(19) United States
(12) Patent Application Publication
(10) Pub. No.: US 2025/0320283 A1
(43) Pub. Date: Oct. 16, 2025

(54) Antibodies Binding to Alpha-Synuclein for Therapy and Diagnosis

(71) Applicant: AC Immune SA, Lausanne (CH)

(72) Inventors: Elpida Tsika, Lausanne (CH); John Warner, Lausanne (CH); Jan Peter Henning Stöhr, Lausanne (CH)

(21) Appl. No.: 18/030,918

(22) PCT Filed: Oct. 15, 2021
(86) PCT No.: PCT/EP2021/078706

§ 371 Date: Apr. 7, 2023

(30) Foreign Application Priority Data
Oct. 16, 2020 (EP) 20202413.9

(51) Int. Cl. C07K 16/18

(52) U.S. Cl. CPC C07K 16/18; C07K 2317/76; C07K 2317/92

(57) ABSTRACT

The present invention relates to novel molecules that can be employed for the prevention, alleviation, treatment and/or diagnosis of diseases, disorders and abnormalities associated with alpha-synuclein (α-synuclein, A-synuclein, aSynuclein, A-syn, α-syn, aSyn, a-syn) aggregates, including, but not limited to, Lewy bodies and/or Lewy neurites, such as Parkinson's disease, Multiple System Atrophy, Lewy Body dementia (LBD; dementia with Lewy bodies (DLB) (“pure” Lewy body dementia), Parkinson's disease dementia (PDD)) or Diffuse Lewy Body Disease. The invention relates to alpha-synuclein binding molecules, in particular to alpha-synuclein antibodies or an antigen-binding fragment or a derivative thereof and uses thereof. The present molecules can also be used for determining a predisposition to such a disorder, disease or abnormality, monitoring residual disorder, disease or abnormality, or predicting the responsiveness of a patient who is suffering from such a disorder, disease or abnormality to treatment with a certain medicament.

### INCORPORATION BY REFERENCE OF SEQUENCE LISTING PROVIDED AS A TEXT FILE

A Sequence Listing is provided herewith in a text file, BOULT-048_SEQ_LIST_ST25, created on Oct. 30, 2023 and having a size of 365,891 bytes. The contents of the text file are incorporated herein by reference in its entirety.

### FIELD OF THE INVENTION

The present invention relates to novel molecules that can be employed for the prevention, alleviation, treatment and/or diagnosis of diseases, disorders and abnormalities associated with alpha-synuclein (α-synuclein, A-synuclein, aSynuclein, A-syn, α-syn, aSyn, a-syn) aggregates including, but not limited to, Lewy bodies and/or Lewy neurites, such as Parkinson's disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. The invention relates to alpha-synuclein binding molecules, in particular to alpha-synuclein antibodies or an antigen-binding fragment thereof or a derivative thereof and uses thereof. The present molecules can also be used for determining a predisposition to such a disorder, disease or abnormality, monitoring residual disorder, disease or abnormality, or predicting the responsiveness of a patient who is suffering from such a disorder, disease or abnormality to the treatment with a certain medicament. The alpha-synuclein binding molecules may be included, according to all aspects of the invention, in mixtures comprising at least two antibodies or functional fragments thereof, in particular where the at least two antibodies or functional fragments thereof are alpha-synuclein binding molecules of the invention.

### BACKGROUND OF THE INVENTION

Many degenerative diseases are associated with extracellular or intracellular deposits of amyloid or amyloid-like proteins that contribute to the pathogenesis as well as to the progression of the disease. The best characterized amyloid protein that forms extracellular aggregates is amyloid beta (Ap).

Amyloid-like proteins that form mainly intracellular aggregates, include, but are not limited to alpha-synuclein, tau, and huntingtin (htt). Diseases involving alpha-synuclein aggregates are generally listed as synucleinopathies (or α-synucleinopathies) and these include, but are not limited to, Parkinson's disease (PD). Synucleinopathies include Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), diffuse Lewy body disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, and Down syndrome. Synucleinopathies with neuronal and glial aggregates of alpha-synuclein include but are not limited to multiple system atrophy (Shy-Drager syndrome, striatonigral degeneration and olivopontocerebellar atrophy). Other diseases that may have alpha-synuclein-immunoreactive lesions include traumatic brain injury, chronic traumatic encephalopathy, dementia puglistica, tauopathies (Pick's disease, frontotemporal dementia, progressive supranuclear palsy, corticobasal degeneration and Niemann-Pick type C1 disease, frontotemporal dementia with Parkinsonism linked to chromosome 17), motor neuron disease, Huntington's disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Creutzfeldt-Jakob disease, ataxia telangiectatica, Meige's syndrome, subacute sclerosing panencephalitis, Gerstmann-Straussler-Scheinker disease, inclusion-body myositis, Gaucher disease, Krabbe disease as well as other lysosomal storage disorders (including Kufor-Rakeb syndrome and Sanfilippo syndrome) and rapid eye movement (REM) sleep behavior disorder (Jellinger, Mov Disord 2003, 18 Suppl. 6, S2-12; Galvin et al., JAMA Neurology 2001, 58 (2), 186-190; Kovari et al., Acta Neuropathol. 2007, 114(3), 295-8; Saito et al., J Neuropathol Exp Neurol. 2004, 63(4), 323-328; McKee et al., Brain, 2013, 136(Pt 1), 43-64; Puschmann et al., Parkinsonism Relat Disord 2012, 18S1, S24-S27; Usenovic et al., J Neurosci. 2012, 32(12), 4240-4246; Winder-Rhodes et al., Mov Disord. 2012, 27(2), 312-315; Ferman et al., J Int Neuropsychol Soc. 2002, 8(7), 907-914; Smith et al., J Pathol. 2014; 232:509-521, Lippa et al., Ann Neurol. 1999 March; 45(3):353-7; Schmitz et al., Mol Neurobiol. 2018 Aug. 22; Charles et al., Neurosci Lett. 2000 Jul. 28; 289(1):29-32; Wilhelmsen et al., Arch Neurol. 2004 March; 61(3):398-406; Yamaguchi et al., J Neuropathol Exp Neurol. 2004, 80^{th} annual meeting, vol. 63; Askanas et al., J Neuropathol Exp Neurol. 2000 July; 59(7):592-8).

Alpha-synuclein is a 140 amino acid long, cytosolic protein abundantly and predominantly expressed in the CNS and localized in pre-synaptic terminals (Burre J., J Parkinsons Dis. 2015; 5(4):699-713). Alpha-synuclein is a natively unfolded protein but adopts secondary structure of mostly helical nature upon association with lipid vesicles or membranes (Iwai et al., Biochemistry 1995, 34(32), 10139-10145). The physiological function of alpha-synuclein remains elusive. Because of the association of alpha-synuclein with synaptic vesicles and its presynaptic localization it is suggested that it regulates synaptic activity and plasticity, neurotransmitter release, dopamine production and metabolism, vesicle trafficking, synaptic vesicle pool maintenance and might also exhibit chaperone-like activity (Cabin et al., J Neurosci. 2002; 22:8797-8807; Chandra et al., Cell. 2005; 123:383-396).

The sequence of alpha-synuclein can be divided into three main domains: 1) the N-terminal region comprising of residues 1-60, which contains 11-mer amphipathic imperfect repeat residues with highly conserved hexamer (KTKEGV). This region has been implicated in regulating alpha-synuclein association to lipid membranes and its internalization; 2) the hydrophobic Non-Amyloid beta Component (NAC) domain spanning residues 61-95; which is essential for alpha-synuclein fibrillization; and 3) the C-terminal region spanning residues 96-140 which is highly acidic and proline-rich, it has no distinct structural propensity.

Alpha-synuclein has been shown to undergo several post translational modifications, including truncations, phosphorylation, ubiquitination, sumoylation, oxidation, nitration, acetylation, glycation, glycosylation, and/or transglutaminase covalent cross linking (Fujiwara et al., Nat Cell Biol 2002, 4(2), 160-164; Hasegawa et al., J Biol Chem 2002, 277(50), 49071-49076; Li et al., Proc Natl Acad Sci USA 2005, 102(6), 2162-2167; Oueslati et al., Prog Brain Res 2010, 183, 115-145; Schmid et al., J Biol Chem 2009, 284(19), 13128-13142; Dorval et al., J Biol Chem. 2006, 281(15):9919-24; Ruzafa et al., PlosOne 2017 12(5):e0178576; Ischiropoulos et al., Ann N Y Acad Sci. 2003, 991, 93-100; Munch et al., J Chem Neuroanat. 2000; 20:253-257; Marotta et al., Chembiochem. 2012; 13:2665-2670). The majority of these modifications involve residues within the C-terminal region.

Several phosphorylation sites have been detected in the carboxyl-terminal region on Tyr-125, -133, and -136, and on Ser-129 (Negro et al., FASEB J 2002, 16(2), 210-212). Extensive and selective phosphorylation of alpha-synuclein at Ser-129 is evident in synucleinopathy lesions, including Lewy bodies (Fujiwara et al., Nat Cell Biol 2002, 4(2); 160-164). Other post-translational modifications in the carboxyl-terminal, including glycosylation on Ser-129 (McLean et al., Neurosci Lett 2002, 323(3), 219-223) and nitration on Tyr-125, -133, and -136 (Takahashi et al., Brain Res 2002, 938(1-2), 73-80), may affect aggregation of alpha-synuclein. Truncation of the carboxyl-terminal region by proteolysis has been reported to play a role in alpha-synuclein fibrillogenesis in various neurodegenerative diseases (Rochet et al., Biochemistry 2000, 39(35), 10619-10626).

Full-length as well as partially truncated and insoluble aggregates of alpha-synuclein have been detected in highly purified Lewy bodies (Crowther et al., FEBS Lett 1998, 436(3), 309-312).

Abnormal protein aggregation is a common feature in aging brain and in several neurodegenerative diseases, even though a clear role in the disease process remains to be defined. In in vitro models, alpha-synuclein readily assembles into filaments resembling those isolated from brain of patients with Lewy Body dementia and familial PD (Crowther et al., FEBS Lett 1998, 436(3), 309-312). Alpha-synuclein and its mutated forms (e.g. A53T and A30P) have a random coil conformation and do not form significant secondary structures in aqueous solution at low concentrations; however, at higher concentrations they are prone to self-aggregate, producing amyloid fibrils (Wood et al., J Biol Chem 1999, 274(28), 19509-19512). Several differences in the aggregation behavior of the PD-linked mutants and the wild-type protein have been documented. Monomeric alpha-synuclein aggregates in vitro form stable fibrils via a metastable oligomeric (i.e., protofibril) state (Volles et al., Biochemistry 2002, 41(14), 4595-4602).

Parkinson's disease (PD) is the most common neurodegenerative motor disorder. PD is mainly an idiopathic disease, although in at least 5% of the PD patients the pathology is linked to mutations in one or several specific genes. Several point mutations have been described in the alpha-synuclein gene (A30P, E46K, H50Q, G51 D, A53T) which cause familial PD with autosomal dominant inheritance. Furthermore, duplications and triplications of the alpha-synuclein gene have been described in patients that developed PD underlining the role of alpha-synuclein in PD pathogenesis (Lesage et al., Hum. Mol. Genet., 2009, 18, R48-59). The pathogenesis of PD remains elusive, however, growing evidence suggests a role for the pathogenic folding of the alpha-synuclein protein that leads to the formation of amyloid-like fibrils. Indeed, the hallmarks of PD are the presence of intracellular alpha-synuclein aggregate structures called Lewy Bodies in the nigral neurons, as well as the death of dopaminergic neurons in the substantia nigra and elsewhere. Alpha-synuclein is a natively unfolded presynaptic protein that can misfold and aggregate into larger oligomeric and fibrillar forms which are linked to the pathogenesis of PD. Studies have implicated small soluble oligomeric and protofibrillar forms of alpha-synuclein as the most neurotoxic species (Lashuel et al., J. Mol. Biol., 2002, 322, 1089-102), however the precise role of alpha-synuclein in the neuronal cell toxicity remains to be clarified (review: Cookson, Annu. Rev. Biochem., 2005, 74, 29-52).

Recent evidence from cellular and animal models suggests that pathological and/or aggregated alpha-synuclein can spread from one neuron to another. Once inside the new cell alpha-synuclein aggregates act as seeds, recruiting endogenous alpha-synuclein and advancing protein aggregation (Luk et al., Science. 2012, 338(6109):949-5; Tran et al., Cell Rep. 2014, 7(6):2054-65). Moreover, the transynaptic spreading of pathological and/or aggregated alpha-synuclein could explain the progressive advancing of Lewy pathology through defined anatomical connected brain areas in PD that was first described by Braak and colleagues (Braak et al., Neurobiol. Aging. 2003; 24:197-211).

Consequently, the cell-to-cell spreading of pathological and/or aggregated alpha-synuclein renders immunotherapy as a compelling target for new therapeutic approaches aiming to alleviate, treat, retard or halt the progression of PD and other synucleinopathies. Antibodies described herein inhibit and/or delay seeded and/or spontaneous alpha-synuclein aggregation, and this functional feature would allow them to bind to alpha-synuclein seeds in the extracellular space to either neutralize the seeds and consequently delay or inhibit the propagation of alpha-synuclein aggregates or facilitate the clearance of these spreading species. The development of such therapies for PD and other synucleinopathies would addresses an unmet medical need since currently only symptomatic treatments are available.

The diagnosis of Parkinson's disease is largely clinical and depends on the presence of a specific set of symptoms and signs (the initial core feature being bradykinesia, rigidity, rest tremor and postural instability), a slowly progressive course, and a response to drug treatment. The final confirmation of the diagnosis is made by post-mortem neuropathological analysis. Strategies are being developed to apply recent advances of the cause of Parkinson's disease to the development of biochemical biomarkers as well as imaging biomarkers (Schapira, Curr Opin Neurol 2013; 26(4):395-400). Such biomarkers that have been investigated in different body fluids (cerebrospinal fluid (CSF), plasma, saliva) include alpha-synuclein levels but also DJ-1, Tau and Abeta, as well as neurofilaments proteins, interleukins, osteopontin and hypocrontin (Schapira, Curr Opin Neurol 2013; 26(4):395-400), but so far none of these biomarkers alone or in combination can be used as a determinant diagnostic test. Antibodies for diagnostic application that selectively recognize and bind to certain pathological structures of alpha-synuclein would have the potential to be used as biomarkers with high sensitivity and specificity. To our knowledge no approved biomarker for monitoring pathological alpha-synuclein levels is currently on the market or available for clinical trials despite a crucial needs for Parkinson's disease research and drug development (Eberling et al., J Parkinsons Dis. 2013; 3(4):565-7).

### PRIOR ART

WO2017/207739 provides antibodies that specifically bind human alpha-synuclein with a high affinity and reduces alpha-synuclein spreading in vivo.

### SUMMARY OF THE INVENTION

It is an object of the present invention to provide alpha-synuclein binding molecules that can be employed to treat, alleviate and/or prevent a disease, disorder or abnormality associated with alpha-synuclein aggregates, such as Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease.

In another aspect, it is an object of the present invention to provide molecules that can be employed to diagnose, monitor disease progression of, and/or monitor drug activity against, a disease, disorder or abnormality associated with alpha-synuclein aggregates including, but not limited to, Lewy bodies, Lewy neurites and/or glial cytoplasmic inclusions, such as Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease.

The invention generally relates to an alpha-synuclein binding molecule, which inhibits and/or delays seeded and/or spontaneous alpha-synuclein aggregation.

In one embodiment, the invention relates to an alpha-synuclein binding molecule, which
(i) inhibits and/or delays seeded and/or spontaneous alpha-synuclein aggregation; and
(ii) is capable of recognizing and binding to pathological and/or aggregated alpha-synuclein, particularly human alpha-synuclein, in vitro and/or in vivo.

Accordingly, the invention relates in its broadest aspect to binding molecules, in particular antibodies or antigen-binding fragments thereof, which bind alpha-synuclein. In a preferred embodiment of the invention, the binding molecules, in particular antibodies or antigen-binding fragments thereof, inhibit and/or delay the aggregation of seeded and/or spontaneous alpha-synuclein aggregation and are capable of recognizing and binding to pathological and/or aggregated alpha-synuclein, particularly human alpha-synuclein, in vitro and/or in vivo. Alpha-synuclein is a soluble protein that has the propensity to spontaneously aggregate and form soluble oligomers or soluble/insoluble protofibrils or mature fibrils or detergent-insoluble aggregates under certain conditions. Seeded alpha-synuclein aggregation is the aggregation accelerated by pathological alpha-synuclein, so called “seeds”. The alpha-synuclein binding molecules may be included in mixtures comprising at least two antibodies or functional fragments thereof, in particular where the at least two antibodies or functional fragments thereof are alpha-synuclein binding molecules of the invention.

The alpha-synuclein binding molecules of the invention, in particular antibodies or antigen-binding fragments thereof, block cell-to-cell spreading and/or delay and/or inhibit the aggregation of alpha-synuclein protein or fragments thereof. Thus, an alpha-synuclein binding molecule within the present invention inhibits and/or delays seeded and/or spontaneous alpha-synuclein aggregation; and is capable of recognizing and binding to pathological and/or aggregated alpha-synuclein, particularly human alpha-synuclein, in vitro and in vivo. An alpha-synuclein binding molecule within the present invention inhibits and/or delays seeded and/or spontaneous alpha-synuclein aggregation; and is capable of recognizing and binding to pathological and/or aggregated alpha-synuclein, particularly human alpha-synuclein, in vitro or in vivo.

In particular alpha-synuclein binding molecules of the invention, in particular antibodies or antigen-binding fragments thereof, inhibit and/or delay aggregation of alpha-synuclein protein or fragments thereof.

In one embodiment, alpha-synuclein binding molecules of the invention, in particular antibodies or antigen-binding fragments thereof, inhibit the formation of alpha-synuclein aggregates, including but not limited to, Lewy Bodies, Lewy Neurites, and/or glial cytoplasmic inclusions.

The alpha-synuclein binding molecules, especially antibodies or antigen-binding fragments thereof, of the invention may selectively bind aggregated alpha-synuclein and/or pathological alpha-synuclein in preference to non-aggregated alpha-synuclein and/or non-pathological alpha-synuclein (such as monomeric alpha-synuclein).

In some embodiments of the invention, the antibody is a monoclonal antibody. In some embodiments, the antibody is a murine, murinized, human, humanized, or chimeric antibody.

In some embodiments of the invention, the antibody, or antigen-binding fragment or derivative thereof having a binding characteristic of an antibody described herein, is an antibody having the variable regions VH and/or VL of the amino acid sequences, respectively, set forth in SEQ ID NO: 610 and SEQ ID NO: 614; SEQ ID NO: 620 and SEQ ID NO: 624; SEQ ID NO: 630 and SEQ ID NO: 634; SEQ ID NO: 640 and SEQ ID NO: 644; SEQ ID NO: 650 and SEQ ID NO: 654; SEQ ID NO: 660 and SEQ ID NO: 664; SEQ ID NO: 670 and SEQ ID NO: 674; SEQ ID NO: 680 and SEQ ID NO: 684; SEQ ID NO: 690 and SEQ ID NO: 694; SEQ ID NO: 700 and SEQ ID NO: 704; SEQ ID NO: 710 and SEQ ID NO: 714; SEQ ID NO: 720 and SEQ ID NO: 724; SEQ ID NO: 730 and SEQ ID NO: 734; SEQ ID NO: 740 and SEQ ID NO: 744; SEQ ID NO: 750 and SEQ ID NO: 754; SEQ ID NO: 760 and SEQ ID NO: 764; SEQ ID NO: 770 and SEQ ID NO: 774; SEQ ID NO: 750 and SEQ ID NO: 784; SEQ ID NO: 790 and SEQ ID NO: 794; SEQ ID NO: 800 and SEQ ID NO: 804; SEQ ID NO: 810 and SEQ ID NO: 814; SEQ ID NO: 820 and SEQ ID NO: 824; SEQ ID NO: 830 and SEQ ID NO: 834; SEQ ID NO: 840 and SEQ ID NO: 844.

The invention therefore also provides an alpha-synuclein binding antibody having the variable regions VH and/or VL of the amino acid sequences, respectively, set forth in SEQ ID NO: 610 and SEQ ID NO: 614; SEQ ID NO: 620 and SEQ ID NO: 624; SEQ ID NO: 630 and SEQ ID NO: 634; SEQ ID NO: 640 and SEQ ID NO: 644; SEQ ID NO: 650 and SEQ ID NO: 654; SEQ ID NO: 660 and SEQ ID NO: 664; SEQ ID NO: 670 and SEQ ID NO: 674; SEQ ID NO: 680 and SEQ ID NO: 684; SEQ ID NO: 690 and SEQ ID NO: 694; SEQ ID NO: 700 and SEQ ID NO: 704; SEQ ID NO: 710 and SEQ ID NO: 714; SEQ ID NO: 720 and SEQ ID NO: 724; SEQ ID NO: 730 and SEQ ID NO: 734; SEQ ID NO: 740 and SEQ ID NO: 744; SEQ ID NO: 750 and SEQ ID NO: 754; SEQ ID NO: 760 and SEQ ID NO: 764; SEQ ID NO: 770 and SEQ ID NO: 774; SEQ ID NO: 750 and SEQ ID NO: 784; SEQ ID NO: 790 and SEQ ID NO: 794; SEQ ID NO: 800 and SEQ ID NO: 804; SEQ ID NO: 810 and SEQ ID NO: 814; SEQ ID NO: 820 and SEQ ID NO: 824; SEQ ID NO: 830 and SEQ ID NO: 834; SEQ ID NO: 840 and SEQ ID NO: 844.

In some embodiments, the antibody comprises:
a) VH-CDR1 comprising the amino acid sequence of SEQ ID NO: 611; VH-CDR2 comprising the amino acid sequence of SEQ ID NO: 612; and VH-CDR3 comprising the amino acid sequence of SEQ ID NO: 613; VL-CDR1 comprising the amino acid sequence of SEQ ID NO: 615; VL-CDR2 comprising the amino acid sequence of SEQ ID NO: 616; and VL-CDR3 comprising the amino acid sequence of SEQ ID NO: 617; or
b) VH-CDR1 comprising the amino acid sequence of SEQ ID NO: 621; VH-CDR2 comprising the amino acid sequence of SEQ ID NO: 622; and VH-CDR3 comprising the amino acid sequence of SEQ ID NO: 623; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
c) VH-CDR1 comprising the amino acid sequence of SEQ ID NO: 631; VH-CDR2 comprising the amino acid sequence of SEQ ID NO: 632; and VH-CDR3 comprising the amino acid sequence of SEQ ID NO: 633; VL-CDR1 comprising the amino acid sequence of SEQ ID NO: 635; VL-CDR2 comprising the amino acid sequence of SEQ ID NO: 616; and VL-CDR3 comprising the amino acid sequence of SEQ ID NO: 637; or
d) VH-CDR1 comprising the amino acid sequence of SEQ ID NO: 641; VH-CDR2 comprising the amino acid sequence of SEQ ID NO: 642; and VH-CDR3 comprising the amino acid sequence of SEQ ID NO: 643; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
e) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 653; VL-CDR1 comprising the amino acid sequence of SEQ ID NO: 655; VL-CDR2 comprising the amino acid sequence of SEQ ID NO: 626; and VL-CDR3 comprising the amino acid sequence of SEQ ID NO: 627; or
f) VH-CDR1 comprising the amino acid sequence of SEQ ID NO: 661; VH-CDR2 comprising the amino acid sequence of SEQ ID NO: 662; and VH-CDR3 comprising the amino acid sequence of SEQ ID NO: 663; VL-CDR1 comprising the amino acid sequence of SEQ ID NO: 665; VL-CDR2 comprising the amino acid sequence of SEQ ID NO: 666; and VL-CDR3 comprising the amino acid sequence of SEQ ID NO: 667; or
g) VH-CDR1 comprising the amino acid sequence of SEQ ID NO: 671; VH-CDR2 comprising the amino acid sequence of SEQ ID NO: 672; and VH-CDR3 comprising the amino acid sequence of SEQ ID NO: 673; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
h) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 683; VL-CDR1 comprising the amino acid sequence of SEQ ID NO: 625; VL-CDR2 comprising the amino acid sequence of SEQ ID NO: 686; and VL-CDR3 comprising the amino acid sequence of SEQ ID NO: 627; or
i) VH-CDR1 comprising the amino acid sequence of SEQ ID NO: 691; VH-CDR2 comprising the amino acid sequence of SEQ ID NO: 692; and VH-CDR3 comprising the amino acid sequence of SEQ ID NO: 693; VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697; or
j) VH-CDR1 comprising the amino acid sequence of SEQ ID NO: 701; VH-CDR2 comprising the amino acid sequence of SEQ ID NO: 702; and VH-CDR3 comprising the amino acid sequence of SEQ ID NO: 703; VL-CDR1 comprising the amino acid sequence of SEQ ID NO: 705; VL-CDR2 comprising the amino acid sequence of SEQ ID NO: 706; and VL-CDR3 comprising the amino acid sequence of SEQ ID NO: 707; or
k) VH-CDR1 comprising the amino acid sequence of SEQ ID NO: 711; VH-CDR2 comprising the amino acid sequence of SEQ ID NO: 712; and VH-CDR3 comprising the amino acid sequence of SEQ ID NO: 713; VL-CDR1 comprising the amino acid sequence of SEQ ID NO: 715; VL-CDR2 comprising the amino acid sequence of SEQ ID NO: 716; and VL-CDR3 comprising the amino acid sequence of SEQ ID NO: 717; or
l) VH-CDR1 comprising the amino acid sequence of SEQ ID NO: 721; VH-CDR2 comprising the amino acid sequence of SEQ ID NO: 722; and VH-CDR3 comprising the amino acid sequence of SEQ ID NO: 723; VL-CDR1 comprising the amino acid sequence of SEQ ID NO: 725; VL-CDR2 comprising the amino acid sequence of SEQ ID NO: 616; and VL-CDR3 comprising the amino acid sequence of SEQ ID NO: 637; or
m) VH-CDR1 comprising the amino acid sequence of SEQ ID NO: 731; VH-CDR2 comprising the amino acid sequence of SEQ ID NO: 722; and VH-CDR3 comprising the amino acid sequence of SEQ ID NO: 733; VL-CDR1 comprising the amino acid sequence of SEQ ID NO: 735; VL-CDR2 comprising the amino acid sequence of SEQ ID NO: 736; and VL-CDR3 comprising the amino acid sequence of SEQ ID NO: 637; or
n) VH-CDR1 comprising the amino acid sequence of SEQ ID NO: 671; VH-CDR2 comprising the amino acid sequence of SEQ ID NO: 742; and VH-CDR3 comprising the amino acid sequence of SEQ ID NO: 743; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
o) VH-CDR1 comprising the amino acid sequence of SEQ ID NO: 751; VH-CDR2 comprising the amino acid sequence of SEQ ID NO: 722; and VH-CDR3 comprising the amino acid sequence of SEQ ID NO: 723; VL-CDR1 comprising the amino acid sequence of SEQ ID NO: 735; VL-CDR2 comprising the amino acid sequence of SEQ ID NO: 616; and VL-CDR3 comprising the amino acid sequence of SEQ ID NO: 637; or
p) VH-CDR1 comprising the amino acid sequence of SEQ ID NO: 761; VH-CDR2 comprising the amino acid sequence of SEQ ID NO: 722; and VH-CDR3 comprising the amino acid sequence of SEQ ID NO: 733; VL-CDR1 comprising the amino acid sequence of SEQ ID NO: 765; VL-CDR2 comprising the amino acid sequence of SEQ ID NO: 616; and VL-CDR3 comprising the amino acid sequence of SEQ ID NO: 637; or
q) VH-CDR1 comprising the amino acid sequence of SEQ ID NO: 771; VH-CDR2 comprising the amino acid sequence of SEQ ID NO: 772; and VH-CDR3 comprising the amino acid sequence of SEQ ID NO: 773; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
r) VH-CDR1 comprising the amino acid sequence of SEQ ID NO: 791; VH-CDR2 comprising the amino acid sequence of SEQ ID NO: 792; and VH-CDR3 comprising the amino acid sequence of SEQ ID NO: 793; VL-CDR1 comprising the amino acid sequence of SEQ ID NO: 795; VL-CDR2 comprising the amino acid sequence of SEQ ID NO: 616; and VL-CDR3 comprising the amino acid sequence of SEQ ID NO: 797; or
s) VH-CDR1 comprising the amino acid sequence of SEQ ID NO: 771; VH-CDR2 comprising the amino acid sequence of SEQ ID NO: 802; and VH-CDR3 comprising the amino acid sequence of SEQ ID NO: 803; VL-CDR1 comprising the amino acid sequence of SEQ ID NO: 805; VL-CDR2 comprising the amino acid sequence of SEQ ID NO: 676; and VL-CDR3 comprising the amino acid sequence of SEQ ID NO: 677; or
t) VH-CDR1 comprising the amino acid sequence of SEQ ID NO: 811; VH-CDR2 comprising the amino acid sequence of SEQ ID NO: 812; and VH-CDR3 comprising the amino acid sequence of SEQ ID NO: 813; VL-CDR1 comprising the amino acid sequence of SEQ ID NO: 815; VL-CDR2 comprising the amino acid sequence of SEQ ID NO: 696; and VL-CDR3 comprising the amino acid sequence of SEQ ID NO: 817; or
u) VH-CDR1 comprising the amino acid sequence of SEQ ID NO: 821; VH-CDR2 comprising the amino acid sequence of SEQ ID NO: 822; and VH-CDR3 comprising the amino acid sequence of SEQ ID NO: 823; VL-CDR1 comprising the amino acid sequence of SEQ ID NO: 825; VL-CDR2 comprising the amino acid sequence of SEQ ID NO: 826; and VL-CDR3 comprising the amino acid sequence of SEQ ID NO: 827; or
v) VH-CDR1 comprising the amino acid sequence of SEQ ID NO: 831; VH-CDR2 comprising the amino acid sequence of SEQ ID NO: 832; and VH-CDR3 comprising the amino acid sequence of SEQ ID NO: 833; VL-CDR1 comprising the amino acid sequence of SEQ ID NO: 835; VL-CDR2 comprising the amino acid sequence of SEQ ID NO: 836; and VL-CDR3 comprising the amino acid sequence of SEQ ID NO: 817; or
w) VH-CDR1 comprising the amino acid sequence of SEQ ID NO: 841; VH-CDR2 comprising the amino acid sequence of SEQ ID NO: 842; and VH-CDR3 comprising the amino acid sequence of SEQ ID NO: 843; VL-CDR1 comprising the amino acid sequence of SEQ ID NO: 845; VL-CDR2 comprising the amino acid sequence of SEQ ID NO: 846; and VL-CDR3 comprising the amino acid sequence of SEQ ID NO: 847.

These alpha-synuclein binding antibodies may constitute separate aspects of the invention. The alpha-synuclein binding molecules may be included in mixtures comprising at least two antibodies or functional fragments thereof, in particular where the at least two antibodies or functional fragments thereof are alpha-synuclein binding molecules of the invention.

In some embodiments, an isolated nucleic acid is provided, wherein the isolated nucleic acid encodes an antibody, or an antigen-binding fragment or derivative thereof, described herein. In some embodiments, a host cell is provided, wherein the host cell comprises an isolated nucleic acid that encodes an antibody, or an antigen-binding fragment or derivative thereof, described herein. In some embodiments, a method of producing an antibody, or an antigen-binding fragment or derivative thereof, is provided, comprising culturing the host cell under conditions suitable for producing the antibody, or the antigen-binding fragment or the derivative thereof.

In some embodiments, an immunoconjugate is provided, wherein the immunoconjugate comprises an isolated antibody, antigen-binding fragment or derivative thereof, described herein and a therapeutic agent. In some embodiments, a labeled antibody, antigen-binding fragment or derivative thereof, is provided, comprising an antibody antigen-binding fragment or derivative thereof, described herein and a detectable label. The immunoconjugates may be included in mixtures comprising at least two immunoconjugates of the invention.

In some embodiments, a pharmaceutical composition is provided, comprising an isolated antibody, antigen-binding fragment or derivative thereof, described herein and a pharmaceutically acceptable carrier and/or excipient. A pharmaceutical composition may comprise, according to the invention, mixtures comprising at least two antibodies or functional fragments thereof, in particular where the at least two antibodies or functional fragments thereof are alpha-synuclein binding molecules of the invention.

As used herein, the term “isolated” means that the chemical compound, e.g. the nucleic acid or antibody, may have been separated and/or recovered from its natural environment. Within the present invention, the chemical compound is preferably chemically synthesized, or synthesized in a cellular system different from the cell from which it naturally originates, and is thus “isolated” from its naturally associated components. The chemical compound may be isolated from its natural environment by e.g. purification or produced by means of a technical process (including but not limited to e.g. gene synthesis, polymerase chain reaction (PCR), vector purification and protein (antibody) purification). Such chemical compound may be, in particular, a nucleic acid, DNA-, RNA-, or cDNA-sequence, or a peptide, antibody or protein.

The present invention is not limited to an isolated antibody in accordance with the above definition, but also relates to an antibody as such irrespective of its origin.

The same applies to peptides, nucleic acids, DNA, RNA and/or cDNA sequences provided by the present invention, which are encompassed in isolated form, as defined above, or in any other form.

In some embodiments, a method of preventing, alleviating and/or treating a disease, disorder or abnormality associated with alpha-synuclein aggregates or pathological alpha-synuclein, such as Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), Diffuse Lewy Body Disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder, is provided. According to one embodiment, the methods of the invention comprise administering an effective concentration or an effective amount of a binding molecule, particularly an antibody, or an antigen-binding fragment or derivative thereof, of the invention binding alpha-synuclein (e.g., a full-length antibody or an alpha-synuclein binding fragment or derivative of an antibody) as described herein to a subject in need thereof. The mixtures of the invention may be used in the methods of the invention.

In some embodiments, a method of retaining motor capabilities or improving motor deficits of a subject suffering from a synucleopathy, including reducing bradykinesia, rigidity, resting tremor or postural instability is provided, comprising administering an antibody, or an antigen-binding fragment or derivative thereof, described herein or a pharmaceutical composition comprising an antibody, or antigen-binding fragment or derivative thereof, described herein to a subject in need thereof.

In some embodiments, a method of retaining or increasing cognitive capacity of a subject suffering from a synucleopathy is provided, comprising administering an antibody, or antigen-binding fragment or derivative thereof, described herein or a pharmaceutical composition comprising an antibody, or antigen-binding fragment or derivative thereof, described herein to a subject in need thereof.

In some embodiments, an isolated antibody, or an antigen-binding fragment or derivative thereof, described herein is provided for use as a medicament. In some embodiments, an isolated antibody, or an antigen-binding fragment or derivative thereof, described herein is provided for use in alleviating, preventing and/or treating a synucleinopathy in a subject. In some embodiments, use of an antibody, or an antigen-binding fragment or derivative thereof, described herein is provided for manufacture of a medicament for preventing, alleviating and/or treating a disease, a disorder and/or abnormality associated with alpha-synuclein aggregates.

In some embodiments, the disease, disorder and/or abnormality associated with alpha-synuclein aggregate is a synucleinopathy. In some embodiments, the synucleinopathy is Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), Diffuse Lewy Body Disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

More particularly, the synucleinopathy is selected from Parkinson's Disease, Multiple System Atrophy, Lewy Body dementia (LBD; dementia with Lewy bodies (DLB) (“pure” Lewy body dementia), Parkinson's disease dementia (PDD)), and Diffuse Lewy Body Disease.

In some embodiments, a method of detecting alpha-synuclein aggregates including, but not limited to, Lewy bodies, Lewy neurites and/or glial cytoplasmic inclusions, is provided, comprising contacting a sample with an antibody, or antigen-binding fragment or derivative thereof, described herein and detecting the presence of aggregates using methods known in the art. In some embodiments, the sample is a brain sample, a cerebrospinal fluid sample, or a blood sample. The mixtures of the invention may be employed in the various detection methods of the invention described herein, including diagnostic, screening and monitoring methods etc.

In some embodiments, a method for evaluating an alpha-synuclein binding molecule for the capability of inhibiting and/or delaying the seeded and/or spontaneous alpha-synuclein aggregation is provided, the method comprising the steps of: bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and determining the time to reach half-maximum signal of the detectable dye, particularly the signal of fluorescent dye, relative to the seeded aggregation in the absence of binding molecule, wherein an increase in time to reach half-maximum signal of the detectable dye in the presence of binding molecule relative to the seeded aggregation in the absence of binding molecule indicates that the alpha-synuclein binding molecule is capable of inhibiting and/or delaying the seeded and/or spontaneous alpha-synuclein aggregation.

In further embodiments, a method for selecting/screening an alpha-synuclein binding molecule capable of inhibiting and/or delaying the seeded and/or spontaneous alpha-synuclein aggregation is provided, the method comprising the steps of bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and selecting the alpha-synuclein binding molecule as being able to inhibit and/or delay seeded and/or spontaneous alpha-synuclein aggregation based on the signal of the detectable dye, in particular the fluorescent dye, determined in the absence and presence of the alpha-synuclein binding molecule.

In some embodiments, the method of evaluating or selecting an alpha-synuclein binding molecule capable of inhibiting and/or delaying the seeded and/or spontaneous alpha-synuclein aggregation is provided, wherein the detectable dye is thioflavin (ThT), which binds to the beta-sheet structure of the aggregated protein.

In some embodiments, the method of evaluating or selecting an alpha-synuclein binding molecule capable of inhibiting and/or delaying the seeded and/or spontaneous alpha-synuclein aggregation is provided, wherein the alpha-synuclein monomeric protein is covalently linked to the detectable dye, in particular the fluorescent dye, and/or wherein the signal of the detectable dye, in particular the fluorescent dye, is quenching of signal/fluorescence emission upon formation of the protein aggregates. Other detection methods are also envisaged within the scope of the present invention, including, for example, fluorescence resonance energy transfer (FRET) assays or the like. Dyes, in particular fluorescent dyes, are known to the person skilled in the art. Examples include for example green fluorescent protein, yellow fluorescent protein and the like.

In some embodiments, an alpha-synuclein binding molecule is evaluated as capable of inhibiting and/or delaying seeded and/or spontaneous alpha-synuclein aggregation or is selected, respectively, if in step d) of the invention the seeded and/or spontaneous alpha-synuclein aggregation is inhibited and/or delayed by at least 10%, 20%, 30%, 40%, 50%, 60%, 70%, 80%, 90%, 100%, 200%, or 300% in the presence of the alpha-synuclein binding molecule as compared to in the absence of the alpha-synuclein binding molecule. Alternatively, an alpha-synuclein binding molecule may be evaluated as capable of inhibiting and/or delaying seeded and/or spontaneous alpha-synuclein aggregation if the alpha-synuclein binding molecule causes an at least 10 percent increase in aggregation half-time (τ½ values) of seeded aggregation relative to the seeded aggregation in the absence of binding molecule.

Within the scope of the present invention, alpha-synuclein may have the sequence of SEQ ID NO: 1. Alpha-synuclein aggregates are multimeric beta-sheet rich assemblies of alpha-synuclein monomers that can form either soluble oligomers or soluble/insoluble protofibrils or mature fibrils which coalesce into intracellular deposits detected as a range of Lewy pathologies in Parkinson's disease and other synucleinopathies. Alpha-synuclein under physiological conditions does not adopt an ordered tertiary structure, rather it is classified as a natively unfolded protein which can exist as a mixture of dynamic and flexible structural conformations.

Misfolded alpha-synuclein can form multimeric intermediate oligomeric structures which eventually assemble into highly-ordered fibrillar aggregates.

The term “aggregated alpha-synuclein” as used herein, refers to insoluble or soluble oligomeric and/or polymeric structures composed of alpha-synuclein misfolded monomers and/or multimers and/or assemblies of monomers.

Pathological alpha-synuclein is misfolded or aggregated or post-translationally modified alpha-synuclein that is the main component of Lewy pathologies; Lewy pathologies can be detected as having the following morphologies: Lewy bodies, Lewy neurites, premature Lewy bodies or pale bodies, perikaryal deposits with diffuse, granular, punctate or pleomorphic patterns. Moreover, pathological alpha-synuclein is the major component of intracellular fibrillary inclusions detected in oligodendrocytes also referred to as glial cytoplasmic inclusions and in neuronal somata, axons and nuclei (referred to as neuronal cytoplasmic inclusions) that are the histological hallmarks of multiple system atrophy. Pathological alpha-synuclein in Lewy pathologies often displays substantial increase in post-translational modifications such as phosphorylation, ubiquitination, nitration, and truncation.

Seeds are multimeric beta-sheet rich structures which are composed of alpha-synuclein, could be also (i.e. in addition to alpha-synuclein) composed of other amyloidogenic proteins (e.g. Tau, Amyloid p), which can accelerate the aggregation kinetics of alpha-synuclein by elongating the growing multimer and/or by acting as templates for the nucleation of monomers on the seed surface.

Spontaneous aggregation of alpha-synuclein is the aggregation process that progresses without the addition of seeds. Alpha-synuclein is a soluble protein that has the propensity to spontaneously aggregate and form soluble oligomers or soluble/insoluble protofibrils or mature fibrils or detergent-insoluble aggregates under certain conditions.

Seeding is the process in which seeds induce the aggregation of the native alpha-synuclein protein.

Lewy bodies are abnormal aggregates of protein that develop inside nerve cells in Parkinson's disease (PD), Lewy body dementia and other synucleinopathies. Lewy bodies appear as spherical masses that displace other cell components. Morphologically, Lewy bodies can be classified as being brainstem or cortical type. Classic brainstem Lewy bodies are eosinophilic cytoplasmic inclusions consisting of a dense core surrounded by a halo of 5-10-nm-wide radiating fibrils, the primary structural component of which is alpha-synuclein; cortical Lewy bodies differ by lacking a halo. The presence of Lewy bodies is a hallmark of Parkinson's disease.

Lewy neurites are abnormal neuronal processes in diseased neurons, containing granular material, abnormal alpha-synuclein filaments similar to those found in Lewy bodies, dot-like, varicose structures and axonal spheroids. Like Lewy bodies, Lewy neurites are a feature of α-synucleinopathies such as dementia with Lewy bodies, Parkinson's disease, and multiple system atrophy.

Glial cytoplasmic inclusions (also referred to as Papp-Lantos inclusions) consist of insoluble alpha-synuclein filamentous aggregates detected in oligodendrocytes in the white matter of multiple system atrophy brains. Alpha-synuclein aggregates in neuronal somata, axons and nuclei, referred to as neuronal cytoplasmic inclusions, are characteristic cytopathological features of multiple system atrophy. The detection of glial cytoplasmic inclusions is considered a hallmark for the neuropathological diagnosis of multiple system atrophy.

An alpha-synuclein binding molecule is a molecule that binds to the pathological and/or aggregated alpha-synuclein protein, such as an alpha-synuclein antibody or fragment thereof, at a specific recognition site, or epitope. Antigen-binding molecules of the invention bind to an epitope within the amino acid sequence of SEQ ID NO: 1. The epitope may be a linear epitope or a non-linear epitope. Preferably antigen-binding molecules of the invention bind to an epitope within amino acids residues 1-15 (SEQ ID NO: 121), 15-45 (SEQ ID NO: 138), 19-33 (SEQ ID NO: 123), 28-50 (SEQ ID NO: 139), 31-60 (SEQ ID NO: 146), 81-120 (SEQ ID NO: 137), 91-105 (SEQ ID NO: 131), 96-140 (SEQ ID NO: 147) or 100-114 (SEQ ID NO: 132) of human alpha-synuclein of SEQ ID NO: 1. More preferably, antigen-binding molecules of the invention bind to an epitope within amino acids residues 28-50 (SEQ ID NO: 139), 31-60 (SEQ ID NO: 146) or 81-120 (SEQ ID NO: 137) of human alpha-synuclein of SEQ ID NO: 1. Even more preferably, antigen-binding molecules of the invention may bind to an epitope comprising amino acids 33-37 or 33-39 or 108-113 of human alpha-synuclein of SEQ ID NO: 1 as critical residues for binding. In another embodiment, antigen-binding molecules of the invention bind to a non-linear epitope within amino acids residues of human alpha-synuclein of SEQ ID NO: 1.

Other alpha-synuclein binding molecules may also include multivalent molecules, multi-specific molecules (e.g., diabodies or biparatopic antibodies), fusion molecules, aptamers, avimers, or other naturally occurring or recombinantly created molecules. Illustrative antigen-binding molecules useful in the present invention include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), Adnectin (WO 2002/032925) and fynomers (WO 2013/135588).

An “antigen binding molecule”, as used herein, is any molecule that can specifically or selectively bind to an antigen. A binding molecule may include or be an antibody or a fragment thereof. An alpha-synuclein binding molecule is a molecule that binds to the alpha-synuclein protein, such as an alpha-synuclein antibody or fragment thereof, at a specific recognition site, epitope.

The terms “alpha-synuclein antibody”, “anti-alpha-synuclein antibody” and “an antibody that binds to pathological and/or aggregated alpha-synuclein” or simply “antibody” as used herein refer to an antibody that is capable of binding pathological alpha-synuclein and/or aggregated alpha-synuclein, including, but not limited to, Lewy bodies, Lewy Neurites or glial cytoplasmic inclusions with sufficient affinity such that the antibody is useful as a therapeutic and/or diagnostic agent in targeting alpha-synuclein. In one embodiment, the extent of binding of an alpha-synuclein antibody of the invention to an unrelated, non-alpha-synuclein protein is less than about 10% of the binding of the antibody to alpha-synuclein as measured, e.g., by a radioimmunoassay (RIA).

In general, the term “antibody” is used herein in the broadest sense and encompasses various antibody structures, including but not limited to monoclonal antibodies, polyclonal antibodies, multispecific antibodies (e.g., bispecific antibodies), fully-human antibodies and antibody fragments so long as they exhibit the desired antigen-binding activity. Antibodies within the present invention may also be chimeric antibodies (especially mouse VH and VL regions fused with human constant domains), recombinant antibodies, antigen-binding fragments of recombinant antibodies, humanized antibodies or antibodies displayed upon the surface of a phage or displayed upon the surface of a chimeric antigen receptor (CAR) T-cell.

An “antigen-binding fragment” of an antibody refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

Accordingly, in context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term “antibody” also comprises but is not limited to fully-human antibodies, chimeric antibodies, humanized antibodies, CDR-grafted antibodies and antibody constructs, like single chain Fvs (scFv) or antibody-fusion proteins.

Humanized antibodies are modified antibodies that are also referred to as reshaped human antibodies. A humanized antibody is constructed by transferring the CDRs of an antibody derived from an immunized animal to the complementarity determining regions of a human antibody. Conventional genetic recombination techniques for such purposes are known (see European Patent Application Publication No. EP 239400; International Publication No. WO 96/02576; Sato K. et al., Cancer Research 1993, 53: 851-856; International Publication No. WO 99/51743).

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. VH-CDR, or CDR-H depicts a CDR region of a variable heavy chain and VL-CDR or CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J., Mol. Biol. 196 (1987), 901-917 or Chothia, Nature 342 (1989), 877-883.

An “Fc” region contains two heavy chain fragments comprising the CH2 and CH3 domains of an antibody. The two heavy chain fragments are held together by two or more disulfide bonds and by hydrophobic interactions of the CH3 domains.

A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′) 2 molecule.

A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains.

The “Fv region” comprises the variable regions from both the heavy and light chains, but lacks the constant regions.

Accordingly, in the context of this invention, antibody molecules or antigen-binding fragments thereof are provided, which are humanized and can successfully be employed in pharmaceutical compositions, including as mixtures of at least two antibody molecules or antigen-binding fragments thereof.

An “antibody that binds to an epitope” within a defined region of a protein is an antibody that requires the presence of one or more of the amino acids within that region for binding to the protein.

In certain embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an “antibody that binds to an epitope” within a defined region of a protein is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody is determined by FACS, WB or by a suitable binding assay such as ELISA.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of alpha-synuclein. Said binding/interaction is also understood to define a “specific recognition”. The term “specifically recognizing” means in accordance with this invention that the antibody is capable of specifically interacting with and/or binding to at least two amino acids of alpha-synuclein as defined herein (also known as “critical residues”), in particular interacting with/binding to at least two amino acids within residues 1-15 (SEQ ID NO: 121), 15-45 (SEQ ID NO: 138), 19-33 (SEQ ID NO: 123), 28-50 (SEQ ID NO: 139), 31-60 (SEQ ID NO: 146), 81-120 (SEQ ID NO: 137), 91-105 (SEQ ID NO: 131), 96-140 (SEQ ID NO: 147) or 100-114 (SEQ ID NO: 132) of human alpha-synuclein of SEQ ID NO: 1. The residues may form a linear or a non-linear epitope. Preferably, antigen-binding molecule of the invention bind to an epitope within amino acids residues 28-50 (SEQ ID NO: 139), 31-60 (SEQ ID NO: 146) or 81-120 (SEQ ID NO: 137) of human alpha-synuclein of SEQ ID NO: 1. Even more preferably, antigen-binding molecules of the invention may bind to an epitope comprising amino acids 33-37 or 33-39 or 108-113 of human alpha-synuclein of SEQ ID NO: 1 as critical residues for binding. The antigen binding molecules of the invention may also bind to a non-linear epitope within amino acids residues of human alpha-synuclein of SEQ ID NO: 1.

Cross-reactivity of antigen-binding molecules, in particular a panel of antibodies or antigen-binding fragments thereof under investigation may be tested, for example, by assessing binding of said panel of antibodies or antigen-binding fragments thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of alpha-synuclein as defined herein, e.g., a specific epitope or (poly)peptide/protein of alpha-synuclein as defined herein but do not or do not essentially bind to any of the other epitope or (poly)peptides of the same alpha-synuclein, are considered specific for the epitope or (poly)peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with BIACORE™), analytical ultracentrifugation, isothermal titration calorimetry, fluorescence anisotropy, fluorescence spectroscopy or by radiolabeled ligand binding assays.

Accordingly, specificity can be determined experimentally by methods known in the art and methods as described herein. Such methods comprise, but are not limited to Western Blots, ELISA-, RIA-, ECL-, IRMA-tests and peptide scans.

It may be understood by a person skilled in the art that the epitopes may be comprised in the alpha-synuclein protein, but may also be comprised in a degradation product thereof or may be a chemically synthesized peptide. The amino acid positions are only indicated to demonstrate the position of the corresponding amino acid sequence in the sequence of the alpha-synuclein protein. The invention encompasses all peptides comprising the epitope. The peptide may be a part of a polypeptide of more than 100 amino acids in length or may be a small peptide of less than 100, preferably less than 50, more preferably less than 25 amino acids, even more preferably less than 18 amino acids. The amino acids of such peptide may be natural amino acids or nonnatural amino acids (e.g., beta-amino acids, gamma-amino acids, D-amino acids) or a combination thereof. Further, the present invention may encompass the respective retro-inverso peptides of the epitopes. The peptide may be unbound or bound. It may be bound, e.g., to a small molecule (e.g., a drug or a fluorophor), to a high-molecular weight polymer (e.g., polyethylene glycol (PEG), polyethylene imine (PEI), hydroxypropylmethacrylate (HPMA), etc.) or to a protein, a fatty acid, a sugar moiety or may be inserted in a membrane. In order to test whether an antibody in question and the antibody of the present invention recognize the same or similar epitope, many assays are known in the art, some of which (e.g. “alanine scanning mutagenesis”) is described in below in example.

Whether an antibody recognizes the same epitope as or an epitope overlapping with an epitope that is recognized by another antibody as provided herein can be confirmed by competition between the two antibodies against the epitope. Competition between the antibodies can be evaluated by competitive binding assays using means such as enzyme-linked immunosorbent assay (ELISA), fluorescence energy transfer method (FRET), and fluorometric microvolume assay technology (FMAT®). The amount of antibodies bound to an antigen indirectly correlate with the binding ability of candidate competitor antibodies (test antibodies) that competitively bind to the same or overlapping epitope. In other words, as the amount of or the affinity of test antibodies against the same or overlapping epitope increases, the amount of antibodies bound to the antigen decreases, and the amount of test antibodies bound to the antigen increases. Specifically, the appropriately labeled antibodies and test antibodies are simultaneously added to the antigens, and then the bound antibodies are detected using the label. The amount of the antibodies bound to the antigen can be easily determined by labeling the antibodies in advance. This label is not particularly limited, and the labeling method is selected according to the assay technique used. Specific examples of the labeling method include fluorescent labeling, radiolabeling, and enzyme labeling.

Herein, the “antibody that binds to the overlapping epitope” or “antibody that binds to the same epitope” refers to a test antibody that can reduce the amount of binding of the labeled antibody by at least 50% at a concentration that is usually 100 times higher, preferably 80 times higher, more preferably 50 times higher, even more preferably 30 times higher, and still more preferably 10 times higher than a concentration of the non-labeled antibody at which binding of the non-labeled antibody reduces the amount of binding of the labeled antibody by 50% (IC50). The epitope recognized by the antibody can be analyzed by methods known to those skilled in the art, and for example, it can be performed by Western blotting and such.

In some embodiments, the antibody comprises:
a) a Heavy Chain Variable Region (VH) comprising the sequence of SEQ ID NO: 610 or a heavy chain variable region (VH) having at least 92%, 93%, 94%, 95%, 96%, 97%, 98%, or 99% sequence identity to the amino acid sequence of SEQ ID NO: 610; or
b) a Heavy Chain Variable Region (VH) comprising the sequence of SEQ ID NO: 620 or a heavy chain variable region (VH) having at least 96%, 97%, 98%, or 99% sequence identity to the amino acid sequence of SEQ ID NO: 620; or
c) a Heavy Chain Variable Region (VH) comprising the sequence of SEQ ID NO: 630 or a heavy chain variable region (VH) having at least 99% sequence identity to the amino acid sequence of SEQ ID NO: 630; or
d) a Heavy Chain Variable Region (VH) comprising the sequence of SEQ ID NO: 640 or a heavy chain variable region (VH) having at least 96%, 97%, 98%, or 99% sequence identity to the amino acid sequence of SEQ ID NO: 640; or
e) a Heavy Chain Variable Region (VH) comprising the sequence of SEQ ID NO: 650 or a heavy chain variable region (VH) having at least 98%, or 99% sequence identity to the amino acid sequence of SEQ ID NO: 650; or
f) a Heavy Chain Variable Region (VH) comprising the sequence of SEQ ID NO: 660 or a heavy chain variable region (VH) having at least 92%, 93%, 94%, 95%, 96%, 97%, 98%, or 99% sequence identity to the amino acid sequence of SEQ ID NO: 660; or
g) a Heavy Chain Variable Region (VH) comprising the sequence of SEQ ID NO: 670 or a heavy chain variable region (VH) having at least 92%, 93%, 94%, 95%, 96%, 97%, 98%, or 99% sequence identity to the amino acid sequence of SEQ ID NO: 670; or
h) a Heavy Chain Variable Region (VH) comprising the sequence of SEQ ID NO: 680 or a heavy chain variable region (VH) having at least 97%, 98%, or 99% sequence identity to the amino acid sequence of SEQ ID NO: 680; or
i) a Heavy Chain Variable Region (VH) comprising the sequence of SEQ ID NO: 690 or a heavy chain variable region (VH) having at least 87%, 88%, 89%, 90%, 91%, 92%, 93%, 94%, 95%, 96%, 97%, 98%, or 99% sequence identity to the amino acid sequence of SEQ ID NO: 690; or
j) a Heavy Chain Variable Region (VH) comprising the sequence of SEQ ID NO: 700 or a heavy chain variable region (VH) having at least 86%, 87%, 88%, 89%, 90%, 91%, 92%, 93%, 94%, 95%, 96%, 97%, 98%, or 99% sequence identity to the amino acid sequence of SEQ ID NO: 700; or
k) a Heavy Chain Variable Region (VH) comprising the sequence of SEQ ID NO: 710 or a heavy chain variable region (VH) having at least 96%, 97%, 98%, or 99% sequence identity to the amino acid sequence of SEQ ID NO: 710; or
l) a Heavy Chain Variable Region (VH) comprising the sequence of SEQ ID NO: 720; or
m) a Heavy Chain Variable Region (VH) comprising the sequence of SEQ ID NO: 730 or a heavy chain variable region (VH) having at least 97%, 98%, or 99% sequence identity to the amino acid sequence of SEQ ID NO: 730; or
n) a Heavy Chain Variable Region (VH) comprising the sequence of SEQ ID NO: 740 or a heavy chain variable region (VH) having at least 88%, 89%, 90%, 91%, 92%, 93%, 94%, 95%, 96%, 97%, 98%, or 99% sequence identity to the amino acid sequence of SEQ ID NO: 740; or
o) a Heavy Chain Variable Region (VH) comprising the sequence of SEQ ID NO: 750 or a heavy chain variable region (VH) having at least 99% sequence identity to the amino acid sequence of SEQ ID NO: 750; or
p) a Heavy Chain Variable Region (VH) comprising the sequence of SEQ ID NO: 760 or a heavy chain variable region (VH) having at least 97%, 98%, or 99% sequence identity to the amino acid sequence of SEQ ID NO: 760; or
q) a Heavy Chain Variable Region (VH) comprising the sequence of SEQ ID NO: 770 or a heavy chain variable region (VH) having at least 93%, 94%, 95%, 96%, 97%, 98%, or 99% sequence identity to the amino acid sequence of SEQ ID NO: 770; or
r) a Heavy Chain Variable Region (VH) comprising the sequence of SEQ ID NO: 790 or a heavy chain variable region (VH) having at least 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 790; or
s) a Heavy Chain Variable Region (VH) comprising the sequence of SEQ ID NO: 800 or a heavy chain variable region (VH) having at least 92%, 93%, 94%, 95%, 96%, 97%, 98%, or 99% sequence identity to the amino acid sequence of SEQ ID NO: 800; or
t) a Heavy Chain Variable Region (VH) comprising the sequence of SEQ ID NO: 810 or a heavy chain variable region (VH) having at least 94%, 95%, 96%, 97%, 98%, or 99% sequence identity to the amino acid sequence of SEQ ID NO: 810; or
u) a Heavy Chain Variable Region (VH) comprising the sequence of SEQ ID NO: 820 or a heavy chain variable region (VH) having at least 98% or 99% sequence identity to the amino acid sequence of SEQ ID NO: 820; or
v) a Heavy Chain Variable Region (VH) comprising the sequence of SEQ ID NO: 830 or a heavy chain variable region (VH) having at least 94%, 95%, 96%, 97%, 98%, or 99% sequence identity to the amino acid sequence of SEQ ID NO: 830; or
w) a Heavy Chain Variable Region (VH) comprising the sequence of SEQ ID NO: 840 or a heavy chain variable region (VH) having at least 90%, 91%, 92%, 93%, 94%, 95%, 96%, 97%, 98%, or 99% sequence identity to the amino acid sequence of SEQ ID NO: 840.

In some embodiments, the antibody comprises:
a) a Light Chain Variable Region (VL) comprising the sequence of SEQ ID NO: 614; or
b) a Light Chain Variable Region (VL) comprising the sequence of SEQ ID NO: 624 or a light chain variable region (VL) having at least 92%, 93%, 94%, 95%, 96%, 97%, 98%, or 99% sequence identity to the amino acid sequence of SEQ ID NO: 624; or
c) a Light Chain Variable Region (VL) comprising the sequence of SEQ ID NO: 634 or a light chain variable region (VL) having at least 99% sequence identity to the amino acid sequence of SEQ ID NO: 634; or
d) a Light Chain Variable Region (VL) comprising the sequence of SEQ ID NO: 644 or a light chain variable region (VL) having at least 96%, 97%, 98%, or 99% sequence identity to the amino acid sequence of SEQ ID NO: 644; or
e) a Light Chain Variable Region (VL) comprising the sequence of SEQ ID NO: 654 or a light chain variable region (VL) having at least 99% sequence identity to the amino acid sequence of SEQ ID NO: 654; or
f) a Light Chain Variable Region (VL) comprising the sequence of SEQ ID NO: 664 or a light chain variable region (VL) having at least 99% sequence identity to the amino acid sequence of SEQ ID NO: 664; or
g) a Light Chain Variable Region (VL) comprising the sequence of SEQ ID NO: 674; or a Light Chain Variable Region (VL) comprising the sequence of SEQ ID NO: 674; or
h) a Light Chain Variable Region (VL) comprising the sequence of SEQ ID NO: 684 or a light chain variable region (VL) having at least 99% sequence identity to the amino acid sequence of SEQ ID NO: 684; or
i) a Light Chain Variable Region (VL) comprising the sequence of SEQ ID NO: 694 or a light chain variable region (VL) having at least 96%, 97%, 98%, or 99% sequence identity to the amino acid sequence of SEQ ID NO: 694; or
j) a Light Chain Variable Region (VL) comprising the sequence of SEQ ID NO: 704 or a light chain variable region (VL) having at least 94%, 95%, 96%, 97%, 98%, or 99% sequence identity to the amino acid sequence of SEQ ID NO: 704; or
k) a Light Chain Variable Region (VL) comprising the sequence of SEQ ID NO: 714 or a light chain variable region (VL) having at least 98%, or 99% sequence identity to the amino acid sequence of SEQ ID NO: 714; or
l) a Light Chain Variable Region (VL) comprising the sequence of SEQ ID NO: 724 or a light chain variable region (VL) having at least 95%, 96%, 97%, 98%, or 99% sequence identity to the amino acid sequence of SEQ ID NO: 724; or
m) a Light Chain Variable Region (VL) comprising the sequence of SEQ ID NO: 734 or a light chain variable region (VL) having at least 97%, 98%, or 99% sequence identity to the amino acid sequence of SEQ ID NO: 734; or
n) a Light Chain Variable Region (VL) comprising the sequence of SEQ ID NO: 744 or a light chain variable region (VL) having at least 99% sequence identity to the amino acid sequence of SEQ ID NO: 744; or
o) a Light Chain Variable Region (VL) comprising the sequence of SEQ ID NO: 754 or a light chain variable region (VL) having at least 97%, 98%, or 99% sequence identity to the amino acid sequence of SEQ ID NO: 754; or
p) a Light Chain Variable Region (VL) comprising the sequence of SEQ ID NO: 764 or a light chain variable region (VL) having at least 98%, or 99% sequence identity to the amino acid sequence of SEQ ID NO: 764; or
q) a Light Chain Variable Region (VL) comprising the sequence of SEQ ID NO: 774 or a light chain variable region (VL) having at least 97%, 98%, or 99% sequence identity to the amino acid sequence of SEQ ID NO: 774; or
r) a Light Chain Variable Region (VL) comprising the sequence of SEQ ID NO: 784 or a light chain variable region (VL) having at least 96%, 97%, 98%, or 99% sequence identity to the amino acid sequence of SEQ ID NO: 784; or
s) a Light Chain Variable Region (VL) comprising the sequence of SEQ ID NO: 794 or a light chain variable region (VL) having at least 95%, 96%, 97%, 98%, or 99% sequence identity to the amino acid sequence of SEQ ID NO: 794; or
t) a Light Chain Variable Region (VL) comprising the sequence of SEQ ID NO: 804 or a light chain variable region (VL) having at least 96%, 97%, 98%, or 99% sequence identity to the amino acid sequence of SEQ ID NO: 804; or
u) a Light Chain Variable Region (VL) comprising the sequence of SEQ ID NO: 814 or a light chain variable region (VL) having at least 99% sequence identity to the amino acid sequence of SEQ ID NO: 814; or
v) a Light Chain Variable Region (VL) comprising the sequence of SEQ ID NO: 824 or a light chain variable region (VL) having at least 98% or 99% sequence identity to the amino acid sequence of SEQ ID NO: 824; or
w) a Light Chain Variable Region (VL) comprising the sequence of SEQ ID NO: 834; or
x) a Light Chain Variable Region (VL) comprising the sequence of SEQ ID NO: 844.

In some embodiments, the antibody comprises:
a) a Heavy Chain Variable Region (VH) comprising the sequence of SEQ ID NO: 610 and a Light Chain Variable Region (VL) comprising the sequence of SEQ ID NO: 614; or
b) a Heavy Chain Variable Region (VH) comprising the sequence of SEQ ID NO: 620 and a Light Chain Variable Region (VL) comprising the sequence of SEQ ID NO: 624; or
c) a Heavy Chain Variable Region (VH) comprising the sequence of SEQ ID NO: 630 and a Light Chain Variable Region (VL) comprising the sequence of SEQ ID NO: 634; or
d) a Heavy Chain Variable Region (VH) comprising the sequence of SEQ ID NO: 640 and a Light Chain Variable Region (VL) comprising the sequence of SEQ ID NO: 644; or
e) a Heavy Chain Variable Region (VH) comprising the sequence of SEQ ID NO: 650 and a Light Chain Variable Region (VL) comprising the sequence of SEQ ID NO: 654; or
f) a Heavy Chain Variable Region (VH) comprising the sequence of SEQ ID NO: 660 and a Light Chain Variable Region (VL) comprising the sequence of SEQ ID NO: 664; or
g) a Heavy Chain Variable Region (VH) comprising the sequence of SEQ ID NO: 670 and a Light Chain Variable Region (VL) comprising the sequence of SEQ ID NO: 674; or
h) a Heavy Chain Variable Region (VH) comprising the sequence of SEQ ID NO: 680 and a Light Chain Variable Region (VL) comprising the sequence of SEQ ID NO: 684; or
i) a Heavy Chain Variable Region (VH) comprising the sequence of SEQ ID NO: 690 and a Light Chain Variable Region (VL) comprising the sequence of SEQ ID NO: 694; or
j) a Heavy Chain Variable Region (VH) comprising the sequence of SEQ ID NO: 700 and a Light Chain Variable Region (VL) comprising the sequence of SEQ ID NO: 704; or
k) a Heavy Chain Variable Region (VH) comprising the sequence of SEQ ID NO: 710 and a Light Chain Variable Region (VL) comprising the sequence of SEQ ID NO: 714; or
l) a Heavy Chain Variable Region (VH) comprising the sequence of SEQ ID NO: 720 and a Light Chain Variable Region (VL) comprising the sequence of SEQ ID NO: 724; or
m) a Heavy Chain Variable Region (VH) comprising the sequence of SEQ ID NO: 730 and a Light Chain Variable Region (VL) comprising the sequence of SEQ ID NO: 734; or
n) a Heavy Chain Variable Region (VH) comprising the sequence of SEQ ID NO: 740 and a Light Chain Variable Region (VL) comprising the sequence of SEQ ID NO: 744; or
o) a Heavy Chain Variable Region (VH) comprising the sequence of SEQ ID NO: 750 and a Light Chain Variable Region (VL) comprising the sequence of SEQ ID NO: 754; or
p) a Heavy Chain Variable Region (VH) comprising the sequence of SEQ ID NO: 760 and a Light Chain Variable Region (VL) comprising the sequence of SEQ ID NO: 764; or
q) a Heavy Chain Variable Region (VH) comprising the sequence of SEQ ID NO: 770 and a Light Chain Variable Region (VL) comprising the sequence of SEQ ID NO: 774; or
r) a Heavy Chain Variable Region (VH) comprising the sequence of SEQ ID NO: 750 and a Light Chain Variable Region (VL) comprising the sequence of SEQ ID NO: 784; or
s) a Heavy Chain Variable Region (VH) comprising the sequence of SEQ ID NO: 790 and a Light Chain Variable Region (VL) comprising the sequence of SEQ ID NO: 794; or
t) a Heavy Chain Variable Region (VH) comprising the sequence of SEQ ID NO: 800 and a Light Chain Variable Region (VL) comprising the sequence of SEQ ID NO: 804; or
u) a Heavy Chain Variable Region (VH) comprising the sequence of SEQ ID NO: 810 and a Light Chain Variable Region (VL) comprising the sequence of SEQ ID NO: 814; or
v) a Heavy Chain Variable Region (VH) comprising the sequence of SEQ ID NO: 820 and a Light Chain Variable Region (VL) comprising the sequence of SEQ ID NO: 824; or
w) a Heavy Chain Variable Region (VH) comprising the sequence of SEQ ID NO: 830 and a Light Chain Variable Region (VL) comprising the sequence of SEQ ID NO: 834; or
x) a Heavy Chain Variable Region (VH) comprising the sequence of SEQ ID NO: 840 and a Light Chain Variable Region (VL) comprising the sequence of SEQ ID NO: 844.

In some embodiments, the antibody comprises:
a. a Heavy Chain Variable Region (VH) comprising the sequence of SEQ ID NO: 610 or a heavy chain variable region (VH) having at least 92%, 93%, 94%, 95%, 96%, 97%, 98%, or 99% sequence identity to the amino acid sequence of SEQ ID NO: 610; and a Light Chain Variable Region (VL) comprising the sequence of SEQ ID NO: 614; or
b. a Heavy Chain Variable Region (VH) comprising the sequence of SEQ ID NO: 620 or a heavy chain variable region (VH) having at least 96%, 97%, 98%, or 99% sequence identity to the amino acid sequence of SEQ ID NO: 620; and a Light Chain Variable Region (VL) comprising the sequence of SEQ ID NO: 624 or a light chain variable region (VL) having at least 92%, 93%, 94%, 95%, 96%, 97%, 98%, or 99% sequence identity to the amino acid sequence of SEQ ID NO: 624; or
c. a Heavy Chain Variable Region (VH) comprising the sequence of SEQ ID NO: 630 or a heavy chain variable region (VH) having at least 99% sequence identity to the amino acid sequence of SEQ ID NO: 630; and a Light Chain Variable Region (VL) comprising the sequence of SEQ ID NO: 634 or a light chain variable region (VL) having at least 99% sequence identity to the amino acid sequence of SEQ ID NO: 634; or
d. a Heavy Chain Variable Region (VH) comprising the sequence of SEQ ID NO: 640 or a heavy chain variable region (VH) having at least 96%, 97%, 98%, or 99% sequence identity to the amino acid sequence of SEQ ID NO: 640; and a Light Chain Variable Region (VL) comprising the sequence of SEQ ID NO: 644 or a light chain variable region (VL) having at least 96%, 97%, 98%, or 99% sequence identity to the amino acid sequence of SEQ ID NO: 644; or
e. a Heavy Chain Variable Region (VH) comprising the sequence of SEQ ID NO: 650 or a heavy chain variable region (VH) having at least 98%, or 99% sequence identity to the amino acid sequence of SEQ ID NO: 650; and a Light Chain Variable Region (VL) comprising the sequence of SEQ ID NO: 654 or a light chain variable region (VL) having at least 99% sequence identity to the amino acid sequence of SEQ ID NO: 654; or
f. a Heavy Chain Variable Region (VH) comprising the sequence of SEQ ID NO: 660 or a heavy chain variable region (VH) having at least 92%, 93%, 94%, 95%, 96%, 97%, 98%, or 99% sequence identity to the amino acid sequence of SEQ ID NO: 660; and a Light Chain Variable Region (VL) comprising the sequence of SEQ ID NO: 664 or a light chain variable region (VL) having at least 99% sequence identity to the amino acid sequence of SEQ ID NO: 664; or
g. a Heavy Chain Variable Region (VH) comprising the sequence of SEQ ID NO: 670 or a heavy chain variable region (VH) having at least 92%, 93%, 94%, 95%, 96%, 97%, 98%, or 99% sequence identity to the amino acid sequence of SEQ ID NO: 670; and a Light Chain Variable Region (VL) comprising the sequence of SEQ ID NO: 674; or
h. a Heavy Chain Variable Region (VH) comprising the sequence of SEQ ID NO: 680 or a heavy chain variable region (VH) having at least 97%, 98%, or 99% sequence identity to the amino acid sequence of SEQ ID NO: 680; and a Light Chain Variable Region (VL) comprising the sequence of SEQ ID NO: 684 or a light chain variable region (VL) having at least 99% sequence identity to the amino acid sequence of SEQ ID NO: 684; or
i. a Heavy Chain Variable Region (VH) comprising the sequence of SEQ ID NO: 690 or a heavy chain variable region (VH) having at least 87%, 88%, 89%, 90%, 91%, 92%, 93%, 94%, 95%, 96%, 97%, 98%, or 99% sequence identity to the amino acid sequence of SEQ ID NO: 690; and a Light Chain Variable Region (VL) comprising the sequence of SEQ ID NO: 694 or a light chain variable region (VL) having at least 96%, 97%, 98%, or 99% sequence identity to the amino acid sequence of SEQ ID NO: 694; or
j. a Heavy Chain Variable Region (VH) comprising the sequence of SEQ ID NO: 700 or a heavy chain variable region (VH) having at least 86%, 87%, 88%, 89%, 90%, 91%, 92%, 93%, 94%, 95%, 96%, 97%, 98%, or 99% sequence identity to the amino acid sequence of SEQ ID NO: 700; and a Light Chain Variable Region (VL) comprising the sequence of SEQ ID NO: 704 or a light chain variable region (VL) having at least 94%, 95%, 96%, 97%, 98%, or 99% sequence identity to the amino acid sequence of SEQ ID NO: 704; or
k. a Heavy Chain Variable Region (VH) comprising the sequence of SEQ ID NO: 710 or a heavy chain variable region (VH) having at least 96%, 97%, 98%, or 99% sequence identity to the amino acid sequence of SEQ ID NO: 710; and a Light Chain Variable Region (VL) comprising the sequence of SEQ ID NO: 714 or a light chain variable region (VL) having at least 98%, or 99% sequence identity to the amino acid sequence of SEQ ID NO: 714; or
l. a Heavy Chain Variable Region (VH) comprising the sequence of SEQ ID NO: 720; and a Light Chain Variable Region (VL) comprising the sequence of SEQ ID NO: 724 or a light chain variable region (VL) having at least 95%, 96%, 97%, 98%, or 99% sequence identity to the amino acid sequence of SEQ ID NO: 724; or
m. a Heavy Chain Variable Region (VH) comprising the sequence of SEQ ID NO: 730 or a heavy chain variable region (VH) having at least 97%, 98%, or 99% sequence identity to the amino acid sequence of SEQ ID NO: 730; and a Light Chain Variable Region (VL) comprising the sequence of SEQ ID NO: 734 or a light chain variable region (VL) having at least 97%, 98%, or 99% sequence identity to the amino acid sequence of SEQ ID NO: 734; or
n. a Heavy Chain Variable Region (VH) comprising the sequence of SEQ ID NO: 740 or a heavy chain variable region (VH) having at least 88%, 89%, 90%, 91%, 92%, 93%, 94%, 95%, 96%, 97%, 98%, or 99% sequence identity to the amino acid sequence of SEQ ID NO: 740; and a Light Chain Variable Region (VL) comprising the sequence of SEQ ID NO: 744 or a light chain variable region (VL) having at least 99% sequence identity to the amino acid sequence of SEQ ID NO: 744; or
o. a Heavy Chain Variable Region (VH) comprising the sequence of SEQ ID NO: 750 or a heavy chain variable region (VH) having at least 99% sequence identity to the amino acid sequence of SEQ ID NO: 750; and a Light Chain Variable Region (VL) comprising the sequence of SEQ ID NO: 754 or a light chain variable region (VL) having at least 97%, 98%, or 99% sequence identity to the amino acid sequence of SEQ ID NO: 754; or
p. a Heavy Chain Variable Region (VH) comprising the sequence of SEQ ID NO: 760 or a heavy chain variable region (VH) having at least 97%, 98%, 99% sequence identity to the amino acid sequence of SEQ ID NO: 760; and a Light Chain Variable Region (VL) comprising the sequence of SEQ ID NO: 764 or a light chain variable region (VL) having at least 98%, or 99% sequence identity to the amino acid sequence of SEQ ID NO: 764; or
q. a Heavy Chain Variable Region (VH) comprising the sequence of SEQ ID NO: 770 or a heavy chain variable region (VH) having at least 93%, 94%, 95%, 96%, 97%, 98%, or 99% sequence identity to the amino acid sequence of SEQ ID NO: 770; and a Light Chain Variable Region (VL) comprising the sequence of SEQ ID NO: 774 or a light chain variable region (VL) having at least 97%, 98%, or 99% sequence identity to the amino acid sequence of SEQ ID NO: 774; or
r. a Heavy Chain Variable Region (VH) comprising the sequence of SEQ ID NO: 750 or a heavy chain variable region (VH) having at least 99% sequence identity to the amino acid sequence of SEQ ID NO: 750; and a Light Chain Variable Region (VL) comprising the sequence of SEQ ID NO: 784 or a light chain variable region (VL) having at least 96%, 97%, 98%, or 99% sequence identity to the amino acid sequence of SEQ ID NO: 784; or
s. a Heavy Chain Variable Region (VH) comprising the sequence of SEQ ID NO: 790 or a heavy chain variable region (VH) having at least 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 790; and a Light Chain Variable Region (VL) comprising the sequence of SEQ ID NO: 794 or a light chain variable region (VL) having at least 95%, 96%, 97%, 98%, or 99% sequence identity to the amino acid sequence of SEQ ID NO: 794; or
t. a Heavy Chain Variable Region (VH) comprising the sequence of SEQ ID NO: 800 or a heavy chain variable region (VH) having at least 92%, 93%, 94%, 95%, 96%, 97%, 98%, or 99% sequence identity to the amino acid sequence of SEQ ID NO: 800; and a Light Chain Variable Region (VL) comprising the sequence of SEQ ID NO: 804 or a light chain variable region (VL) having at least 96%, 97%, 98%, or 99% sequence identity to the amino acid sequence of SEQ ID NO: 804; or
u. a Heavy Chain Variable Region (VH) comprising the sequence of SEQ ID NO: 810 or a heavy chain variable region (VH) having at least 94%, 95%, 96%, 97%, 98%, or 99% sequence identity to the amino acid sequence of SEQ ID NO: 810; and a Light Chain Variable Region (VL) comprising the sequence of SEQ ID NO: 814 or a light chain variable region (VL) having at least 99% sequence identity to the amino acid sequence of SEQ ID NO: 814; or
v. a Heavy Chain Variable Region (VH) comprising the sequence of SEQ ID NO: 820 or a heavy chain variable region (VH) having at least 98% or 99% sequence identity to the amino acid sequence of SEQ ID NO: 820; and a Light Chain Variable Region (VL) comprising the sequence of SEQ ID NO: 824 or a light chain variable region (VL) having at least 98% or 99% sequence identity to the amino acid sequence of SEQ ID NO: 824; or
w. a Heavy Chain Variable Region (VH) comprising the sequence of SEQ ID NO: 830 or a heavy chain variable region (VH) having at least 94%, 95%, 96%, 97%, 98%, or 99% sequence identity to the amino acid sequence of SEQ ID NO: 830; and a Light Chain Variable Region (VL) comprising the sequence of SEQ ID NO: 834; or
x. a Heavy Chain Variable Region (VH) comprising the sequence of SEQ ID NO: 840 or a heavy chain variable region (VH) having at least 90%, 91%, 92%, 93%, 94%, 95%, 96%, 97%, 98%, or 99% sequence identity to the amino acid sequence of SEQ ID NO: 840; and a Light Chain Variable Region (VL) comprising the sequence of SEQ ID NO: 844.

In some embodiments, the antibody comprises:
a) VH-CDR1 comprising the amino acid sequence of SEQ ID NO: 611; VH-CDR2 comprising the amino acid sequence of SEQ ID NO: 612; and VH-CDR3 comprising the amino acid sequence of SEQ ID NO: 613; or
b) VH-CDR1 comprising the amino acid sequence of SEQ ID NO: 621; VH-CDR2 comprising the amino acid sequence of SEQ ID NO: 622; and VH-CDR3 comprising the amino acid sequence of SEQ ID NO: 623; or
c) VH-CDR1 comprising the amino acid sequence of SEQ ID NO: 631; VH-CDR2 comprising the amino acid sequence of SEQ ID NO: 632; and VH-CDR3 comprising the amino acid sequence of SEQ ID NO: 633; or
d) VH-CDR1 comprising the amino acid sequence of SEQ ID NO: 641; VH-CDR2 comprising the amino acid sequence of SEQ ID NO: 642; and VH-CDR3 comprising the amino acid sequence of SEQ ID NO: 643; or
e) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 653; or
f) VH-CDR1 comprising the amino acid sequence of SEQ ID NO: 661; VH-CDR2 comprising the amino acid sequence of SEQ ID NO: 662; and VH-CDR3 comprising the amino acid sequence of SEQ ID NO: 663; or
g) VH-CDR1 comprising the amino acid sequence of SEQ ID NO: 671; VH-CDR2 comprising the amino acid sequence of SEQ ID NO: 672; and VH-CDR3 comprising the amino acid sequence of SEQ ID NO: 673; or
h) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 683; or
i) VH-CDR1 comprising the amino acid sequence of SEQ ID NO: 691; VH-CDR2 comprising the amino acid sequence of SEQ ID NO: 692; and VH-CDR3 comprising the amino acid sequence of SEQ ID NO: 693; or
j) VH-CDR1 comprising the amino acid sequence of SEQ ID NO: 701; VH-CDR2 comprising the amino acid sequence of SEQ ID NO: 702; and VH-CDR3 comprising the amino acid sequence of SEQ ID NO: 703; or
k) VH-CDR1 comprising the amino acid sequence of SEQ ID NO: 711; VH-CDR2 comprising the amino acid sequence of SEQ ID NO: 712; and VH-CDR3 comprising the amino acid sequence of SEQ ID NO: 713; or
l) VH-CDR1 comprising the amino acid sequence of SEQ ID NO: 721; VH-CDR2 comprising the amino acid sequence of SEQ ID NO: 722; and VH-CDR3 comprising the amino acid sequence of SEQ ID NO: 723; or
m) VH-CDR1 comprising the amino acid sequence of SEQ ID NO: 731; VH-CDR2 comprising the amino acid sequence of SEQ ID NO: 722; and VH-CDR3 comprising the amino acid sequence of SEQ ID NO: 733; or
n) VH-CDR1 comprising the amino acid sequence of SEQ ID NO: 671; VH-CDR2 comprising the amino acid sequence of SEQ ID NO: 742; and VH-CDR3 comprising the amino acid sequence of SEQ ID NO: 743; or
o) VH-CDR1 comprising the amino acid sequence of SEQ ID NO: 751; VH-CDR2 comprising the amino acid sequence of SEQ ID NO: 722; and VH-CDR3 comprising the amino acid sequence of SEQ ID NO: 723; or
p) VH-CDR1 comprising the amino acid sequence of SEQ ID NO: 761; VH-CDR2 comprising the amino acid sequence of SEQ ID NO: 722; and VH-CDR3 comprising the amino acid sequence of SEQ ID NO: 733; or
q) VH-CDR1 comprising the amino acid sequence of SEQ ID NO: 771; VH-CDR2 comprising the amino acid sequence of SEQ ID NO: 772; and VH-CDR3 comprising the amino acid sequence of SEQ ID NO: 773; or
r) VH-CDR1 comprising the amino acid sequence of SEQ ID NO: 791; VH-CDR2 comprising the amino acid sequence of SEQ ID NO: 792; and VH-CDR3 comprising the amino acid sequence of SEQ ID NO: 793; or
s) VH-CDR1 comprising the amino acid sequence of SEQ ID NO: 771; VH-CDR2 comprising the amino acid sequence of SEQ ID NO: 802; and VH-CDR3 comprising the amino acid sequence of SEQ ID NO: 803; or
t) VH-CDR1 comprising the amino acid sequence of SEQ ID NO: 811; VH-CDR2 comprising the amino acid sequence of SEQ ID NO: 812; and VH-CDR3 comprising the amino acid sequence of SEQ ID NO: 813; or
u) VH-CDR1 comprising the amino acid sequence of SEQ ID NO: 821; VH-CDR2 comprising the amino acid sequence of SEQ ID NO: 822; and VH-CDR3 comprising the amino acid sequence of SEQ ID NO: 823; or
v) VH-CDR1 comprising the amino acid sequence of SEQ ID NO: 831; VH-CDR2 comprising the amino acid sequence of SEQ ID NO: 832; and VH-CDR3 comprising the amino acid sequence of SEQ ID NO: 833; or
w) VH-CDR1 comprising the amino acid sequence of SEQ ID NO: 841; VH-CDR2 comprising the amino acid sequence of SEQ ID NO: 842; and VH-CDR3 comprising the amino acid sequence of SEQ ID NO: 843.

In some embodiments, the antibody comprises:
a) VL-CDR1 comprising the amino acid sequence of SEQ ID NO: 615; VL-CDR2 comprising the amino acid sequence of SEQ ID NO: 616; and VL-CDR3 comprising the amino acid sequence of SEQ ID NO: 617; or
b) VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
c) VL-CDR1 comprising the amino acid sequence of SEQ ID NO: 635; VL-CDR2 comprising the amino acid sequence of SEQ ID NO: 616; and VL-CDR3 comprising the amino acid sequence of SEQ ID NO: 637; or
d) VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
e) VL-CDR1 comprising the amino acid sequence of SEQ ID NO: 655; VL-CDR2 comprising the amino acid sequence of SEQ ID NO: 626; and VL-CDR3 comprising the amino acid sequence of SEQ ID NO: 627; or
f) VL-CDR1 comprising the amino acid sequence of SEQ ID NO: 665; VL-CDR2 comprising the amino acid sequence of SEQ ID NO: 666; and VL-CDR3 comprising the amino acid sequence of SEQ ID NO: 667; or
g) VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
h) VL-CDR1 comprising the amino acid sequence of SEQ ID NO: 625; VL-CDR2 comprising the amino acid sequence of SEQ ID NO: 686; and VL-CDR3 comprising the amino acid sequence of SEQ ID NO: 627; or
i) VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697; or
j) VL-CDR1 comprising the amino acid sequence of SEQ ID NO: 705; VL-CDR2 comprising the amino acid sequence of SEQ ID NO: 706; and VL-CDR3 comprising the amino acid sequence of SEQ ID NO: 707; or
k) VL-CDR1 comprising the amino acid sequence of SEQ ID NO: 715; VL-CDR2 comprising the amino acid sequence of SEQ ID NO: 716; and VL-CDR3 comprising the amino acid sequence of SEQ ID NO: 717; or
l) VL-CDR1 comprising the amino acid sequence of SEQ ID NO: 725; VL-CDR2 comprising the amino acid sequence of SEQ ID NO: 616; and VL-CDR3 comprising the amino acid sequence of SEQ ID NO: 637; or
m) VL-CDR1 comprising the amino acid sequence of SEQ ID NO: 735; VL-CDR2 comprising the amino acid sequence of SEQ ID NO: 736; and VL-CDR3 comprising the amino acid sequence of SEQ ID NO: 637; or
n) VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
o) VL-CDR1 comprising the amino acid sequence of SEQ ID NO: 735; VL-CDR2 comprising the amino acid sequence of SEQ ID NO: 616; and VL-CDR3 comprising the amino acid sequence of SEQ ID NO: 637; or
p) VL-CDR1 comprising the amino acid sequence of SEQ ID NO: 765; VL-CDR2 comprising the amino acid sequence of SEQ ID NO: 616; and VL-CDR3 comprising the amino acid sequence of SEQ ID NO: 637; or
q) VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
r) VL-CDR1 comprising the amino acid sequence of SEQ ID NO: 795; VL-CDR2 comprising the amino acid sequence of SEQ ID NO: 616; and VL-CDR3 comprising the amino acid sequence of SEQ ID NO: 797; or
s) VL-CDR1 comprising the amino acid sequence of SEQ ID NO: 805; VL-CDR2 comprising the amino acid sequence of SEQ ID NO: 676; and VL-CDR3 comprising the amino acid sequence of SEQ ID NO: 677; or
t) VL-CDR1 comprising the amino acid sequence of SEQ ID NO: 815; VL-CDR2 comprising the amino acid sequence of SEQ ID NO: 696; and VL-CDR3 comprising the amino acid sequence of SEQ ID NO: 817; or
u) VL-CDR1 comprising the amino acid sequence of SEQ ID NO: 825; VL-CDR2 comprising the amino acid sequence of SEQ ID NO: 826; and VL-CDR3 comprising the amino acid sequence of SEQ ID NO: 827; or
v) VL-CDR1 comprising the amino acid sequence of SEQ ID NO: 835; VL-CDR2 comprising the amino acid sequence of SEQ ID NO: 836; and VL-CDR3 comprising the amino acid sequence of SEQ ID NO: 817; or
w) VL-CDR1 comprising the amino acid sequence of SEQ ID NO: 845; VL-CDR2 comprising the amino acid sequence of SEQ ID NO: 846; and VL-CDR3 comprising the amino acid sequence of SEQ ID NO: 847

In some embodiments, the antibody comprises:
a) VH-CDR1 comprising the amino acid sequence having at least 80%, 90%, 95% or 100% sequence identity to SEQ ID NO: 611; VH-CDR2 comprising the amino acid sequence having at least 80%, 90%, 95% or 100% sequence identity to SEQ ID NO: 612; and VH-CDR3 comprising the amino acid sequence having at least 80%, 90%, 95% or 100% sequence identity to SEQ ID NO: 613; VL-CDR1 comprising the amino acid sequence of SEQ ID NO: 615; VL-CDR2 comprising the amino acid sequence of SEQ ID NO: 616; and VL-CDR3 comprising the amino acid sequence of SEQ ID NO: 617; or
b) VH-CDR1 comprising the amino acid sequence having at least 80%, 90%, 95% or 100% sequence identity to SEQ ID NO: 621; VH-CDR2 comprising the amino acid sequence having at least 80%, 90%, 95% or 100% sequence identity to SEQ ID NO: 622; and VH-CDR3 comprising the amino acid sequence having at least 80%, 90%, 95% or 100% sequence identity to SEQ ID NO: 623; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
c) VH-CDR1 comprising the amino acid sequence having at least 80%, 90%, 95% or 100% sequence identity to SEQ ID NO: 631; VH-CDR2 comprising the amino acid sequence having at least 80%, 90%, 95% or 100% sequence identity to SEQ ID NO: 632; and VH-CDR3 comprising the amino acid sequence having at least 80%, 90%, 95% or 100% sequence identity to SEQ ID NO: 633; VL-CDR1 comprising the amino acid sequence of SEQ ID NO: 635; VL-CDR2 comprising the amino acid sequence of SEQ ID NO: 616; and VL-CDR3 comprising the amino acid sequence of SEQ ID NO: 637; or
d) VH-CDR1 comprising the amino acid sequence having at least 80%, 90%, 95% or 100% sequence identity to SEQ ID NO: 641; VH-CDR2 comprising the amino acid sequence having at least 80%, 90%, 95% or 100% sequence identity to SEQ ID NO: 642; and VH-CDR3 comprising the amino acid sequence having at least 80%, 90%, 95% or 100% sequence identity to SEQ ID NO: 643; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
e) VH-CDR1 comprising the amino acid sequence having at least 80%, 90%, 95% or 100% sequence identity to SEQ ID NO: 621; VH-CDR2 comprising the amino acid sequence having at least 80%, 90%, 95% or 100% sequence identity to SEQ ID NO: 642; and VH-CDR3 comprising the amino acid sequence having at least 80%, 90%, 95% or 100% sequence identity to SEQ ID NO: 653; VL-CDR1 comprising the amino acid sequence of SEQ ID NO: 655; VL-CDR2 comprising the amino acid sequence of SEQ ID NO: 626; and VL-CDR3 comprising the amino acid sequence of SEQ ID NO: 627; or
f) VH-CDR1 comprising the amino acid sequence having at least 80%, 90%, 95% or 100% sequence identity to SEQ ID NO: 661; VH-CDR2 comprising the amino acid sequence having at least 80%, 90%, 95% or 100% sequence identity to SEQ ID NO: 662; and VH-CDR3 comprising the amino acid sequence having at least 80%, 90%, 95% or 100% sequence identity to SEQ ID NO: 663; VL-CDR1 comprising the amino acid sequence of SEQ ID NO: 665; VL-CDR2 comprising the amino acid sequence of SEQ ID NO: 666; and VL-CDR3 comprising the amino acid sequence of SEQ ID NO: 667; or
g) VH-CDR1 comprising the amino acid sequence having at least 80%, 90%, 95% or 100% sequence identity to SEQ ID NO: 671; VH-CDR2 comprising the amino acid sequence having at least 80%, 90%, 95% or 100% sequence identity to SEQ ID NO: 672; and VH-CDR3 comprising the amino acid sequence having at least 80%, 90%, 95% or 100% sequence identity to SEQ ID NO: 673; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
h) VH-CDR1 comprising the amino acid sequence having at least 80%, 90%, 95% or 100% sequence identity to SEQ ID NO: 621; VH-CDR2 comprising the amino acid sequence having at least 80%, 90%, 95% or 100% sequence identity to SEQ ID NO: 642; and VH-CDR3 comprising the amino acid sequence having at least 80%, 90%, 95% or 100% sequence identity to SEQ ID NO: 683; VL-CDR1 comprising the amino acid sequence of SEQ ID NO: 625; VL-CDR2 comprising the amino acid sequence of SEQ ID NO: 686; and VL-CDR3 comprising the amino acid sequence of SEQ ID NO: 627; or
i) VH-CDR1 comprising the amino acid sequence having at least 80%, 90%, 95% or 100% sequence identity to SEQ ID NO: 691; VH-CDR2 comprising the amino acid sequence having at least 80%, 90%, 95% or 100% sequence identity to SEQ ID NO: 692; and VH-CDR3 comprising the amino acid sequence having at least 80%, 90%, 95% or 100% sequence identity to SEQ ID NO: 693; VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697; or
j) VH-CDR1 comprising the amino acid sequence having at least 80%, 90%, 95% or 100% sequence identity to SEQ ID NO: 701; VH-CDR2 comprising the amino acid sequence having at least 80%, 90%, 95% or 100% sequence identity to SEQ ID NO: 702; and VH-CDR3 comprising the amino acid sequence having at least 80%, 90%, 95% or 100% sequence identity to SEQ ID NO: 703; VL-CDR1 comprising the amino acid sequence of SEQ ID NO: 705; VL-CDR2 comprising the amino acid sequence of SEQ ID NO: 706; and VL-CDR3 comprising the amino acid sequence of SEQ ID NO: 707; or
k) VH-CDR1 comprising the amino acid sequence having at least 80%, 90%, 95% or 100% sequence identity to SEQ ID NO: 711; VH-CDR2 comprising the amino acid sequence having at least 80%, 90%, 95% or 100% sequence identity to SEQ ID NO: 712; and VH-CDR3 comprising the amino acid sequence having at least 80%, 90%, 95% or 100% sequence identity to SEQ ID NO: 713; VL-CDR1 comprising the amino acid sequence of SEQ ID NO: 715; VL-CDR2 comprising the amino acid sequence of SEQ ID NO: 716; and VL-CDR3 comprising the amino acid sequence of SEQ ID NO: 717; or
l) VH-CDR1 comprising the amino acid sequence having at least 80%, 90%, 95% or 100% sequence identity to SEQ ID NO: 721; VH-CDR2 comprising the amino acid sequence having at least 80%, 90%, 95% or 100% sequence identity to SEQ ID NO: 722; and VH-CDR3 comprising the amino acid sequence having at least 80%, 90%, 95% or 100% sequence identity to SEQ ID NO: 723; VL-CDR1 comprising the amino acid sequence of SEQ ID NO: 725; VL-CDR2 comprising the amino acid sequence of SEQ ID NO: 616; and VL-CDR3 comprising the amino acid sequence of SEQ ID NO: 637; or
m) VH-CDR1 comprising the amino acid sequence having at least 80%, 90%, 95% or 100% sequence identity to SEQ ID NO: 731; VH-CDR2 comprising the amino acid sequence having at least 80%, 90%, 95% or 100% sequence identity to SEQ ID NO: 722; and VH-CDR3 comprising the amino acid sequence having at least 80%, 90%, 95% or 100% sequence identity to SEQ ID NO: 733; VL-CDR1 comprising the amino acid sequence of SEQ ID NO: 735; VL-CDR2 comprising the amino acid sequence of SEQ ID NO: 736; and VL-CDR3 comprising the amino acid sequence of SEQ ID NO: 637; or
n) VH-CDR1 comprising the amino acid sequence having at least 80%, 90%, 95% or 100% sequence identity to SEQ ID NO: 671; VH-CDR2 comprising the amino acid sequence having at least 80%, 90%, 95% or 100% sequence identity to SEQ ID NO: 742; and VH-CDR3 comprising the amino acid sequence having at least 80%, 90%, 95% or 100% sequence identity to SEQ ID NO: 743; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
o) VH-CDR1 comprising the amino acid sequence having at least 80%, 90%, 95% or 100% sequence identity to SEQ ID NO: 751; VH-CDR2 comprising the amino acid sequence having at least 80%, 90%, 95% or 100% sequence identity to SEQ ID NO: 722; and VH-CDR3 comprising the amino acid sequence having at least 80%, 90%, 95% or 100% sequence identity to SEQ ID NO: 723; VL-CDR1 comprising the amino acid sequence of SEQ ID NO: 735; VL-CDR2 comprising the amino acid sequence of SEQ ID NO: 616; and VL-CDR3 comprising the amino acid sequence of SEQ ID NO: 637; or
p) VH-CDR1 comprising the amino acid sequence having at least 80%, 90%, 95% or 100% sequence identity to SEQ ID NO: 761; VH-CDR2 comprising the amino acid sequence having at least 80%, 90%, 95% or 100% sequence identity to SEQ ID NO: 722; and VH-CDR3 comprising the amino acid sequence having at least 80%, 90%, 95% or 100% sequence identity to SEQ ID NO: 733; VL-CDR1 comprising the amino acid sequence of SEQ ID NO: 765; VL-CDR2 comprising the amino acid sequence of SEQ ID NO: 616; and VL-CDR3 comprising the amino acid sequence of SEQ ID NO: 637; or
q) VH-CDR1 comprising the amino acid sequence having at least 80%, 90%, 95% or 100% sequence identity to SEQ ID NO: 771; VH-CDR2 comprising the amino acid sequence having at least 80%, 90%, 95% or 100% sequence identity to SEQ ID NO: 772; and VH-CDR3 comprising the amino acid sequence having at least 80%, 90%, 95% or 100% sequence identity to SEQ ID NO: 773; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
r) VH-CDR1 comprising the amino acid sequence having at least 80%, 90%, 95% or 100% sequence identity to SEQ ID NO: 791; VH-CDR2 comprising the amino acid sequence having at least 80%, 90%, 95% or 100% sequence identity to SEQ ID NO: 792; and VH-CDR3 comprising the amino acid sequence having at least 80%, 90%, 95% or 100% sequence identity to SEQ ID NO: 793; VL-CDR1 comprising the amino acid sequence of SEQ ID NO: 795; VL-CDR2 comprising the amino acid sequence of SEQ ID NO: 616; and VL-CDR3 comprising the amino acid sequence of SEQ ID NO: 797; or
s) VH-CDR1 comprising the amino acid sequence having at least 80%, 90%, 95% or 100% sequence identity to SEQ ID NO: 771; VH-CDR2 comprising the amino acid sequence having at least 80%, 90%, 95% or 100% sequence identity to SEQ ID NO: 802; and VH-CDR3 comprising the amino acid sequence having at least 80%, 90%, 95% or 100% sequence identity to SEQ ID NO: 803; VL-CDR1 comprising the amino acid sequence of SEQ ID NO: 805; VL-CDR2 comprising the amino acid sequence of SEQ ID NO: 676; and VL-CDR3 comprising the amino acid sequence of SEQ ID NO: 677; or
t) VH-CDR1 comprising the amino acid sequence having at least 80%, 90%, 95% or 100% sequence identity to SEQ ID NO: 811; VH-CDR2 comprising the amino acid sequence having at least 80%, 90%, 95% or 100% sequence identity to SEQ ID NO: 812; and VH-CDR3 comprising the amino acid sequence having at least 80%, 90%, 95% or 100% sequence identity to SEQ ID NO: 813; VL-CDR1 comprising the amino acid sequence of SEQ ID NO: 815; VL-CDR2 comprising the amino acid sequence of SEQ ID NO: 696; and VL-CDR3 comprising the amino acid sequence of SEQ ID NO: 817; or
u) VH-CDR1 comprising the amino acid sequence having at least 80%, 90%, 95% or 100% sequence identity to SEQ ID NO: 821; VH-CDR2 comprising the amino acid sequence having at least 80%, 90%, 95% or 100% sequence identity to SEQ ID NO: 822; and VH-CDR3 comprising the amino acid sequence having at least 80%, 90%, 95% or 100% sequence identity to SEQ ID NO: 823; VL-CDR1 comprising the amino acid sequence of SEQ ID NO: 825; VL-CDR2 comprising the amino acid sequence of SEQ ID NO: 826; and VL-CDR3 comprising the amino acid sequence of SEQ ID NO: 827; or
v) VH-CDR1 comprising the amino acid sequence having at least 80%, 90%, 95% or 100% sequence identity to SEQ ID NO: 831; VH-CDR2 comprising the amino acid sequence having at least 80%, 90%, 95% or 100% sequence identity to SEQ ID NO: 832; and VH-CDR3 comprising the amino acid sequence having at least 80%, 90%, 95% or 100% sequence identity to SEQ ID NO: 833; VL-CDR1 comprising the amino acid sequence of SEQ ID NO: 835; VL-CDR2 comprising the amino acid sequence of SEQ ID NO: 836; and VL-CDR3 comprising the amino acid sequence of SEQ ID NO: 817; or
w) VH-CDR1 comprising the amino acid sequence having at least 80%, 90%, 95% or 100% sequence identity to SEQ ID NO: 841; VH-CDR2 comprising the amino acid sequence having at least 80%, 90%, 95% or 100% sequence identity to SEQ ID NO: 842; and VH-CDR3 comprising the amino acid sequence having at least 80%, 90%, 95% or 100% sequence identity to SEQ ID NO: 843; VL-CDR1 comprising the amino acid sequence of SEQ ID NO: 845; VL-CDR2 comprising the amino acid sequence of SEQ ID NO: 846; and VL-CDR3 comprising the amino acid sequence of SEQ ID NO: 847.

In some embodiments, the antibody comprises:
a) VH-CDR1 comprising the amino acid sequence of SEQ ID NO: 611; VH-CDR2 comprising the amino acid sequence of SEQ ID NO: 612; and VH-CDR3 comprising the amino acid sequence of SEQ ID NO: 613; VL-CDR1 comprising the amino acid sequence having at least 80%, 90%, 95% or 100% sequence identity to SEQ ID NO: 615; VL-CDR2 comprising the amino acid sequence having at least 80%, 90%, 95% or 100% sequence identity to SEQ ID NO: 616; and VL-CDR3 comprising the amino acid sequence having at least 80%, 90%, 95% or 100% sequence identity to SEQ ID NO: 617; or
b) VH-CDR1 comprising the amino acid sequence of SEQ ID NO: 621; VH-CDR2 comprising the amino acid sequence of SEQ ID NO: 622; and VH-CDR3 comprising the amino acid sequence of SEQ ID NO: 623; VL-CDR1 comprising the amino acid sequence having at least 80%, 90%, 95% or 100% sequence identity to SEQ ID NO: 625; VL-CDR2 comprising the amino acid sequence having at least 80%, 90%, 95% or 100% sequence identity to SEQ ID NO: 626; and VL-CDR3 comprising the amino acid sequence having at least 80%, 90%, 95% or 100% sequence identity to SEQ ID NO: 627; or
c) VH-CDR1 comprising the amino acid sequence of SEQ ID NO: 631; VH-CDR2 comprising the amino acid sequence of SEQ ID NO: 632; and VH-CDR3 comprising the amino acid sequence of SEQ ID NO: 633; VL-CDR1 comprising the amino acid sequence having at least 80%, 90%, 95% or 100% sequence identity to SEQ ID NO: 635; VL-CDR2 comprising the amino acid sequence having at least 80%, 90%, 95% or 100% sequence identity to SEQ ID NO: 616; and VL-CDR3 comprising the amino acid sequence having at least 80%, 90%, 95% or 100% sequence identity to SEQ ID NO: 637; or
d) VH-CDR1 comprising the amino acid sequence of SEQ ID NO: 641; VH-CDR2 comprising the amino acid sequence of SEQ ID NO: 642; and VH-CDR3 comprising the amino acid sequence of SEQ ID NO: 643; VL-CDR1 comprising the amino acid sequence having at least 80%, 90%, 95% or 100% sequence identity to SEQ ID NO: 625; VL-CDR2 comprising the amino acid sequence having at least 80%, 90%, 95% or 100% sequence identity to SEQ ID NO: 626; and VL-CDR3 comprising the amino acid sequence having at least 80%, 90%, 95% or 100% sequence identity to SEQ ID NO: 627; or
e) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 653; VL-CDR1 comprising the amino acid sequence having at least 80%, 90%, 95% or 100% sequence identity to SEQ ID NO: 655; VL-CDR2 comprising the amino acid sequence having at least 80%, 90%, 95% or 100% sequence identity to SEQ ID NO: 626; and VL-CDR3 comprising the amino acid sequence having at least 80%, 90%, 95% or 100% sequence identity to SEQ ID NO: 627; or
f) VH-CDR1 comprising the amino acid sequence of SEQ ID NO: 661; VH-CDR2 comprising the amino acid sequence of SEQ ID NO: 662; and VH-CDR3 comprising the amino acid sequence of SEQ ID NO: 663; VL-CDR1 comprising the amino acid sequence having at least 80%, 90%, 95% or 100% sequence identity to SEQ ID NO: 665; VL-CDR2 comprising the amino acid sequence having at least 80%, 90%, 95% or 100% sequence identity to SEQ ID NO: 666; and VL-CDR3 comprising the amino acid sequence having at least 80%, 90%, 95% or 100% sequence identity to SEQ ID NO: 667; or
g) VH-CDR1 comprising the amino acid sequence of SEQ ID NO: 671; VH-CDR2 comprising the amino acid sequence of SEQ ID NO: 672; and VH-CDR3 comprising the amino acid sequence of SEQ ID NO: 673; VL-CDR1 comprising the amino acid sequence having at least 80%, 90%, 95% or 100% sequence identity to SEQ ID NO: 675; VL-CDR2 comprising the amino acid sequence having at least 80%, 90%, 95% or 100% sequence identity to SEQ ID NO: 676; and VL-CDR3 comprising the amino acid sequence having at least 80%, 90%, 95% or 100% sequence identity to SEQ ID NO: 677; or
h) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 683; VL-CDR1 comprising the amino acid sequence having at least 80%, 90%, 95% or 100% sequence identity to SEQ ID NO: 625; VL-CDR2 comprising the amino acid sequence having at least 80%, 90%, 95% or 100% sequence identity to SEQ ID NO: 686; and VL-CDR3 comprising the amino acid sequence having at least 80%, 90%, 95% or 100% sequence identity to SEQ ID NO: 627; or
i) VH-CDR1 comprising the amino acid sequence of SEQ ID NO: 691; VH-CDR2 comprising the amino acid sequence of SEQ ID NO: 692; and VH-CDR3 comprising the amino acid sequence of SEQ ID NO: 693; VL-CDR1 comprising the amino acid sequence having at least 80%, 90%, 95% or 100% sequence identity to SEQ ID NO: 695; VL-CDR2 comprising the amino acid sequence having at least 80%, 90%, 95% or 100% sequence identity to SEQ ID NO: 696; and VL-CDR3 comprising the amino acid sequence having at least 80%, 90%, 95% or 100% sequence identity to SEQ ID NO: 697; or
j) VH-CDR1 comprising the amino acid sequence of SEQ ID NO: 701; VH-CDR2 comprising the amino acid sequence of SEQ ID NO: 702; and VH-CDR3 comprising the amino acid sequence of SEQ ID NO: 703; VL-CDR1 comprising the amino acid sequence having at least 80%, 90%, 95% or 100% sequence identity to SEQ ID NO: 705; VL-CDR2 comprising the amino acid sequence having at least 80%, 90%, 95% or 100% sequence identity to SEQ ID NO: 706; and VL-CDR3 comprising the amino acid sequence having at least 80%, 90%, 95% or 100% sequence identity to SEQ ID NO: 707; or
k) VH-CDR1 comprising the amino acid sequence of SEQ ID NO: 711; VH-CDR2 comprising the amino acid sequence of SEQ ID NO: 712; and VH-CDR3 comprising the amino acid sequence of SEQ ID NO: 713; VL-CDR1 comprising the amino acid sequence having at least 80%, 90%, 95% or 100% sequence identity to SEQ ID NO: 715; VL-CDR2 comprising the amino acid sequence having at least 80%, 90%, 95% or 100% sequence identity to SEQ ID NO: 716; and VL-CDR3 comprising the amino acid sequence having at least 80%, 90%, 95% or 100% sequence identity to SEQ ID NO: 717; or
l) VH-CDR1 comprising the amino acid sequence of SEQ ID NO: 721; VH-CDR2 comprising the amino acid sequence of SEQ ID NO: 722; and VH-CDR3 comprising the amino acid sequence of SEQ ID NO: 723; VL-CDR1 comprising the amino acid sequence having at least 80%, 90%, 95% or 100% sequence identity to SEQ ID NO: 725; VL-CDR2 comprising the amino acid sequence having at least 80%, 90%, 95% or 100% sequence identity to SEQ ID NO: 616; and VL-CDR3 comprising the amino acid sequence having at least 80%, 90%, 95% or 100% sequence identity to SEQ ID NO: 637; or
m) VH-CDR1 comprising the amino acid sequence of SEQ ID NO: 731; VH-CDR2 comprising the amino acid sequence of SEQ ID NO: 722; and VH-CDR3 comprising the amino acid sequence of SEQ ID NO: 733; VL-CDR1 comprising the amino acid sequence having at least 80%, 90%, 95% or 100% sequence identity to SEQ ID NO: 735; VL-CDR2 comprising the amino acid sequence having at least 80%, 90%, 95% or 100% sequence identity to SEQ ID NO: 736; and VL-CDR3 comprising the amino acid sequence having at least 80%, 90%, 95% or 100% sequence identity to SEQ ID NO: 637; or
n) VH-CDR1 comprising the amino acid sequence of SEQ ID NO: 671; VH-CDR2 comprising the amino acid sequence of SEQ ID NO: 742; and VH-CDR3 comprising the amino acid sequence of SEQ ID NO: 743; VL-CDR1 comprising the amino acid sequence having at least 80%, 90%, 95% or 100% sequence identity to SEQ ID NO: 675; VL-CDR2 comprising the amino acid sequence having at least 80%, 90%, 95% or 100% sequence identity to SEQ ID NO: 676; and VL-CDR3 comprising the amino acid sequence having at least 80%, 90%, 95% or 100% sequence identity to SEQ ID NO: 677; or
o) VH-CDR1 comprising the amino acid sequence of SEQ ID NO: 751; VH-CDR2 comprising the amino acid sequence of SEQ ID NO: 722; and VH-CDR3 comprising the amino acid sequence of SEQ ID NO: 723; VL-CDR1 comprising the amino acid sequence having at least 80%, 90%, 95% or 100% sequence identity to SEQ ID NO: 735; VL-CDR2 comprising the amino acid sequence having at least 80%, 90%, 95% or 100% sequence identity to SEQ ID NO: 616; and VL-CDR3 comprising the amino acid sequence having at least 80%, 90%, 95% or 100% sequence identity to SEQ ID NO: 637; or
p) VH-CDR1 comprising the amino acid sequence of SEQ ID NO: 761; VH-CDR2 comprising the amino acid sequence of SEQ ID NO: 722; and VH-CDR3 comprising the amino acid sequence of SEQ ID NO: 733; VL-CDR1 comprising the amino acid sequence having at least 80%, 90%, 95% or 100% sequence identity to SEQ ID NO: 765; VL-CDR2 comprising the amino acid sequence having at least 80%, 90%, 95% or 100% sequence identity to SEQ ID NO: 616; and VL-CDR3 comprising the amino acid sequence having at least 80%, 90%, 95% or 100% sequence identity to SEQ ID NO: 637; or
q) VH-CDR1 comprising the amino acid sequence of SEQ ID NO: 771; VH-CDR2 comprising the amino acid sequence of SEQ ID NO: 772; and VH-CDR3 comprising the amino acid sequence of SEQ ID NO: 773; VL-CDR1 comprising the amino acid sequence having at least 80%, 90%, 95% or 100% sequence identity to SEQ ID NO: 675; VL-CDR2 comprising the amino acid sequence having at least 80%, 90%, 95% or 100% sequence identity to SEQ ID NO: 676; and VL-CDR3 comprising the amino acid sequence having at least 80%, 90%, 95% or 100% sequence identity to SEQ ID NO: 677; or
r) VH-CDR1 comprising the amino acid sequence of SEQ ID NO: 791; VH-CDR2 comprising the amino acid sequence of SEQ ID NO: 792; and VH-CDR3 comprising the amino acid sequence of SEQ ID NO: 793; VL-CDR1 comprising the amino acid sequence having at least 80%, 90%, 95% or 100% sequence identity to SEQ ID NO: 795; VL-CDR2 comprising the amino acid sequence having at least 80%, 90%, 95% or 100% sequence identity to SEQ ID NO: 616; and VL-CDR3 comprising the amino acid sequence having at least 80%, 90%, 95% or 100% sequence identity to SEQ ID NO: 797; or
s) VH-CDR1 comprising the amino acid sequence of SEQ ID NO: 771; VH-CDR2 comprising the amino acid sequence of SEQ ID NO: 802; and VH-CDR3 comprising the amino acid sequence of SEQ ID NO: 803; VL-CDR1 comprising the amino acid sequence having at least 80%, 90%, 95% or 100% sequence identity to SEQ ID NO: 805; VL-CDR2 comprising the amino acid sequence having at least 80%, 90%, 95% or 100% sequence identity to SEQ ID NO: 676; and VL-CDR3 comprising the amino acid sequence having at least 80%, 90%, 95% or 100% sequence identity to SEQ ID NO: 677; or
t) VH-CDR1 comprising the amino acid sequence of SEQ ID NO: 811; VH-CDR2 comprising the amino acid sequence of SEQ ID NO: 812; and VH-CDR3 comprising the amino acid sequence of SEQ ID NO: 813; VL-CDR1 comprising the amino acid sequence having at least 80%, 90%, 95% or 100% sequence identity to SEQ ID NO: 815; VL-CDR2 comprising the amino acid sequence having at least 80%, 90%, 95% or 100% sequence identity to SEQ ID NO: 696; and VL-CDR3 comprising the amino acid sequence having at least 80%, 90%, 95% or 100% sequence identity to SEQ ID NO: 817; or
u) VH-CDR1 comprising the amino acid sequence of SEQ ID NO: 821; VH-CDR2 comprising the amino acid sequence of SEQ ID NO: 822; and VH-CDR3 comprising the amino acid sequence of SEQ ID NO: 823; VL-CDR1 comprising the amino acid sequence having at least 80%, 90%, 95% or 100% sequence identity to SEQ ID NO: 825; VL-CDR2 comprising the amino acid sequence having at least 80%, 90%, 95% or 100% sequence identity to SEQ ID NO: 826; and VL-CDR3 comprising the amino acid sequence having at least 80%, 90%, 95% or 100% sequence identity to SEQ ID NO: 827; or
v) VH-CDR1 comprising the amino acid sequence of SEQ ID NO: 831; VH-CDR2 comprising the amino acid sequence of SEQ ID NO: 832; and VH-CDR3 comprising the amino acid sequence of SEQ ID NO: 833; VL-CDR1 comprising the amino acid sequence having at least 80%, 90%, 95% or 100% sequence identity to SEQ ID NO: 835; VL-CDR2 comprising the amino acid sequence having at least 80%, 90%, 95% or 100% sequence identity to SEQ ID NO: 836; and VL-CDR3 comprising the amino acid sequence having at least 80%, 90%, 95% or 100% sequence identity to SEQ ID NO: 817; or
w) VH-CDR1 comprising the amino acid sequence of SEQ ID NO: 841; VH-CDR2 comprising the amino acid sequence of SEQ ID NO: 842; and VH-CDR3 comprising the amino acid sequence of SEQ ID NO: 843; VL-CDR1 comprising the amino acid sequence having at least 80%, 90%, 95% or 100% sequence identity to SEQ ID NO: 845; VL-CDR2 comprising the amino acid sequence having at least 80%, 90%, 95% or 100% sequence identity to SEQ ID NO: 846; and VL-CDR3 comprising the amino acid sequence having at least 80%, 90%, 95% or 100% sequence identity to SEQ ID NO: 847.

In some embodiments, an alpha-synuclein antibody comprises at least one, two, three, four, five, or six CDRs selected from (a) VH-CDR1 comprising the amino acid sequence of SEQ ID NO: 611; (b) VH-CDR2 comprising the amino acid sequence of SEQ ID NO: 612; and (c) VH-CDR3 comprising the amino acid sequence of SEQ ID NO: 613; (d) VL-CDR1 comprising the amino acid sequence of SEQ ID NO: 615; (e) VL-CDR2 comprising the amino acid sequence of SEQ ID NO: 616; and (f) VL-CDR3 comprising the amino acid sequence of SEQ ID NO: 617.

In some embodiments, an alpha-synuclein antibody comprises at least one, two, three, four, five, or six CDRs selected from (a) VH-CDR1 comprising the amino acid sequence of SEQ ID NO: 621; (b) VH-CDR2 comprising the amino acid sequence of SEQ ID NO: 622; and (c) VH-CDR3 comprising the amino acid sequence of SEQ ID NO: 623; (d) VL-CDR1 comprising the amino acid sequence of SEQ ID NO: 625; (e) VL-CDR2 comprising the amino acid sequence of SEQ ID NO: 626; and (f) VL-CDR3 comprising the amino acid sequence of SEQ ID NO: 627.

In some embodiments, an alpha-synuclein antibody comprises at least one, two, three, four, five, or six CDRs selected from (a) VH-CDR1 comprising the amino acid sequence of SEQ ID NO: 631; (b) VH-CDR2 comprising the amino acid sequence of SEQ ID NO: 632; and (c) VH-CDR3 comprising the amino acid sequence of SEQ ID NO: 633; (d) VL-CDR1 comprising the amino acid sequence of SEQ ID NO: 635; (e) VL-CDR2 comprising the amino acid sequence of SEQ ID NO: 616; and (f) VL-CDR3 comprising the amino acid sequence of SEQ ID NO: 637.

In some embodiments, an alpha-synuclein antibody comprises at least one, two, three, four, five, or six CDRs selected from (a) VH-CDR1 comprising the amino acid sequence of SEQ ID NO: 641; (b) VH-CDR2 comprising the amino acid sequence of SEQ ID NO: 642; and (c) VH-CDR3 comprising the amino acid sequence of SEQ ID NO: 643; (d) VL-CDR1 comprising the amino acid sequence of SEQ ID NO: 625; (e) VL-CDR2 comprising the amino acid sequence of SEQ ID NO: 626; and (f) VL-CDR3 comprising the amino acid sequence of SEQ ID NO: 627.

In some embodiments, an alpha-synuclein antibody comprises at least one, two, three, four, five, or six CDRs selected from (a) VH-CDR1 comprising the amino acid sequence of SEQ ID NO: 621; (b) VH-CDR2 comprising the amino acid sequence of SEQ ID NO: 642; and (c) VH-CDR3 comprising the amino acid sequence of SEQ ID NO: 653; (d) VL-CDR1 comprising the amino acid sequence of SEQ ID NO: 655; (e) VL-CDR2 comprising the amino acid sequence of SEQ ID NO: 626; and (f) VL-CDR3 comprising the amino acid sequence of SEQ ID NO: 627.

In some embodiments, an alpha-synuclein antibody comprises at least one, two, three, four, five, or six CDRs selected from (a) VH-CDR1 comprising the amino acid sequence of SEQ ID NO: 661; (b) VH-CDR2 comprising the amino acid sequence of SEQ ID NO: 662; and (c) VH-CDR3 comprising the amino acid sequence of SEQ ID NO: 663; (d) VL-CDR1 comprising the amino acid sequence of SEQ ID NO: 665; (e) VL-CDR2 comprising the amino acid sequence of SEQ ID NO: 666; and (f) VL-CDR3 comprising the amino acid sequence of SEQ ID NO: 667.

In some embodiments, an alpha-synuclein antibody comprises at least one, two, three, four, five, or six CDRs selected from (a) VH-CDR1 comprising the amino acid sequence of SEQ ID NO: 671; (b) VH-CDR2 comprising the amino acid sequence of SEQ ID NO: 672; and (c) VH-CDR3 comprising the amino acid sequence of SEQ ID NO: 673; (d) VL-CDR1 comprising the amino acid sequence of SEQ ID NO: 675; (e) VL-CDR2 comprising the amino acid sequence of SEQ ID NO: 676; and (f) VL-CDR3 comprising the amino acid sequence of SEQ ID NO: 677.

In some embodiments, an alpha-synuclein antibody comprises at least one, two, three, four, five, or six CDRs selected from (a) VH-CDR1 comprising the amino acid sequence of SEQ ID NO: 621; (b) VH-CDR2 comprising the amino acid sequence of SEQ ID NO: 642; and (c) VH-CDR3 comprising the amino acid sequence of SEQ ID NO: 683; (d) VL-CDR1 comprising the amino acid sequence of SEQ ID NO: 625; (e) VL-CDR2 comprising the amino acid sequence of SEQ ID NO: 686; and (f) VL-CDR3 comprising the amino acid sequence of SEQ ID NO: 627.

In some embodiments, an alpha-synuclein antibody comprises at least one, two, three, four, five, or six CDRs selected from (a) VH-CDR1 comprising the amino acid sequence of SEQ ID NO: 691; (b) VH-CDR2 comprising the amino acid sequence of SEQ ID NO: 692; and (c) VH-CDR3 comprising the amino acid sequence of SEQ ID NO: 693; (d) VL-CDR1 comprising the amino acid sequence of SEQ ID NO: 695; (e) VL-CDR2 comprising the amino acid sequence of SEQ ID NO: 696; and (f) VL-CDR3 comprising the amino acid sequence of SEQ ID NO: 697.

In some embodiments, an alpha-synuclein antibody comprises at least one, two, three, four, five, or six CDRs selected from (a) VH-CDR1 comprising the amino acid sequence of SEQ ID NO: 701; (b) VH-CDR2 comprising the amino acid sequence of SEQ ID NO: 702; and (c) VH-CDR3 comprising the amino acid sequence of SEQ ID NO: 703; (d) VL-CDR1 comprising the amino acid sequence of SEQ ID NO: 705; (e) VL-CDR2 comprising the amino acid sequence of SEQ ID NO: 706; and (f) VL-CDR3 comprising the amino acid sequence of SEQ ID NO: 707.

In some embodiments, an alpha-synuclein antibody comprises at least one, two, three, four, five, or six CDRs selected from (a) VH-CDR1 comprising the amino acid sequence of SEQ ID NO: 711; (b) VH-CDR2 comprising the amino acid sequence of SEQ ID NO: 712; and (c) VH-CDR3 comprising the amino acid sequence of SEQ ID NO: 713; (d) VL-CDR1 comprising the amino acid sequence of SEQ ID NO: 715; (e) VL-CDR2 comprising the amino acid sequence of SEQ ID NO: 716; and (f) VL-CDR3 comprising the amino acid sequence of SEQ ID NO: 717.

In some embodiments, an alpha-synuclein antibody comprises at least one, two, three, four, five, or six CDRs selected from (a) VH-CDR1 comprising the amino acid sequence of SEQ ID NO: 721; (b) VH-CDR2 comprising the amino acid sequence of SEQ ID NO: 722; and (c) VH-CDR3 comprising the amino acid sequence of SEQ ID NO: 723; (d) VL-CDR1 comprising the amino acid sequence of SEQ ID NO: 725; (e) VL-CDR2 comprising the amino acid sequence of SEQ ID NO: 616; and (f) VL-CDR3 comprising the amino acid sequence of SEQ ID NO: 637.

In some embodiments, an alpha-synuclein antibody comprises at least one, two, three, four, five, or six CDRs selected from (a) VH-CDR1 comprising the amino acid sequence of SEQ ID NO: 731; (b) VH-CDR2 comprising the amino acid sequence of SEQ ID NO: 722; and (c) VH-CDR3 comprising the amino acid sequence of SEQ ID NO: 733; (d) VL-CDR1 comprising the amino acid sequence of SEQ ID NO: 735; (e) VL-CDR2 comprising the amino acid sequence of SEQ ID NO: 736; and (f) VL-CDR3 comprising the amino acid sequence of SEQ ID NO: 637.

In some embodiments, an alpha-synuclein antibody comprises at least one, two, three, four, five, or six CDRs selected from (a) VH-CDR1 comprising the amino acid sequence of SEQ ID NO: 671; (b) VH-CDR2 comprising the amino acid sequence of SEQ ID NO: 742; and (c) VH-CDR3 comprising the amino acid sequence of SEQ ID NO: 743; (d) VL-CDR1 comprising the amino acid sequence of SEQ ID NO: 675; (e) VL-CDR2 comprising the amino acid sequence of SEQ ID NO: 676; and (f) VL-CDR3 comprising the amino acid sequence of SEQ ID NO: 677.

In some embodiments, an alpha-synuclein antibody comprises at least one, two, three, four, five, or six CDRs selected from (a) VH-CDR1 comprising the amino acid sequence of SEQ ID NO: 751; (b) VH-CDR2 comprising the amino acid sequence of SEQ ID NO: 722; and (c) VH-CDR3 comprising the amino acid sequence of SEQ ID NO: 723; (d) VL-CDR1 comprising the amino acid sequence of SEQ ID NO: 735; (e) VL-CDR2 comprising the amino acid sequence of SEQ ID NO: 616; and (f) VL-CDR3 comprising the amino acid sequence of SEQ ID NO: 637.

In some embodiments, an alpha-synuclein antibody comprises at least one, two, three, four, five, or six CDRs selected from (a) VH-CDR1 comprising the amino acid sequence of SEQ ID NO: 761; (b) VH-CDR2 comprising the amino acid sequence of SEQ ID NO: 722; and (c) VH-CDR3 comprising the amino acid sequence of SEQ ID NO: 733; (d) VL-CDR1 comprising the amino acid sequence of SEQ ID NO: 765; (e) VL-CDR2 comprising the amino acid sequence of SEQ ID NO: 616; and (f) VL-CDR3 comprising the amino acid sequence of SEQ ID NO: 637.

In some embodiments, an alpha-synuclein antibody comprises at least one, two, three, four, five, or six CDRs selected from (a) VH-CDR1 comprising the amino acid sequence of SEQ ID NO: 771; (b) VH-CDR2 comprising the amino acid sequence of SEQ ID NO: 772; and (c) VH-CDR3 comprising the amino acid sequence of SEQ ID NO: 773; (d) VL-CDR1 comprising the amino acid sequence of SEQ ID NO: 675; (e) VL-CDR2 comprising the amino acid sequence of SEQ ID NO: 676; and (f) VL-CDR3 comprising the amino acid sequence of SEQ ID NO: 677.

In some embodiments, an alpha-synuclein antibody comprises at least one, two, three, four, five, or six CDRs selected from (a) VH-CDR1 comprising the amino acid sequence of SEQ ID NO: 791; (b) VH-CDR2 comprising the amino acid sequence of SEQ ID NO: 792; and (c) VH-CDR3 comprising the amino acid sequence of SEQ ID NO: 793; (d) VL-CDR1 comprising the amino acid sequence of SEQ ID NO: 795; (e) VL-CDR2 comprising the amino acid sequence of SEQ ID NO: 616; and (f) VL-CDR3 comprising the amino acid sequence of SEQ ID NO: 797.

In some embodiments, an alpha-synuclein antibody comprises at least one, two, three, four, five, or six CDRs selected from (a) VH-CDR1 comprising the amino acid sequence of SEQ ID NO: 771; (b) VH-CDR2 comprising the amino acid sequence of SEQ ID NO: 802; and (c) VH-CDR3 comprising the amino acid sequence of SEQ ID NO: 803; (d) VL-CDR1 comprising the amino acid sequence of SEQ ID NO: 805; (e) VL-CDR2 comprising the amino acid sequence of SEQ ID NO: 676; and (f) VL-CDR3 comprising the amino acid sequence of SEQ ID NO: 677.

In some embodiments, an alpha-synuclein antibody comprises at least one, two, three, four, five, or six CDRs selected from (a) VH-CDR1 comprising the amino acid sequence of SEQ ID NO: 811; (b) VH-CDR2 comprising the amino acid sequence of SEQ ID NO: 812; and (c) VH-CDR3 comprising the amino acid sequence of SEQ ID NO: 813; (d) VL-CDR1 comprising the amino acid sequence of SEQ ID NO: 815; (e) VL-CDR2 comprising the amino acid sequence of SEQ ID NO: 696; and (f) VL-CDR3 comprising the amino acid sequence of SEQ ID NO: 817.

In some embodiments, an alpha-synuclein antibody comprises at least one, two, three, four, five, or six CDRs selected from (a) VH-CDR1 comprising the amino acid sequence of SEQ ID NO: 821; (b) VH-CDR2 comprising the amino acid sequence of SEQ ID NO: 822; and (c) VH-CDR3 comprising the amino acid sequence of SEQ ID NO: 823; (d) VL-CDR1 comprising the amino acid sequence of SEQ ID NO: 825; (e) VL-CDR2 comprising the amino acid sequence of SEQ ID NO: 826; and (f) VL-CDR3 comprising the amino acid sequence of SEQ ID NO: 827.

In some embodiments, an alpha-synuclein antibody comprises at least one, two, three, four, five, or six CDRs selected from (a) VH-CDR1 comprising the amino acid sequence of SEQ ID NO: 831; (b) VH-CDR2 comprising the amino acid sequence of SEQ ID NO: 832; and (c) VH-CDR3 comprising the amino acid sequence of SEQ ID NO: 833; (d) VL-CDR1 comprising the amino acid sequence of SEQ ID NO: 835; (e) VL-CDR2 comprising the amino acid sequence of SEQ ID NO: 836; and (f) VL-CDR3 comprising the amino acid sequence of SEQ ID NO: 817.

In some embodiments, an alpha-synuclein antibody comprises at least one, two, three, four, five, or six CDRs selected from (a) VH-CDR1 comprising the amino acid sequence of SEQ ID NO: 841; (b) VH-CDR2 comprising the amino acid sequence of SEQ ID NO: 842; and (c) VH-CDR3 comprising the amino acid sequence of SEQ ID NO: 843; (d) VL-CDR1 comprising the amino acid sequence of SEQ ID NO: 845; (e) VL-CDR2 comprising the amino acid sequence of SEQ ID NO: 846; and (f) VL-CDR3 comprising the amino acid sequence of SEQ ID NO: 847.

In some embodiments, an alpha-synuclein antibody comprises at least one, two, or three CDRs selected from (a) VH-CDR1 comprising the amino acid sequence selected from SEQ ID NO: 611, 621, 631, 641, 661, 671, 691, 701, 711, 721, 731, 751, 761, 771, 791, 811, 821, 831 or 841, (b) VH-CDR2 comprising the amino acid sequence selected from SEQ ID NO: 612, 622, 632, 642, 662, 672, 692, 702, 712, 722, 742, 772, 792, 802, 812, 822, 832 or 842, (c) VH-CDR3 comprising the amino acid sequence selected from SEQ ID NO: 613, 623, 633, 643, 653, 663, 673, 683, 693, 703, 713, 723, 733, 743, 773, 793, 803, 813, 823, 833 or 843.

In some embodiments, an alpha-synuclein antibody comprises at least one, two, or three CDRs selected from (a) VL-CDR1 comprising the amino acid sequence selected from SEQ ID NO: 615, 625, 635, 655, 665, 675, 695, 705, 715, 725, 735, 765, 795, 805, 815, 825, 835 or 845, (b) VL-CDR2 comprising the amino acid sequence selected from SEQ ID NO: 616, 626, 666, 676, 686, 696, 706, 716, 736, 826, 836 or 846, (c) VL-CDR3 comprising the amino acid sequence selected from SEQ ID NO: 617, 627, 637, 667, 677, 697, 707, 717, 797, 817, 827 or 847.

In another embodiment, the alpha-synuclein antibody comprises a heavy chain variable domain (VH) selected from SEQ ID NO: 610, 620, 630, 640, 650, 660, 670, 680, 690, 700, 710, 720, 730, 740, 750, 760, 770, 790, 800, 810, 820, 830 or 840 including post-translational modifications of that sequence.

In a particular embodiment, the heavy chain variable domain (VH) comprises at least one, two, or three CDRs selected from (a) VH-CDR1 comprising the amino acid sequence selected from SEQ ID NO:611, 621, 631, 641, 661, 671, 691, 701, 711, 721, 731, 751, 761, 771, 791, 811, 821, 831 or 841, (b) VH-CDR2 comprising the amino acid sequence selected from SEQ ID NO: 612, 622, 632, 642, 662, 672, 692, 702, 712, 722, 742, 772, 792, 802, 812, 822, 832 or 842, (c) VH-CDR3 comprising the amino acid sequence selected from SEQ ID NO: 613, 623, 633, 643, 653, 663, 673, 683, 693, 703, 713, 723, 733, 743, 773, 793, 803, 813, 823, 833 or 843.

In another embodiment, the alpha-synuclein antibody comprises a light chain variable domain (VL) selected from SEQ ID NO: 614, 624, 634, 644, 654, 664, 674, 684, 694, 704, 714, 724, 734, 744, 754, 764, 774, 784, 794, 804, 814, 824, 834 or 844 including post-translational modifications of that sequence.

In a particular embodiment, the light chain variable domain (VL) comprises at least one, two, or three CDRs selected from (a) VL-CDR1 comprising the amino acid sequence selected from SEQ ID NO: 615, 625, 635, 655, 665, 675, 695, 705, 715, 725, 735, 765, 795, 805, 815, 825, 835 or 845, (b) VL-CDR2 comprising the amino acid sequence selected from SEQ ID NO: 616, 626, 666, 676, 686, 696, 706, 716, 736, 826, 836 or 846, (c) VL-CDR3 comprising the amino acid sequence selected from SEQ ID NO: 617, 627, 637, 667, 677, 697, 707, 717, 797, 817, 827 or 847.

In some embodiments, the invention relates to an antibody selected from ACI-7079-3101 E3-Ab1, ACI-7079-3103D9-Ab1, ACI-7079-3103G12-Ab2, ACI-7079-3104F12-Ab2, ACI-7079-3106C5-Ab1, ACI-7079-3106F2-Ab1, ACI-7079-3112H1-Ab1, ACI-7079-3107E6-Ab1, ACI-7079-3108C10-Ab2, ACI-8030-6106F5-Ab1, ACI-8031-6207G10-Ab1, ACI-8032-6301A10-Ab2, ACI-8032-6301C8-Ab2, ACI-8032-6301 G2-Ab2, ACI-8032-6304F3-Ab1, ACI-8032-6307F1-Ab2, ACI-8032-6313G2-Ab1, ACI-8032-6314A3-Ab3, ACI-8033-6401 F2-Ab1, ACI-8033-6402E2-Ab2, ACI-8033-6402E10-Ab1, ACI-8033-6403A4-Ab1, ACI-8033-6403E11-Ab2 or ACI-7067-4813-R4A-G7-rec1. In certain preferred embodiments, the antibody may be selected from ACI-7079-3108C10-Ab2, ACI-8032-6301A10-Ab2, ACI-8032-6301G2-Ab2, ACI-7067-4813-R4A-G7-rec1 or ACI-8033-6401 F2-Ab1. They all delay seeded alpha-synuclein aggregation to particularly high levels (although all of the antibodies described herein provide highly significant delays in aggregation). In some embodiments, the antibody is selected from ACI-7067-4813-R4A-G7-rec1, ACI-7079-3101 E3-Ab1, ACI-8032-6301 A10-Ab2, ACI-7079-3106F2-Ab1 and ACI-8033-6403A4-Ab1 (as further described and defined herein). These antibodies demonstrate a binding preference to fibrillar alpha-synuclein and display significantly slower dissociation rates (kd) from fibrillar alpha-synuclein compared to monomeric alpha-synuclein. In some embodiments, the antibody is selected from ACI-7079-3108C10-Ab2 and ACI-8033-6401F2-Ab1 (as further described and defined herein). These antibodies selectively bind only to fibrillar alpha-synuclein.

In some embodiments, the antibody or an antibody-binding fragment thereof comprises:
a) VH-CDR1 comprising the amino acid sequence of SEQ ID NO: 691; VH-CDR2 comprising the amino acid sequence of SEQ ID NO: 692; and VH-CDR3 comprising the amino acid sequence of SEQ ID NO: 693; VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697; or
b) VH-CDR1 comprising the amino acid sequence of SEQ ID NO: 721; VH-CDR2 comprising the amino acid sequence of SEQ ID NO: 722; and VH-CDR3 comprising the amino acid sequence of SEQ ID NO: 723; VL-CDR1 comprising the amino acid sequence of SEQ ID NO: 725; VL-CDR2 comprising the amino acid sequence of SEQ ID NO: 616; and VL-CDR3 comprising the amino acid sequence of SEQ ID NO: 637; or
c) VH-CDR1 comprising the amino acid sequence of SEQ ID NO: 671; VH-CDR2 comprising the amino acid sequence of SEQ ID NO: 742; and VH-CDR3 comprising the amino acid sequence of SEQ ID NO: 743; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
d) VH-CDR1 comprising the amino acid sequence of SEQ ID NO: 791; VH-CDR2 comprising the amino acid sequence of SEQ ID NO: 792; and VH-CDR3 comprising the amino acid sequence of SEQ ID NO: 793; VL-CDR1 comprising the amino acid sequence of SEQ ID NO: 795; VL-CDR2 comprising the amino acid sequence of SEQ ID NO: 616; and VL-CDR3 comprising the amino acid sequence of SEQ ID NO: 797.
e) VH-CDR1 comprising the amino acid sequence of SEQ ID NO: 841; VH-CDR2 comprising the amino acid sequence of SEQ ID NO: 842; and VH-CDR3 comprising the amino acid sequence of SEQ ID NO: 843; VL-CDR1 comprising the amino acid sequence of SEQ ID NO: 845; VL-CDR2 comprising the amino acid sequence of SEQ ID NO: 846; and VL-CDR3 comprising the amino acid sequence of SEQ ID NO: 847.

In some embodiments, the antibody or antibody-binding fragment thereof comprises:
a. a Heavy Chain Variable Region (VH) comprising the sequence of SEQ ID NO: 690 and a Light Chain Variable Region (VL) comprising the sequence of SEQ ID NO: 694; or
b. a Heavy Chain Variable Region (VH) comprising the sequence of SEQ ID NO: 720 and a Light Chain Variable Region (VL) comprising the sequence of SEQ ID NO: 724; or
c. a Heavy Chain Variable Region (VH) comprising the sequence of SEQ ID NO: 740 and a Light Chain Variable Region (VL) comprising the sequence of SEQ ID NO: 744; or
d. a Heavy Chain Variable Region (VH) comprising the sequence of SEQ ID NO: 790 and a Light Chain Variable Region (VL) comprising the sequence of SEQ ID NO: 794; or
e. a Heavy Chain Variable Region (VH) comprising the sequence of SEQ ID NO: 840 and a Light Chain Variable Region (VL) comprising the sequence of SEQ ID NO: 844

In some embodiments, an antibody binds to the same or similar epitope (totally or partially overlapping epitope) as an antibody selected from ACI-7079-3101 E3-Ab1, ACI-7079-3103D9-Ab1, ACI-7079-3103G12-Ab2, ACI-7079-3104F12-Ab2, ACI-7079-3106C5-Ab1, ACI-7079-3106F2-Ab1, ACI-7079-3112H1-Ab1, ACI-7079-3107E6-Ab1, ACI-7079-3108C10-Ab2, ACI-8030-6106F5-Ab1, ACI-8031-6207G10-Ab1, ACI-8032-6301A10-Ab2, ACI-8032-6301C8-Ab2, ACI-8032-6301G2-Ab2, ACI-8032-6304F3-Ab1, ACI-8032-6307F1-Ab2, ACI-8032-6313G2-Ab1, ACI-8032-6314A3-Ab3, ACI-8033-6401F2-Ab1, ACI-8033-6402E2-Ab2, ACI-8033-6402E10-Ab1, ACI-8033-6403A4-Ab1, ACI-8033-6403E11-Ab2 or ACI-7067-4813-R4A-G7-rec1. In certain preferred embodiments, the antibody binds to the same or similar epitope (totally or partially overlapping epitope) as an antibody selected from ACI-7079-3108C10-Ab2, ACI-8032-6301 A10-Ab2, ACI-8032-6301G2-Ab2 or ACI-8033-6401 F2-Ab1. They all delay seeded alpha-synuclein aggregation.

In some embodiments, an isolated antibody is provided, wherein the isolated antibody binds to the same or similar epitope comprising, consisting essentially of or consisting of the sequence SEQ ID NO: 121. In some embodiments, an isolated antibody is provided, wherein the isolated antibody binds to the same or similar epitope comprising, consisting essentially of or consisting of the sequence SEQ ID NO: 123. In some embodiments, an isolated antibody is provided, wherein the isolated antibody binds to the same or similar epitope comprising, consisting essentially of or consisting of the sequence SEQ ID NO: 131. In some embodiments, an isolated antibody is provided, wherein the isolated antibody binds to the same or similar epitope comprising, consisting essentially of or consisting of the sequence SEQ ID NO: 132. In some embodiments, an isolated antibody is provided, wherein the isolated antibody binds to the same or similar epitope comprising, consisting essentially of or consisting of the sequence SEQ ID NO: 137. In some embodiments, an isolated antibody is provided, wherein the isolated antibody binds to the same or similar epitope comprising, consisting essentially of or consisting of the sequence SEQ ID NO: 138. In some embodiments, an isolated antibody is provided, wherein the isolated antibody binds to the same or similar epitope comprising, consisting essentially of or consisting of the sequence SEQ ID NO: 139. In some embodiments, an isolated antibody is provided, wherein the isolated antibody binds to the same or similar epitope comprising, consisting essentially of or consisting of the sequence SEQ ID NO: 146. In some embodiments, an isolated antibody is provided, wherein the isolated antibody binds to the same or similar epitope comprising, consisting essentially of or consisting of the sequence SEQ ID NO: 147. In some embodiments, an isolated antibody is provided wherein the isolated antibody binds to the same or similar non-linear epitope within amino acids residues of human alpha-synuclein of SEQ ID NO: 1. The term “the same or similar epitope” references any antibody provided herein.

Antibodies binding the same epitope as any of the antibodies provided herein are also part of the invention. In some embodiments, an isolated antibody is provided, wherein the isolated antibody binds to the same epitope comprising, consisting essentially of or consisting of the sequence SEQ ID NO: 121. In some embodiments, an isolated antibody is provided, wherein the isolated antibody binds to the same epitope comprising, consisting essentially of or consisting of the sequence SEQ ID NO: 123. In some embodiments, an isolated antibody is provided, wherein the isolated antibody binds to the same epitope comprising, consisting essentially of or consisting of the sequence SEQ ID NO: 131. In some embodiments, an isolated antibody is provided, wherein the isolated antibody binds to the same epitope comprising, consisting essentially of or consisting of the sequence SEQ ID NO: 132. In some embodiments, an isolated antibody is provided, wherein the isolated antibody binds to the same epitope comprising, consisting essentially of or consisting of the sequence SEQ ID NO: 137. In some embodiments, an isolated antibody is provided, wherein the isolated antibody binds to the same epitope comprising, consisting essentially of or consisting of the sequence SEQ ID NO: 138. In some embodiments, an isolated antibody is provided, wherein the isolated antibody binds to the same epitope comprising, consisting essentially of or consisting of the sequence SEQ ID NO: 139. In some embodiments, an isolated antibody is provided, wherein the isolated antibody binds to the same epitope comprising, consisting essentially of or consisting of the sequence SEQ ID NO: 146. In some embodiments, an isolated antibody is provided, wherein the isolated antibody binds to the same epitope comprising, consisting essentially of or consisting of the sequence SEQ ID NO: 147. In some embodiments, an isolated antibody is provided wherein the isolated antibody binds to the same non-linear epitope within amino acids residues of human alpha-synuclein of SEQ ID NO: 1. The term “the same epitope” references any antibody provided herein.

In accordance with the above, in certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

In certain embodiments, one or more amino acid modifications may be introduced into the Fc region of an antibody provided herein, thereby generating an Fc region variant. The Fc region variant may comprise a murine Fc region sequence (e.g.: IgG1, IgG2a or IgG2b) comprising an amino acid modification (e.g. substitution) at one or more amino acid positions. The Fc region variant may comprise a human Fc region sequence (e.g., a human IgG1, IgG2, IgG3 or IgG4 Fc region) comprising an amino acid modification (e.g. a substitution) at one or more amino acid positions (e.g. an IgG4 isotype including the S228P mutation).

In certain embodiments, the Fc region is mutated to increase its affinity to FcRn at pH 6.0 and consequently extend the antibody half-life. Antibodies with enhanced affinity to FcRn include those with substitution of one or more of Fc region residues 252, 253, 254, 256, 428, 434, including the so called YTE mutation with substitution M252Y/S254T/T256E (Dall' Acqua et al, J Immunol. 169:5171-5180 (2002)) or LS mutation M428L/N434S (Zalevsky et al, Nat Biotechnol. 28(2): 157-159 (2010)).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. The accessible sites may be on the antibody surface. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541 and in Bhakta S., Raab H., Junutula J. R. (2013) Engineering THIOMABs for Site-Specific Conjugation of Thiol-Reactive Linkers. In: Ducry L. (eds) Antibody-Drug Conjugates. Methods in Molecular Biology (Methods and Protocols), vol 1045. Humana Press, Totowa, NJ. https://doi.org/10.1007/978-1-62703-541-5_11.

In certain embodiments, an antibody provided herein may be further modified to contain additional nonproteinaceous moieties. Suitable nonproteinaceous moieties are known in the art and readily available. Moieties suitable for derivatization of the antibody include but are not limited to water soluble polymers. Nonlimiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-, vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, the invention contemplates an antibody variant that possesses some but not all effector functions, which make it a desirable candidate for applications in which the half life of the antibody in vivo is important yet certain effector functions (such as complement activation and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes and microglia express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). This discussion applies mutatis mutandis to mixtures of the invention.

Antibodies with reduced effector function include those with substitution of one or more of Fc region residues 234, 235, 238, 265, 269, 270, 297, 327 and 329 (U.S. Pat. No. 6,737,056). Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581) or the so-called “DANG” Fc mutant with substitution of residues 265 to alanine and 297 to Glycine. Alternatively, antibodies with reduced effector function include those with substitution of one or more of Fc region residues 234, 235 and 329, so-called “PG-LALA” Fc mutant with substitution of residues 234 and 235 to alanine and 329 to glycine (Lo, M. et al., Journal of Biochemistry, 292, 3900-3908). Other known mutations at position 234, 235 and 321, the so called TM mutant containing mutations L234F/L235E/P331S in the CH2 domain, can be used (Oganesyan et al. Acta Cryst. D 64, 700-704. (2008)). Antibodies from the human IgG4 isotype include mutations S228P/L235E to stabilize the hinge and to reduce FcR binding (Schlothauer et al, PEDS, 29 (10):457-466).

Other Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821.

Antibodies may be produced using recombinant methods and compositions, e.g., as described in U.S. Pat. No. 4,816,567. An isolated nucleic acid encoding an alpha-synuclein antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20). In one embodiment, a method of making an anti-apha-synuclein antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an alpha-synuclein antibody, nucleic acid encoding an antibody, e.g., as described above, is isolated and inserted into one or more vectors for further cloning and/or expression in a host cell.

Suitable host cells for cloning or expression of antibody-encoding vectors include prokaryotic or eukaryotic cells described herein.

The present invention also relates to the production of specific antibodies against native polypeptides and recombinant polypeptides of alpha-synuclein. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen polypeptide of alpha-synuclein can be subcloned into an appropriate vector, wherein the recombinant polypeptide is to be expressed in an organism being suitable for its expression, for example in bacteria. Thus, the expressed recombinant protein can be injected into a mice and the resulting specific antibody can be, for example, obtained from the mice serum being provided by intra-cardiac blood puncture. Many other strategies are known in the art, such as the use of DNA vaccine strategies which is well-known in the art and encompass liposome-mediated delivery, by gene gun or jet injection and intramuscular or intradermal injection. Thus, antibodies directed against a polypeptide or a protein or an epitope of alpha-synuclein, in particular the epitope of the antibodies provided herein, can be obtained by directly immunizing the animal by directly injecting intramuscularly the vector expressing the desired polypeptide or a protein or an epitope of alpha-synuclein. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

Accordingly, antibodies of the present invention can be produced by methods known to those skilled in the art. Specifically, DNA encoding the antibody of interest is inserted into an expression vector. Insertion into an expression vector is carried out such that the expression will take place under the control of expression regulatory regions such as enhancers and promoters. Next, host cells are transformed using this expression vector to express the antibodies. Appropriate combinations of the host and expression vector can be used in this step.

Examples of the vectors include M13 series vectors, pUC series vectors, pBR322, pBluescript, and pCR-Script. In addition to these vectors, for example, pGEM-T, pDIRECT, or pT7 can also be used for the purpose of cDNA subcloning and excision.

Particularly, expression vectors are useful for the purpose of producing the antibody. For example, when the host is E. coli such as JM109, DH5a, HB101, or XL1-Blue, the expression vectors indispensably have a promoter that permits efficient expression in E. coli, for example, lacZ promoter (Ward et al., Nature (1989) 341, 544-546; and FASEB J (1992) 6, 2422-2427), araB promoter (Better et al., Science (1988) 240, 1041-1043), or T7 promoter. Examples of such vectors include the vectors mentioned above as well as pGEX-5X-1 (manufactured by Pharmacia), “QIAexpress system” (manufactured by QIAGEN), pEGFP, and pET (in this case, the host is preferably BL21 expressing T7 RNA polymerase).

The vectors may contain a signal sequence for polypeptide secretion. In the case of production in the periplasm of E. coli, pelB signal sequence (Lei, S. P. et al., J. Bacteriol. (1987) 169, 4397) can be used as the signal sequence for polypeptide secretion. The vectors can be transferred to the host cells using, for example, calcium chloride methods or electroporation methods.

In addition to the E. coli expression vectors, examples of the vectors for producing the antibody of the present invention include mammal-derived expression vectors (e.g., pcDNA3 (manufactured by Invitrogen Corp.), pEGF-BOS (Nucleic Acids. Res. 1990, 18(17), p5322), pEF, and pCDM8), insect cell-derived expression vectors (e.g., “Bac-to-BAC baculovirus expression system” (manufactured by GIBCO BRL), and pBacPAK8), plant-derived expression vectors (e.g., pMH1 and pMH2), animal virus-derived expression vectors (e.g., pHSV, pMV, and pAdexLcw), retrovirus-derived expression vectors (e.g., pZlPneo), yeast-derived expression vectors (e.g., “Pichia Expression Kit” (manufactured by Invitrogen Corp.), pNV11, and SP-Q01), and Bacillus subtilis-derived expression vectors (e.g., pPL608 and pKTH50).

For the purpose of expression in animal cells such as CHO cells, COS cells, or NIH3T3 cells, the vectors indispensably have a promoter necessary for intracellular expression, for example, SV40 promoter (Mulligan et al., Nature (1979) 277, 108), MMTV-LTR promoter, EF1a promoter (Mizushima et al., Nucleic Acids Res (1990) 18, 5322), CAG promoter (Gene (1991) 108, 193), or CMV promoter and, more preferably, have a gene for screening for transformed cells (e.g., a drug resistance gene that can work as a marker by a drug (neomycin, G418, etc.)). Examples of the vectors having such properties include pMAM, pDR2, pBK-RSV, pBK-CMV, pOPRSV, and pOP13.

An exemplary method intended to stably express the gene and increase the number of intracellular gene copies involves transfecting CHO cells deficient in nucleic acid synthesis pathway with vectors having a DHFR gene serving as a complement thereto (e.g., pCHOI) and using methotrexate (MTX) in the gene amplification. An exemplary method intended to transiently express the gene involves using COS cells having a gene which expresses an SV40 T antigen on their chromosomes to transform the cells with vectors having a replication origin of SV40 (pcD, etc.). Also, a replication origin derived from polyomavirus, adenovirus, bovine papillomavirus (BPV), or the like may be used. The expression vectors for increasing the number of gene copies in a host cell system can additionally contain a selection marker such as an aminoglycoside transferase (APH) gene, a thymidine kinase (TK) gene, an E. coli xanthine guanine phosphoribosyltransferase (Ecogpt) gene, or a dihydrofolate reductase (dhfr) gene.

The antibodies of the present invention obtained by the methods described above can be isolated from inside host cells or from outside of the cells (the medium, or such), and purified to practically pure and homogeneous antibodies. The antibodies can be separated and purified by methods routinely used for separating and purifying antibodies, and the type of method is not limited. For example, the antibodies can be separated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectrofocusing, dialysis, recrystallization, and such.

The chromatographies include, for example, affinity chromatography, ion exchange chromatography, hydrophobic chromatography, gel filtration, reverse phase chromatography, and adsorption chromatography (Strategies for Protein Purification and Characterization: A Laboratory Course Manual. Ed Daniel R. Marshak et al., Cold Spring Harbor Laboratory Press, 1996). The chromatographic methods described above can be conducted using liquid-chromatography, for example, HPLC and FPLC. Columns used for affinity chromatography include protein A columns and protein G columns. Columns using protein A include, for example, Hyper D, POROS, and Sepharose FF (GE Amersham Biosciences). The present invention includes antibodies that are highly purified using these purification methods.

The obtained antibodies can be purified to homogeneity. Separation and purification of the antibodies can be performed using separation and purification methods generally used for protein separation and purification. For example, the antibodies can be separated and purified by appropriately selecting and combining column chromatography such as affinity chromatography, filtration, ultrafiltration, salting-out, dialysis, SDS-polyacrylamide gel electrophoresis, isoelectric focusing, and such, without limitation (Antibodies: A Laboratory Manual. Ed Harlow and David Lane, Cold Spring Harbor Laboratory, 1988). Columns used for affinity chromatography include, for example, protein A columns and protein G columns.

Several known approaches exist for delivering molecules across the blood brain barrier (BBB) and may be employed according to the invention. Non-limiting examples include alteration of the administration route, disruption of the BBB and alteration of its permeability, nanoparticle delivery, Trojan horse approaches, receptor-mediated transport, and cell and gene therapy.

Alteration of the administration route can be achieved by direct injection into the brain (see, e.g., Papanastassiou et al., Gene Therapy 9: 398-406(2002)), implanting a delivery device in the brain (see, e.g., Gillet al., Nature Med. 9: 589-595 (2003); and Gliadel Wafers™, Guildford Pharmaceutical), and intranasal administration to bypass the BBB (Mittal et al, Drug Deliv. 21(2):75-86. (2014))

Methods of barrier disruption include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416).

Methods of altering the BBB permeability include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), and inhibiting ABC drug transporters (see, e.g., U.S. Patent Application Publication No. 2003/0073713).

Trojan horse delivery methods of delivering the humanized antibody or humanized antibody fragment thereof across the blood brain barrier include, but are not limited to, cationizing the antibodies (see, e.g., U.S. Pat. No. 5,004,697), and the use of cell-penetration peptides such as Tat peptides to gain entry into the CNS. (see, e.g. Dietz et al., J. Neurochem. 104:757-765 (2008)).

Nanoparticle delivery methods of delivering the antibody or antigen-binding fragment thereof across the blood brain barrier include, but are not limited to, encapsulating the antibody or antigen-binding fragment thereof in delivery vehicles such as liposomes, or extracellular vesicles or exosomes, that are coupled to antibody or antigen-binding fragments or, alternatively, peptides that bind to receptors on the vascular endothelium of the blood-brain barrier (see, e.g., U.S. Patent Application Publication No. 20020025313), and coating the antibody or antigen-binding fragment thereof in low-density lipoprotein particles (see, e.g., U.S. Patent Application Publication No. 20040204354) or apolipoprotein E (see, e.g., U.S. Patent Application Publication No. 20040131692).

Alpha-synuclein antibodies of the invention can be further modified to enhance blood brain barrier penetration.

The alpha-synuclein antibody or antigen-binding fragment thereof of the invention can be fused to a polypeptide binding to a blood-brain barrier receptor. BBB receptors include, but are not limited to, transferrin receptor, insulin receptor or low-density lipoprotein receptor. The polypeptide can be any suitable polypeptide. It may, for example, comprise a peptide, a receptor ligand, a single domain antibody (VHH), a scFv or a Fab fragment.

The alpha-synuclein antibodies of the invention can also be delivered as a corresponding nucleic acid encoding the alpha-synuclein antibody. Such nucleic acid molecule may be a part of a viral vector for targeted delivery to the blood brain barrier or any other cell type in the CNS. A viral vector may be a recombinant adeno-associated viral vectors (rAAV) selected from any AAV serotype known in the art, including, without limitation, from AAV1 to AAV12 to enable the alpha-synuclein antibody or alpha-synuclein antibody fragment or alpha-synuclein antibody derivatives to be expressed intracellularly or into the brain parenchyma.

Cell therapy methods of delivering the alpha-synuclein antibody of the invention or the alpha-synuclein antibody fragment or alpha-synuclein antibody derivatives across the blood brain barrier include, but are not limited to, the use of the homing capacity of Endothelial Progenitor Cells (EPCs) transfected ex vivo with suitable vectors and the secretion and delivery of antibodies or antibody fragments to the brain by these cells (see, e.g., Heller et al., J Cell Mol Med. 00:1-7 (2020)), or the use of polymeric cell implant devices loaded with genetically engineered cells, to secrete antibodies or antibody fragments (see, e.g. Marroquin Belaunzaran et al. PLoS ONE 6(4): e18268 (2011)).

Alpha-synuclein antibodies provided herein may be identified, screened for, or characterized for their physical/chemical properties and/or biological activities by various assays known in the art.

In one aspect, an antibody of the invention is tested for its antigen binding activity, e.g., by known methods such as ELISA, BIACore®, FACS, immunofluorescence or immunohistochemistry.

In another aspect, competition assays may be used to identify an antibody that competes with any of the antibodies described herein for binding to aggregated or pathlological alpha-synuclein. In certain embodiments, such a competing antibody binds to the same or similar epitope (e.g., a linear or a conformational epitope with total or partial overlap) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, NJ).

The invention also provides immunoconjugates comprising an alpha-synuclein antibody provided herein conjugated to one or more therapeutic agents, such as chemotherapeutic agents or drugs, growth inhibitory agents, toxins (e.g., protein toxins, enzymatically active toxins of bacterial, fungal, plant, or animal origin, or fragments thereof), radioactive isotopes (i.e., a radioconjugate), blood brain barrier penetration moieties or detectable labels. Various techniques exist for improving drug delivery across the blood-brain barrier (BBB) as discussed herein, which discussion applies mutatis mutandis. Non-invasive techniques include the so-called “Trojan horse approach” in which conjugated molecules deliver the binding molecules of the invention by binding to BBB receptors and mediating transport. Suitable molecules may comprise endogenous ligands or antibodies, in particular monoclonal antibodies, that bind specific epitopes on the BBB receptor.

In some embodiments, a conjugated binding molecule, in particular antibody or antigen-binding fragment thereof, is provided, comprising a binding molecule, in particular an antibody or antigen-binding fragment thereof, described herein and a conjugated molecule. Conjugates of the invention may be referred to as immunoconjugates. Any suitable conjugated molecule may be employed according to the invention. Suitable examples include, but are not limited to enzymes (e.g. alkaline phosphatase or horseradish peroxidase), avidin, streptavidin, biotin, Protein A/G, magnetic beads, fluorophores, radioactive isotopes (i.e., radioconjugates), nucleic acid molecules, detectable labels, therapeutic agents, toxins and blood brain barrier penetration moieties. Conjugation methods are well known in the art and several technologies are commercially available for conjugating antibodies to a label or other molecule. Conjugation is typically through amino acid residues contained within the binding molecules of the invention (such as lysine, histidine or cysteine). They may rely upon methods such as the NHS (Succinimidyl) ester method, isothiocyanate method, carbodiimide method and periodate method. Conjugation may be achieved through creation of fusion proteins for example. This is appropriate where the binding molecule is conjugated with another protein molecule. Thus, suitable genetic constructs may be formed that permit the expression of a fusion of the binding molecule of the invention with the label or other molecule. Conjugation may be via a suitable linker moiety to ensure suitable spatial separation of the antibody and conjugated molecule, such as detectable label. However, a linker may not be required in all instances.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease or disorder or abnormality, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease, disorder or abnormality. In particular embodiments, the binding molecules of the invention are for preventing, slowing down, halting, retaining and/or improving the motor capabilities or motor deficits, cognitive capabilities or cognitive deficits, or behavioral impairments of a subject suffering from a synucleopathy. In further particular embodiments, the binding molecules of the invention are for improving motor capabilities, in particular facial expression, speech, ocular motor dysfunction, tremor at rest, action tremor, increased tone, rapid alternating movement of hands, finger tapping, leg agility, Heel-Shin test, arising from chair, posture, body sway and/or gait; improving cognitive deficits, in particular as measured by MoCA (Montreal Cognitive Assessment) or Addenbrookes Cognitive Examination; and/or improving behavioral impairments, in particular using NPI scale, wherein the synucleopathy is multiple system atrophy (MSA).

In a further embodiment, when the synucleopathy is Parkinson's disease, Lewy Body dementia (LBD; dementia with Lewy bodies (DLB) (“pure” Lewy body dementia), Parkinson's disease dementia (PDD)), or Diffuse Lewy Body Disease, the binding molecules of the invention are for: (i) improving motor capabilities, in particular activities of daily living (speech, salivation, swallowing, handwriting, cutting food and handling utensils, dressing, hygiene, turning in bed and adjusting bed clothes, falling, freezing when walking, walking, tremor, sensory complaints related to Parkinsonism), motor examination (speech, facial expression, tremor at rest, action or postural tremor of hands, rigidity, finger taps, hand movements, rapid alternating movements of hands, leg agility, arising from chair, posture, gait, postural stability, body bradykinesia and hypokinesia, dyskinesias, clinical fluctuations), symptomatic orthostatis, repeated falls and syncope, and/or transient unexplained loss of consciousness; and/or (ii) improving cognitive deficits; and/or (iii) improving behavioral impairments, in particular behavior and mood (intellectual Impairment, thought disorder, depression, motivation/initiative), delusions, hallucinations, agitation/aggression, depression/dysphoria, anxiety, elation/euphoria, apathy/indifference, irritability/lability, motor disturbance, nighttime behavior, and/or appetite/eating, deficits of attention, executive functions, visuospatial ability, visual hallucination; and/or (iv) improving rapid eye movement (REM) sleep disorders, in particular insomnia, hypersomnolence.

In one embodiment, a pharmaceutical composition is provided comprising the antibody, antigen-binding fragment thereof or derivative thereof, as an active ingredient and a pharmaceutically acceptable carrier and/or excipient. For example, the antibody, antigen-binding fragment thereof or derivative thereof may be combined, as appropriate, with pharmaceutically acceptable carriers or media such as sterilized water or saline solution, vegetable oils, emulsifiers, suspensions, surfactants, stabilizers, flavoring agents, excipients, vehicles, preservatives, and binders, for example, and formulated into a pharmaceutical preparation. Examples of carriers include light anhydrous silicie acid, lactose, crystalline cellulose, mannitol, starch, cannellose calcium, carmellose sodium, hydroxypropylcellulose, hydroxypropylmethylcellulose, polyvinylacetal diethylaminoacetate, polyvinyl pyrrolidone, gelatin, medium chain fatty acid triglycerides, polyoxyethylene hydrogenated castor oil 60, sucrose, carboxymethyl cellulose, corn starch, and inorganic salts.

The amount of the active ingredient in these preparations can be set as appropriate within the designated range of doses.

In another embodiment, the present disclosure provides a product comprising at least (i) a container (e.g., an injection); (ii) a pharmaceutical composition comprising the antibody, antigen-binding fragment thereof or derivative thereof as an active ingredient within the container; and (iii) a document instructing that the antibody, antigen-binding fragment thereof or derivative thereof be administered according to a desired dosage regimen. Additionally, a label, a syringe, an injection needle, a pharmacologically acceptable medium, an alcohol cotton cloth, plaster, and the like may be additionally packaged, as appropriate, with this product. The container may be a bottle, a glass bottle, or a syringe, for example, and may be made of any of various materials such as glass and plastics. The container contains the pharmaceutical composition, and has an outlet sealed with a rubber stopper, for example. The container is provided with, for example, a label indicating that the pharmaceutical composition is for use in preventing or treating a selected pathological condition. In some cases, this label may describe the embodiment where the antibody, antigen-binding fragment thereof or derivative thereof is used in combination with an additional medicament.

An antibody, immunoconjugate, pharmaceutical composition of the invention (and any additional therapeutic agent) can be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies, immunoconjugates, pharmaceutical compositions of the invention may be formulated, dosed, and administered in a fashion consistent with good medical practice. Factors for consideration in this context include the particular disease or disorder or abnormality being treated, the particular subject being treated, the clinical condition of the individual patient, the cause of the disease or disorder or abnormality, the site of delivery of the agent, the method of administration, the scheduling of administration, and other factors known to medical practitioners. The antibody or immunoconjugate need not be, but is optionally formulated with one or more agents currently used to prevent or treat the disease or disorder or abnormality in question. The effective amount of such other agents depends on the amount of antibody or immunoconjugate present in the formulation, the type of disease, or disorder or abnormality or treatment, and other factors discussed above. These are generally used in the same dosages and with administration routes as described herein, or in any dosage and by any route that is empirically/clinically determined to be appropriate.

It is understood that any of the above formulations or therapeutic methods may be carried out using both an immunoconjugate of the invention and an alpha-synuclein antibody, or using mixtures of the invention.

In some embodiments, an isolated nucleic acid is provided, wherein the isolated nucleic acid encodes an antibody described herein.

In some embodiments, an isolated nucleic acid is provided, wherein the isolated nucleic acid comprises SEQ ID NO: 618 encoding an alpha-synuclein antibody. In some embodiments, an isolated nucleic acid is provided, wherein the isolated nucleic acid comprises SEQ ID NO: 628 encoding an alpha-synuclein antibody. In some embodiments, an isolated nucleic acid is provided, wherein the isolated nucleic acid comprises SEQ ID NO: 638 encoding an alpha-synuclein antibody. In some embodiments, an isolated nucleic acid is provided, wherein the isolated nucleic acid comprises SEQ ID NO: 648 encoding an alpha-synuclein antibody. In some embodiments, an isolated nucleic acid is provided, wherein the isolated nucleic acid comprises SEQ ID NO: 658 encoding an alpha-synuclein antibody. In some embodiments, an isolated nucleic acid is provided, wherein the isolated nucleic acid comprises SEQ ID NO: 668 encoding an alpha-synuclein antibody. In some embodiments, an isolated nucleic acid is provided, wherein the isolated nucleic acid comprises SEQ ID NO: 678 encoding an alpha-synuclein antibody. In some embodiments, an isolated nucleic acid is provided, wherein the isolated nucleic acid comprises SEQ ID NO: 688 encoding an alpha-synuclein antibody. In some embodiments, an isolated nucleic acid is provided, wherein the isolated nucleic acid comprises SEQ ID NO: 698 encoding an alpha-synuclein antibody. In some embodiments, an isolated nucleic acid is provided, wherein the isolated nucleic acid comprises SEQ ID NO: 708 encoding an alpha-synuclein antibody. In some embodiments, an isolated nucleic acid is provided, wherein the isolated nucleic acid comprises SEQ ID NO: 718 encoding an alpha-synuclein antibody. In some embodiments, an isolated nucleic acid is provided, wherein the isolated nucleic acid comprises SEQ ID NO: 728 encoding an alpha-synuclein antibody. In some embodiments, an isolated nucleic acid is provided, wherein the isolated nucleic acid comprises SEQ ID NO: 738 encoding an alpha-synuclein antibody. In some embodiments, an isolated nucleic acid is provided, wherein the isolated nucleic acid comprises SEQ ID NO: 748 encoding an alpha-synuclein antibody. In some embodiments, an isolated nucleic acid is provided, wherein the isolated nucleic acid comprises SEQ ID NO: 758 encoding an alpha-synuclein antibody. In some embodiments, an isolated nucleic acid is provided, wherein the isolated nucleic acid comprises SEQ ID NO: 768 encoding an alpha-synuclein antibody. In some embodiments, an isolated nucleic acid is provided, wherein the isolated nucleic acid comprises SEQ ID NO: 778 encoding an alpha-synuclein antibody. In some embodiments, an isolated nucleic acid is provided, wherein the isolated nucleic acid comprises SEQ ID NO: 798 encoding an alpha-synuclein antibody. In some embodiments, an isolated nucleic acid is provided, wherein the isolated nucleic acid comprises SEQ ID NO: 808 encoding an alpha-synuclein antibody. In some embodiments, an isolated nucleic acid is provided, wherein the isolated nucleic acid comprises SEQ ID NO: 818 encoding an alpha-synuclein antibody. In some embodiments, an isolated nucleic acid is provided, wherein the isolated nucleic acid comprises SEQ ID NO: 828 encoding an alpha-synuclein antibody. In some embodiments, an isolated nucleic acid is provided, wherein the isolated nucleic acid comprises SEQ ID NO: 838 encoding an alpha-synuclein antibody. In some embodiments, an isolated nucleic acid is provided, wherein the isolated nucleic acid comprises SEQ ID NO: 848 encoding an alpha-synuclein antibody.

In some embodiments, an isolated nucleic acid is provided, wherein the isolated nucleic acid comprises SEQ ID NO: 619 encoding an alpha-synuclein antibody. In some embodiments, an isolated nucleic acid is provided, wherein the isolated nucleic acid comprises SEQ ID NO: 629 encoding an alpha-synuclein antibody. In some embodiments, an isolated nucleic acid is provided, wherein the isolated nucleic acid comprises SEQ ID NO: 639 encoding an alpha-synuclein antibody. In some embodiments, an isolated nucleic acid is provided, wherein the isolated nucleic acid comprises SEQ ID NO: 649 encoding an alpha-synuclein antibody. In some embodiments, an isolated nucleic acid is provided, wherein the isolated nucleic acid comprises SEQ ID NO: 659 encoding an alpha-synuclein antibody. In some embodiments, an isolated nucleic acid is provided, wherein the isolated nucleic acid comprises SEQ ID NO: 669 encoding an alpha-synuclein antibody. In some embodiments, an isolated nucleic acid is provided, wherein the isolated nucleic acid comprises SEQ ID NO: 679 encoding an alpha-synuclein antibody. In some embodiments, an isolated nucleic acid is provided, wherein the isolated nucleic acid comprises SEQ ID NO: 689 encoding an alpha-synuclein antibody. In some embodiments, an isolated nucleic acid is provided, wherein the isolated nucleic acid comprises SEQ ID NO: 699 encoding an alpha-synuclein antibody. In some embodiments, an isolated nucleic acid is provided, wherein the isolated nucleic acid comprises SEQ ID NO: 709 encoding an alpha-synuclein antibody. In some embodiments, an isolated nucleic acid is provided, wherein the isolated nucleic acid comprises SEQ ID NO: 719 encoding an alpha-synuclein antibody. In some embodiments, an isolated nucleic acid is provided, wherein the isolated nucleic acid comprises SEQ ID NO: 729 encoding an alpha-synuclein antibody. In some embodiments, an isolated nucleic acid is provided, wherein the isolated nucleic acid comprises SEQ ID NO: 739 encoding an alpha-synuclein antibody. In some embodiments, an isolated nucleic acid is provided, wherein the isolated nucleic acid comprises SEQ ID NO: 749 encoding an alpha-synuclein antibody. In some embodiments, an isolated nucleic acid is provided, wherein the isolated nucleic acid comprises SEQ ID NO: 759 encoding an alpha-synuclein antibody. In some embodiments, an isolated nucleic acid is provided, wherein the isolated nucleic acid comprises SEQ ID NO: 769 encoding an alpha-synuclein antibody. In some embodiments, an isolated nucleic acid is provided, wherein the isolated nucleic acid comprises SEQ ID NO: 779 encoding an alpha-synuclein antibody. In some embodiments, an isolated nucleic acid is provided, wherein the isolated nucleic acid comprises SEQ ID NO: 789 encoding an alpha-synuclein antibody. In some embodiments, an isolated nucleic acid is provided, wherein the isolated nucleic acid comprises SEQ ID NO: 799 encoding an alpha-synuclein antibody. In some embodiments, an isolated nucleic acid is provided, wherein the isolated nucleic acid comprises SEQ ID NO: 809 encoding an alpha-synuclein antibody. In some embodiments, an isolated nucleic acid is provided, wherein the isolated nucleic acid comprises SEQ ID NO: 819 encoding an alpha-synuclein antibody. In some embodiments, an isolated nucleic acid is provided, wherein the isolated nucleic acid comprises SEQ ID NO: 829 encoding an alpha-synuclein antibody. In some embodiments, an isolated nucleic acid is provided, wherein the isolated nucleic acid comprises SEQ ID NO: 839 encoding an alpha-synuclein antibody. In some embodiments, an isolated nucleic acid is provided, wherein the isolated nucleic acid comprises SEQ ID NO: 849 encoding an alpha-synuclein antibody.

In certain embodiments, a binding molecule or an antibody provided herein has a dissociation constant (KD) of between 0.04 nM and 3500 nm in particular with respect to binding alpha-synuclein, in particular aggregated alpha-synuclein and/or pathological alpha-synuclein. In certain embodiments, a binding molecule or an antibody provided herein has a dissociation constant (KD) of ≤4 μM, ≤100 nM, ≤10 nM, ≤1 nM, ≤0.1 nM, or ≤0.05 nM, preferably ≤0.04 nM (e.g. 4×10⁻⁶ M or less, e.g. from 4×10⁻⁶ M to 10⁻¹² M, e.g., from 10⁻⁹ M to 10⁻¹² M), in particular with respect to binding alpha-synuclein, in particular aggregated alpha-synuclein and/or pathological alpha-synuclein.

In certain embodiments, a binding molecule or an antibody provided herein has a dissociation constant (KD) of between 0.04 nM and 3500 nm in particular with respect to binding pathological and/or aggregated alpha-synuclein, including but not limited to protofibrils, fibrils, oligomers, Lewy Body, Lewy neurites and/or glial cytoplasmic inclusions. In certain embodiments, a binding molecule or an antibody provided herein has a dissociation constant (KD) of ≤4 μM, ≤100 nM, ≤10 nM, ≤1 nM, ≤0.1 nM, or ≤0.05 nM, preferably ≤0.04 nM (e.g. 4×10⁻⁶ M or less, e.g. from 4×10⁻⁶ M to 10⁻¹² M, e.g., from 10⁻⁹ M to 10⁻¹² M), in particular with respect to binding pathological and/or aggregated alpha-synuclein, including but not limited to protofibrils, fibrils, oligomers, Lewy Body, Lewy neurites and/or glial cytoplasmic inclusions.

In one embodiment, KD is measured using surface plasmon resonance assays using a BIACORE®-2000 or a BIACORE®-3000 (BIAcore, Inc., Piscataway, NJ) at 25° C. with immobilized antigen CM5 chips at −10 response units (RU).

In some embodiments, an antibody, particularly an isolated antibody of the invention as described herein that binds human alpha-synuclein is provided, wherein the antibody binds aggregated alpha-synuclein and/or pathological alpha-synuclein with a KD of less than 100 nM, less than 10 nM, less than 1 nM, less than 200 μM, less than 100 μM, or less than 10 μM. Preferably, the antibody of the invention binds aggregated alpha-synuclein and/or pathological alpha-synuclein with a KD of less than 100 nM, less than 10 nM, less than 1 nM, less than 200 μM, less than 100 μM, or less than 10 μM.

The binding molecules, especially antibodies, of the invention may selectively bind aggregated alpha-synuclein and/or pathological alpha-synuclein in preference to non-aggregated alpha-synuclein and/or non-pathological alpha-synuclein (such as monomeric alpha-synuclein). This selectivity may be measured in terms of dissociation (or “off”) rates (kd). Thus, the binding molecules, especially antibodies, of the invention may display slower, preferably significantly slower, dissociation rates (kd) from aggregated alpha-synuclein and/or pathological alpha-synuclein (such as fibrillar alpha-synuclein) compared to non-aggregated alpha-synuclein and/or non-pathological alpha-synuclein (such as monomeric alpha-synuclein). For example, the binding molecules, especially antibodies, of the invention may display at least 10-fold, preferably at least 100-fold, and more preferably at least 1000-fold slower dissociation rates (kd) from aggregated alpha-synuclein and/or pathological alpha-synuclein (such as fibrillar alpha-synuclein) compared to non-aggregated alpha-synuclein and/or non-pathological alpha-synuclein (such as monomeric alpha-synuclein). This selectivity may be measured in terms of relative dissociation constant (KD). Thus, the binding molecules, especially antibodies, of the invention may display lower, preferably significantly lower, dissociation constants (KD) with respect to aggregated alpha-synuclein and/or pathological alpha-synuclein (such as fibrillar alpha-synuclein) compared to non-aggregated alpha-synuclein and/or non-pathological alpha-synuclein (such as monomeric alpha-synuclein). For example, the binding molecules, especially antibodies, of the invention may display at least 10-fold, more preferably at least 20-fold, and more preferably at least 100-fold lower dissociation constants (KD) with respect to aggregated alpha-synuclein and/or pathological alpha-synuclein (such as fibrillar alpha-synuclein) compared to non-aggregated alpha-synuclein and/or non-pathological alpha-synuclein (such as monomeric alpha-synuclein). KD and kd may be measured using surface plasmon resonance assays using a BIACORE®-2000 or a BIACORE®-3000 (BIAcore, Inc., Piscataway, NJ) at 25° C. with immobilized antigen CM5 chips at −10 response units (RU). Specific methodology is described in the Examples section herein (see “Affinity measurements on alpha-synuclein monomers and alpha-synuclein fibrils by SPR′”), which may be applied according to the invention as a reference method.

The binding molecules, especially antibodies, of the invention may inhibit and/or delay seeded and/or spontaneous alpha-synuclein aggregation with an IC₅₀ of ≤1 μM, ≤100 nM, ≤10 nM, ≤1 nM or ≤0.1 nM. The IC₅₀ may be obtained by measuring the percentage of de novo alpha-synuclein aggregates formed, relative to conditions in the absence of antibody, as a function of antibody concentration. Dose-response curves may be plotted and IC₅₀ values obtained using Equation 6. See the Examples describing the in vitro cellular model, which methodology applies mutatis mutandis. Alternatively, dose-response curves may be plotted and IC₅₀ values obtained using Equation 7. See the Examples describing the mouse primary cortical neuron experiments, which methodology applies mutatis mutandis.

The binding molecules, especially antibodies, of the invention may inhibit and/or delay seeded and/or spontaneous alpha-synuclein aggregation as quantified by a percent change in the aggregation half-time (T_{1/2}). Suitable methodology for measuring the aggregation half-time is provided herein, see the Examples “Inhibition or delay of seeded alpha-synuclein aggregation”, which description can be applied mutatis mutandis. Antibodies of the invention significantly increase, such as at least a 10% increase in, T_{1/2} values, as normalized to aggregation in the absence of antibody.

In some embodiments, an antibody, antigen-binding fragment thereof or derivative thereof, is provided which binds to human alpha-synuclein within an epitope comprised in SEQ ID NO: 1. In some embodiments, an antibody is provided which binds to a non-linear epitope within amino acids residues of human alpha-synuclein of SEQ ID NO: 1. More preferably, antigen-binding molecule of the invention bind to an epitope within amino acids residues 1-15 (SEQ ID NO: 121), 15-45 (SEQ ID NO: 138), 19-33 (SEQ ID NO: 123), 28-50 (SEQ ID NO: 139), 31-60 (SEQ ID NO: 146), 81-120 (SEQ ID NO: 137), 91-105 (SEQ ID NO: 131), 96-140 (SEQ ID NO: 147) or 100-114 (SEQ ID NO: 132) of human alpha-synuclein of SEQ ID NO: 1. More preferably, antigen-binding molecules of the invention bind to an epitope within amino acids residues 28-50 (SEQ ID NO: 139), 31-60 (SEQ ID NO: 146) or 81-120 (SEQ ID NO: 137) of human alpha-synuclein of SEQ ID NO: 1. Even more preferably, antigen-binding molecules of the invention may bind to an epitope comprising amino acids 33-37 or 33-39 or 108-113 of human alpha-synuclein of SEQ ID NO: 1 as critical residues for binding.

In some embodiments, an isolated antibody that binds to human alpha-synuclein is provided, wherein the antibody binds extracellular or cytoplasmic alpha-synuclein. In some embodiments an isolated antibody that binds to monomeric or aggregated alpha-synuclein. In some embodiments of the invention, the monomeric, oligomeric or aggregated alpha-synuclein is post-translationally modified, e.g. phosphorylated or nitrosylated. The invention also relates to compositions comprising a binding molecule, particularly an antibody of the invention (including alpha-synuclein antibody fragments and derivatives) as described herein and to therapeutic and diagnostic methods using such compositions in the prevention, diagnosis or treatment of a synucleopathy, wherein an effective amount of the binding molecule is administered to a patient in need thereof.

In certain embodiments, the alpha-synuclein antibodies described herein, including in the form of mixtures, are useful for detecting the presence of alpha-synuclein in a biological sample. Such methods (specific examples of which are described herein) are typically performed in vitro using an isolated sample. However, they may be performed in vivo in some circumstances, where appropriate. In particular embodiments, the alpha-synuclein antibodies described herein are useful for detecting the presence of aggregated and/or pathological alpha-synuclein, including but not limited to Lewy bodies, Lewy neurites and/or glial cytoplasmic inclusions in a biological sample. The term “detecting” as used herein encompasses quantitative or qualitative detection.

The biological sample (in all methods reliant upon such detecting) is typically a clinical sample from a mammalian, in particular human, subject. In certain embodiments, a biological sample comprises a cell or tissue, such as cerebrospinal fluid (CSF), a cell or tissue of the brain (e.g., brain cortex or hippocampus), or blood. In some embodiments, a biological sample is cerebrospinal fluid.

In some embodiments, an alpha-synuclein antibody described herein for use in a method of diagnosis or detection is provided. In a further aspect, a method of detecting the presence of alpha-synuclein in a biological sample is provided. In certain embodiments, the method comprises contacting the biological sample with an alpha-synuclein antibody as described herein under conditions permissive for binding of the alpha-synuclein antibody to alpha-synuclein, and detecting whether a complex is formed between the alpha-synuclein antibody and alpha-synuclein. Such method may be an in vitro and/or in vivo method. Further, the complex formed between the alpha-synuclein antibody and alpha-synuclein in a test biological sample can be compared to the complex formed in a control biological sample (e.g., a biological sample from a healthy subject or subjects). The amount of the complex formed between the alpha-synuclein antibody and alpha-synuclein in a test biological sample can also be quantified and compared to the amount of the complex formed in a control biological sample (e.g., a biological sample from a healthy subject or subjects) or to the average amount of the complex known to be formed in healthy subjects. Mixtures of the invention may be employed in such methods.

In certain embodiments, an alpha-synuclein binding molecule, in particular an alpha-synuclein antibody or antigen-binding fragment thereof, of the invention and as provided herein is useful for detecting the presence of alpha-synuclein in a biological sample. In particular embodiments, the alpha-synuclein binding molecule, in particular an alpha-synuclein antibody or antigen-binding fragment thereof, of the invention and as provided herein is useful as an assay reagent, positive control, biomarker detection reagent and/or calibrator for an immunoassay, (including, but not limited to an ELISA, MSD (Meso Scale Discovery Inc., USA), Luminex (Luminex Corp., USA), Alphalisa (PerkinElmer, Inc., USA), Gyrolab (Gyros Protein Technologies AB, Sweden), Simoa (Quanterix Corp., USA), Gyros™ (Given et al., 2012), Singulex Erenna (EMD Millipore, Corp., USA), iR-SENSE/Immuno-InfraRed assay (Nabers et al, 2016), MITOMI (Piraino et al, 2016), Immunoprecipitation combined with liquid chromatography mass spectrometry (IP LC-MS/MS; Shimadzu, Germany), Surface plasmon resonance (SPR; Cytiva Europe, Switzerland), Atomic force microscope (AFM) (Kiio and Park, 2020) or any other assay technology or kit that relies on antibodies for target immunocapture and/or detection). As such, the alpha-synuclein binding molecule, in particular the alpha-synuclein antibody or antigen-binding fragments thereof, may be used in assays for validating/screening alpha-synuclein binding molecules, alpha-synuclein antibodies or antigen-binding fragments thereof. The alpha-synuclein binding molecules, in particular an alpha-synuclein antibody or antigen-binding fragment thereof, of the invention may be used as detection tools and/or positive controls as they bind to all alpha-synuclein species in the sample in selective fashion. Diagnostic compositions of the invention may be used in such methods. Mixtures of the invention may be employed in such methods.

The invention therefore provides a method of detecting alpha-synuclein in a sample obtained from a subject, the method comprising contacting the sample with a binding molecule, in particular an antibody or antigen-binding fragment of the invention and detecting binding of the antibody or antigen-binding fragment thereof in order to detect alpha-synuclein in the sample. Similarly, the invention provides a method of quantifying alpha-synuclein in a sample obtained from a subject, the method comprising contacting the sample with a binding molecule, in particular an antibody or antigen-binding fragment of the invention and performing quantification based on the binding of the binding molecule to alpha-synuclein. This method may comprise comparing the alpha-synuclein levels in the sample to those in a control sample or samples. The levels in control samples represent known levels against which the levels in the test sample may be determined. The control samples are not, therefore, necessarily tested at the same time as the method of quantification is performed. However, in some embodiments, reference levels are determined in parallel with the test sample. For example, a quantitative ELISA, ELISA, MSD (Meso Scale Discovery Inc., USA), Luminex (Luminex Corp., USA), Alphalisa (PerkinElmer, Inc., USA), Gyrolab (Gyros Protein Technologies AB, Sweden), Simoa (Quanterix Corp., USA), Gyros™ (Given et al., 2012), Singulex Erenna (EMD Millipore, Corp., USA), iR-SENSE/Immuno-InfraRed assay (Nabers et al, 2016), MITOMI (Piraino et al, 2016), Immunoprecipitation combined with liquid chromatography mass spectrometry (IP LC-MS/MS; Shimadzu, Germany), Surface plasmon resonance (SPR; Cytiva Europe, Switzerland), Atomic force microscope (AFM) (Kiio and Park, 2020) may be performed. A standard curve may be generated to permit quantification based on a dilution series (serial dilution) of alpha-synuclein. Diagnostic compositions of the invention may be used in such methods. Sandwich immunoassays, incorporating a suitable capture and detection antibody or antigen binding fragment thereof, may be used in the methods of quantifying alpha-synuclein in a sample obtained from a subject. Mixtures of the invention may be employed in such methods.

The invention also provides a method for diagnosing a disease, disorder and/or condition associated with alpha-synuclein comprising contacting the sample with a binding molecule, in particular an antibody or antigen-binding fragment of the invention and comparing the alpha-synuclein levels in the sample to those in a control sample or samples. Higher levels of alpha-synuclein in the sample compared with a control level based on healthy subjects are indicative of a disease, disorder and/or condition associated with alpha-synuclein. Additionally or alternatively similar or higher levels of alpha-synuclein in the sample compared with a diseased control (i.e. one or more samples from a subject having the disease, disorder and/or condition associated with alpha-synuclein) are indicative of a disease, disorder and/or condition associated with alpha-synuclein. Diagnostic compositions of the invention may be used in such methods. Sandwich immunoassays, incorporating a suitable capture and detection antibody or antigen binding fragment thereof, may be used in the methods of diagnosing a disease, disorder and/or condition associated with amyloid-beta. Mixtures of the invention may be employed in such methods.

The binding molecules of the invention are also useful in classification methods, for example, to indicate the relative stage of the disease, disorder and/or condition associated with alpha-synuclein. The invention therefore also provides a method for classifying a disease, disorder and/or condition associated with alpha-synuclein comprising contacting a sample from a subject with a binding molecule, in particular an antibody or antigen-binding fragment of the invention and comparing the alpha-synuclein levels in the sample to those in a control sample or samples in order to classify the disease. A range of controls representative of different classes of disease may be employed in order to classify the sample. The test sample may be classified based on the best match to the control samples. Higher levels of alpha-synuclein in the sample compared with a control level based on healthy subjects are indicative of a disease, disorder and/or condition associated with alpha-synuclein. Similar or higher levels of alpha-synuclein in the sample compared with a diseased control at a certain stage of disease are indicative of that stage of disease, disorder and/or condition associated with alpha-synuclein. Such methods may be performed in relation to subjects known to have the disease, disorder and/or condition associated with alpha-synuclein and/or in relation to subjects not already known to have the disease, disorder and/or condition associated with alpha-synuclein. Diagnostic compositions of the invention may be used in such methods. Sandwich immunoassays, incorporating a suitable capture and detection antibody or antigen binding fragment thereof, may be used in the classification methods of the invention. Mixtures of the invention may be employed in such methods.

The invention also provides a method for monitoring a disease, disorder and/or condition associated with alpha-synuclein at two or more time points using samples from a subject, the method comprising contacting the samples with a binding molecule, in particular an antibody or antigen-binding fragment of the invention and comparing the alpha-synuclein levels in the samples, wherein higher levels of alpha-synuclein in the later sample compared with one or more earlier samples are indicative of progression of a disease, disorder and/or condition associated with alpha-synuclein. Similarly, the invention provides a method for monitoring a disease, disorder and/or condition associated with alpha-synuclein at two or more time points using samples from a subject, the method comprising contacting the samples with a binding molecule, in particular an antibody or antigen-binding fragment of the invention and comparing the alpha-synuclein levels in the samples, wherein lower levels of alpha-synuclein in the later sample compared with one or more earlier samples are indicative of regression of a disease, disorder and/or condition associated with alpha-synuclein. These methods also permit a lack of progression of the disease to be monitored, where there is no significant change in levels of alpha-synuclein in the later sample compared with one or more earlier samples. Such methods are typically performed in relation to subjects known to have the disease, disorder and/or condition associated with alpha-synuclein. Diagnostic compositions of the invention may be used in such methods. Sandwich immunoassays, incorporating a suitable capture and detection antibody or antigen binding fragment thereof, may be used in the monitoring methods of the invention. Mixtures of the invention may be employed in such methods.

Monitoring methods are useful to determine whether a particular therapy is successful or otherwise. The invention therefore also provides a method for monitoring a disease, disorder and/or condition associated with alpha-synuclein at two or more time points using samples from a subject, the method comprising contacting the samples with a binding molecule, in particular an antibody or antigen-binding fragment of the invention, wherein lower levels of alpha-synuclein in the later sample compared with one or more earlier samples are indicative of successful treatment of a disease, disorder and/or condition associated with alpha-synuclein. The therapy may be any suitable candidate therapeutic agent, such as an antibody or small molecule therapeutic. These methods also permit a lack of progression of the disease to be monitored, where there is no significant change in levels of alpha-synuclein in the later sample compared with one or more earlier samples. This may also be considered successful treatment in some circumstances. Indeed, a decline in the rate of increase of alpha-synuclein levels between samples, compared with the rate of increase prior to therapy, may also be considered indicative of successful treatment. Such methods are typically performed in relation to subjects known to have the disease, disorder and/or condition associated with alpha-synuclein. Unsuccessful treatment may be determined where the treatment provides no decline in the rate of increase of alpha-synuclein levels between samples, compared with the rate of increase prior to therapy. Diagnostic compositions of the invention may be used in such methods. Sandwich immunoassays, incorporating a suitable capture and detection antibody or antigen binding fragment thereof, may be used in the methods of monitoring therapy of the invention. Mixtures of the invention may be employed in such methods.

The binding molecules of the invention may also be used to assist with therapy selection. Thus, the invention provides a method for selecting a therapy for treatment of a disease, disorder and/or condition associated with alpha-synuclein, the method comprising contacting samples taken before and after treatment with a binding molecule, in particular an antibody or antigen-binding fragment of the invention, wherein lower levels of alpha-synuclein in the sample taken after treatment compared with the sample taken before treatment are indicative of successful treatment of a disease, disorder and/or condition associated with alpha-synuclein and thus the therapy is selected for treatment. The therapy may be any suitable candidate therapeutic agent, such as an antibody or small molecule therapeutic. A therapy halting progression of the disease may also be selected, where there is no significant change in levels of alpha-synuclein in the later sample compared with one or more earlier samples. This may also be considered successful treatment in some circumstances. Indeed, a decline in the rate of increase of alpha-synuclein levels between samples, compared with the rate of increase prior to therapy, may also be considered indicative of successful treatment and therefore result in selection of the particular therapy. Such methods are typically performed in relation to subjects known to have the disease, disorder and/or condition associated with alpha-synuclein. Unsuccessful treatment may be determined where the treatment provides no decline in the rate of increase of alpha-synuclein levels between samples, compared with the rate of increase prior to therapy. Such therapy is not selected for treatment. Alternatively, higher levels of alpha-synuclein in the sample taken after treatment compared with the sample taken before may be indicative of unsuccessful treatment of a disease, disorder and/or condition associated with alpha-synuclein and thus the therapy is not selected for treatment. Diagnostic compositions of the invention may be used in such methods. Sandwich immunoassays, incorporating a suitable capture and detection antibody or antigen binding fragment thereof, may be used in the therapy selection methods of the invention (as applied to individual subjects). Mixtures of the invention may be employed in such methods.

Methods of the invention are also useful to determine whether a particular therapy is successful or otherwise in the context of a larger, controlled study, such as a clinical trial. Thus, these methods are typically applied to a treatment group of subjects that is compared with a group of subjects not treated with the therapy. In such a context, control samples not treated with the therapy are also available for comparative purposed (placebo group). The invention therefore also provides a method for assessing a candidate therapy for a disease, disorder and/or condition associated with alpha-synuclein, the method comprising, following treatment of one or more subjects, contacting samples from the one or more treated subjects with a binding molecule, in particular an antibody or antigen-binding fragment of the invention, wherein lower levels of alpha-synuclein in the samples compared with levels in corresponding samples from subjects not treated with the therapy are indicative of successful treatment of a disease, disorder and/or condition associated with alpha-synuclein. The methods are typically performed in relation to a plurality (i.e. at least two) treated subjects and a plurality of control subjects. The treated and control groups may or may not be of the same size. They may each comprise 3 or more, 4 or more, 5 or more, 10 or more, 20 or more, 50 or more subjects in some embodiments. The therapy may be any suitable candidate therapeutic agent, such as a biologic, in particular an antibody, a vaccine or small molecule therapeutic. The methods may be performed at multiple time points in matched samples between the treatment and placebo groups in order to monitor the effectiveness of the candidate therapy over a defined time period. An initial pre-therapy sample is typically also taken. Thus, the methods may comprise contacting samples from the one or more treated subjects and the subjects not treated with the therapy with a binding molecule, in particular an antibody or antigen-binding fragment of the invention prior to treatment to determine base levels of amyloid-beta. “Prior to treatment” means prior to administration of the therapy or the placebo depending upon the subject group. The binding molecules of the invention may therefore also be used to assist with assessment of candidate therapies in the context of clinical trials. Candidate therapies providing successful treatment may be selected and, ultimately, approved for marketing. Diagnostic compositions of the invention may be used in such methods. Sandwich immunoassays, incorporating a suitable capture and detection antibody or antigen binding fragment thereof, may be used in the therapy selection methods of the invention (as applied to clinical trials). Mixtures of the invention may be employed in such methods.

In some embodiments, an alpha-synuclein antibody described herein is used to select subjects eligible for therapy, including therapy with an alpha-synuclein antibody, e.g. where alpha-synuclein is a biomarker for selection of patients. For example, in some embodiments, an alpha-synuclein antibody is used to detect whether the subject has a disease, disorder or abnormality associated with alpha-synuclein aggregates including but not limited, Lewy bodies, Lewy neurites and/or Glial cytoplasic inclusions, or whether the subject is at high risk (or predisposed to) a disease or disorder or abnormality associated with alpha-synuclein aggregates including but not limited, Lewy bodies, Lewy neurites and/or Glial cytoplasic inclusions.

Exemplary diseases or disorders or abnormality that may be diagnosed using an antibody of the invention include diseases or disorders or abnormalities associated with alpha-synuclein aggregates including, but not limited, Lewy bodies, Lewy neurites and/or Glial cytoplasic inclusions, that are manifested in a cognitive deficit or behavioral impairment, or motor deficit or impairment such as bradykinesia, rigidity, resting tremor or postural instability. In particular, diseases or disorders or abnormality that may be diagnosed using an antibody, antigen-binding fragment thereof or derivative thereof, of the invention include synucleinopathies such as Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease.

Exemplary diseases or disorders or abnormality that may be prevented or treated using an antibody of the invention include diseases, disorders or abnormalities associated with alpha-synuclein aggregates including, but not limited, Lewy bodies, Lewy neurites and/or Glial cytoplasic inclusions, that are manifested in a cognitive deficit or behavioral impairment, or motor deficit or impairment such as bradykinesia, rigidity, resting tremor or postural instability. In particular, diseases or disorders or abnormality that may be diagnosed using an antibody, antigen-binding fragment thereof or derivative thereof, of the invention include synucleinopathies such as Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease.

In some embodiments, an immunoconjugate is provided, wherein the immunoconjugate comprises an isolated antibody described herein and a therapeutic agent.

In some embodiments, a labeled antibody is provided, comprising an antibody described herein and a detectable label.

In some embodiments the alpha-synuclein binding molecule of the present invention is linked to a detectable label.

In some embodiments the alpha-synuclein binding molecule is part of an immunoconjugate wherein the alpha-synuclein binding molecule is covalently linked to another suitable therapeutic agent.

In some embodiments an alpha-synuclein binding molecule is part of a pharmaceutical composition comprising an alpha-synuclein binding molecule, or an immunoconjugate wherein the alpha-synuclein binding molecule is covalently linked to another suitable therapeutic agent, or a composition comprising an alpha-synuclein specific binding molecule combined with a pharmaceutically acceptable carrier and/or excipient.

In some embodiments an alpha-synuclein binding molecule is part of a diagnostic kit comprising an alpha-synuclein specific binding molecule, or an immunoconjugate wherein the alpha-synuclein specific binding molecule is covalently linked to another suitable therapeutic agent, or a composition comprising an alpha-synuclein specific binding molecule.

In some embodiments an alpha-synuclein binding molecule is used in an immunodiagnostic method for use in the prevention, diagnosis, alleviation of symptoms associated with, or treatment of a disease or disorder or abnormality associated with alpha-synuclein aggregates including, but not limited to, Lewy bodies, Lewy neurites, and/or glial cytoplasmic inclusions.

In some embodiments, a diagnostic composition is provided, comprising an isolated alpha-synuclein binding molecule, in particular an alpha-synuclein antibody or antigen-binding fragment thereof, described herein and a pharmaceutically acceptable carrier and/or excipient. Mixtures of the invention may be employed in such diagnostic compositions.

Pharmaceutical formulations of an alpha-synuclein antibody or diagnostic composition as described herein are prepared by mixing such antibody or diagnostic composition having the desired degree of purity with one or more optional pharmaceutically acceptable carriers and/or excipients and/or diluents (Remington's Pharmaceutical Sciences 16th edition, Osol, A. Ed. (1980)). Typically, the antibody or fragment therefor is prepared as a lyophilized formulation or aqueous solution. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases. Pharmaceutically acceptable excipients that may be used to formulate the compositions include, but are not limited to: ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and lanolin. Diluents may be buffers. They may comprise a salt selected from the group consisting of phosphate, acetate, citrate, succinate and tartrate, and/or wherein the buffer comprises histidine, glycine, TRIS glycine, Tris, or mixtures thereof. It is further envisaged in the context of the present invention that the diluent is a buffer selected from the group consisting of potassium phosphate, acetic acid/sodium acetate, citric acid/sodium citrate, succinic acid/sodium succinate, tartaric acid/sodium tartrate, and histidine/histidine HCl or mixtures thereof.

In some embodiments an alpha-synuclein binding molecule is part of an immunotherapeutic method for the prevention, alleviation of symptoms associated with, or treatment of a synucleinopathy, wherein an effective amount of the alpha-synuclein binding molecule, or an immunoconjugate wherein the alpha-synuclein binding molecule is covalently linked to another suitable therapeutic agent, or a composition comprising an alpha-synuclein binding molecule is administered to a patient in need thereof. Mixtures of the invention may be employed in such methods.

In some embodiments the alpha-synuclein binding molecule, or an immunoconjugate wherein the alpha-synuclein binding molecule is covalently linked to another suitable therapeutic agent, or a composition comprising an alpha-synuclein binding molecule is administered to a patient in need thereof is used to diagnose, prevent, alleviate, delay, inhibit or treat a disease, disorder or abnormality associated with alpha-synuclein aggregates, such as Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. Mixtures of the invention may be employed in such methods.

In some embodiments the alpha-synuclein binding molecule, or an immunoconjugate wherein the alpha-synuclein specific binding molecule is covalently linked to another suitable therapeutic agent, or a composition comprising an alpha-synuclein binding molecule is administered to a patient in need thereof is used in a method for diagnosing or monitoring a disease, disorder or abnormality associated with alpha-synuclein aggregates such as Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), Diffuse Lewy Body Disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder. Mixtures of the invention may be employed in such methods.

In some embodiments an alpha-synuclein binding molecule is used in a method for diagnosing presymptomatic disease or disorder or abnormality, or for monitoring disease or disorder or abnormality progression and therapeutic efficacy of a drug, or for predicting responsiveness, or for selecting patients which are likely to respond to the treatment with an alpha-synuclein binding molecule. Said method is preferably performed using a sample of human blood or urine. Most preferably the method involves an ELISA-based or surface adapted assay. Mixtures of the invention may be employed in such methods.

In some embodiments an alpha-synuclein binding molecule is used in a method wherein an alpha-synuclein binding molecule of the present invention is contacted with a sample (e.g., blood, cerebrospinal fluid, or brain tissue) to detect, diagnose or monitor Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. Mixtures of the invention may be employed in such methods.

In some embodiments an alpha-synuclein binding molecule is used in a method wherein an alpha-synuclein specific binding molecule of the present invention is contacted with a sample (e.g., blood, cerebrospinal fluid, or brain tissue) to detect, diagnose a disease or disorder or abnormality associated with alpha-synuclein aggregates, such as Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), Diffuse Lewy Body Disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder. Mixtures of the invention may be employed in such methods.

In some embodiments an alpha-synuclein binding molecule, or an immunoconjugate wherein the alpha-synuclein binding molecule is covalently linked to another suitable therapeutic agent, or a composition comprising an alpha-synuclein binding molecule is administered to a patient in need thereof is used for preventing, alleviating or treating a disease, disorder or abnormality associated with apha-synuclein aggregates or a synucleinoptahy or Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. Mixtures of the invention may be employed in such methods.

In some embodiments an alpha-synuclein binding molecule, or an immunoconjugate wherein the alpha-synuclein binding molecule is covalently linked to another suitable therapeutic agent, or a composition comprising an alpha-synuclein binding molecule is administered to a patient in need thereof is used for treating a disease or disorder or abnormality associated with alpha-synuclein aggregates, such as Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder. Mixtures of the invention may be employed in such methods.

In some embodiments an alpha-synuclein binding molecule, or an immunoconjugate wherein the alpha-synuclein binding molecule is covalently linked to another suitable therapeutic agent, or a composition comprising an alpha-synuclein specific binding molecule is administered to a patient in need thereof is used for manufacturing a medicament for treating a disease, disorder or abnormality associated with alpha-synuclein aggregates, or a synucleinoptahy or Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. Mixtures of the invention may be employed in such methods.

In some embodiments, an alpha-synuclein antibody or immunoconjugate for use as a medicament is provided. In some embodiments, an alpha-synuclein antibody or immunoconjugate for use in a method of treatment is provided. In certain embodiments, an anti-alpha-synuclein antibody or immunoconjugate for use in the prevention, diagnosis and/or treatment of a synucleinopathy is provided. In a preferred embodiment of the invention, an alpha-synuclein antibody or immunoconjugate is provided for use in the prevention, diagnosis and/or treatment of a disease, disorder or abnormality associated with alpha-synuclein aggregates, such as Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. Mixtures of the invention may be employed in such methods.

In some embodiments, the invention describes the use of an alpha-synuclein antibody or immunoconjugate in the manufacture or preparation of a medicament. In one such embodiment, the method further comprises administering to the individual an effective amount of at least one additional therapeutic agent. Mixtures of the invention may be employed in such uses.

Antibodies or immunoconjugates of the invention can be used either alone or in combination with other agents in a therapy. For instance, an antibody or immunoconjugate of the invention may be co-administered with at least one additional therapeutic agent. Mixtures of the invention may be employed.

In another aspect of the invention, an article of manufacture containing materials useful for the treatment, prevention and/or diagnosis of the disease or disorders or abnormality described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disease, disorder or abnormality and may have a sterile access port (for example the container may be an intravenous solution bag or a vial having a stopper pierceable by a hypodermic injection needle). At least one active agent in the composition is an antibody or immunoconjugate of the invention. The label or package insert indicates that the composition is used for treating the condition of choice. Moreover, the article of manufacture may comprise (a) a first container with a composition contained therein, wherein the composition comprises an antibody or immunoconjugate of the invention; and (b) a second container with a composition contained therein, wherein the composition comprises a further therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes. Mixtures of the invention may be employed.

The methods of the invention may comprise administering at least one additional therapy, preferably wherein the additional therapy is selected from, but not limited to, neurological drugs, levodopa (e.g. Sinemet®), catechol-O-methyl transferase inhibitors (e.g. entacapone, tolcapone), dopamine agonists, monoamine oxidase B inhibitors (e.g. rasagiline, selegiline) Amantadine, anticholinergic medication, anti-abeta antibodies, anti-Tau antibodies, Tau aggregation inhibitors, beta-amyloid aggregation inhibitors, anti-BACE1 antibodies, and BACE1 inhibitors.

The invention furthermore relates to a method of detecting aggregated and/or pathological alpha-synuclein, including, but not limited to Lewy neurites, Lewy Bodies and/or Glial cytoplasmic inclusions, comprising contacting a sample with the binding molecule of the invention, preferably wherein the sample is a brain sample, a cerebrospinal fluid sample, urine sample or a blood sample. Mixtures of the invention may be employed.

In some embodiments, the invention encompasses alpha-synuclein binding molecules, particularly antibodies of the invention as described herein that binds aggregated and/or pathological alpha-synuclein and the use of these molecules to diagnose, prevent, alleviate or treat a disease, disorder or abnormality associated with alpha-synuclein aggregates such as Parkinson's Disease, Multiple System Atrophy, Lewy Body dementia (LBD; dementia with Lewy bodies (DLB) (“pure” Lewy body dementia), Parkinson's disease dementia (PDD)), or Diffuse Lewy Body Disease. Mixtures of the invention may be employed.

In another embodiment, a binding molecule, particularly an antibody of the invention as described herein specific for alpha-synuclein is administered to prevent, alleviate or treat a disease, disorder or abnormality associated with alpha-synuclein aggregates selected from Parkinson's Disease, Multiple System Atrophy, Lewy Body dementia (LBD; dementia with Lewy bodies (DLB) (“pure” Lewy body dementia), Parkinson's disease dementia (PDD)), and Diffuse Lewy Body Disease. Mixtures of the invention may be employed.

In another embodiment, a binding molecule, in particular antibodies or antigen-binding fragments thereof as described herein, binding aggregated and/or pathological alpha-synuclein is contacted with a sample to detect, diagnose or monitor a disease, disorder or abnormality associated with alpha-synuclein aggregates selected from Parkinson's disease (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), Diffuse Lewy Body Disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17, and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder. Mixtures of the invention may be employed.

The invention furthermore relates to methods for evaluating an alpha-synuclein binding molecule for the capability of inhibiting and/or delaying the seeded and/or spontaneous alpha-synuclein aggregation, comprising the steps of bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and determining the time to reach half-maximum signal of the detectable dye, particularly the signal of fluorescent dye, relative to the seeded aggregation in the absence of binding molecule. In an alternative or additional embodiment, the method for evaluating an alpha-synuclein binding molecule for the capability of inhibiting and/or delaying the seeded and/or spontaneous alpha-synuclein aggregation, may comprise the steps of bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and determining the time to reach half-maximum signal of the detectable dye, particularly the signal of fluorescent dye, wherein an increase in time to reach half-maximum signal of the detectable dye in the presence of binding molecule relative to the seeded aggregation in the absence of binding molecule indicates that the alpha-synuclein binding molecule is capable of inhibiting and/or delaying the seeded and/or spontaneous alpha-synuclein aggregation. In a further alternative or additional embodiment, the method for evaluating an alpha-synuclein binding molecule for the capability of inhibiting and/or delaying the seeded and/or spontaneous alpha-synuclein aggregation, may comprise the steps of bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and determining the time to reach half-maximum signal of the detectable dye, particularly the signal of fluorescent dye, and detecting the increase in time to reach half-maximum signal of the detectable dye in the presence of binding molecule relative to the seeded aggregation in the absence of binding molecule, indicating that the alpha-synuclein binding molecule inhibits and/or delays the seeded and/or spontaneous alpha-synuclein aggregation. In a yet further alternative or additional embodiment the method for evaluating an alpha-synuclein binding molecule for the capability of inhibiting and/or delaying the seeded and/or spontaneous alpha-synuclein aggregation, may comprise the steps of bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and measuring the increase in time to reach half-maximum signal of the detectable dye in the presence of the alpha-synuclein binding molecule relative to the seeded aggregation in the absence of binding molecule, as an indication of the binding molecule having capability of inhibiting and/or delaying the seeded and/or spontaneous alpha-synuclein aggregation.

The invention furthermore relates to a method for screening an alpha-synuclein binding molecule capable of inhibiting and/or delaying the seeded and/or spontaneous alpha-synuclein aggregation, comprising the steps of bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and selecting the alpha-synuclein binding molecule as being able to inhibit and/or delay seeded and/or spontaneous alpha-synuclein aggregation based on the signal of the detectable dye, in particular the fluorescent dye, determined in the absence and presence of the alpha-synuclein binding molecule.

The screening or evaluation methods provided herein may further comprise a step of providing alpha-synuclein binding molecules to be screened/evaluated. The binding molecules may for example be provided in form of a library, in particular an antibody library. The skilled person is well-aware of methods for providing binding molecule libraries and in particular antibody libraries. Alternatively, libraries may be obtained commercially before evaluation/screening.

The invention furthermore relates to an in vitro assay for screening for alpha-synuclein binding molecules for the capability of inhibiting and/or delaying the seeded and/or spontaneous alpha-synuclein aggregation, said assay comprising the steps of bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and selecting the alpha-synuclein binding molecule as being able to inhibit and/or delay seeded and/or spontaneous alpha-synuclein aggregation based on the signal of the detectable dye, in particular the fluorescent dye, determined in the absence and presence of the alpha-synuclein binding molecule. In an alternative or additional embodiment, the invention relates to an in vitro assay for evaluating an alpha-synuclein binding molecule for the capability of inhibiting and/or delaying the seeded and/or spontaneous alpha-synuclein aggregation, said assay comprising the steps of: bringing an alpha-synuclein binding molecule in contact with alpha-synuclein aggregates (seeds); allowing the alpha-synuclein binding molecule to bind to alpha-synuclein aggregates, to form an immunological complex; adding alpha-synuclein monomeric protein and a detectable dye, in particular a fluorescent dye, to the immunological complex; and determining the time to reach half-maximum signal of the detectable dye, particularly the signal of fluorescent dye, wherein an increase in time to reach half-maximum signal of the detectable dye in the presence of binding molecule relative to the seeded aggregation in the absence of binding molecule indicates that the alpha-synuclein binding molecule is capable of inhibiting and/or delaying the seeded and/or spontaneous alpha-synuclein aggregation. In a particular embodiment, the fluorescent dye is thioflavin.

The invention also relates to kits for use in screening or evaluating alpha-synuclein binding molecules, in particular antibodies. Such kits may comprise all necessary components for performing the herein provided methods and/or assays, such as, for example, buffers, detectable dyes, laboratory equipment, reaction containers, instructions and the like.

The invention also relates to methods for the prevention, alleviation or treatment of diseases, disorders and/or abnormalities associated with alpha-synuclein, particularly with pathological alpha-synuclein and/or aggregated alpha-synuclein, comprising administering an effective amount of an alpha-synuclein binding molecule, in particular an antibody, of the invention to a subject in need thereof. Mixtures of the invention may be employed.

### FIGURES

FIG. 1: Effect of mAbs on aggregation half-times in seeded a-syn aggregation. Effect of mAbs on aggregation half-times in seeded a-syn aggregation. (A) Change in aggregation half-times (τ½), relative to the control in the absence of mAb. (B) Percent increases of τ½ values, relative to the control in the absence of mAb, are plotted for the seeded aggregations in the presence of the indicated mAb. Error bars represent the propagation of error (Equation 5). Significance was determined using a one-way ANOVA (Dunnett's multiple comparisons test) versus aggregation in the absence of mAb (n.s. not significant; (*) P<0.033; (**) P<0.002, (***) P<0.0002), (****) P<0.0001.

FIG. 2. Effect of mAbs on aggregation half-times in seeded a-syn aggregation. Effect of mAbs on aggregation half-times in seeded a-syn aggregation. (A) Change in aggregation half-times (τ½), relative to the control in the absence of mAb. (B) Percent increases of τ½ values, relative to the control in the absence of mAb, are plotted for the seeded aggregations in the presence of the indicated mAb. Error bars represent the propagation of error (Equation 5). Significance was determined using a one-way ANOVA (Dunnett's multiple comparisons test) versus aggregation in the absence of mAb (n.s. not significant; (**) P<0.002; (****) P<0.0001).

FIG. 3: Effect of mAbs on aggregation half-times in seeded a-syn aggregation. (A) Change in aggregation half-time (τ½), relative to the control in the absence of mAb. An antibody not binding to a-syn was used as isotype control (B) Percent increase of τ½ values, relative to the control in the absence of mAb, is plotted for the seeded aggregation in the presence of the indicated mAbs. Error bars represent the propagation of error (Equation 5). Significance was determined using a one-way ANOVA (Dunnett's multiple comparisons test) versus aggregation in the absence of mAb (****) P<0.0001).

The invention will be further understood with reference to the following non-limiting examples:

### EXAMPLES

### Preparation of an Alpha-Synuclein Liposomal Vaccine Composition

The liposome-based antigenic constructs were prepared according to the protocols published in WO2012/055933. The liposomal vaccine with human full-length alpha-synuclein protein as antigen was used for antibody generation (Table 2, SEQ ID NO: 1) or liposomal vaccine with alpha-synuclein peptide as antigen was used for antibody generation.

TABLE 2
antigen description

| | Definition | Amino acid sequence (1-letter code) |
|---|---|---|
| SEQ ID NO: 1 | FL-alpha- | MDVFMKGLSKAKEGVVAAAEKTKQGVAEAAGKTKEG |
| | synuclein (140 aa) | VLYVGSKTKEGVVHGVATVAEKTKEQVTNVGGAVVT |
| | | GVTAVAQKTVEGAGSIAAATGFVKKDQLGKNEEGAP |
| | | QEGILEDMPVDPDNEAYEMPSEEGYQDYEPEA |

### Mouse Immunization

Female C57BL/6JOlaHsd and BALB/cOlaHsd mice (Envigo, USA) were vaccinated at 10 weeks of age. C57BL/6JOlaHsd substrain is known to have a spontaneous deletion of the alpha-synuclein gene. Mice were vaccinated with vaccine containing human full-length alpha-synuclein protein or alpha-synuclein peptide presented on the surface of liposomes in the presence of synthetic monophosphoryl hexa-acyl Lipid A 3-deacyl (3D-(6-acyl) PHAD®) (Avanti Polar Lipids, USA) as adjuvant.

Mice were vaccinated by subcutaneous injection (s.c.) on days 0, 7, 27, 70, and in some cases on day 14, 28, 63, and 73. Mice were bled and heparinized plasma prepared 7 days before immunization (pre-immune plasma) and on days 14, 37, 73 and in some cases on day 7, 21, 35, 35, 70, 73, after first immunization. Mice used for myeloma fusion or phage display were additionally vaccinated with three or four daily booster injections by intraperitoneal injection (i.p.) of liposomal vaccines without adjuvant. Very high antigen-specific IgG responses were obtained in all immunized mice.

### Isolation of Clonal Mouse Hybridoma Cell Lines Producing Specific and High-Affinity Monoclonal Antibodies

Mice were euthanized and fusion with PAI myeloma cells was performed using splenocytes from immunized mice. For screening fusion products, cell culture supernatant was diluted 1:50 and analysed using Luminex bead-based multiplex assay (Luminex, The Netherlands). Luminex beads were conjugated to either full-length alpha-synuclein, alpha-synuclein peptide 1-60aa, alpha-synuclein peptide 1-95aa, alpha-synuclein peptide 61-140aa, or full-length beta-synuclein (irrelevant target), and with capturing IgGs with anti-mouse IgG-Fc antibodies specific for the IgG1, IgG2a, IgG2b, IgG2c, and IgG3 subclasses (Jackson Immunoresearch, USA). Luminex assay results binding to full-length alpha-synuclein identified 92 hits. In a second round of fusion of immunized mice splenocytes and PAI myeloma cells, 400 hits were identified by Luminex assay binding to full-length alpha-synuclein. Viable hybridomas were grown using serum-containing selection media, and the best hybridomas binding to full-length alpha-synuclein were then selected for subcloning. Following limiting dilution, the clonal hybridomas were grown in low immunoglobulin containing medium and stable colonies were selected for antibody screening and selection.

In another round of fusion of immunized mice splenocytes or lymph nodes (popliteals, axial, brachials, and inguinals) and X63/AG.8653 myeloma cells, 279 hits were identified by ELISA assay binding to alpha-synuclein peptide 1-120aa. Viable hybridomas were grown using serum-containing selection media, and the best hybridomas binding to alpha-synuclein peptide were then selected for subcloning. Following limiting dilution, the clonal hybridomas were grown in low immunoglobulin containing medium and stable colonies were selected for antibody screening and selection.

### Isolation of Alpha Synuclein Antibodies by Phage Display

Some antibodies were generated by phage display using the same group of immunized mice previously described. RT-PCR was performed on mRNA isolated from splenocytes. VH and VL region were assembled as scFv and cloned into phagemid vectors resulting in a phage display library of 1×10⁷ clones. Several rounds of panning were performed either against full length human alpha synuclein or against alpha synuclein fragments, amino acids 1-60 (SEQ ID NO: 850), amino acids 61-95 (SEQ ID NO: 851), or amino acids 96-140 (SEQ ID NO: 147) (Table 3).

Positive clones were sequenced and expressed recombinantly as murine IgG2a for characterization.

### Epitope Mapping on Alpha-Synuclein

Serum-free supernatants were harvested from stable hybridomas. The supernatants containing antibodies of interest were then screened by an indirect ELISA assay to determine epitopes. Epitopes were first determined using a library of 15-mer peptides covering the entire sequence of human alpha-synuclein protein, spanning amino acids (aa) 1-140 with 9aa offset and 6aa overlap. All peptides were synthesized biotinylated at N-terminus with aminohexanoic acid spacer except the N-terminal peptide 1-14aa (SEQ ID NO: 120) which was synthesized biotinylated at the C-terminus. Briefly, streptavidin-coated ELISA plates were blocked overnight at 4° C. (PBS/0.05% Tween®20/1% BSA) and then incubated for 1 hour at 25° C. with 0.25 μM of biotinylated full-length alpha-synuclein protein or biotinylated 15-mer peptides. Peptide sequences are provided in Table 3. Plates were washed with PBS/0.05% Tween®20 and then incubated with the hybridoma supernatants at 1/100 dilution for 1 hour at 25° C. Next, plates were washed with PBS/0.05% Tween®20 and incubated for 1 hour at 25° C. with the detection antibody, anti-mouse IgG conjugated to alkaline phosphatase (Jackson Immunoresearch Laboratories Inc., 115-055-164,) at 1:1000 dilution. After final wash, plates were incubated 2 hours at 25° C. with alkaline phosphatase substrate (p-nitrophenyl phosphate disodium hexahydrate; pNPP, S0942, Sigma) and read the absorbance optical density (O.D.) signal at 405 nm using an ELISA plate reader (Tecan, Switzerland). Tested antibodies were found to bind to one or more of the following peptides:

TABLE 3
Library of peptides used for epitope mapping

| | | aa alpha- |
|---|---|---|
| SEQ ID | | synuclein |
| NO: | Sequence | sequence |
| 120 | MDVFMKGLSKAKEG | 1-14* |
| 121 | MDVFMKGLSKAKEGV | 1-15 |
| 122 | KAKEGVVAAAEKTKQ | 10-24 |
| 123 | AEKTKQGVAEAAGKT | 19-33 |
| 124 | EAAGKTKEGVLYVGS | 28-42 |
| 125 | VLYVGSKTKEGVVHG | 37-51 |
| 126 | EGVVHGVATVAEKTK | 46-60 |
| 127 | VAEKTKEQVTNVGGA | 55-69 |
| 128 | TNVGGAVVTGVTAVA | 64-78 |
| 129 | GVTAVAQKTVEGAGS | 73-87 |
| 130 | VEGAGSIAAATGFVK | 82-96 |
| 131 | ATGFVKKDQLGKNEE | 91-105 |
| 132 | LGKNEEGAPQEGILE | 100-114 |
| 133 | QEGILEDMPVDPDNE | 109-123 |
| 134 | VDPDNEAYEMPSEEG | 118-132 |
| 135 | MPSEEGYQDYEPEA | 127-140 |
| 137 | TVEGAGSIAAATGFVKKDQLGKNEEGAPQEGILEDMPVDP | 81-120 |
| 850 | MDVFMKGLSKAKEGVVAAAEKTKQGVAEAAGKTKEGVLY | 1-60 |
| | VGSKTKEGVVHGVATVAEKTK | |
| 851 | EQVTNVGGAVVTGVTAVAQKTVEGAGSIAAATGFV | 61-95 |
| 147 | KKDQLGKNEEGAPQEGILEDMPVDPDNEAYEMPSEEGYQ | 96-140 |
| | DYEPEA | |

*Peptide biotinylated at C-terminus

Epitopes were further determined using a library of 8-mer peptides covering the alpha-synuclein sequences previously identified by indirect ELISA on a library of 15-mer peptides. The 8-mer peptides were designed with 1 aa offset and 7aa overlap. Finally, for determining the critical residues for antibody binding an Alanine scanning library of peptides was utilized covering the alpha-synuclein sequences previously identified with the library of 1 5-mer peptides. The peptides of the Alanine scanning library were from 15 to 30 residues in length and synthesized with an alanine residue in each position substituting the natural residue in the sequence (except when the natural residue is alanine). All peptides were synthesized biotinylated at N-terminus with aminohexanoic acid spacer. For the indirect ELISA, streptavidin-coated ELISA plates were blocked overnight at 4° C. (PBS/0.05% Tween®20/1% BSA) and then incubated for 1 hour at 25° C. with 0.25 μM of biotinylated peptides. Plates were washed with PBS/0.05% Tween®20 and then incubated with the hybridoma supernatants at 1/100 dilution for 1 hour at 2500. Next, plates were washed with PBS/0.05% Tween®20 and incubated for 1 hour at 25° C. with the detection antibody, anti-mouse IgG conjugated to alkaline phosphatase (Jackson Immunoresearch Laboratories Inc., 115-055-164,) at 1:1000 dilution. After final wash, plates were incubated 2 hours at 25° C. with alkaline phosphatase substrate (p-nitrophenyl phosphate disodium hexahydrate; pNPP, S0942, Sigma) and read the absorbance optical density (O.D.) signal at 405 nm using an ELISA plate reader (Tecan, Switzerland). Where indicated, binding epitopes were confirmed using recombinantly produced antibodies. Variable domain sequences were cloned into mammalian cell expression vectors and transiently transfected into CHO cells. Antibodies were purified from cell culture supernatant by standard protein A purification and were buffer exchanged in 1×PBS, prior to being tested for binding. The binding epitopes for the recombinantly produced antibodies are shown in Table 4. In the event of inconsistency between the results obtained using recombinant proteins and the results obtained from hybridoma supernatants the recombinant protein result is accepted (because there is some risk of contamination when diluting hybridoma supernatants). The binding epitopes for the antibodies are shown in Table 4.

TABLE 4
Antibody binding epitopes

| | | | Critical residues (aa) - |
|---|---|---|---|
| Antibody Code | Hybridoma Code | Epitope (aa) | Alanine scanning library |
| ACI-7067-4813-R4A-G7- | 4813-R4A-G7 | 96-140 (SEQ ID | N.D. |
| rec1 | | NO: 147) | |
| ACI-7079-3101E3-Ab1 | 3101E3C9 | 100-114 (SEQ | 108-114 |
| | | ID NO: 132) | |
| ACI-7079-3103D9-Ab1 | 3103D9C9 | 28-50 (SEQ ID | 33-37 |
| | | NO: 139) | |
| ACI-7079-3103G12-Ab2 | 3103G12D2B10 | 19-33 (SEQ ID | N.D. |
| | | NO: 123) | |
| ACI-7079-3104F12-Ab2 | 3104F12F2H7 | 1-15 (SEQ ID | 33-37 |
| | | NO: 121), 28-50 | |
| | | (SEQ ID NO: | |
| | | 139) | |
| ACI-7079-3104F12-Ab2* | 3104F12F2H7 | 28-50 (SEQ ID | 33-37 |
| | | NO: 139) | |
| ACI-7079-3106C5-Ab1 | 3106C5B7 | 28-50 (SEQ ID | 33-37 |
| | | NO: 139) | |
| ACI-7079-3106F2-Ab1 | 3106F2C8 | 1-15 (SEQ ID | N.D. |
| | | NO: 121) | |
| ACI-7079-3107E6-Ab1 | 3107E6A3 | 28-50 (SEQ ID | 33-37 |
| | | NO: 139) | |
| ACI-7079-3108C10-Ab2 | 3108C10G6 | 28-50 (SEQ ID | 33-37 |
| | | NO: 139) | |
| ACI-7079-3108C10-Ab2 ** | 3108C10G6 | 100-114 (SEQ | N.D. |
| | | ID NO: 132) | |
| ACI-7079-3112H1-Ab1 | 3112H1F3 | 91-105 (SEQ ID | 99-101 |
| | | NO: 131) | |
| ACI-8031-6207G10-Ab1 | 6207G10E6 | 15-45 (SEQ ID | 30-40 |
| | | NO: 138) | |
| ACI-8032-6301A10-Ab2 | 6301A10E12 | 31-60 (SEQ ID | 33-39 |
| | | NO: 146) | |
| ACI-8032-6301C8-Ab2 | 6301C8G12 | 31-60 (SEQ ID | 33-39 |
| | | NO: 146) | |
| ACI-8032-6304F3-Ab1 | 6304F3D12 | 31-60 (SEQ ID | 33-39 |
| | | NO: 146) | |
| ACI-8032-6307F1-Ab2 | 6307F1H10 | 31-60 (SEQ ID | N.D. |
| | | NO: 146) | |
| ACI-8032-6314A3-Ab3 | 6314A3E4 | 31-60 (SEQ ID | 33-39 |
| | | NO: 146) | |
| ACI-8033-6401F2-Ab1 | 6401F2E3 | 81-120 (SEQ ID | 108-113 |
| | | NO: 137) | |
| ACI-8033-6402E2-Ab2 | 6402E2E4 | 81-120 (SEQ ID | 99-103 |
| | | NO: 137) | |
| ACI-8033-6402E10-Ab1 | 6402E10B3 | 81-120 (SEQ ID | 92-96 |
| | | NO: 137) | |
| ACI-8033-6403A4-Ab1 | 6403A4A3 | 81-120 (SEQ ID | 116-120 |
| | | NO: 137) | |
| ACI-8033-6403E11-Ab2 | 6403E11B4 | 81-120 (SEQ ID | 92-96 |
| | | NO: 137) | |
| ACI-8032-6313G2-Ab1 | 6313G2A10 | N.D. | N.D. |
| ACI-8030-6106F5-Ab1 | 6106F5B10 | N.D. | N.D. |
| ACI-8032-6301G2-Ab2 | 6301G2D11 | N.D. | N.D. |

N.D.: Not determined;
*according to revised data;
** data obtained from recombinantly produced antibodies.

### Inhibition or Delay of Seeded Alpha-Synuclein Aggregation

Monoclonal anti-alpha-synuclein antibodies were evaluated for their ability to inhibit the aggregation of alpha-synuclein in vitro. The presence of alpha-synuclein pre-formed aggregates (seeds) increases the de novo aggregation propensity of monomeric a-synuclein. Alpha-synuclein antibodies were incubated with alpha-synuclein seeds prior to adding the monomeric alpha-synuclein for the aggregation assay. Kinetics of alpha-synuclein aggregation were monitored by thioflavin T (ThT) fluorescence. The ability of alpha-synuclein antibodies to inhibit the seeded aggregation was quantified by a percent change in the aggregation half-time (time to reach half-maximum ThT fluorescence signal).

Alpha-synuclein recombinant protein (rPeptide, S-1001-4) at concentration of 5 mg/mL was re-suspended and dialyzed against DPBS (Slide-A-Lyzer Mini Dialysis 10K MWCO, ThermoScientific, 88404) four times of 60 minutes each at 4° C. Higher molecular weight species were then removed by centrifugal filtration (Microcon DNA Fast Flow Centrifugal Filter Unit with Ultracel membrane, Sigma, MRCFOR100). Sonicated alpha-synuclein fibrils were diluted with PBS to a final concentration of 1.0 mg/mL. Aggregations were assembled in low-binding 96-well plates (ThermoScientific, 278752), in triplicate for each condition. Alpha-synuclein seeds were used at 1% the final concentration of monomeric alpha-synuclein (14 μM).

Alpha-synuclein seeds (34.5 pmoles) were incubated with alpha-synuclein antibodies (787 pmoles, ˜22.8 equivalents) for 1 hour at at 25° C. As a reference control, alpha-synuclein seeds were incubated without the addition of alpha-synuclein antibodies. The Syn303 antibody (BioLegend, 824301) was used as a reference standard (Tran et al., Cell Rep. 2014, 7(6):2054-65). To control for any non-alpha-synuclein specific effect from the antibodies, the mouse isotype control (IgG2a) was produced recombinantly or purchased (ThermoFisher, 02-6200) and was used as a negative control.

Monomeric aSyn and ThT (3 mM stock solution, Sigma, D8537) were added to reach a final concentration of 14 μM and 46 μM respectively. Each aggregation was then aliquoted into 3 separate wells (65 μL/well) of the 96-well plates. Kinetic measurements were performed using an M200 Infinite Pro Microplate Reader (Tecan, Switzerland).

ThT fluorescent measurements were obtained in triplicate for each aggregation condition (technical repeats) and run twice on independent days (for a total of N=6). A baseline correction was performed by subtraction of the initial ThT value (t=0) and data was then normalized as a percent maximum ThT signal (see Equation 1). Aggregation half-times (τ½) were calculated from non-linear regressions using either a sigmoidal dose-response (see Equation 2) or a one-phase association (see Equation 3) (GraphPad Prism 7) and represent the time taken to reach half the maximum ThT signal.

$$\% ThT ( x ) = \frac{( ThT ( x ) ) - ( ThT ( {x}_{0} ) )}{( ThT ( {x}_{max} ) ) - ( ThT ( {x}_{0} ) )} * 100 \qquad Equation 1$$

Where % ThT(x) is the percent ThT signal at time t=x, ThT(x₀) is the ThT signal at t=0 and ThT(xₘₐₓ) is the maximum ThT signal.

$$\% ThT ( x ) = Bottom + \frac{( Top - Bottom )}{( 1 + 1 {0}^{( LogEC 50 - X ) - HillSlope} )} \qquad Equation 2$$

Where Bottom is a fit of the minimum ThT signal, Top is a fit of the maximum ThT signal, EC50 is the x value when the ThT signal is halfway between Bottom and Top, and the HillSlope is the steepness of the curve. Here, the aggregation half-time (τ_{1/2}) is obtained directly from EC50.

$$\% ThT ( x ) = ThT ( {x}_{0} ) + ( ( Plateau - ThT ( {x}_{0} ) ) * ( 1 - exp ( - K * x ) ) \qquad Equation 3$$

Where ThT(x₀) is the initial ThT signal, Plateau is the fit of the maximum ThT signal, and K is the rate constant. Here, the aggregation half-time (τ_{1/2}) is calculated from ln(2)/K.

$$\% Increase {τ}_{1 / 2} = \frac{{τ}_{m A b} - {τ}_{no mAb}}{{τ}_{no mAb}} * 100 \qquad Equation 4$$

Where τ_{no mab} is the aggregation half-time in the absence of antibody (mAb) and T_{mab} is the aggregation half-time in the presence of the indicated antibody.

$$Propagation of Error = \% {τ}_{m A b} * \sqrt{{( \frac{{SEM}_{mAb}}{{τ}_{mAb}} )}^{2} + {( \frac{{SEM}_{no mAb}}{{τ}_{no mAb}} )}^{2}} \qquad Equation 5$$

Where % τ_{mAb} is the percent increase in τ_{1/2} from Equation 4, τ_{no mab} is the aggregation half-time in the absence of mAb, T_{mab} is the aggregation half-time in the presence of the indicated mAb, and SEM is the standard error (calculations resulting from fitting of Equations 2 and 3).

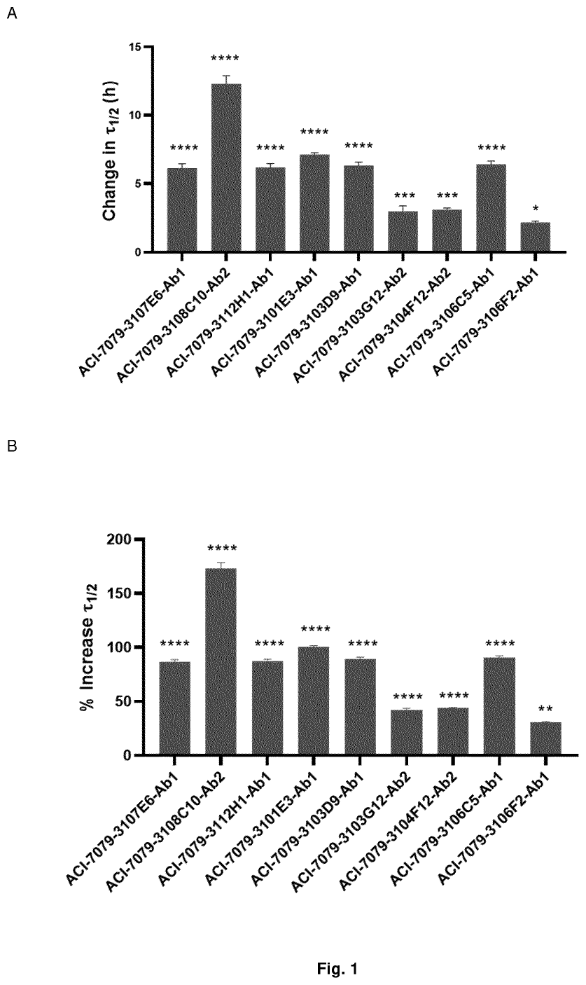

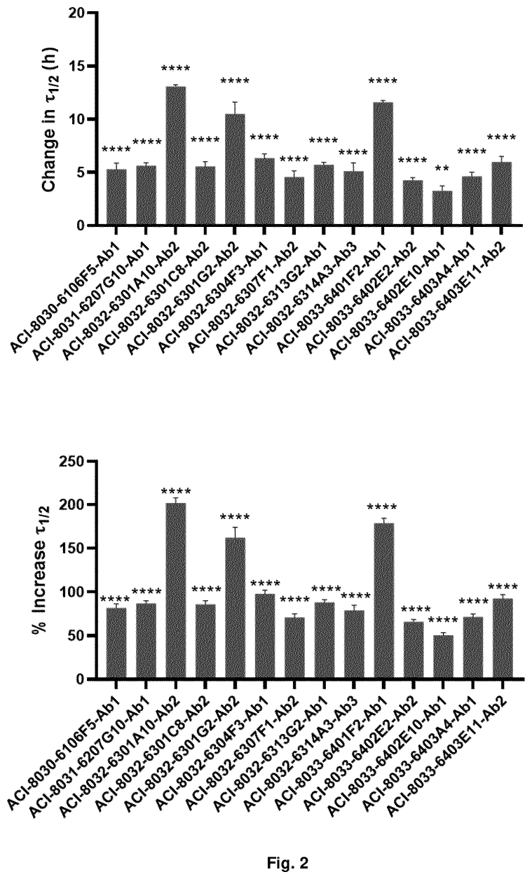

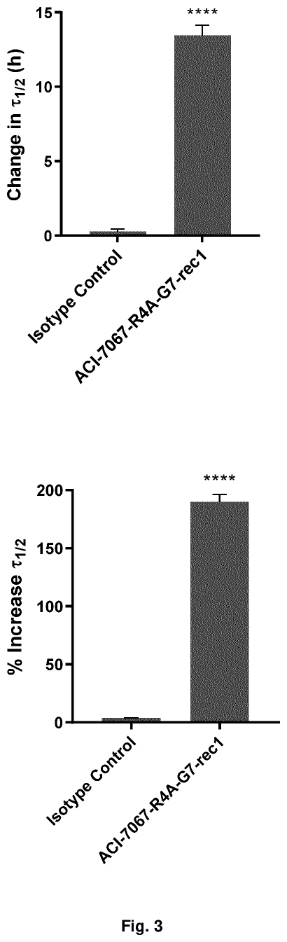

Aggregation half-times (τ_{1/2}) were obtained using either a sigmoidal fit (Equation 2) or an exponential fit (Equation 3) dependent upon the kinetic profile and best fit. Varied time frames were used to obtain optimal fitting as ThT signals can decrease following completion of aggregation. Change in τ_{1/2} values, in the presence of the indicated antibodies, were normalized relative to the τ_{1/2} value in the absence of antibody. FIG. 1A and FIG. 2A and FIG. 3A show the comparison of changes in τ_{1/2} values as normalized to the aggregation in the absence of antibody. Significant increases in τ_{1/2} values were observed for all antibodies proving the good efficacy of antibodies in delaying the seeded and/or spontaneous aggregation of alpha-synuclein. The percent increase in τ_{1/2} values were calculated relative to the seeded aggregation in the absence of antibody (see Equation 4). FIG. 1B and FIG. 2B and FIG. 3B show the calculated percent increase in τ_{1/2} values upon pre-incubation of alpha-synuclein seeds with the indicated antibodies proving the good efficacy of antibodies in delaying the seeded and/or spontaneous aggregation of alpha-synuclein. ACI-8032-6301A10-Ab2 demonstrated the largest increase in τ_{1/2} values, closely followed by ACI-8033-6401F2-Ab1, ACI-7079-3108C10-Ab2 and ACI-8032-6301G2-Ab2. Similar results were obtained with ACI-7067-4813-R4A-G7-rec1 (FIG. 3). Relative to the control condition, aggregation in the absence of antibody, pre-incubation of alpha-synuclein seeds with all antibodies of the present invention showed a significant percent increase in τ_{1/2} values.

### Affinity Measurements on Alpha-Synuclein Monomers and Alpha-Synuclein Fibrils by SPR

Affinity measurements were performed on an surface plasmon resonance (SPR) instrument (Biacore T200, GE Healthcare Life Sciences) using CM5 Series S sensor chips (GE Healthcare, BR-1005-30). Flow channels (Fc) 1-4 were activated with a fresh solution of EDC/NHS (Amine Coupling Kit, 1:1 ratio of both reagents, GE Healthcare, BR-1006-33). The goat anti-mouse antibody (GE Healthcare, BR-1008-38) was captured at a concentration of 30pg/mL diluted in 10 mM sodium acetate (pH 5.0). Following, all unreacted activated ester groups were capped with 1 M ethanolamine (GE Healthcare, BR-1006-33). Any non-covalently bound antibodies were removed by three successive regenerations of 10 mM Glycine pH 1.7 (GE Healthcare, 28-9950-84). Immobilization levels were evaluated following ethanolamine capping (Bound) and finally following regeneration (Final). Non-covalent immobilization of alpha-synuclein antibodies was performed using a target immobilization method of 2000 response units (RU). Antibodies were diluted in 10 mM sodium acetate pH 5.5 (GE Healthcare, BR-1003-52) to a final concentration of 5 μg/mL.

Binding affinity of alpha-synuclein antibodies to monomeric or fibrillar alpha-synuclein species was performed using a single-cycle kinetics method. The instrument was primed with 1×HBS-P+ buffer (10× stock from GE Healthcare, BR-1003-52 diluted in Milli-Q water). Injections of monomeric alpha-synuclein (aSyn) (Boston Biochem, SP-485), increasing in concentration from 0.62-50 nM prepared from serial 2-fold dilutions, were performed with contact times of 300 sec/injection at a flow rate of 30 μL/min. A dissociation phase of 900 sec followed the final 50 nM injection. Regeneration of the sensor to the goat anti-mouse antibody layer was achieved using 3 regenerations of 10 mM Glycine pH 1.7. Injections of alpha-synuclein fibrils of increasing concentration from 5.56-450 nM prepared from serial 2-fold dilutions, were performed with contact times of 300 sec/injection at a flow rate of 30 μL/min. A dissociation phase of 900 sec followed the final 450 nM injection. Regeneration of the sensor to the goat anti-mouse antibody layer was achieved using 3 regenerations of 10 mM Glycine pH 1.7. Results obtained from single-cycle kinetics were evaluated by Biacore T200 evaluation software with 1:1 binding homogenous Langmuir model or a heterogeneous ligand model (with a global Rmax) with Cycle 5 as a blank subtraction. The following kinetic parameters were obtained: on-rate (ka), off-rate (kd), affinity constant (KD, ratio of kd by ka), maximum response (Rmax), and goodness of fit (Chi2). Non-covalent capture of the alpha-synuclein antibodies was performed in three separate runs.

Capture levels ranged from ˜1800 to ˜2100 RU based on the target immobilization level of 2000 RU. The kinetic fitting parameters from single-cycle kinetics affinity measurements by SPR are shown in Table 5. ACI-7067-4813-R4A-G7-rec1, ACI-7079-3101 E3-Ab1, ACI-8032-6301A10-Ab2, ACI-7079-3106F2-Ab1, ACI-8033-6403A4-Ab1 demonstrate a binding preference to fibrillar alpha-synuclein and display significantly slower dissociation rates (kd) from fibrillar alpha-synuclein compared to monomeric alpha-synuclein. Moreover, ACI-7079-3108C10-Ab2 and ACI-8033-6401 F2-Ab1 selectively bind only to fibrillar alpha-synuclein.

TABLE 5
Affinity measurements obtained by SPR

| | | Alpha-synuclein monomers | | | Alpha-synuclein fibrils | | |
|---|---|---|---|---|---|---|---|
| Antibody | Hybridoma | | | KD | | | KD |
| Code | Code | ka (1/Ms) | kd (1/s) | (nM) | ka (1/Ms) | kd (1/s) | (nM) |
| ACI-7067- | 4813-R4A- | 7.35E+05 | 7.37E−02 | 97.0 | 4760.667 | 1.34E−07 | 0.041 |
| 4813-R4A- | G7 | | | | | | |
| G7-rec1 | | | | | | | |
| ACI-7079- | 3101E3C9 | 1.90E+05 | 5.69E−02 | 300 | 9.38E+03 | 1.29E−04 | 13.7 |
| 3101E3- | | | | | | | |
| Ab1 | | | | | | | |
| ACI-7079- | 3103D9C9 | 5.98E+05 | 4.37E−04 | 0.73 | 4.48E+04 | 4.13E−04 | 9.20 |
| 3103D9- | | | | | | | |
| Ab1 | | | | | | | |
| ACI-7079- | 3103G12D2B10 | 5.80E+05 | 1.40E−03 | 2.40 | 2.50E+04 | 5.63E−04 | 22.5 |
| 3103G12- | | | | | | | |
| Ab2 | | | | | | | |
| ACI-7079- | 3104F12F2H7 | 8.92E+05 | 4.52E−04 | 0.51 | 7.18E+04 | 2.68E−04 | 3.73 |
| 3104F12- | | | | | | | |
| Ab2 | | | | | | | |
| ACI-7079- | 3106C5B7 | 7.92E+05 | 3.25E−03 | 4.10 | 2.50E+04 | 2.37E−04 | 9.49 |
| 3106C5- | | | | | | | |
| Ab1 | | | | | | | |
| ACI-7079- | 3106F2C8 | 3.44E+05 | 1.35E−01 | 392 | 1.17E+04 | 3.67E−04 | 31.5 |
| 3106F2- | | | | | | | |
| Ab1 | | | | | | | |
| ACI-7079- | 3107E6A3 | 7.65E+05 | 8.59E−04 | 1.12 | 5.51E+04 | 1.46E−04 | 2.66 |
| 3107E6- | | | | | | | |
| Ab1 | | | | | | | |
| ACI-7079- | 3108C10G6 | Non- | Non- | Non- | 9.84E+03 | 6.66E−05 | 6.77 |
| 3108C10- | | binding | binding | binding | | | |
| Ab2 | | | | | | | |
| ACI-7079- | 3112H1F3 | 2.14E+05 | 2.25E−03 | 10.5 | 1.12E+04 | 2.16E−04 | 19.3 |
| 3112H1- | | | | | | | |
| Ab1 | | | | | | | |
| ACI-8030- | 6106F5B10 | 1.18E+05 | 2.30E−04 | 1.95 | 1.39E+04 | 2.32E−04 | 16.7 |
| 6106F5- | | | | | | | |
| Ab1 | | | | | | | |
| ACI-8031- | 6207G10E6 | 8.53E+04 | 3.14E−04 | 3.67 | 1.13E+04 | 1.50E−04 | 13.3 |
| 6207G10- | | | | | | | |
| Ab1 | | | | | | | |
| ACI-8032- | 6301A10E12 | 4.50E+05 | 5.17E−03 | 11.5 | 7.18E+03 | 1.93E−04 | 2.69 |
| 6301A10- | | | | | | | |
| Ab2 | | | | | | | |
| ACI-8032- | 6301C8G12 | 3.76E+05 | 3.29E−03 | 8.76 | 8.30E+04 | 3.55E−03 | 42.8 |
| 6301C8- | | | | | | | |
| Ab2 | | | | | | | |
| ACI-8032- | 6301G2D11 | 3.90E+02 | 2.37E−04 | 606 | 1.05E+02 | 3.53E−04 | 3350 |
| 6301G2- | | | | | | | |
| Ab2 | | | | | | | |
| ACI-8032- | 6304F3D12 | 3.37E+06 | 1.60E−02 | 4.75 | 1.30E+04 | 5.27E−04 | 40.6 |
| 6304F3- | | | | | | | |
| Ab1 | | | | | | | |
| ACI-8032- | 6307F1H10 | 1.30E+06 | 2.48E−02 | 19.0 | 2.65E+04 | 6.91E−04 | 26.0 |
| 6307F1- | | | | | | | |
| Ab2 | | | | | | | |
| ACI-8032- | 6313G2A10 | 3.35E+04 | 5.41E−04 | 16.2 | 3.38E+02 | 8.29E−04 | 2460 |
| 6313G2- | | | | | | | |
| Ab1 | | | | | | | |
| ACI-8032- | 6314A3E4 | 2.28E+06 | 3.82E−02 | 16.7 | 7.83E+03 | 3.25E−04 | 41.5 |
| 6314A3- | | | | | | | |
| Ab3 | | | | | | | |
| ACI-8033- | 6401F2E3 | Non- | Non- | Non- | 1.31E+04 | 1.60E−04 | 12.2 |
| 6401F2- | | binding | binding | binding | | | |
| Ab1 | | | | | | | |
| ACI-8033- | 6402E2E4 | 2.00E+05 | 6.51E−04 | 3.26 | 7.35E+03 | 9.87E−04 | 134 |
| 6402E2- | | | | | | | |
| Ab2 | | | | | | | |
| ACI-8033- | 6402E10B3 | 2.05E+04 | 5.80E−04 | 28.3 | 4.06E+03 | 1.52E−04 | 37.4 |
| 6402E10- | | | | | | | |
| Ab1 | | | | | | | |
| ACI-8033- | 6403A4A3 | 4.57E+05 | 4.40E−02 | 96.4 | 1.58E+04 | 5.99E−04 | 37.9 |
| 6403A4- | | | | | | | |
| Ab1 | | | | | | | |
| ACI-8033- | 6403E11B4 | 7.52E+04 | 2.93E−04 | 3.90 | 6.11E+03 | 2.73E−04 | 44.6 |
| 6403E11- | | | | | | | |
| Ab2 | | | | | | | |

### Antibody Variable Region Gene Sequencing

Clonal hybridoma cell lysates were used for variable region gene sequencing. Mouse hybridomas were harvested and lysed using a lysis buffer containing guanidinium salts that deactivates RNases. Genomic DNA was then eliminated by RNase-free DNase, and RNA was purified with a silica-based affinity column using multiple washes and eluted from the column using RNase-free water. Once the RNA was extracted, its purity and concentration was measured spectrophotometrically. The integrity of the RNA was assessed on a denaturing agarose gel and RNA was reverse transcribed into cDNA using reverse transcriptase (RT). Before adding the reaction mixture, the RNA was heated to 70° C. for 10 min in order to disrupt RNA secondary structures. The RT products were directly used for PCR amplification. For high-fidelity PCR amplification of the cDNA, each of the variable region primers corresponding to the different gene families encoding for antibodies were individually mixed with the constant primer, for variable heavy chain domain (VH) and variable light chain domain (VL) separately. In first intention, a degenerate primer pool was used (12 for VH and 12 for VL) and, depending on the results, a second pool was used to obtain PCR products. After the PCR reaction, the products were analyzed by gel electrophoresis on 2% agarose gels stained with ethidium bromide. The PCR products for VL and VH were individually purified on an agarose gel using tris-acetate-EDTA (TAE). The purified fragments excised from the gel were then sequenced using the dye-terminator sequencing method. The same primers as those used for PCR were used for the sequencing reaction. Sequencing was carried out in both directions to provide overlap at both ends. Sequencing data were analyzed on the Ig Blast/Kabat database. Nucleotide sequences for VH and VL are shown in Table 6. Protein sequences for VH and VL, and their complementarity-determining regions (CDRs) are shown in Table 7.

TABLE 6
Nucleotide sequence of the heavy chain and light chain variable domains (VH and VL)

| Antibody | Hybridoma | | |
|---|---|---|---|
| Code | Code | VH | VL |
| ACI-7079- | 3101E3C9 | CAGGTGCAGCTGAAGGAGTCAGGACCTGGCC | GATGTTTTGATGACCCAAACTCCACTCTCCCTGC |
| 3101E3- | | TGGTGGCGCCCTCACAGAGCCTGTCCATCACA | CTGTCAGTCTTGGAGATCAAGCCTCCATCTCTTG |
| Ab1 | | TGCACTGTCTCTGGCTTCTCATTAACCAGGTAT | CAGATCTAGTCAGACCATTGTACATAGTAATGGAA |
| | | ATTATAAATTGGGTTCGCCAGCCACCAGGAAA | ACACCTATTTAGAATGGTACCTGCAGAAACCAGG |
| | | GGGTCTGGAGTGGCTTGGAGTAATCTGGACTG | CCAGTCTCCAAAGCTCCTGATCTACAAAGTTTCCA |
| | | GTGGAGGCACAGATTATAATTCAGCTCTCAAAT | ACCGATTTTCTGGGGTCCCAGACAGGTTCAGTGG |
| | | CCAGACTGAGCATCAGCAAAGACAACTCCAAG | CAGTGGATCAGGGACAGATTTCACACTCAAGATC |
| | | AGTCAAGTTTTCTTAAAAATGAACAGTCTGCAA | AGCAGAGTGGAGGCTGAGGATCTGGGAGTTTATT |
| | | ACTGATGACACAGCCAGGTACTACTGTGCCAG | ACTGCTTTCAAGGTTCACATGTTCCGTACACGTTC |
| | | AGAGTATGGTTACGACGGGGCCTGGTTTGCTT | GGAGGGGGGACCAAGCTGGAAATAAAA (SEQ ID |
| | | ACTGGGGCCAAGGGACTCTTGTCACTGTCTCT | NO: 619) |
| | | GCA (SEQ ID NO: 618) | |
| ACI-7079- | 3103D9C9 | CAGGTTTCTCTGAAAGAGTCTGGCCCTGGGAT | GACATTGTGATGACACAGTCTCCATCCTCCCTGA |
| 3103D9- | | ATTGCAGCCCTCCCAGACCCTCAGTCTGACTT | CTATGTCACTAGGACAGACGGTCACTATGAGCTG |
| Ab1 | | GCTCTTTCTCTGGGTTTTCACTGAGCACTTTTG | CAAGTCCAGTCAGAGCCTTTTAAATAGTAGCAATC |
| | | GTATGGGTGTAGGCTGGATTCGTCAGCCTTCA | AAAAGAACCATCTGGCCTGGTACCAGCAGAGACC |
| | | GGGAAGGGTCTGGACTGGCTGACACACATTTG | AGGACAGTCTCCCAAACTTCTGGTATACTTTGCAT |
| | | GTGGGATGATGATAAGTAC | CCACTAGGGAATCTGGGGTCCCTGAGCGCTTCAT |
| | | TATAATCCAGGCCTGAAGAGTCGCCTCACAAT | AGGCGGTGGATCTGGGACAGATTTCACTCTTACC |
| | | CTCCAAGGATACCTCCAAAAACCAGGTATTCC | ATCAGCAATGTGCAGTCTGAAGACCTGACAGATT |
| | | TCGAGATCGCCAATGTGGACACTGCAGATACT | ACTTCTGTCAGCAACATTATAGCACTCCGTACACG |
| | | GCCACATATTACTGTGCGCGAATAGGAGATTA | TTCGGAGGGGGGACCAAGCTGGAAATAAAA (SEQ |
| | | CTACGGTACTAGAGGGTACTTCGATGTCTGGG | ID NO: 629) |
| | | GCACAGGGACCACGGTCACCGTCTCCTCA | |
| | | (SEQ ID NO: 628) | |
| ACI-7079- | 3103G12D | GATGTGCAGCTTCAGGAGTCAGGACCTGGCAT | GATGTTTTGATGACCCAAGCTCCACTCTCCCTGC |
| 3103G12- | 2B10 | GGTGAAACCTTCTCAGTCACTTTCCCTCACCT | CTGTCAGTCTTGGAGATCAAGCCTCCATCTCGTG |
| Ab2 | | GCACTGTCACTGGCTACTCCATCACCAGTGGT | CAGATCTAGTCAGAGCATTGTACATAGTAATGGA |
| | | TATGACTGGCACTGGATCCGACATTTTCCAGG | GACACCTATTTAGAATGGTACCTGCAGAAACCAG |
| | | AAACAAACTGGAGTGGATGGGCTACATAACCT | GCCAGTCTCCAAAGCTCCTGATCTACAAAGTTTC |
| | | ACAGTGGTAGCACTAACTACAACCCATCCCTC | CAACCGATTTTCTGGGGTCCCAGACAGGTTCAGT |
| | | AAAAGTCGAATCTCCATCACTCATGACACATCT | GGCAGTGGATCAGGGACAGATTTCACACTCAAGA |
| | | AAGAACCATTTCTTCCTGAAGTTGACTTCTGTG | TCAGCAGAGTGGAGGCTGAGGATCTGGGAGTTTA |
| | | ACTACTGAGGACACAGCCACATATTACTGTGC | TTACTGCTTTCAAGGTTCACATGTTCCTCCGACGT |
| | | AAGAGACACGGTAGACGCCTGGTTTGCTTACT | TCGGTGGAGGCACCAAGCTGGAAATCAAA (SEQ |
| | | GGGGCCAGGGGACTCTGGTCACTGTCTCTGC | ID NO: 639) |
| | | A (SEQ ID NO: 638) | |
| ACI-7079- | 3104F12F2 | CAGGTTACTCTGAAAGAGTCTGGCCCTGGAAT | GACATTGTGATGACACAGTCTCCATCCTCCCTGG |
| 3104F12- | H7 | AGTGCAGCCCTCCCAGACCCTCAGTCTGACTT | CTATGTCAGTTGGACAGAAGGTCACTTTGAGGTG |
| Ab2 | | GCTCTTTCTCTGGATTTTCACTGAACACTTTTG | CAAGTCCAGTCAGAGCCTTTTAAATAGTAGTAATC |
| | | GTTTGGGTGTAGGCTGGATTCGTCAGCCTTCA | AAAAGAACCATTTGGCCTGGTACCAGCAGAAACC |
| | | GGGAAGGGTCTGGACTGGCTGACACACATTTG | AGGACAGTCTCCTAAACTTCTGCTATGCTTTGCAT |
| | | GTGGGATGATGATAAGTACTATAATCCAGCCC | CCACTAGGGAATCTGGGGTCCCTGATCGCTTCAT |
| | | TGAAGAGTCGGCTCACAATCTCCAAGGATACC | AGGCAGTGGATCTGGGACAGATTTCACTCTTACC |
| | | TCCAAAAACCAGGTATTCCTCAAGATCGCCAAT | ATCAGTAGTGTGCAGGCTGAAGACCTGGCAGATT |
| | | GTGGACACTGCAGATACTGCCACATACTACTG | ACTTCTGTCAGCAACATTATAGCACTCCGTACACG |
| | | TGCTCGAATAGGCGATTTCTACGGTAGTAGAG | TTCGGAGGGGGGACCAAGCTGGAAATAAAA (SEQ |
| | | GATATTTCGATGTCTGGGGCACAGGGACCACG | ID NO: 649) |
| | | GTCACCGTCTCCTCA (SEQ ID NO: 648) | |
| ACI-7079- | 3106C5B7 | CAGGTTACTCTGAAAGAGTCTGGCCCTGGGAT | GACATTGTGATGACACAGTCTCCATCCTCCCTGG |
| 3106C5- | | ATTGCAGCCCTCCCAGACCCTCAGTCTGACTT | CTATGTCAGTAGGACAGAAGGTCACTATGAGCTG |
| Ab1 | | GTTCTTTCTCTGGGTTTTCACTGAGTACTTTTG | CAAGTCCAGGCAGAGCCTTTTAAATAGTAGTAATC |
| | | GTATGGGTGTAGGCTGGATTCGTCAGCCTTCA | AAAAGAATCATTTGGCCTGGTACCAGCAGAAACC |
| | | GGGAAGGGTCTGGAGTGGCTAACACACATTTG | AGGACAGTCTCCTAAACTTCTGGTATACTTTGCAT |
| | | GTGGGATGATGATAAGTACTATAACCCAGCCC | CCACTAGGGAATCTGGGGTCCCTGATCGCTTCAT |
| | | TGAAGAGTCGGCTCACAATCTCCAGGGATCCC | AGGCAGTGGATCTGGGACAGATTTCACTCTTACC |
| | | TCCAAAAACCAGGTATTCTCAAGATCGCCAAT | ATCAGCAGTGTGCAGGCTGAAGACCTGGCAGATT |
| | | GTGGACACTGCAGATACTGCCACATACTACTG | ACTTCTGTCAGCAACATTATAGCACTCCGTACACG |
| | | TGCTCGAATAGGGGATTACTACGGTAGTAGAG | TTCGGGGGGGGGACCAAGCTGGAAATAAAA |
| | | GATACTTCGATGTCTGGGGCACAGGGACCAC | (SEQ ID NO: 659) |
| | | GGTCACCGTCTCCTCA (SEQ ID NO: 658) | |
| ACI-7079- | 3106F2C8 | GAGGTTCAGCTGCAGCAGTCTGGGGCTGAGC | GACATTGTGATGACCCAGTCTCAAAAATTCATGTC |
| 3106F2- | | TTGTGAGGCCAGGGGCCTCAGTCAAGTTGTCC | CACATCAGTAGGAGACAGGGTCAGCGTCACCTG |
| Ab1 | | TGCACAGCTTCTGGCTTTAACGTTAAAGACGA | CAAGGCCAGTCAGAATGTGGGAACTAATGTAGCC |
| | | CTATATGCACTGGGTGAAGCAGAGGCCTGAAC | TGGTATCAACAGAAAGTAGGACAATCTCCTAAAG |
| | | AGGGCCTGGAGTGGATTGGATGGATTGATCCT | CACTGATTTACTCGGCATCCTACCGGTACAGTGG |
| | | GAGAATGGTGATACTGAATATGCCTCGAAGTT | AGTCCCTGATCGCTTCACAGGCAGTGGATCTGGG |
| | | CCAGGGCAAGGCCACTTTAACAGCAGACACAT | ACAGATTTCACTCTCACCATCAGCAATGTGCAGTC |
| | | CCTCCAACACAGCCTACCTGCAGCTCAGCGGC | TGAAGACTTGGCAGACTATTTCTGTCAGCAATATA |
| | | CTGACATCTGTGGACACTGCCGTCTATTACTG | ACAGCTATCCTCTCACGTTCGGCTCGGGGACAAA |
| | | TACTACAGCCGGTACTAGCCCGTACTACTTTG | GTTGGAAATAAAA (SEQ ID NO: 669) |
| | | ACTACTGGGGCCAAGGCACCACTCTCACAGTC | |
| | | TCCTCC (SEQ ID NO: 668) | |
| ACI-7079- | 3112H1F3 | GAGGTCCAGCTGCAACAATCTGGACCTGAACT | GACATTGTGATGTCACAGTCTCCATCCTCCCTGG |
| 3112H1- | | GGTGAAGCCTGGGGCTTCAGTGAAGATATCCT | CTGTGTCAGCAGGAGAGAAGGTCACTATGAGCTG |
| Ab1 | | GTAAGGCTTCTGGATACACGTTCAGTGACTAC | CAAATCCAGTCAGAGTCTGCTCAACAGTAGAACC |
| | | TTCATGAACTGGGTGAAGCAGAGTCATGGAAA | CGGAAGAACTACTTGGCTTGGTACCAGCAGAAAC |
| | | AAGCCTTGAGTGGATTGGAGATATTAATCCTAA | CAGGACAGTCTCCTAAACTGCTGATCTACTGGGC |
| | | CAATGGTGGTTCTAGCTACATCCAGAAGTTCA | ATCCACTAGGGAATCTGGGGTCCCTGATCGCTTC |
| | | AGGGCAAGGCCACATTGACTGTAGACAAGTCC | ACAGGCAGTGGATCTGGGACAGATTTCACTCTCA |
| | | TCCACCACAGCCTATATGGAGCTCCGCAGCCT | CCATCAGCAGTGTGCAGGCTGAAGACCTGGCAG |
| | | GACATCTGAGGACTCAGCAGTCTATTACTGTG | TTTATTACTGCAAGCAATCTTATGATCTGTGGACG |
| | | CAAGAGGTAGCAACTATGCTATGGACTCCTGG | TTCGGTGGGGGCACCAAGCTGGAAATCAAA (SEQ |
| | | GGTCAGGGAACCTCAGTCACCGTCTCCTCA | ID NO: 679) |
| | | (SEQ ID NO: 678) | |
| ACI-7079- | 3107E6A3 | CAGGTTACTCTGAAAGAGTCTGGCCCTGGGAT | GACATTGTGATGACACAGTCTCCATCCTCCCTGG |
| 3107E6- | | ATTGCAGCCCTCCCAGACCCTCAGTCTGACTT | CCATGTCAGTAGGACAGAAGGTCACTATGAGCTG |
| Ab1 | | GTTCTTTCTCTGGGTTTTCACTGAGCACTTTTG | CAAGTCCAGTCAGAGCCTTTTAAATAGTAGCAATC |
| | | GTATGGGTGTAGGCTGGATTCGTCAGCCTTCA | AAAAGAACCATTTGGCCTGGTATCAGCAGAAACC |
| | | GGGAAGGGTCTGGAGTGGCTGACACACATTT | AGGACAGTCTCCTAAACTTCTGGTATACTTTGCAT |
| | | GGTGGGATGATGATAAATACTATAACCCAGCC | CCACTAGGGACTCTGGGGTCCCTGATCGCTTCAT |
| | | CTTAAGAGTCGGCTCACAATCTCCAAGGATGC | AGGCAGTGGATCTGGGACAGATTTCACTCTTACC |
| | | CTCCAAAAACCAGATGTTCCTCAAGATCGCCA | ATCAGCAGTGTGCAGGCTGAGGACCTGGCAGATT |
| | | ATGTGGACACTGCAGATACTGCCACATACTTC | ACTTCTGTCAGCAACATTATAGCACTCCGTACACG |
| | | TGTGCTCGAATAGGGGATTACTACGGTAGTAG | TTCGGAGGGGGGACCAAGCTGGAAATAAAA (SEQ |
| | | AGGGTTCTTCGATGTCTGGGGCACAGGGACC | ID NO: 689) |
| | | ACGGTCACCGTCTCCTCA (SEQ ID | |
| | | NO: 688) | |
| ACI-7079- | 3108C10G | GAACTGCAGCTGGTGGAGTCTGGGGGAGGCT | GATGTTGTGATGACCCAGACTCCACGCACTTTGT |
| 3108C10- | 6 | TAGTGAGGCCTGGAGGGTCCCTGAAACTCTCC | CGGTAATCATTGGACAACCAGCCTCCATCTCTTG |
| Ab2 | | TGTGCAACCTCTGGATTCACTTTCAGAAACTAT | CAAGTCAAGTCAGAGCCTCTTAGATAGTGATGGA |
| | | GCCATGTCTTGGGTTCGCCAGACTCCGGCAAA | AAGACATACTTGAGTTGGTTGTTACAGAGGCCAG |
| | | GAGGCTGGAGTGGGTCGCAACCATTAGTGAT | GCCAGTCTCCAAAGCGCCTAATCTATCTGGTGTC |
| | | GGTGGTACTTACACCTTCTATTCAGACAATGTA | TAAAGTGGACTCTGGAGTCCCTGACAGGTTCACT |
| | | AAGGGCCGATTCACCATCTCCAGAGACAATGT | GGCAGTGGATCAGGGACAGATTTCACACTGAAAA |
| | | CAAGAACACCTTGTACCTTCAAATGAGCCATCT | TCAGCAGAGTGGAGGCTGAGGATTTGGGAGTTTA |
| | | GAAGTCTGAGGACACAGCCATGTATTACTGTT | TTATTGCTGGCAAGGTACATATTTTCCTCGGACGT |
| | | CAAGAGCCGGCTCTGGCCACTGGGGCCAAGG | TCGGTGGAGGCACCAAGCTGGAAATCAAA (SEQ |
| | | CACCACTCTCACAGTCTCCTCA (SEQ ID NO: | ID NO: 699) |
| | | 698) | |
| ACI-8030- | 6106F5B10 | CAGGTCCAGCTGCAGCAGTCTGGACCTGAACT | GACATTGTGCTGACCCAATCTCCAGCTTCTTTGG |
| 6106F5- | | GGTGAAGCCTGGGGCTTCAGTGAAGATATCCT | CTGTGTCTCTAGGGCAGAGGGCCACCATCTCCTG |
| Ab1 | | GCAAGGCTTCTGGCTACAGCTTCACAACCTAC | CAAGGCCAGCCAAAGTGTTGATTATGGTGGTGAT |
| | | TATATACACTGGATGAAGCAGAGGCCTGGACA | AGTTGGCTGAACTGGTACCAACAGAAACCAGGAC |
| | | GGGACCTGAGTGGATTGGATGGATTTATCCTG | AGCCACCCAAACTCCTCATCTATGCTGCATCCAAT |
| | | GAAGGGGTTATACTAAGTACAATGAGAAGTTC | CTAGATTCTGGGATCCCAGCCAGGTTTGGTGGCA |
| | | AAGGACAAGGCCGCACAGACGGCAGACACAT | GTGGGTCTGGGACAGACTTCACCCTCAACATCCA |
| | | CCTCCAACACTGCCTACATGCAGCTCAGCAGC | TCCTGTGGAGGAGGAGGATGCTGCAACCTATTAC |
| | | CTAAGATCTGAGGACTCTGCGGTCTATTACTG | TGTCAACAAAGTGGAGAGGATCCGTTCACGTTCG |
| | | TGCAAGAGACTGGGAAACCGGATTTCCTTACT | GAGGGGGGACCAATCTGGAAATAAAA (SEQ ID |
| | | GGGGCCAAGGTACTCTGGTCACTGTCTCTGCA | NO: 709) |
| | | (SEQ ID NO: 708) | |
| ACI-8031- | 6207G10E6 | CAGGTTCAGCTGCAGCAGTCTGGGGCTGAGC | GACATCTTGCTGACTCAGTCTCCAGCCATCCTGT |
| 6207G10- | | TGGTGAAGCCTGGGGCCTCAGTGAAGATTTCC | CTGTGAGTCCAGGAGATAGAGTCAGTTTCTCCTG |
| Ab1 | | TGCAAAGCTTCTGGCTACGCATTCAGTAGCTA | CAGGGCCAGTCAGAGCATTGGCACAAGGATACA |
| | | CTGGATGAACTGGGTGAAGCAGAGGCCTGGA | CTGGTATCAGCAAAGAACAAATGGTTCTCCAAGG |
| | | AAGGGTCTTGAGTGGATTGGACAGATTTATCC | CTTCTCATAAAGTATGCTTCTGAGTCTATCTCTGG |
| | | TGGAGATGGTGATACTAACTACAACGGAAAGT | GACCCCTTCCAGGTTTAGTGGCAGTGGATCAGGG |
| | | TCAAGGGCAAGGCCACACTGACTGCAGACAG | ACAGATTTTACTCTTAGCATCAACAGTGTGGAGTC |
| | | ATCCTCCAGCACAGCCTACATGCAGCTCAGCA | TGAAGATATTGCAGATTATTACTGTCAACAAAGTA |
| | | GCCTGACCTCTGAGGACTCTGCGGTCTATTTC | ATAGTTGGCCACTCACGTTCGGTGCTGGGACCAA |
| | | TGTGCAATTACGCACTATGACTACTGGGGCCA | GCTGGAGCTGAAA (SEQ ID NO: 719) |
| | | AGGCACCACTCTCACAGTCTCCTCA (SEQ ID | |
| | | NO: 718) | |
| ACI-8032- | 6301A10E1 | CAGGTTCAGCTGCAGCAGTCTGGAGCTGAACT | GATGTTTTGATGACCCAAACTCCACTCTCCCTGC |
| 6301A10- | 2 | GGCGAGGCCTGGGGCTTCAGTGAAGCTGTCC | CTGTCAGTCTTGGAGATCAAGCCTCCATCTCTTG |
| Ab2 | | TGCAAGGCTTCTGGCTACACCTTCACAAGCTA | CAGATCTAGTCACAGCATTGAACATAGTAATGGAA |
| | | TGGTATCAGCTGGGTGAAGCAGAGAACTGGAC | ACACCTATTTAGAATGGTGCCTGCAGAAACCAGG |
| | | AGGGCCTTGAGTGGATTGGAGAGATTTATCCT | CCAGCCTCCAAAGTTCCTGATCTACAAAGTTTCCA |
| | | AGAAGTGGTAATACTTACTACAATGAGAAGTTC | ACCGATTTTCTGGGGTCCCAGACAGGTTCAGTGG |
| | | AAGGGCAAGGCCACACTGACTGCAGACAAATC | CAGTGGATCAGGGACAGATTTCACACTCAGGATC |
| | | CTCCAGCACAGCGTACATGGAGCTCCGCAGC | AGTAGAGTGGAGGCTGAGGATCTGGGAGTTTATT |
| | | CTGACATCTGAGGACTCTGCGGTCTATTTCTG | ACTGCTTTCAAGGTTCACATGTTCCTCCGACGTTC |
| | | TGCAAGTGAGAGCTACTATGGTGACTACGACT | GGTGGAGGCACCAAGCTGGAAATCAAA (SEQ ID |
| | | GGTACTTCGATGTCTGGGGCACTGGGACCAC | NO: 729) |
| | | GGTCACCGTCTCCTCA (SEQ ID NO: 728) | |
| ACI-8032- | 6301C8G12 | CAGGTTCAGCTGCAGCAGTCTGGAGCTGAACT | GATGTTTTGATGACCCAAATTCCACTCTCCCTGCC |
| 6301C8- | | GGCGAGGCCTGGGACTTCAGTGAAGCTGTCC | TGTCAGTCTTGGAGATCAAGCCTCCATCTCTTGC |
| Ab2 | | TGCAAGGCTTCTGGCTACACCTTCACAACCTA | AGATCTAGTCAGAGCATTGTACATAGTAATGGAAA |
| | | TGGTATAAGCTGGGTGAAGCAGAGAACTGGAC | CACCTATTTAGAATGGCACCTGCAGAAACCAGGC |
| | | AGGGCCTTGAGTGGATTGGAGAGATTTATCCT | CAGTCTCCAAAGCTCCTGATCTACGAAGTTTCCAA |
| | | AGAAGTGGTAATACTTACTACAATGAGAAATTC | CCGATTTTCTGGGGTCCCAGACAGGTTCAGTGGC |
| | | AAGGGCAAGGCCACACTGACTGCAGACAAGC | AGTGGATCAGGGACAGATTTCACACTCAATATCA |
| | | CCTCCAGCACAGCGTACATGGAGCTCCGCAG | GCAGAGTGGAGGCTGAGGATCTGGGAGTTTATTA |
| | | CCTGACATCTGAGGACTCTGCGGTCTATTTCT | CTGCTTTCAAGGTTCACATGTTCCTCCGACGTTC |
| | | GTGCAAGTGAGAGTTACTATGGTGACTACGCC | GGTGGAGGCACCAAGCTGGAAATCAAA (SEQ ID |
| | | TGGTACTTCGATGTCTGGGGCACAGGGACCAC | NO: 739) |
| | | GGTCACCGTCTCCTCA (SEQ ID NO: 738) | |
| ACI-8032- | 6301G2D11 | GAGGTCCAACTGCAACAATCTGGACCTGAGCT | GACATTGTGATGTCACAGTCTCCATCCTCCCTGG |
| 6301G2- | | GGTGAAGCCTGGGGCTTCAGTGAAGATATCCT | CTGTGTCAGCAGGAGAGAAGGTCACTATGAGCTG |
| Ab2 | | GTAAGGCTTCTGGATACACGTTCACTGACTAC | CAAATCCAGTCAGAGTCTGCTCAACAGTAGAACC |
| | | TTCATGAACTGGGTGAAGCAGAGCCATGGAAA | CGAAAGAACTACTTGGCTTGGTACCAGCAGAAAC |
| | | GAGCCTTGAGTGGATTGGAGATATTAATCCTA | CAGGGCAGTCTCCTAAACTGCTGATCTACTGGGC |
| | | ACATTGGTGTTACTAACTACAACCCGAAGTTCA | ATCCACTAGGGAATCTGGGGTCCCTGATCGCTTC |
| | | AGGACAGGGCCACATTGACTGTAGACAAGTCC | ACAGGCAGTGGATCTGGGACAGATTTCACTCTCA |
| | | TCCAGCTCAGCCTACATGGAGCTCCGCAGCCT | CCATCAGCAGTGTGCAGGCTGAAGACCTGGCAG |
| | | GACATCTGAGGACTCTACAGTCTATTACTGTG | TTTATTTCTGCAAGCAATCTTATGATCTGTGGACG |
| | | CAAGAGGGGGAGGGAGTAATCCCCTTTACTAT | TTCGGTGGAGGCACCAAGCTGGAAATCAAA (SEQ |
| | | GCTATGGACTACTGGGGTCAAGGAACCTCAGT | ID NO: 749) |
| | | CACCGTCTCCTCA (SEQ ID NO: 748) | |
| ACI-8032- | 6304F3D12 | CAGGTTCAGCTGCAGCAGTCTGGAGCTGAGCT | GATGTTTTGATGACCCAAATTCCACTCTCCCTGCC |
| 6304F3- | | GGCGAGACCTGGGGCTTCAGTGAAGCTGTCC | TGTCAGTCCTGGAGATCAAGTTTCCATCTCTTGCA |
| Ab1 | | TGCAAGGCTTCTGGCTACACCTTCACAAGCTA | GATCTAGTCAGAGCATTGTACATAGTAATGGAAAC |
| | | TGGTGTAAGCTGGGTGAAGCAGAGAACTGGA | ACCTATTTAGAATGGTACCTGCAGAAACCAGGCC |
| | | CAGGGCCTTGAGTGGATTGGAGAGATTTATCC | AGTCTCCAAAGCCCCTGATCTACAAAGTTTCCAAC |
| | | TAGAAGTGGTAATACTTACTACAATGAGAAGTT | CGATTTTCTGGGGTCCCAGACAGGTTCAGTGGCA |
| | | CAAGGGCAAGGCCACACTGACTGCAGACAAAT | GTGGATCAGGGACAGATTTCACACTCAAGATCAG |
| | | CCTCCAGCACAGCGTACATGGAGCTCCGCAG | CAGAGTGGAGGCTGAGGATCTGGGAGTTTATTAC |
| | | CCTGACATCTGAGGACTCTGCGGTCTATTTCT | TGCTTTCAAGGTTCACATGTTCCTCCGACGTTCG |
| | | GTGCAAGTGAGAGCTACTATGGTGACTACGAC | GTGGAGGCACCAAGCTGGAAATCAAA (SEQ ID |
| | | TGGTATTTCGATGTCTGGGGCACAGGGACCAC | NO: 759) |
| | | GGTCACCGTCTCCTCA (SEQ ID NO: 758) | |
| ACI-8032- | 6307F1H10 | CAGGTTCAGCTGCAGCAGTCTGGAGCTGAGCT | GATGTTTTGATGACCCAAACTCCACTCTCCCTGC |
| 6307F1- | | GGCGAGGCCTGGGGCTTCAGTGAAGCTGTCC | CTGTCAGTCTTGGAGATCAAGCCTCCATCTCTTG |
| Ab2 | | TGCAAGGCTTCTGGCTACACCTTCACAAGCTA | CAGATCTAGTCAGAGCGTTGTACATAGTAATGGA |
| | | TGGTATGAGTTGGGTGAAACAGAGAACTGGAC | ATCACCTATTTAGAATGGTACCTGCAGAAACCAG |
| | | AGGGCCTTGAGTGGATTGGAGAGATTTATCCT | GCCAGTCTCCAAAGCTCCTGATCTACAAAGTTTC |
| | | AGAAGTGGTAATACTTACTACAATGAGAAGTTC | CAACCGATTTTCTGGGGTCCCAGACAGGTTCAGT |
| | | AAGGGCAAGGCCACACTGACTTCAGACAAATC | GGCAGTGGATCAGGGACATATTTCACACTCAAGA |
| | | CTCCAGCACATCATACATGGAGCTCCGCAGCC | TCAGCAGAGTGGAGGCTGAGGATCTTGGAGTTTA |
| | | TGACATCTGAGGACTCTGCGGTCTATTTCTGT | TTACTGCTTTCAAGGTTCACATGTTCCTCCGACGT |
| | | GCAAGTGAGAGCTACTATGGTGACTACGCCTG | TCGGTGGAGGCACCAAGCTGGAAATCAAA (SEQ |
| | | GTACTTCGATGTCTGGGGCACAGGGACCACG | ID NO: 769) |
| | | GTCACCGTCTCCTCA (SEQ ID NO: 768) | |
| ACI-8032- | 6313G2A10 | GAGGTCCAGCTGCAACAATCTGGACCTGAGCT | GACATTGTGATGTCACAGTCGCCATCCTCCCTGG |
| 6313G2- | | GGTGAAGCCTGGGGCTTCAGTGAAGATATCCT | CTGTGTCAGCAGGAGAGAAGGTCACTATGAACTG |
| Ab1 | | GTAAGGCTTCTGGATACACGTTCACTGACTAC | CAAATCCAGTCAGAGTCTGCTCAACAGTAGAACC |
| | | TACATGAACTGGGTGAAGCAGAGCCATGGAAA | CGAAAGAACTACTTGGCTTGGTACCAGCAGAAAC |
| | | GAGCCTTGAGTGGATTGGAGATATTGATCCTA | CAGGGCAGTCTCCAAAACTACTGATCTACTGGGC |
| | | ACAATGGTGTTACTAGTTACAGCCAGAAGTTCA | ATCCACTAGGGAATCTGGGGTCCCTGATCGCTTC |
| | | AGGGCAAGGCCACATTGACTGTAGACAAGTCC | ATAGGCAGTGGATCTGGGACAGGTTTCACTCTCA |
| | | TCCAGCACAGCCTACATGGAGCTCCGCAGCCT | CCATTAGCAGTGTGCAGGCTGAAGACCTGGCAGT |
| | | GACATCTGAAGACTCTGCAGTCTATTACTGTG | TTATTACTGCAAGCAATCTTATGATCTGTGGACGT |
| | | CAAGGACGGGGTACTACGGTACTTCTCTATAC | TCGGTGGAGGCACCAAGCTGGAAATCAAA (SEQ |
| | | TATGCTATGGACTACTGGGGTCAAGGAACCTC | ID NO: 779) |
| | | AGTCACCGTCTCCTCA (SEQ ID NO: 778) | |
| ACI-8032- | 6314A3E4 | CAGGTTCAGCTGCAGCAGTCTGGAGCTGAGCT | GATGTTTTGATGACCCAAATTCCACTCTCCCTGCC |
| 6314A3- | | GGCGAGACCTGGGGCTTCAGTGAAGCTGTCC | TGTCAGTCCTGGAGATCAAGTTTTCATCTCTTGCA |
| Ab3 | | TGCAAGGCTTCTGGCTACACCTTCACAAGCTA | GATCTAGTCAGAGCATTGTACATAGTAATGGAAAC |
| | | TGGTGTAAGCTGGGTGAAGCAGAGAACTGGA | ACCTATTTAGAATGGTACCTGCAGAAACCAGGCC |
| | | CAGGGCCTTGAGTGGATTGGAGAGATTTATCC | AGTCTCCAAAGCCCCTGATCTACAAAGTTTCCAAC |
| | | TAGAAGTGGTAATACTTACTACAATGAGAAGTT | CGATTTTCTGGGGTCCCAGACAGGTTCAGTGGCA |
| | | CAAGGGCAAGGCCACACTGACTGCAGACAAAT | GTGGATCAGGGACAGATTTCACACTCAAGATCAG |
| | | CCTCCAGCACAGCGTACATGGAGCTCCGCAG | CAGAGTGGAGGCTGAGGATCTGGGAGTTTATTAC |
| | | CCTGACATCTGAGGACTCTGCGGTCTATTTCT | TGCTTTCAAGGTTCACATGTTCCTCCGACGTTCG |
| | | GTGCAAGTGAGAGCTACTATGGTGACTACGAC | GTGGAGGCACCAAGCTGGAAATCAAA (SEQ ID |
| | | TGGTATTTCGATGTCTGGGGCACAGGGACCAC | NO: 789) |
| | | GGTCACCGTCTCCTCA (SEQ ID NO: 758) | |
| ACI-8033- | 6401F2E3 | CAGGTCCAACTGCAGCAGCCTGGGGCTGAGC | GATGTTTTGATGACCCAAACTCCACTCTCCCTGC |
| 6401F2- | | TTGTGAAGCCTGGGGCTTCAGTGAAGATGTCC | CTGTCAGTCTTGGAGATCAAGCCTCCATCTCTTG |
| Ab1 | | TGCAAGGCTTCTGGCTACACCTTCACCAACTA | CAGATCTAGTCAGAGTGTTGTGCATGTTAATGGA |
| | | CTGGATTACCTGGGTAAAACAGAGGCCTGGAC | AACACCTATTTAGATTGGTACCTGCAGAAACCAG |
| | | AAGGCCTTGAGTGGATTGGGGATATTTATCCT | GCCAGTCTCCAAAGCTCCTGATCTACAAAGTTTC |
| | | GGTAATGGTGATGTTGACTACAGTGAGATCTT | CAACCGATTTTCTGGGGTCCCAGACAGGTTCAGT |
| | | CAAGAATAAGGCCAAAATGACTGTAGACACAT | GGCAGTGGATCAGGGACAGATTTCACGCTCAGG |
| | | CCTCCACCACAGCCTACATGCAGCTCAGCAGC | ATCACCAGAGTGGAGGCTGAGGATCTGGGAATTT |
| | | CTGACATCTGAGGACTCTGCGGTCTATCACTG | ATTACTGTTTTCAAGGTTCACATGTTCCTCGGACG |
| | | TGTCTTGGGTCAGACTTCCTTTGGTCATTGGG | TTCGGTGGAGGCACCAAGCTGGAAATCAAA (SEQ |
| | | GCCAAGGGACTCTGGTCACTGTCTCTGCA | ID NO: 799) |
| | | (SEQ ID NO: 798) | |
| ACI-8033- | 6402E2E4 | GAGGTCCAGCTGCAACATTCTGGACCTGACCT | GACATTGTGATGTCACAGTCTCCATCCTCCCTGG |
| 6402E2- | | GGTGAAGCCTGGGGCTTCAGTGAAGATATCCT | CTGTGTCAGCAGGAGAGAGGGTCACTATGAGCT |
| Ab2 | | GTAAGGCTTCTGGATACACGTTCACTGACTAC | GCAAATCCAGTCAGAGTCTGTTCAACAGTAGAAC |
| | | TACATGAACTGGGTGAAACAGAGCCATGGAAA | CCGAAAGAACTACTTGGCTTGGTACCAGCAGAAA |
| | | GAGCCTTGAGTGGATTGGAGATATTAATCCTA | CCAGGGCAGTCTCCTAAACTGCTGATCTACTGGG |
| | | ACAATGGTGATACTACCTACAACCAGAAGTTCA | CATCCACTAGGGAATCTGGGGTCCCTGATCGCTT |
| | | AGGGCAGGGCCACATTGACTGTAGACAAGTCC | CACAGGCAGTGGATCTGGGACAGATTTCAGTCTC |
| | | TCCAGCACAGCCTACATGGAGCTCCGCAGCCT | AACATCAGCAGTGTGCAGGCTGAAGACCTGGCA |
| | | GACATCTGAGGACTCTGCAGTCTATTACTGTG | GTTTATTACTGCAAGCAATCTTATGATCTGTGGAC |
| | | CAAGATCGGGGAGTAATTACTATGCTATGGAC | GTTCGGTGGAGGCACCAAGCTGGAAATCAAA |
| | | TCCTGGGGTCAAGGAACCTCAGTCACCGTCTC | (SEQ ID NO: 809) |
| | | CTCA (SEQ ID NO: 808) | |
| ACI-8033- | 6402E10B3 | GATGTACAGCTTCAGGAGTCAGGACCTGGCCT | GATGTTGTGATGACCCAGACTCCACTCACTTTGT |
| 6402E10- | | CGTGAAACCTGTTCAGTCTTTGTCTCTCACCTG | CGGTTACCATTGGACAACCAGCCTCCATCTCTTG |
| Ab1 | | CTCTGTCACTGGCTACTCCATCACCAGTGGTT | CAAGTCAAGTCAGAGCCTCTTAGATAGTGATGGA |
| | | ATTACTGGAACTGGATCCGGCAGTTTCCAGGA | GAGACATATTTGAATTGGTTGTTACAGAGGCCAG |
| | | AACAAACTGGAATGGATGGGCTACATAAGCTC | GCCAGTCTCCAAAGCGCCTAATCTATCTGGTGTC |
| | | CGATGGTAGCAATAACTACAACCCATCTCTCAA | TAAAGTGGACTCTGGAGTCCCTGACAGGTTCACT |
| | | AAATCGAATCTCCATCTCTCGTGACACATCTAA | GGCAGAGGATCAGGGACAGATTTCACACTGAAAA |
| | | GAACCAGTTTTTCCTGAAGTTGGATTCTGTGAC | TCAGCAGAGTGGAGGCTGAGGATTTGGGAGTTTA |
| | | TACTGAGGACACAGCCACATATTACTGTGTAA | TTATTGCTGGCAAGGCACACATTTTCCTCAGACGT |
| | | GAGCGGACAACTACTGGGGCCAAGGCACCAC | TCGGTGGAGGCACCAAGCTGGAAATCAAG (SEQ |
| | | TCTCACAGTCTCCTCG (SEQ ID NO: 818) | ID NO: 819) |
| ACI-8033- | 6403A4A3 | GAGGTGCAGCTTGTTGAGTCTGGTGGAGGATT | CAAATTGTTCTCACCCAGTCTCCAGCAATCATGTC |
| 6403A4- | | GGTGCAGCCTAAAGGGTCATTGAAACTCTCAT | TGCATCTCTAGGGGAGGAGATCACCCTAACCTGC |
| Ab1 | | GTGCAGCCTCTGGATTCAGTTTCAATACCTAC | AGTGCCAGTTCGAGTATAAGTTACATGCACTGGT |
| | | GCCATGAACTGGGTCCGCCAGGCTCCAGGAA | ACCAGCAGAAGTCAGGCACTTCTCCCAAACTCTT |
| | | AGGGTTTGGAATGGGTTGCTCGCATAAGAAGT | GATTCATAGCACATTCAACCTGGCTTCTGGAGTC |
| | | AAAAGTAATAATTATACAACATATTATGCCGATT | CCTTCTCGCTTCAGTGGCAGTGGGTCTGGGACCT |
| | | CAGTGAAAGACAGATTCACCATCTCCAGAGAT | TTTATTCTCTCACAATCAGCAGTGTGGAGGCTGAA |
| | | GATTCAGAAAGCATGCTCTATCTGCAAATGAAC | GATGCTGCCGATTATTACTGCCATCAGTGGAGTA |
| | | AACTTGAAAACTGAGGACACAGCCATGTATTA | GTTATTACACGTTCGGAGGGGGGACCAAGCTGG |
| | | CTGTGTGAGAACTATGGACTACTGGGGTCAAG | AAATAAAA (SEQ ID NO: 829) |
| | | GAACCTCAGTCACCGTCTCCTCA (SEQ ID | |
| | | NO: 828) | |
| ACI-8033- | 6403E11B4 | GATGTACAGCTTCAGGAGTCAGGACCTGGCCT | GATGTTGTGATGACCCAGACTCCACTCACTTTGT |
| 6403E11- | | CGTGAAACCTTCTCAGTCTCTGTCTCTCACCTG | CGGTCACCATTGGACAACCAGCCTCCATCTCTTG |
| Ab2 | | CTCTGTCACTGGCTACTCCATCACCAGTGCTT | CAAGTCAAGTCAGAGCCTCTTAGATGGTGATGGA |
| | | ATTACTGGAACTGGATCCGGCAGTTTCCAGGA | GAGACATATTTGAATTGGTTGTTACAGAGGCCAG |
| | | AACAAACTGGAATGGATGGGCTACATAAGCTA | GCCAGTCTCCAAAGCGCCTAATCTATCTGGTATC |
| | | CGATGGTAGCAATAACTACATCCCTTCTCTCAA | TAAACTGGACTCTGGAGTCCCTGACAGGTTCACT |
| | | AAATCGAATCTCCATCAGTCGTGACACATCTAA | GGCAGTGGATCAGGGACAGATTTCACACTGAAAA |
| | | GAACCAGTTTTTCCTGAAGTTGAATTCTGTGAC | TCAGCAGAGTGGAGGCTGAGGATTTGGGAGTTTA |
| | | TACTGAGGACACAGCCACATATTACTGTATAAG | TTATTGCTGGCAAGGTACACATTTTCCTCAGACGT |
| | | AGGGGATGTTAACTGGGGCCAAGGCACCACT | TCGGTGGAGGCACCAAGCTGGAAATCAAA (SEQ |
| | | CTCACAGTCTCCTCA (SEQ ID NO: 838) | ID NO: 839) |
| ACI-7067- | 4813-R4A- | GAGGTGCAGCTGGAGGAGTCTGGTGGAGGAT | CAGGCTGTTGTGACTCAGGAATCTGCACTCACCA |
| 4813-R4A- | G7 | TGGTGCACCCTAAAGGATCATTGAAACTCTCAT | CATCACCTGGTGAAACAGTCACACTCACTTGTCG |
| G7-rec1 | | GTGCCGCCTCTGGTTTCACCTTCAATTCCTATG | CTCAAGTACTGGGGCTGTTACAACTAGTAACTAT |
| | | CCATGCACTGGGTCCGCCAGGCTCCAGGAAA | GCCAACTGGGTCCAAGAAAAACCAGATCATTTATT |
| | | GGGTTTGGAATGGGTTGCTCGCATAAGAAGTA | CACTGGTCTAATAGGTGGTACCAACAACCGAGCT |
| | | AAAGAAGTAATTATGCAACATATTATGCCGATT | CCAGGTGTTCCTGCCAGATTCTCAGGCTCCCTGA |
| | | CAGTGAAAGACAGATTCACCATCTCCAGAGAT | TTGGAGACAAGGCTGCCCTCACCATCACAGGGG |
| | | GATTCACAAAGCATGGTCTATCTGGAAATGAA | CACAGACTGAGGATGAGGCAATATATTTCTGTGC |
| | | CAACCTGGAAGCTGAGGACACAGCCACGTATT | TCTATGGTACAGCAACCATTGGGTGTTCGGTGGA |
| | | ACTGTGTGAGAGGGGGACGAGAGGCTATGGA | GGAACCAAACTGACTGTCCTA (SEQ ID |
| | | CTTCTGGGGTCAAGGAACCTCAGTCACCGTCT | NO: 849) |
| | | CCTCA (SEQ ID NO: 848) | |

TABLE 7
Amino acid sequence of the heavy chain and light chain variable domains (VH and VL) and their CDRs

| Antibody | Hybridoma | | VH | VH | VH | | VL | VL | VL |
|---|---|---|---|---|---|---|---|---|---|
| Code | Code | VH | CDR1 | CDR2 | CDR3 | VL | CDR1 | CDR2 | CDR3 |
| ACI-7079- | 3101E3C9 | QVQLKESGPGL | RYIIN | VIWTGG | EYGYD | DVLMTQTPLSL | RSSQTIV | KVSNRF | FQGSH |
| 3101E3- | | VAPSQSLSITCT | (SEQ ID | GTDYNS | GAWFA | PVSLGDQASIS | HSNGNT | S (SEQ | VPYT |
| Ab1 | | VSGFSLTRYIIN | NO: | ALKS | Y (SEQ | CRSSQTIVHSN | YLE | ID NO: | (SEQ ID |
| | | WVRQPPGKGLE | 611) | (SEQ ID | ID NO: | GNTYLEWYLQK | (SEQ ID | 616) | NO: 617) |
| | | WLGVIWTGGGT | | NO: 612) | 613) | PGQSPKLLIYKV | NO: 615) | | |
| | | DYNSALKSRLSI | | | | SNRFSGVPDRF | | | |
| | | SKDNSKSQVFL | | | | SGSGSGTDFTL | | | |
| | | KMNSLQTDDTA | | | | KISRVEAEDLG | | | |
| | | RYYCAREYGYD | | | | VYYCFQGSHVP | | | |
| | | GAWFAYWGQG | | | | YTFGGGTKLEIK | | | |
| | | TLVTVSA (SEQ | | | | (SEQ ID NO: | | | |
| | | ID NO: 610) | | | | 614) | | | |
| ACI-7079- | 3103D9C9 | QVSLKESGPGIL | TFGMG | HIWWD | IGDYYG | DIVMTQSPSSL | KSSQSLL | FASTRE | QQHYS |
| 3103D9- | | QPSQTLSLTCSF | VG | DDKYYN | TRGYFD | TMSLGQTVTMS | NSSNQK | S (SEQ | TPYT |
| Ab1 | | SGFSLSTFGMG | (SEQ ID | PGLKS | V (SEQ | CKSSQSLLNSS | NHLA | ID NO: | (SEQ ID |
| | | VGWIRQPSGKG | NO: | (SEQ ID | ID NO: | NQKNHLAWYQ | (SEQ ID | 626) | NO: 627) |
| | | LDWLTHIWWDD | 621) | NO: 622) | 623) | QRPGQSPKLLV | NO: 625) | | |
| | | DKYYNPGLKSR | | | | YFASTRESGVP | | | |
| | | LTISKDTSKNQV | | | | ERFIGGGSGTD | | | |
| | | FLEIANVDTADT | | | | FTLTISNVQSED | | | |
| | | ATYYCARIGDYY | | | | LTDYFCQQHYS | | | |
| | | GTRGYFDVWGT | | | | TPYTFGGGTKL | | | |
| | | GTTVTVSS (SEQ | | | | EIK (SEQ ID NO: | | | |
| | | ID NO: 620) | | | | 624) | | | |
| ACI-7079- | 3103G12D2B10 | DVQLQESGPGM | SGYDW | YITYSG | DTVDA | DVLMTQAPLSL | RSSQSIV | KVSNRF | FQGSH |
| 3103G12- | | VKPSQSLSLTCT | H (SEQ | STNYNP | WFAY | PVSLGDQASIS | HSNGDT | S (SEQ | VPPT |
| Ab2 | | VTGYSITSGYD | ID NO: | SLKS | (SEQ ID | CRSSQSIVHSN | YLE | ID NO: | (SEQ ID |
| | | WHWIRHFPGNK | 631) | (SEQ ID | NO: 633) | GDTYLEWYLQK | (SEQ ID | 616) | NO: 637) |
| | | LEWMGYITYSG | | NO: 632) | | PGQSPKLLIYKV | NO: 635) | | |
| | | STNYNPSLKSRI | | | | SNRFSGVPDRF | | | |
| | | SITHDTSKNHFF | | | | SGSGSGTDFTL | | | |
| | | LKLTSVTTEDTA | | | | KISRVEAEDLG | | | |
| | | TYYCARDTVDA | | | | VYYCFQGSHVP | | | |
| | | WFAYWGQGTLV | | | | PTFGGGTKLEIK | | | |
| | | TVSA (SEQ ID | | | | (SEQ ID NO: | | | |
| | | NO: 630) | | | | 634) | | | |
| ACI-7079- | 3104F12F2H7 | QVTLKESGPGIV | TFGLGV | HIWWD | IGDFYG | DIVMTQSPSSL | KSSQSLL | FASTRE | QQHYS |
| 3104F12- | | QPSQTLSLTCSF | G (SEQ | DDKYYN | SRGYFD | AMSVGQKVTLR | NSSNQK | S (SEQ | TPYT |
| Ab2 | | SGFSLNTFGLGV | ID NO: | PALKS | V (SEQ | CKSSQSLLNSS | NHLA | ID NO: | (SEQ ID |
| | | GWIRQPSGKGL | 641) | (SEQ ID | ID NO: | NQKNHLAWYQ | (SEQ ID | 626) | NO: 627) |
| | | DWLTHIWWDDD | | NO: 642) | 643) | QKPGQSPKLLL | NO: 625) | | |
| | | KYYNPALKSRLT | | | | CFASTRESGVP | | | |
| | | ISKDTSKNQVFL | | | | DRFIGSGSGTD | | | |
| | | KIANVDTADTAT | | | | FTLTISSVQAED | | | |
| | | YYCARIGDFYGS | | | | LADYFCQQHYS | | | |
| | | RGYFDVWGTGT | | | | TPYTFGGGTKL | | | |
| | | TVTVSS (SEQ ID | | | | EIK (SEQ ID NO: | | | |
| | | NO: 640) | | | | 644) | | | |
| ACI-7079- | 3106C5B7 | QVTLKESGPGIL | TFGMG | HIWWD | IGDYYG | DIVMTQSPSSL | KSRQSL | FASTRE | QQHYS |
| 3106C5- | | QPSQTLSLTCSF | VG | DDKYYN | SRGYFD | AMSVGQKVTM | LNSSNQ | S (SEQ | TPYT |
| Ab1 | | SGFSLSTFGMG | (SEQ ID | PALKS | V (SEQ | SCKSRQSLLNS | KNHLA | ID NO: | (SEQ ID |
| | | VGWIRQPSGKG | NO: | (SEQ ID | ID NO: | SNQKNHLAWY | (SEQ ID | 626) | NO: 627) |
| | | LEWLTHIWWDD | 621) | NO: 642) | 653) | QQKPGQSPKLL | NO: 655) | | |
| | | DKYYNPALKSRL | | | | VYFASTRESGV | | | |
| | | TISRDPSKNQVF | | | | PDRFIGSGSGT | | | |
| | | LKIANVDTADTA | | | | DFTLTISSVQAE | | | |
| | | TYYCARIGDYYG | | | | DLADYFCQQHY | | | |
| | | SRGYFDVWGTG | | | | STPYTFGGGTK | | | |
| | | TTVTVSS (SEQ | | | | LEIK (SEQ ID | | | |
| | | ID NO: 650) | | | | NO: 654) | | | |
| ACI-7079- | 3106F2C8 | EVQLQQSGAEL | DDYMH | WIDPEN | AGTSPY | DIVMTQSQKFM | KASQNV | SASYRY | QQYNS |
| 3106F2- | | VRPGASVKLSC | (SEQ ID | GDTEYA | YFDY | STSVGDRVSVT | GTNVA | S (SEQ | YPLT |
| Ab1 | | TASGFNVKDDY | NO: | SKFQG | (SEQ ID | CKASQNVGTNV | (SEQ ID | ID NO: | (SEQ ID |
| | | MHWVKQRPEQ | 661) | (SEQ ID | NO: 663) | AWYQQKVGQS | NO: 665) | 666) | NO: 667) |
| | | GLEWIGWIDPEN | | NO: 662) | | PKALIYSASYRY | | | |
| | | GDTEYASKFQG | | | | SGVPDRFTGSG | | | |
| | | KATLTADTSSNT | | | | SGTDFTLTISNV | | | |
| | | AYLQLSGLTSVD | | | | QSEDLADYFCQ | | | |
| | | TAVYYCTTAGTS | | | | QYNSYPLTFGS | | | |
| | | PYYFDYWGQGT | | | | GTKLEIK (SEQ | | | |
| | | TLTVSS (SEQ ID | | | | ID NO: 664) | | | |
| | | NO: 660) | | | | | | | |
| ACI-7079- | 3112H1F3 | EVQLQQSGPEL | DYFMN | DINPNN | GSNYA | DIVMSQSPSSL | KSSQSLL | WASTR | KQSYDL |
| 3112H1- | | VKPGASVKISCK | (SEQ ID | GGSSYI | MDS | AVSAGEKVTMS | NSRTRK | ES (SEQ | WT |
| Ab1 | | ASGYTFSDYFM | NO: | QKFKG | (SEQ ID | CKSSQSLLNSR | NYLA | ID NO: | (SEQ ID |
| | | NWVKQSHGKSL | 671) | (SEQ ID | N: O673) | TRKNYLAWYQ | (SEQ ID | 676) | NO: 677) |
| | | EWIGDINPNNG | | NO: 672) | | QKPGQSPKLLI | NO: 675) | | |
| | | GSSYIQKFKGKA | | | | YWASTRESGV | | | |
| | | TLTVDKSSTTAY | | | | PDRFTGSGSGT | | | |
| | | MELRSLTSEDSA | | | | DFTLTISSVQAE | | | |
| | | VYYCARGSNYA | | | | DLAVYYCKQSY | | | |
| | | MDSWGQGTSV | | | | DLWTFGGGTKL | | | |
| | | TVSS (SEQ ID | | | | EIK (SEQ ID NO: | | | |
| | | NO: 670) | | | | 674) | | | |
| ACI-7079- | 3107E6A3 | QVTLKESGPGIL | TFGMG | HIWWD | IGDYYG | DIVMTQSPSSL | KSSQSLL | FASTRD | QQHYS |
| 3107E6- | | QPSQTLSLTCSF | VG | DDKYYN | SRGFFD | AMSVGQKVTM | NSSNQK | S (SEQ | TPYT |
| Ab1 | | SGFSLSTFGMG | (SEQ ID | PALKS | V (SEQ | SCKSSQSLLNS | NHLA | ID NO: | (SEQ ID |
| | | VGWIRQPSGKG | NO: | (SEQ ID | ID NO: | SNQKNHLAWY | (SEQ ID | 686) | NO: 627) |
| | | LEWLTHIWWDD | 621) | NO: 642) | 683) | QQKPGQSPKLL | NO: 625) | | |
| | | DKYYNPALKSRL | | | | VYFASTRDSGV | | | |
| | | TISKDASKNQMF | | | | PDRFIGSGSGT | | | |
| | | LKIANVDTADTA | | | | DFTLTISSVQAE | | | |
| | | TYFCARIGDYYG | | | | DLADYFCQQHY | | | |
| | | SRGFFDVWGTG | | | | STPYTFGGGTK | | | |
| | | TTVTVSS (SEQ | | | | LEIK (SEQ ID | | | |
| | | ID NO: 680) | | | | NO: 684) | | | |
| ACI-7079- | 3108C10G6 | ELQLVESGGGL | NYAMS | TISDGG | AGSGH | DVVMTQTPRTL | KSSQSLL | LVSKVD | WQGTY |
| 3108C10- | | VRPGGSLKLSC | (SEQ ID | TYTFYS | (SEQ ID | SVIIGQPASISC | DSDGKT | S (SEQ | FPRT |
| Ab2 | | ATSGFTFRNYA | NO: | DNVKG | NO: 693) | KSSQSLLDSDG | YLS | ID NO: | (SEQ ID |
| | | MSWVRQTPAKR | 691) | (SEQ ID | | KTYLSWLLQRP | (SEQ ID | 696) | NO: 697) |
| | | LEWVATISDGGT | | NO: 692) | | GQSPKRLIYLVS | NO: 695) | | |
| | | YTFYSDNVKGR | | | | KVDSGVPDRFT | | | |
| | | FTISRDNVKNTL | | | | GSGSGTDFTLK | | | |
| | | YLQMSHLKSED | | | | ISRVEAEDLGV | | | |
| | | TAMYYCSRAGS | | | | YYCWQGTYFP | | | |
| | | GHWGQGTTLTV | | | | RTFGGGTKLEI | | | |
| | | SS (SEQ ID NO: | | | | K (SEQ ID NO: | | | |
| | | 690) | | | | 694) | | | |
| ACI-8030- | 6106F5B10 | QVQLQQSGPEL | TYYIH | WIYPGR | DWETG | DIVLTQSPASLA | KASQSV | AASNLD | QQSGE |
| 6106F5- | | VKPGASVKISCK | (SEQ ID | GYTKYN | FPY | VSLGQRATISC | DYGGDS | S (SEQ | DPFT |
| Ab1 | | ASGYSFTTYYIH | NO: | EKFKD | (SEQ ID | KASQSVDYGG | WLN | ID NO: | (SEQ ID |
| | | WMKQRPGQGP | 701) | (SEQ ID | NO: 703) | DSWLNWYQQK | (SEQ ID | 706) | NO: 707) |
| | | EWIGWIYPGRG | | NO: 702) | | PGQPPKLLIYAA | NO: 705) | | |
| | | YTKYNEKFKDKA | | | | SNLDSGIPARF | | | |
| | | AQTADTSSNTA | | | | GGSGSGTDFTL | | | |
| | | YMQLSSLRSED | | | | NIHPVEEEDAA | | | |
| | | SAVYYCARDWE | | | | TYYCQQSGED | | | |
| | | TGFPYWGQGTL | | | | PFTFGGGTNLEI | | | |
| | | VTVSA (SEQ ID | | | | K (SEQ ID NO: | | | |
| | | NO: 700) | | | | 704) | | | |
| ACI-8031- | 6207G10E6 | QVQLQQSGAEL | SYWMN | QIYPGD | THYDY | DILLTQSPAILSV | RASQSIG | YASESI | QQSNS |
| 6207G10- | | VKPGASVKISCK | (SEQ ID | GDTNY | (SEQ ID | SPGDRVSFSCR | TRIH | S (SEQ | WPLT |
| Ab1 | | ASGYAFSSYWM | NO: | NGKFK | NO: 713) | ASQSIGTRIHW | (SEQ ID | ID NO: | (SEQ ID |
| | | NWVKQRPGKGL | 711) | G (SEQ | | YQQRTNGSPRL | NO: 715) | 716) | NO: 717) |
| | | EWIGQIYPGDG | | ID NO: | | LIKYASESISGT | | | |
| | | DTNYNGKFKGK | | 712) | | PSRFSGSGSGT | | | |
| | | ATLTADRSSSTA | | | | DFTLSINSVESE | | | |
| | | YMQLSSLTSED | | | | DIADYYCQQSN | | | |
| | | SAVYFCAITHYD | | | | SWPLTFGAGTK | | | |
| | | YWGQGTTLTVS | | | | LELK (SEQ ID | | | |
| | | S (SEQ ID NO: | | | | NO: 714) | | | |
| | | 710) | | | | | | | |
| ACI-8032- | 6301A10E12 | QVQLQQSGAEL | SYGIS | EIYPRS | ESYYGD | DVLMTQTPLSL | RSSHSIE | KVSNRF | FQGSH |
| 6301A10- | | ARPGASVKLSC | (SEQ ID | GNTYYN | YDWYF | PVSLGDQASIS | HSNGNT | S (SEQ | VPPT |
| Ab2 | | KASGYTFTSYGI | NO: | EKFKG | DV (SEQ | CRSSHSIEHSN | YLE | ID NO: | (SEQ ID |
| | | SWVKQRTGQGL | 721) | (SEQ ID | ID NO: | GNTYLEWCLQK | (SEQ ID | 616) | NO: 637) |
| | | EWIGEIYPRSGN | | NO: 722) | 723) | PGQPPKFLIYKV | NO: 725) | | |
| | | TYYNEKFKGKAT | | | | SNRFSGVPDRF | | | |
| | | LTADKSSSTAYM | | | | SGSGSGTDFTL | | | |
| | | ELRSLTSEDSAV | | | | RISRVEAEDLG | | | |
| | | YFCASESYYGD | | | | VYYCFQGSHVP | | | |
| | | YDWYFDVWGT | | | | PTFGGGTKLEIK | | | |
| | | GTTVTVSS (SEQ | | | | (SEQ ID NO: | | | |
| | | ID NO: 720) | | | | 724) | | | |
| ACI-8032- | 6301C8G12 | QVQLQQSGAEL | TYGIS | EIYPRS | ESYYGD | DVLMTQIPLSLP | RSSQSIV | EVSNRF | FQGSH |
| 6301C8- | | ARPGTSVKLSC | (SEQ ID | GNTYYN | YAWYF | VSLGDQASISC | HSNGNT | S (SEQ | VPPT |
| Ab2 | | KASGYTFTTYGI | NO: | EKFKG | DV (SEQ | RSSQSIVHSNG | YLE | ID NO: | (SEQ ID |
| | | SWVKQRTGQGL | 731) | (SEQ ID | ID NO: | NTYLEWHLQKP | (SEQ ID | 736) | NO: 637) |
| | | EWIGEIYPRSGN | | NO: 722) | 733) | GQSPKLLIYEVS | NO: 735) | | |
| | | TYYNEKFKGKAT | | | | NRFSGVPDRFS | | | |
| | | LTADKPSSTAYM | | | | GSGSGTDFTLN | | | |
| | | ELRSLTSEDSAV | | | | ISRVEAEDLGV | | | |
| | | YFCASESYYGD | | | | YYCFQGSHVPP | | | |
| | | YAWYFDVWGT | | | | TFGGGTKLEIK | | | |
| | | GTTVTVSS (SEQ | | | | (SEQ ID NO: | | | |
| | | ID NO: 730) | | | | 734) | | | |
| ACI-8032- | 6301G2D11 | EVQLQQSGPEL | DYFMN | DINPNIG | GGGSN | DIVMSQSPSSL | KSSQSLL | WASTR | KQSYDL |
| 6301G2- | | VKPGASVKISCK | (SEQ ID | VTNYNP | PLYYAM | AVSAGEKVTMS | NSRTRK | ES (SEQ | WT |
| Ab2 | | ASGYTFTDYFM | NO: | KFKD | DY (SEQ | CKSSQSLLNSR | NYLA | ID NO: | (SEQ ID |
| | | NWVKQSHGKSL | 671) | (SEQ ID | ID NO: | TRKNYLAWYQ | (SEQ ID | 676) | NO: 677) |
| | | EWIGDINPNIGV | | NO: 742) | 743) | QKPGQSPKLLI | NO: 675) | | |
| | | TNYNPKFKDRA | | | | YWASTRESGV | | | |
| | | TLTVDKSSSSAY | | | | PDRFTGSGSGT | | | |
| | | MELRSLTSEDST | | | | DFTLTISSVQAE | | | |
| | | VYYCARGGGSN | | | | DLAVYFCKQSY | | | |
| | | PLYYAMDYWGQ | | | | DLWTFGGGTKL | | | |
| | | GTSVTVSS | | | | EIK (SEQ ID NO: | | | |
| | | (SEQ ID NO: | | | | 744) | | | |
| | | 740) | | | | | | | |
| ACI-8032- | 6304F3D12 | QVQLQQSGAEL | SYGVS | EIYPRS | ESYYGD | DVLMTQIPLSLP | RSSQSIV | KVSNRF | FQGSH |
| 6304F3- | | ARPGASVKLSC | (SEQ ID | GNTYYN | YDWYF | VSPGDQVSISC | HSNGNT | S (SEQ | VPPT |
| Ab1 | | KASGYTFTSYG | NO: | EKFKG | DV (SEQ | RSSQSIVHSNG | YLE | ID NO: | (SEQ ID |
| | | VSWVKQRTGQ | 751) | (SEQ ID | ID NO: | NTYLEWYLQKP | (SEQ ID | 616) | NO: 637) |
| | | GLEWIGEIYPRS | | NO: 722) | 723) | GQSPKPLIYKV | NO: 735) | | |
| | | GNTYYNEKFKG | | | | SNRFSGVPDRF | | | |
| | | KATLTADKSSST | | | | SGSGSGTDFTL | | | |
| | | AYMELRSLTSED | | | | KISRVEAEDLG | | | |
| | | SAVYFCASESYY | | | | VYYCFQGSHVP | | | |
| | | GDYDWYFDVW | | | | PTFGGGTKLEIK | | | |
| | | GTGTTVTVSS | | | | (SEQ ID NO: | | | |
| | | (SEQ ID NO: | | | | 754) | | | |
| | | 750) | | | | | | | |
| ACI-8032- | 6307F1H10 | QVQLQQSGAEL | SYGMS | EIYPRS | ESYYGD | DVLMTQTPLSL | RSSQSV | KVSNRF | FQGSH |
| 6307F1- | | ARPGASVKLSC | (SEQ ID | GNTYYN | YAWYF | PVSLGDQASIS | VHSNGIT | S (SEQ | VPPT |
| Ab2 | | KASGYTFTSYG | NO: | EKFKG | DV (SEQ | CRSSQSVVHSN | YLE | ID NO: | (SEQ ID |
| | | MSWVKQRTGQ | 761) | (SEQ ID | ID NO: | GITYLEWYLQK | (SEQ ID | 616) | NO: 637) |
| | | GLEWIGEIYPRS | | NO: 722) | 733) | PGQSPKLLIYKV | NO: 765) | | |
| | | GNTYYNEKFKG | | | | SNRFSGVPDRF | | | |
| | | KATLTSDKSSST | | | | SGSGSGTYFTL | | | |
| | | SYMELRSLTSED | | | | KISRVEAEDLG | | | |
| | | SAVYFCASESYY | | | | VYYCFQGSHVP | | | |
| | | GDYAWYFDVW | | | | PTFGGGTKLEIK | | | |
| | | GTGTTVTVSS | | | | (SEQ ID NO: | | | |
| | | (SEQ ID NO: | | | | 764) | | | |
| | | 760) | | | | | | | |
| ACI-8032- | 6313G2A10 | EVQLQQSGPEL | DYYMN | DIDPNN | TGYYGT | DIVMSQSPSSL | KSSQSLL | WASTR | KQSYDL |
| 6313G2- | | VKPGASVKISCK | (SEQ ID | GVTSYS | SLYYAM | AVSAGEKVTMN | NSRTRK | ES (SEQ | WT |
| Ab1 | | ASGYTFTDYYM | NO: | QKFKG | DY (SEQ | CKSSQSLLNSR | NYLA | ID NO: | (SEQ ID |
| | | NWVKQSHGKSL | 771) | (SEQ ID | ID NO: | TRKNYLAWYQ | (SEQ ID | 676) | NO: 677) |
| | | EWIGDIDPNNGV | | NO: 772) | 773) | QKPGQSPKLLI | NO: 675) | | |
| | | TSYSQKFKGKA | | | | YWASTRESGV | | | |
| | | TLTVDKSSSTAY | | | | PDRFIGSGSGT | | | |
| | | MELRSLTSEDSA | | | | GFTLTISSVQAE | | | |
| | | VYYCARTGYYG | | | | DLAVYYCKQSY | | | |
| | | TSLYYAMDYWG | | | | DLWTFGGGTKL | | | |
| | | QGTSVTVSS | | | | EIK (SEQ ID NO: | | | |
| | | (SEQ ID NO: | | | | 774) | | | |
| | | 770) | | | | | | | |
| ACI-8032- | 6314A3E4 | QVQLQQSGAEL | SYGVS | EIYPRS | ESYYGD | DVLMTQIPLSLP | RSSQSIV | KVSNRF | FQGSH |
| 6314A3- | | ARPGASVKLSC | (SEQ ID | GNTYYN | YDWYF | VSPGDQVFISC | HSNGNT | S (SEQ | VPPT |
| Ab3 | | KASGYTFTSYG | NO: | EKFKG | DV (SEQ | RSSQSIVHSNG | YLE | ID NO: | (SEQ ID |
| | | VSWVKQRTGQ | 751) | (SEQ ID | ID NO: | NTYLEWYLQKP | (SEQ ID | 616) | NO: 637) |
| | | GLEWIGEIYPRS | | NO: 722) | 723) | GQSPKPLIYKV | NO: 735) | | |
| | | GNTYYNEKFKG | | | | SNRFSGVPDRF | | | |
| | | KATLTADKSSST | | | | SGSGSGTDFTL | | | |
| | | AYMELRSLTSED | | | | KISRVEAEDLG | | | |
| | | SAVYFCASESYY | | | | VYYCFQGSHVP | | | |
| | | GDYDWYFDVW | | | | PTFGGGTKLEIK | | | |
| | | GTGTTVTVSS | | | | (SEQ ID NO: | | | |
| | | (SEQ ID NO: | | | | 784) | | | |
| | | 750) | | | | | | | |
| ACI-8033- | 6401F2E3 | QVQLQQPGAEL | NYWIT | DIYPGN | GQTSF | DVLMTQTPLSL | RSSQSV | KVSNRF | FQGSH |
| 6401F2- | | VKPGASVKMSC | (SEQ ID | GDVDY | GH | PVSLGDQASIS | VHVNGN | S (SEQ | VPRT |
| Ab1 | | KASGYTFTNYWI | NO: | SEIFKN | (SEQ ID | CRSSQSVVHVN | TYLD | ID NO: | (SEQ ID |
| | | TWVKQRPGQGL | 791) | (SEQ ID | NO: 793) | GNTYLDWYLQK | (SEQ ID | 616) | NO: 797) |
| | | EWIGDIYPGNGD | | NO: 792) | | PGQSPKLLIYKV | NO: 795) | | |
| | | VDYSEIFKNKAK | | | | SNRFSGVPDRF | | | |
| | | MTVDTSSTTAY | | | | SGSGSGTDFTL | | | |
| | | MQLSSLTSEDS | | | | RITRVEAEDLGI | | | |
| | | AVYHCVLGQTS | | | | YYCFQGSHVPR | | | |
| | | FGHWGQGTLVT | | | | TFGGGTKLEIK | | | |
| | | VSA (SEQ ID | | | | (SEQ ID NO: | | | |
| | | NO: 790) | | | | 794) | | | |
| ACI-8033- | 6402E2E4 | EVQLQHSGPDL | DYYMN | DINPNN | SGSNYY | DIVMSQSPSSL | KSSQSL | WASTR | KQSYDL |
| 6402E2- | | VKPGASVKISCK | (SEQ ID | GDTTYN | AMDS | AVSAGERVTMS | FNSRTR | ES (SEQ | WT |
| Ab2 | | ASGYTFTDYYM | NO: | QKFKG | (SEQ ID | CKSSQSLFNSR | KNYLA | ID NO: | (SEQ ID |
| | | NWVKQSHGKSL | 771) | (SEQ ID | NO: 803) | TRKNYLAWYQ | (SEQ ID | 676) | NO: 677) |
| | | EWIGDINPNNGD | | NO: 802) | | QKPGQSPKLLI | NO: 805) | | |
| | | TTYNQKFKGRA | | | | YWASTRESGV | | | |
| | | TLTVDKSSSTAY | | | | PDRFTGSGSGT | | | |
| | | MELRSLTSEDSA | | | | DFSLNISSVQAE | | | |
| | | VYYCARSGSNY | | | | DLAVYYCKQSY | | | |
| | | YAMDSWGQGT | | | | DLWTFGGGTKL | | | |
| | | SVTVSS (SEQ ID | | | | EIK (SEQ ID NO: | | | |
| | | NO: 800) | | | | 804) | | | |
| ACI-8033- | 6402E10B3 | DVQLQESGPGL | SGYYW | YISSDG | ADNY | DVVMTQTPLTL | KSSQSLL | LVSKVD | WQGTH |
| 6402E10- | | VKPVQSLSLTCS | N (SEQ | SNNYNP | (SEQ ID | SVTIGQPASISC | DSDGET | S (SEQ | FPQT |
| Ab1 | | VTGYSITSGYYW | ID NO: | SLKN | NO: 813) | KSSQSLLDSDG | YLN | ID NO: | (SEQ ID |
| | | NWIRQFPGNKL | 811) | (SEQ ID | | ETYLNWLLQRP | (SEQ ID | 696) | NO: 817) |
| | | EWMGYISSDGS | | NO: 812) | | GQSPKRLIYLVS | NO: 815) | | |
| | | NNYNPSLKNRIS | | | | KVDSGVPDRFT | | | |
| | | ISRDTSKNQFFL | | | | GRGSGTDFTLK | | | |
| | | KLDSVTTEDTAT | | | | ISRVEAEDLGV | | | |
| | | YYCVRADNYWG | | | | YYCWQGTHFP | | | |
| | | QGTTLTVSS | | | | QTFGGGTKLEI | | | |
| | | (SEQ ID NO: | | | | K (SEQ ID NO: | | | |
| | | 810) | | | | 814) | | | |
| ACI-8033- | 6403A4A3 | EVQLVESGGGL | TYAMN | RIRSKS | TMDY | QIVLTQSPAIMS | SASSSIS | STFNLA | HQWSS |
| 6403A4- | | VQPKGSLKLSC | (SEQ ID | NNYTTY | (SEQ ID | ASLGEEITLTCS | YMH | S (SEQ | YYT |
| Ab1 | | AASGFSFNTYA | NO: | YADSVK | NO: 823) | ASSSISYMHWY | (SEQ ID | ID NO: | (SEQ ID |
| | | MNWVRQAPGK | 821) | D (SEQ | | QQKSGTSPKLLI | NO: 825) | 826) | NO: 827) |
| | | GLEWVARIRSKS | | ID NO: | | HSTFNLASGVP | | | |
| | | NNYTTYYADSV | | 822) | | SRFSGSGSGTF | | | |
| | | KDRFTISRDDSE | | | | YSLTISSVEAED | | | |
| | | SMLYLQMNNLK | | | | AADYYCHQWS | | | |
| | | TEDTAMYYCVR | | | | SYYTFGGGTKL | | | |
| | | TMDYWGQGTS | | | | EIK (SEQ ID NO: | | | |
| | | VTVSS (SEQ ID | | | | 824) | | | |
| | | NO: 820) | | | | | | | |
| ACI-8033- | 6403E11B4 | DVQLQESGPGL | SAYYW | YISYDG | GDVN | DVVMTQTPLTL | KSSQSLL | LVSKLD | WQGTH |
| 6403E11- | | VKPSQSLSLTCS | N (SEQ | SNNYIP | (SEQ ID | SVTIGQPASISC | DGDGET | S (SEQ | FPQT |
| Ab2 | | VTGYSITSAYYW | ID NO: | SLKN | NO: 833) | KSSQSLLDGDG | YLN | ID NO: | (SEQ ID |
| | | NWIRQFPGNKL | 831) | (SEQ ID | | ETYLNWLLQRP | (SEQ ID | 836) | NO: 817) |
| | | EWMGYISYDGS | | NO: 832) | | GQSPKRLIYLVS | NO: 835) | | |
| | | NNYIPSLKNRISI | | | | KLDSGVPDRFT | | | |
| | | SRDTSKNQFFLK | | | | GSGSGTDFTLK | | | |
| | | LNSVTTEDTATY | | | | ISRVEAEDLGV | | | |
| | | YCIRGDVNWGQ | | | | YYCWQGTHFP | | | |
| | | GTTLTVSS (SEQ | | | | QTFGGGTKLEI | | | |
| | | ID NO: 830) | | | | K (SEQ ID NO: | | | |
| | | | | | | 834) | | | |
| ACI-7067- | 4813- | EVQLEESGGGL | SYAMH | RIRSKR | GGREA | QAVVTQESALT | RSSTGA | GTNNR | ALWYS |
| 4813-R4A- | R4A-G7 | VHPKGSLKLSCA | (SEQ ID | SNYATY | MDF | TSPGETVTLTC | VTTSNY | AP (SEQ | NH |
| G7-rec1 | | ASGFTFNSYAM | NO: | YADSVK | (SEQ ID | RSSTGAVTTSN | AN (SEQ | ID NO: | (SEQ ID |
| | | HWVRQAPGKGL | 841) | D (SEQ | NO: 843) | YANWVQEKPD | ID NO: | 846) | NO: 847) |
| | | EWVARIRSKRS | | ID NO: | | HLFTGLIGGTN | 845) | | |
| | | NYATYYADSVK | | 842) | | NRAPGVPARFS | | | |
| | | DRFTISRDDSQS | | | | GSLIGDKAALTI | | | |
| | | MVYLEMNNLEA | | | | TGAQTEDEAIY | | | |
| | | EDTATYYCVRG | | | | FCALWYSNHW | | | |
| | | GREAMDFWGQ | | | | VFGGGTKLTVL | | | |
| | | GTSVTVSS | | | | (SEQ ID NO: | | | |
| | | (SEQ ID NO: | | | | 844) | | | |
| | | 840) | | | | | | | |

Inhibiting a-Syn Propagation in Cells

Monoclonal anti-alpha-synuclein antibodies are being evaluated for their ability to inhibit the uptake and seeding of alpha-synuclein aggregation in an in vitro cellular model that is susceptible to alpha-synuclein seeding and in mouse primary cortical neurons. The addition of alpha-synuclein seeds to the cellular model or primary neurons initiates the de novo aggregation of monomeric a-synuclein. The formation of de novo a-syn aggregates or de novo pathological alpha-synuclein (phosphorylated alpha-synuclein) is being assessed in the presence or absence of alpha-synuclein antibodies relative to an isotype control antibody. The ability of alpha-synuclein antibodies to inhibit uptake or seeded aggregation is being quantified as a percent change in the number of alpha-synuclein aggregates observed.

For the in vitro cellular model, alpha-synuclein antibodies of the invention or an isotype control antibody are incubated with 0.4 μL/well Ab-DeliverIN™ Transfection Reagent (OZ Biosciences, A121000) for 30 min at room temperature in low-binding 96-well plates (Eppendorf Microplate 96/V-PP, Sigma, EP951040227). Antibodies/Ab-DeliverIN are then added to the cells, plated at a density of 8,000 cells/well 24 hours prior to treatment, and placed back in the incubator (at 37° C. with 5% CO₂) for 5 hours. Alpha-synuclein seeds (0.05 μg/well) are diluted in a reduced-serum medium (Opti-MEM™, Life Technologies, 31985070) and incubated with 0.2 μL/well Lipofectamine™ 2000 Transfection Reagent (Life Technologies, 11668019) for 30 min at 25° C. in a low-binding 96-well plate. Alpha-synuclein seeds/lipofectamine are then added to cells. As a reference control, cells are also transduced with lipofectamine without alpha-synuclein seeds. Cells are placed back in the incubator (at 37° C. with 5% CO₂). Cells are then supplemented at 24 hours post-transduction with 100 μL of DMEM/glutamax (Gibco, 31966-021), supplemented with 5% Fetal Bovine Serum (qualified and heat inactivated; Gibco, 10500-064) and 1% Penicillin-Streptomycin (10,000 U/mL; Gibco, 15140-122). At 96 hours, post initial transduction, cells are fixed with an equal volume of cold 2% Triton X-100, 8% PFA in PBS, and Hoechst 33342 (1:10,000). Media is removed and washed three times with PBS, fixed cells are left in PBS, kept protected from light, and high-content imaging analysis is performed to detect and quantify the formation of de novo alpha-synuclein aggregates. Use of an intrinsically fluorescent reporter protein allows for the detection of de novo alpha-synuclein aggregates. The percent aggregates formed is then calculated relative to conditions in the absence of antibodies. IC50 values is obtained from fitting using Equation 6 (GraphPad Prism 7).

$$Y = \frac{Bottom + ( Top - Bottom )}{1 + 1 {0}^{( {logIC}_{50} - X ) * Hill Slope}} \qquad Equation 6$$

For the mouse primary cortical neurons, cells are cultured in 384-well plates. At 6 days in vitro (DIV), alpha-synuclein antibodies of the invention or an isotype control antibody are added to cells plated at a density of 40,000 cells/well and incubated for 30 min. Alpha-synuclein seeds (8 μg) are then added to the cells. At 13 DIV (7 days after alpha-synuclein seed addition) the cells are fixated with PFA and stained with an antibody directed against phosphorylated alpha-synuclein (EP1536Y) and Hoechst stain. High-content image analysis is performed to detect and quantify the formation of de novo alpha-synuclein aggregates/cell. The percent aggregates formed is then calculated relative to conditions in the absence of antibodies. Data is combined from three independent experiments and IC₅₀ values are obtained from fitting using Equation 7 (GraphPad Prism 7).

$$Y = \frac{100}{1 + {( \frac{{IC}_{50}}{X} )}^{Hill Slope}} \qquad Equation 7$$

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

1.-71. (canceled)

72. An alpha-synuclein binding molecule, which comprises:
a) VH-CDR1 comprising the amino acid sequence of SEQ ID NO: 691; VH-CDR2 comprising the amino acid sequence of SEQ ID NO: 692; and VH-CDR3 comprising the amino acid sequence of SEQ ID NO: 693; VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697; or
b) VH-CDR1 comprising the amino acid sequence of SEQ ID NO: 721; VH-CDR2 comprising the amino acid sequence of SEQ ID NO: 722; and VH-CDR3 comprising the amino acid sequence of SEQ ID NO: 723; VL-CDR1 comprising the amino acid sequence of SEQ ID NO: 725; VL-CDR2 comprising the amino acid sequence of SEQ ID NO: 616; and VL-CDR3 comprising the amino acid sequence of SEQ ID NO: 637; or
c) VH-CDR1 comprising the amino acid sequence of SEQ ID NO: 671; VH-CDR2 comprising the amino acid sequence of SEQ ID NO: 742; and VH-CDR3 comprising the amino acid sequence of SEQ ID NO: 743; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
d) VH-CDR1 comprising the amino acid sequence of SEQ ID NO: 791; VH-CDR2 comprising the amino acid sequence of SEQ ID NO: 792; and VH-CDR3 comprising the amino acid sequence of SEQ ID NO: 793; VL-CDR1 comprising the amino acid sequence of SEQ ID NO: 795; VL-CDR2 comprising the amino acid sequence of SEQ ID NO: 616; and VL-CDR3 comprising the amino acid sequence of SEQ ID NO: 797; or
e) VH-CDR1 comprising the amino acid sequence of SEQ ID NO: 841; VH-CDR2 comprising the amino acid sequence of SEQ ID NO: 842; and VH-CDR3 comprising the amino acid sequence of SEQ ID NO: 843; VL-CDR1 comprising the amino acid sequence of SEQ ID NO: 845; VL-CDR2 comprising the amino acid sequence of SEQ ID NO: 846; and VL-CDR3 comprising the amino acid sequence of SEQ ID NO: 847; or
f) VH-CDR1 comprising the amino acid sequence of SEQ ID NO: 611; VH-CDR2 comprising the amino acid sequence of SEQ ID NO: 612; and VH-CDR3 comprising the amino acid sequence of SEQ ID NO: 613; VL-CDR1 comprising the amino acid sequence of SEQ ID NO: 615; VL-CDR2 comprising the amino acid sequence of SEQ ID NO: 616; and VL-CDR3 comprising the amino acid sequence of SEQ ID NO: 617; or
g) VH-CDR1 comprising the amino acid sequence of SEQ ID NO: 621; VH-CDR2 comprising the amino acid sequence of SEQ ID NO: 622; and VH-CDR3 comprising the amino acid sequence of SEQ ID NO: 623; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
h) VH-CDR1 comprising the amino acid sequence of SEQ ID NO: 631; VH-CDR2 comprising the amino acid sequence of SEQ ID NO: 632; and VH-CDR3 comprising the amino acid sequence of SEQ ID NO: 633; VL-CDR1 comprising the amino acid sequence of SEQ ID NO: 635; VL-CDR2 comprising the amino acid sequence of SEQ ID NO: 616; and VL-CDR3 comprising the amino acid sequence of SEQ ID NO: 637; or
i) VH-CDR1 comprising the amino acid sequence of SEQ ID NO: 641; VH-CDR2 comprising the amino acid sequence of SEQ ID NO: 642; and VH-CDR3 comprising the amino acid sequence of SEQ ID NO: 643; VL-CDR1 comprising the amino acid sequence of SEQ ID NO: 625; VL-CDR2 comprising the amino acid sequence of SEQ ID NO: 626; and VL-CDR3 comprising the amino acid sequence of SEQ ID NO: 627; or
j) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 653; VL-CDR1 comprising the amino acid sequence of SEQ ID NO: 655; VL-CDR2 comprising the amino acid sequence of SEQ ID NO: 626; and VL-CDR3 comprising the amino acid sequence of SEQ ID NO: 627; or
k) VH-CDR1 comprising the amino acid sequence of SEQ ID NO: 661; VH-CDR2 comprising the amino acid sequence of SEQ ID NO: 662; and VH-CDR3 comprising the amino acid sequence of SEQ ID NO: 663; VL-CDR1 comprising the amino acid sequence of SEQ ID NO: 665; VL-CDR2 comprising the amino acid sequence of SEQ ID NO: 666; and VL-CDR3 comprising the amino acid sequence of SEQ ID NO: 667; or
l) VH-CDR1 comprising the amino acid sequence of SEQ ID NO: 671; VH-CDR2 comprising the amino acid sequence of SEQ ID NO: 672; and VH-CDR3 comprising the amino acid sequence of SEQ ID NO: 673; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
m) VH-CDR1 comprising the amino acid sequence of SEQ ID NO: 621; VH-CDR2 comprising the amino acid sequence of SEQ ID NO: 642; and VH-CDR3 comprising the amino acid sequence of SEQ ID NO: 683; VL-CDR1 comprising the amino acid sequence of SEQ ID NO: 625; VL-CDR2 comprising the amino acid sequence of SEQ ID NO: 686; and VL-CDR3 comprising the amino acid sequence of SEQ ID NO: 627; or
n) VH-CDR1 comprising the amino acid sequence of SEQ ID NO: 701; VH-CDR2 comprising the amino acid sequence of SEQ ID NO: 702; and VH-CDR3 comprising the amino acid sequence of SEQ ID NO: 703; VL-CDR1 comprising the amino acid sequence of SEQ ID NO: 705; VL-CDR2 comprising the amino acid sequence of SEQ ID NO: 706; and VL-CDR3 comprising the amino acid sequence of SEQ ID NO: 707; or
o) VH-CDR1 comprising the amino acid sequence of SEQ ID NO: 711; VH-CDR2 comprising the amino acid sequence of SEQ ID NO: 712; and VH-CDR3 comprising the amino acid sequence of SEQ ID NO: 713; VL-CDR1 comprising the amino acid sequence of SEQ ID NO: 715; VL-CDR2 comprising the amino acid sequence of SEQ ID NO: 716; and VL-CDR3 comprising the amino acid sequence of SEQ ID NO: 717; or
p) VH-CDR1 comprising the amino acid sequence of SEQ ID NO: 731; VH-CDR2 comprising the amino acid sequence of SEQ ID NO: 722; and VH-CDR3 comprising the amino acid sequence of SEQ ID NO: 733; VL-CDR1 comprising the amino acid sequence of SEQ ID NO: 735; VL-CDR2 comprising the amino acid sequence of SEQ ID NO: 736; and VL-CDR3 comprising the amino acid sequence of SEQ ID NO: 637; or
q) VH-CDR1 comprising the amino acid sequence of SEQ ID NO: 751; VH-CDR2 comprising the amino acid sequence of SEQ ID NO: 722; and VH-CDR3 comprising the amino acid sequence of SEQ ID NO: 723; VL-CDR1 comprising the amino acid sequence of SEQ ID NO: 735; VL-CDR2 comprising the amino acid sequence of SEQ ID NO: 616; and VL-CDR3 comprising the amino acid sequence of SEQ ID NO: 637; or
r) VH-CDR1 comprising the amino acid sequence of SEQ ID NO: 761; VH-CDR2 comprising the amino acid sequence of SEQ ID NO: 722; and VH-CDR3 comprising the amino acid sequence of SEQ ID NO: 733; VL-CDR1 comprising the amino acid sequence of SEQ ID NO: 765; VL-CDR2 comprising the amino acid sequence of SEQ ID NO: 616; and VL-CDR3 comprising the amino acid sequence of SEQ ID NO: 637; or
s) VH-CDR1 comprising the amino acid sequence of SEQ ID NO: 771; VH-CDR2 comprising the amino acid sequence of SEQ ID NO: 772; and VH-CDR3 comprising the amino acid sequence of SEQ ID NO: 773; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
t) VH-CDR1 comprising the amino acid sequence of SEQ ID NO: 771; VH-CDR2 comprising the amino acid sequence of SEQ ID NO: 802; and VH-CDR3 comprising the amino acid sequence of SEQ ID NO: 803; VL-CDR1 comprising the amino acid sequence of SEQ ID NO: 805; VL-CDR2 comprising the amino acid sequence of SEQ ID NO: 676; and VL-CDR3 comprising the amino acid sequence of SEQ ID NO: 677; or
u) VH-CDR1 comprising the amino acid sequence of SEQ ID NO: 811; VH-CDR2 comprising the amino acid sequence of SEQ ID NO: 812; and VH-CDR3 comprising the amino acid sequence of SEQ ID NO: 813; VL-CDR1 comprising the amino acid sequence of SEQ ID NO: 815; VL-CDR2 comprising the amino acid sequence of SEQ ID NO: 696; and VL-CDR3 comprising the amino acid sequence of SEQ ID NO: 817; or
v) VH-CDR1 comprising the amino acid sequence of SEQ ID NO: 821; VH-CDR2 comprising the amino acid sequence of SEQ ID NO: 822; and VH-CDR3 comprising the amino acid sequence of SEQ ID NO: 823; VL-CDR1 comprising the amino acid sequence of SEQ ID NO: 825; VL-CDR2 comprising the amino acid sequence of SEQ ID NO: 826; and VL-CDR3 comprising the amino acid sequence of SEQ ID NO: 827; or
w) VH-CDR1 comprising the amino acid sequence of SEQ ID NO: 831; VH-CDR2 comprising the amino acid sequence of SEQ ID NO: 832; and VH-CDR3 comprising the amino acid sequence of SEQ ID NO: 833; VL-CDR1 comprising the amino acid sequence of SEQ ID NO: 835; VL-CDR2 comprising the amino acid sequence of SEQ ID NO: 836; and VL-CDR3 comprising the amino acid sequence of SEQ ID NO: 817.

73. The alpha-synuclein binding molecule of claim 72, which:
(i) inhibits or delays seeded or spontaneous alpha-synuclein aggregation; and
(ii) is capable of recognizing and binding to pathological or aggregated alpha-synuclein, particularly human alpha-synuclein, in vitro or in vivo.

74. The alpha-synuclein binding molecule of claim 72, which is an antibody or an antibody-binding fragment thereof comprising:
a) VH-CDR1 comprising the amino acid sequence of SEQ ID NO: 691; VH-CDR2 comprising the amino acid sequence of SEQ ID NO: 692; and VH-CDR3 comprising the amino acid sequence of SEQ ID NO: 693; VL-CDR1 comprising the amino acid sequence of SEQ ID NO: 695; VL-CDR2 comprising the amino acid sequence of SEQ ID NO: 696; and VL-CDR3 comprising the amino acid sequence of SEQ ID NO: 697; or
b) VH-CDR1 comprising the amino acid sequence of SEQ ID NO: 721; VH-CDR2 comprising the amino acid sequence of SEQ ID NO: 722; and VH-CDR3 comprising the amino acid sequence of SEQ ID NO: 723; VL-CDR1 comprising the amino acid sequence of SEQ ID NO: 725; VL-CDR2 comprising the amino acid sequence of SEQ ID NO: 616; and VL-CDR3 comprising the amino acid sequence of SEQ ID NO: 637; or
c) VH-CDR1 comprising the amino acid sequence of SEQ ID NO: 671; VH-CDR2 comprising the amino acid sequence of SEQ ID NO: 742; and VH-CDR3 comprising the amino acid sequence of SEQ ID NO: 743; VL-CDR1 comprising the amino acid sequence of SEQ ID NO: 675; VL-CDR2 comprising the amino acid sequence of SEQ ID NO: 676; and VL-CDR3 comprising the amino acid sequence of SEQ ID NO: 677; or
d) VH-CDR1 comprising the amino acid sequence of SEQ ID NO: 791; VH-CDR2 comprising the amino acid sequence of SEQ ID NO: 792; and VH-CDR3 comprising the amino acid sequence of SEQ ID NO: 793; VL-CDR1 comprising the amino acid sequence of SEQ ID NO: 795; VL-CDR2 comprising the amino acid sequence of SEQ ID NO: 616; and VL-CDR3 comprising the amino acid sequence of SEQ ID NO: 797; or
e) VH-CDR1 comprising the amino acid sequence of SEQ ID NO: 841; VH-CDR2 comprising the amino acid sequence of SEQ ID NO: 842; and VH-CDR3 comprising the amino acid sequence of SEQ ID NO: 843; VL-CDR1 comprising the amino acid sequence of SEQ ID NO: 845; VL-CDR2 comprising the amino acid sequence of SEQ ID NO: 846; and VL-CDR3 comprising the amino acid sequence of SEQ ID NO: 847.

75. The alpha-synuclein binding molecule of claim 72, which is an antibody or an antibody-binding fragment thereof comprising:
a. a Heavy Chain Variable Region (VH) comprising the sequence of SEQ ID NO: 690 or a heavy chain variable region (VH) having at least 87%, 88%, 89%, 90%, 91%, 92%, 93%, 94%, 95%, 96%, 97%, 98%, or 99% sequence identity to the amino acid sequence of SEQ ID NO: 690; and a Light Chain Variable Region (VL) comprising the sequence of SEQ ID NO: 694 or a light chain variable region (VL) having at least 96%, 97%, 98%, or 99% sequence identity to the amino acid sequence of SEQ ID NO: 694; or
b. a Heavy Chain Variable Region (VH) comprising the sequence of SEQ ID NO: 720; and a Light Chain Variable Region (VL) comprising the sequence of SEQ ID NO: 724 or a light chain variable region (VL) having at least 95%, 96%, 97%, 98%, or 99% sequence identity to the amino acid sequence of SEQ ID NO: 724; or
c. a Heavy Chain Variable Region (VH) comprising the sequence of SEQ ID NO: 740 or a heavy chain variable region (VH) having at least 88%, 89%, 90%, 91%, 92%, 93%, 94%, 95%, 96%, 97%, 98%, or 99% sequence identity to the amino acid sequence of SEQ ID NO: 740; and a Light Chain Variable Region (VL) comprising the sequence of SEQ ID NO: 744 or a light chain variable region (VL) having at least 99% sequence identity to the amino acid sequence of SEQ ID NO: 744; or
d. a Heavy Chain Variable Region (VH) comprising the sequence of SEQ ID NO: 790 or a heavy chain variable region (VH) having at least 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 790; and a Light Chain Variable Region (VL) comprising the sequence of SEQ ID NO: 794 or a light chain variable region (VL) having at least 95%, 96%, 97%, 98%, or 99% sequence identity to the amino acid sequence of SEQ ID NO: 794; or
e. a Heavy Chain Variable Region (VH) comprising the sequence of SEQ ID NO: 840 or a heavy chain variable region (VH) having at least 90%, 91%, 92%, 93%, 94%, 95%, 96%, 97%, 98%, or 99% sequence identity to the amino acid sequence of SEQ ID NO: 840; and a Light Chain Variable Region (VL) comprising the sequence of SEQ ID NO: 844; or
f. a Heavy Chain Variable Region (VH) comprising the sequence of SEQ ID NO: 610 or a heavy chain variable region (VH) having at least 92%, 93%, 94%, 95%, 96%, 97%, 98%, or 99% sequence identity to the amino acid sequence of SEQ ID NO: 610; and a Light Chain Variable Region (VL) comprising the sequence of SEQ ID NO: 614; or
g. a Heavy Chain Variable Region (VH) comprising the sequence of SEQ ID NO: 620 or a heavy chain variable region (VH) having at least 96%, 97%, 98%, or 99% sequence identity to the amino acid sequence of SEQ ID NO: 620; and a Light Chain Variable Region (VL) comprising the sequence of SEQ ID NO: 624 or a light chain variable region (VL) having at least 92%, 93%, 94%, 95%, 96%, 97%, 98%, or 99% sequence identity to the amino acid sequence of SEQ ID NO: 624; or
h. a Heavy Chain Variable Region (VH) comprising the sequence of SEQ ID NO: 630 or a heavy chain variable region (VH) having at least 99% sequence identity to the amino acid sequence of SEQ ID NO: 630; and a Light Chain Variable Region (VL) comprising the sequence of SEQ ID NO: 634 or a light chain variable region (VL) having at least 99% sequence identity to the amino acid sequence of SEQ ID NO: 634; or
i. a Heavy Chain Variable Region (VH) comprising the sequence of SEQ ID NO: 640 or a heavy chain variable region (VH) having at least 96%, 97%, 98%, or 99% sequence identity to the amino acid sequence of SEQ ID NO: 640; and a Light Chain Variable Region (VL) comprising the sequence of SEQ ID NO: 644 or a light chain variable region (VL) having at least 96%, 97%, 98%, or 99% sequence identity to the amino acid sequence of SEQ ID NO: 644; or
j. a Heavy Chain Variable Region (VH) comprising the sequence of SEQ ID NO: 650 or a heavy chain variable region (VH) having at least 98%, or 99% sequence identity to the amino acid sequence of SEQ ID NO: 650; and a Light Chain Variable Region (VL) comprising the sequence of SEQ ID NO: 654 or a light chain variable region (VL) having at least 99% sequence identity to the amino acid sequence of SEQ ID NO: 654; or
k. a Heavy Chain Variable Region (VH) comprising the sequence of SEQ ID NO: 660 or a heavy chain variable region (VH) having at least 92%, 93%, 94%, 95%, 96%, 97%, 98%, or 99% sequence identity to the amino acid sequence of SEQ ID NO: 660; and a Light Chain Variable Region (VL) comprising the sequence of SEQ ID NO: 664 or a light chain variable region (VL) having at least 99% sequence identity to the amino acid sequence of SEQ ID NO: 664; or
l. a Heavy Chain Variable Region (VH) comprising the sequence of SEQ ID NO: 670 or a heavy chain variable region (VH) having at least 92%, 93%, 94%, 95%, 96%, 97%, 98%, or 99% sequence identity to the amino acid sequence of SEQ ID NO: 670; and a Light Chain Variable Region (VL) comprising the sequence of SEQ ID NO: 674; or
m. a Heavy Chain Variable Region (VH) comprising the sequence of SEQ ID NO: 680 or a heavy chain variable region (VH) having at least 97%, 98%, or 99% sequence identity to the amino acid sequence of SEQ ID NO: 680; and a Light Chain Variable Region (VL) comprising the sequence of SEQ ID NO: 684 or a light chain variable region (VL) having at least 99% sequence identity to the amino acid sequence of SEQ ID NO: 684; or
n. a Heavy Chain Variable Region (VH) comprising the sequence of SEQ ID NO: 700 or a heavy chain variable region (VH) having at least 86%, 87%, 88%, 89%, 90%, 91%, 92%, 93%, 94%, 95%, 96%, 97%, 98%, or 99% sequence identity to the amino acid sequence of SEQ ID NO: 700; and a Light Chain Variable Region (VL) comprising the sequence of SEQ ID NO: 704 or a light chain variable region (VL) having at least 94%, 95%, 96%, 97%, 98%, or 99% sequence identity to the amino acid sequence of SEQ ID NO: 704; or
o. a Heavy Chain Variable Region (VH) comprising the sequence of SEQ ID NO: 710 or a heavy chain variable region (VH) having at least 96%, 97%, 98%, or 99% sequence identity to the amino acid sequence of SEQ ID NO: 710; and a Light Chain Variable Region (VL) comprising the sequence of SEQ ID NO: 714 or a light chain variable region (VL) having at least 98%, or 99% sequence identity to the amino acid sequence of SEQ ID NO: 714; or
p. a Heavy Chain Variable Region (VH) comprising the sequence of SEQ ID NO: 730 or a heavy chain variable region (VH) having at least 97%, 98%, or 99% sequence identity to the amino acid sequence of SEQ ID NO: 730; and a Light Chain Variable Region (VL) comprising the sequence of SEQ ID NO: 734 or a light chain variable region (VL) having at least 97%, 98%, or 99% sequence identity to the amino acid sequence of SEQ ID NO: 734; or
q. a Heavy Chain Variable Region (VH) comprising the sequence of SEQ ID NO: 750 or a heavy chain variable region (VH) having at least 99% sequence identity to the amino acid sequence of SEQ ID NO: 750; and a Light Chain Variable Region (VL) comprising the sequence of SEQ ID NO: 754 or a light chain variable region (VL) having at least 97%, 98%, or 99% sequence identity to the amino acid sequence of SEQ ID NO: 754; or
r. a Heavy Chain Variable Region (VH) comprising the sequence of SEQ ID NO: 760 or a heavy chain variable region (VH) having at least 97%, 98%, 99% sequence identity to the amino acid sequence of SEQ ID NO: 760; and a Light Chain Variable Region (VL) comprising the sequence of SEQ ID NO: 764 or a light chain variable region (VL) having at least 98%, or 99% sequence identity to the amino acid sequence of SEQ ID NO: 764; or
s. a Heavy Chain Variable Region (VH) comprising the sequence of SEQ ID NO: 770 or a heavy chain variable region (VH) having at least 93%, 94%, 95%, 96%, 97%, 98%, or 99% sequence identity to the amino acid sequence of SEQ ID NO: 770; and a Light Chain Variable Region (VL) comprising the sequence of SEQ ID NO: 774 or a light chain variable region (VL) having at least 97%, 98%, or 99% sequence identity to the amino acid sequence of SEQ ID NO: 774; or
t. a Heavy Chain Variable Region (VH) comprising the sequence of SEQ ID NO: 750 or a heavy chain variable region (VH) having at least 99% sequence identity to the amino acid sequence of SEQ ID NO: 750; and a Light Chain Variable Region (VL) comprising the sequence of SEQ ID NO: 784 or a light chain variable region (VL) having at least 96%, 97%, 98%, or 99% sequence identity to the amino acid sequence of SEQ ID NO: 784; or
u. a Heavy Chain Variable Region (VH) comprising the sequence of SEQ ID NO: 800 or a heavy chain variable region (VH) having at least 92%, 93%, 94%, 95%, 96%, 97%, 98%, or 99% sequence identity to the amino acid sequence of SEQ ID NO: 800; and a Light Chain Variable Region (VL) comprising the sequence of SEQ ID NO: 804 or a light chain variable region (VL) having at least 96%, 97%, 98%, or 99% sequence identity to the amino acid sequence of SEQ ID NO: 804; or
v. a Heavy Chain Variable Region (VH) comprising the sequence of SEQ ID NO: 810 or a heavy chain variable region (VH) having at least 94%, 95%, 96%, 97%, 98%, or 99% sequence identity to the amino acid sequence of SEQ ID NO: 810; and a Light Chain Variable Region (VL) comprising the sequence of SEQ ID NO: 814 or a light chain variable region (VL) having at least 99% sequence identity to the amino acid sequence of SEQ ID NO: 814; or
w. a Heavy Chain Variable Region (VH) comprising the sequence of SEQ ID NO: 820 or a heavy chain variable region (VH) having at least 98% or 99% sequence identity to the amino acid sequence of SEQ ID NO: 820; and a Light Chain Variable Region (VL) comprising the sequence of SEQ ID NO: 824 or a light chain variable region (VL) having at least 98% or 99% sequence identity to the amino acid sequence of SEQ ID NO: 824; or
x. a Heavy Chain Variable Region (VH) comprising the sequence of SEQ ID NO: 830 or a heavy chain variable region (VH) having at least 94%, 95%, 96%, 97%, 98%, or 99% sequence identity to the amino acid sequence of SEQ ID NO: 830; and a Light Chain Variable Region (VL) comprising the sequence of SEQ ID NO: 834.

76. The alpha-synuclein binding molecule of claim 72, which is an antibody or an antibody-binding fragment thereof comprising:
a. a Heavy Chain Variable Region (VH) comprising the sequence of SEQ ID NO: 690 or a heavy chain variable region (VH) having at least 87%, 88%, 89%, 90%, 91%, 92%, 93%, 94%, 95%, 96%, 97%, 98%, or 99% sequence identity to the amino acid sequence of SEQ ID NO: 690; and a Light Chain Variable Region (VL) comprising the sequence of SEQ ID NO: 694 or a light chain variable region (VL) having at least 96%, 97%, 98%, or 99% sequence identity to the amino acid sequence of SEQ ID NO: 694; or
b. a Heavy Chain Variable Region (VH) comprising the sequence of SEQ ID NO: 720; and a Light Chain Variable Region (VL) comprising the sequence of SEQ ID NO: 724 or a light chain variable region (VL) having at least 95%, 96%, 97%, 98%, or 99% sequence identity to the amino acid sequence of SEQ ID NO: 724; or
c. a Heavy Chain Variable Region (VH) comprising the sequence of SEQ ID NO: 740 or a heavy chain variable region (VH) having at least 88%, 89%, 90%, 91%, 92%, 93%, 94%, 95%, 96%, 97%, 98%, or 99% sequence identity to the amino acid sequence of SEQ ID NO: 740; and a Light Chain Variable Region (VL) comprising the sequence of SEQ ID NO: 744 or a light chain variable region (VL) having at least 99% sequence identity to the amino acid sequence of SEQ ID NO: 744; or
d. a Heavy Chain Variable Region (VH) comprising the sequence of SEQ ID NO: 790 or a heavy chain variable region (VH) having at least 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 790; and a Light Chain Variable Region (VL) comprising the sequence of SEQ ID NO: 794 or a light chain variable region (VL) having at least 95%, 96%, 97%, 98%, or 99% sequence identity to the amino acid sequence of SEQ ID NO: 794; or
e. a Heavy Chain Variable Region (VH) comprising the sequence of SEQ ID NO: 840 or a heavy chain variable region (VH) having at least 90%, 91%, 92%, 93%, 94%, 95%, 96%, 97%, 98%, or 99% sequence identity to the amino acid sequence of SEQ ID NO: 840; and a Light Chain Variable Region (VL) comprising the sequence of SEQ ID NO: 844.

77. The alpha-synuclein binding molecule of claim 72, which is an antibody or an antibody-binding fragment thereof comprising:
a. a Heavy Chain Variable Region (VH) comprising the sequence of SEQ ID NO: 690 and a Light Chain Variable Region (VL) comprising the sequence of SEQ ID NO: 694; or
b. a Heavy Chain Variable Region (VH) comprising the sequence of SEQ ID NO: 720 and a Light Chain Variable Region (VL) comprising the sequence of SEQ ID NO: 724; or
c. a Heavy Chain Variable Region (VH) comprising the sequence of SEQ ID NO: 740 and a Light Chain Variable Region (VL) comprising the sequence of SEQ ID NO: 744; or
d. a Heavy Chain Variable Region (VH) comprising the sequence of SEQ ID NO: 790 and a Light Chain Variable Region (VL) comprising the sequence of SEQ ID NO: 794; or
e. a Heavy Chain Variable Region (VH) comprising the sequence of SEQ ID NO: 840 and a Light Chain Variable Region (VL) comprising the sequence of SEQ ID NO: 844; or
f. a Heavy Chain Variable Region (VH) comprising the sequence of SEQ ID NO: 610 and a Light Chain Variable Region (VL) comprising the sequence of SEQ ID NO: 614; or
g. a Heavy Chain Variable Region (VH) comprising the sequence of SEQ ID NO: 620 and a Light Chain Variable Region (VL) comprising the sequence of SEQ ID NO: 624; or
h. a Heavy Chain Variable Region (VH) comprising the sequence of SEQ ID NO: 630 and a Light Chain Variable Region (VL) comprising the sequence of SEQ ID NO: 634; or
i. a Heavy Chain Variable Region (VH) comprising the sequence of SEQ ID NO: 640 and a Light Chain Variable Region (VL) comprising the sequence of SEQ ID NO: 644; or
j. a Heavy Chain Variable Region (VH) comprising the sequence of SEQ ID NO: 650 and a Light Chain Variable Region (VL) comprising the sequence of SEQ ID NO: 654; or
k. a Heavy Chain Variable Region (VH) comprising the sequence of SEQ ID NO: 660 and a Light Chain Variable Region (VL) comprising the sequence of SEQ ID NO: 664; or
l. a Heavy Chain Variable Region (VH) comprising the sequence of SEQ ID NO: 670 and a Light Chain Variable Region (VL) comprising the sequence of SEQ ID NO: 674; or
m. a Heavy Chain Variable Region (VH) comprising the sequence of SEQ ID NO: 680 and a Light Chain Variable Region (VL) comprising the sequence of SEQ ID NO: 684; or
n. a Heavy Chain Variable Region (VH) comprising the sequence of SEQ ID NO: 700 and a Light Chain Variable Region (VL) comprising the sequence of SEQ ID NO: 704; or
o. a Heavy Chain Variable Region (VH) comprising the sequence of SEQ ID NO: 710 and a Light Chain Variable Region (VL) comprising the sequence of SEQ ID NO: 714; or
p. a Heavy Chain Variable Region (VH) comprising the sequence of SEQ ID NO: 730 and a Light Chain Variable Region (VL) comprising the sequence of SEQ ID NO: 734; or
q. a Heavy Chain Variable Region (VH) comprising the sequence of SEQ ID NO: 750 and a Light Chain Variable Region (VL) comprising the sequence of SEQ ID NO: 754; or
r. a Heavy Chain Variable Region (VH) comprising the sequence of SEQ ID NO: 760 and a Light Chain Variable Region (VL) comprising the sequence of SEQ ID NO: 764; or
s. a Heavy Chain Variable Region (VH) comprising the sequence of SEQ ID NO: 770 and a Light Chain Variable Region (VL) comprising the sequence of SEQ ID NO: 774; or
t. a Heavy Chain Variable Region (VH) comprising the sequence of SEQ ID NO: 750 and a Light Chain Variable Region (VL) comprising the sequence of SEQ ID NO: 784; or
u. a Heavy Chain Variable Region (VH) comprising the sequence of SEQ ID NO: 800 and a Light Chain Variable Region (VL) comprising the sequence of SEQ ID NO: 804; or
v. a Heavy Chain Variable Region (VH) comprising the sequence of SEQ ID NO: 810 and a Light Chain Variable Region (VL) comprising the sequence of SEQ ID NO: 814; or
w. a Heavy Chain Variable Region (VH) comprising the sequence of SEQ ID NO: 820 and a Light Chain Variable Region (VL) comprising the sequence of SEQ ID NO: 824; or
x. a Heavy Chain Variable Region (VH) comprising the sequence of SEQ ID NO: 830 and a Light Chain Variable Region (VL) comprising the sequence of SEQ ID NO: 834.

78. The alpha-synuclein binding molecule of claim 72, which binds to an epitope within amino acids residues 1-15 (SEQ ID NO: 121), 15-45 (SEQ ID NO: 138), 19-33 (SEQ ID NO: 123), 28-50 (SEQ ID NO: 139), 31-60 (SEQ ID NO: 146), 81-120 (SEQ ID NO: 137), 91-105 (SEQ ID NO: 131), 96-140 (SEQ ID NO: 147) or 100-114 (SEQ ID NO: 132) of human alpha-synuclein of SEQ ID NO: 1, or
to a non-linear epitope within human alpha-synuclein of SEQ ID NO: 1.

79. The alpha-synuclein binding molecule of claim 72, which:
a. delays the aggregation and seeding of pathological alpha-synuclein in vivo and/or in vitro;
b. delays or inhibits the propagation of alpha-synuclein aggregates; or
c. upon pre-incubation with alpha-synuclein seeds causes an at least 10 percent increase in aggregation half-time (τ½ values) of seeded aggregation relative to the seeded aggregation in the absence of binding molecule, optionally wherein alpha-synuclein aggregation is monitored by a thioflavin T (ThT) fluorescence assay.

80. The alpha-synuclein binding molecule of claim 72, which:
a. binds (in a Parkinson's disease (PD) context) to pathological and/or aggregated alpha-synuclein in Lewy bodies and Lewy neurites; or
b. binds (in a multiple system atrophy (MSA) context) to pathological and/or aggregated alpha-synuclein in glial cytoplasmic inclusions.

81. The alpha-synuclein binding molecule of claim 72, which is:
a. an antibody or an antigen-binding fragment thereof;
b. a monoclonal antibody or an antigen-binding fragment thereof;
c. a murine, chimeric, humanized or a human antibody or an antigen-binding fragment thereof;
d. an IgA, IgD, IgE, IgM, IgG1, IgG2, IgG2a, IgG2b, IgG3 or IgG4 antibody or antigen-binding fragment thereof; or
e. the binding molecule is an IgG4 isotype including the S228P mutation.

82. An immunoconjugate comprising the alpha-synuclein binding molecule according to claim 72, optionally wherein the immunoconjugate crosses the blood brain barrier using a delivery vehicle or a blood brain barrier moiety, optionally wherein:
a. the delivery vehicle comprises a liposome or extracellular vesicle;
b. the alpha-synuclein binding molecule is linked to a blood brain barrier moiety;
c. the blood brain barrier moiety is a polypeptide or a small molecule, preferably, a peptide, a receptor ligand, a single domain antibody (VHH), a scFv or a Fab fragment; or
d. wherein the blood brain barrier moiety binds a blood brain barrier receptor, preferably a transferrin receptor, insulin receptor or low-density lipoprotein receptor.

83. A method for preventing, alleviating or treating diseases, disorders and abnormalities associated with alpha-synuclein, particularly with pathological alpha-synuclein or aggregated alpha-synuclein by administering the alpha synuclein binding molecule of claim 72 to a subject, optionally wherein the aggregated alpha-synuclein is in the form of Lewy bodies, Lewy neurites or glial cytoplasmic inclusions, preferably wherein the disease, disorder or abnormality is:
a. a synucleinopathy, or
b. Parkinson's disease (PD) (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), Diffuse Lewy Body Disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (MSA) (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder, more preferably wherein the disease, disorder or abnormality is selected from the group consisting of Parkinson's Disease, Multiple System Atrophy, Lewy Body dementia (LBD; dementia with Lewy bodies (DLB) (“pure” Lewy body dementia), Parkinson's disease dementia (PDD)) and Diffuse Lewy Body Disease.

84. The method according to claim 83 for preventing, slowing down, halting, retaining or improving the motor capabilities or motor deficits, cognitive capabilities or cognitive deficits, behavioral impairments or REM sleep disorders of a subject suffering from a synucleinopathy, wherein the synucleinopathy is multiple system atrophy (MSA) Parkinson's Disease, Lewy Body dementia (LBD; dementia with Lewy bodies (DLB) (“pure” Lewy body dementia), Parkinson's disease dementia (PDD)) or Diffuse Lewy Body Disease.

85. A method for diagnosing diseases, disorders and abnormalities associated with alpha-synuclein, particularly with pathological alpha-synuclein or aggregated alpha-synuclein, using the alpha-synuclein binding molecule according to claim 72.

86. A method for:
a. detecting alpha-synuclein in a sample obtained from a subject, the method comprising contacting the sample with the alpha-synuclein binding antibody or antigen-binding fragment thereof of claim 72 and detecting binding of the antibody or antigen-binding fragment thereof in order to detect alpha-synuclein in the sample, or
b. quantifying alpha-synuclein in a sample obtained from a subject, the method comprising contacting the sample with the alpha-synuclein binding antibody or antigen-binding fragment thereof of claim 72 and quantifying alpha-synuclein in a sample based on the level of binding of the antibody or antigen-binding fragment thereof to alpha-synuclein.

87. A method for diagnosing a disease, disorder or condition associated with alpha-synuclein comprising performing the quantifying method of claim 86 wherein:
a. higher levels of alpha-synuclein in the sample compared with a control level based on healthy subjects are indicative of a disease, disorder or condition associated with alpha-synuclein; or
b. similar or higher levels of alpha-synuclein in the sample compared with a diseased control level are indicative of a disease, disorder or condition associated with alpha-synuclein; or
a method for classifying a disease, disorder or condition associated with alpha-synuclein comprising:
a. performing the quantifying method of claim 86, wherein
i. higher levels of alpha-synuclein in the sample compared with a control level based on healthy subjects are indicative of a disease, disorder or condition associated with alpha-synuclein; or
ii. similar or higher levels of alpha-synuclein in the sample compared with a diseased control at a certain stage of disease are indicative of that stage of disease, disorder or condition associated with alpha-synuclein; and
b. classifying the disease, disorder or condition associated with alpha-synuclein.

88. A method for monitoring a disease, disorder or condition associated with alpha-synuclein at two or more time points using samples from a subject comprising contacting the samples with an alpha-synuclein binding antibody or antigen-binding fragment thereof of claim 72, wherein:
a. higher levels of alpha-synuclein in the later sample compared with one or more earlier samples are indicative of progression of a disease, disorder and/or condition associated with alpha-synuclein;
b. lower levels of alpha-synuclein in the later sample compared with one or more earlier samples are indicative of regression of a disease, disorder and/or condition associated with alpha-synuclein; or
c. no significant change of levels of alpha-synuclein in the later sample compared with one or more earlier samples are indicative of lack of progression of a disease, disorder or condition associated with alpha-synuclein; or
a method for selecting a therapy for treatment of a disease, disorder or condition associated with alpha-synuclein comprising contacting samples taken before and after treatment with the therapy with an alpha-synuclein binding antibody or antigen-binding fragment thereof of claim 72, wherein:
a. lower levels of alpha-synuclein in the sample taken after treatment compared with the sample taken before treatment are indicative of successful treatment of a disease, disorder or condition associated with alpha-synuclein and thus the therapy is selected for treatment;
b. no significant change of levels of alpha-synuclein in the sample taken after treatment compared with the sample taken before treatment are indicative of successful treatment of a disease, disorder or condition associated with alpha-synuclein and thus the therapy is selected for treatment;
c. a decline in the rate of increase of levels of alpha-synuclein between samples taken during treatment compared with samples taken before treatment are indicative of successful treatment of a disease, disorder or condition associated with alpha-synuclein and thus the therapy is selected for treatment;
d. higher levels of alpha-synuclein in the sample taken after treatment compared with the sample taken before treatment are indicative of unsuccessful treatment of a disease, disorder or condition associated with alpha-synuclein and thus the therapy is not selected for treatment; or
e. no decline in the rate of increase of levels of alpha-synuclein between samples taken during treatment compared with samples taken before treatment are indicative of unsuccessful treatment of a disease, disorder or condition associated with alpha-synuclein and thus the therapy is not selected for treatment.

89. A method for assessing a candidate therapy for a disease, disorder or condition associated with alpha-synuclein, the method comprising, following treatment of one or more subjects, contacting samples from the one or more treated subjects with an antibody or antigen-binding fragment of claim 72, wherein lower levels of alpha-synuclein in the samples compared with levels in corresponding samples from subjects not treated with the therapy are indicative of successful treatment of a disease, disorder or condition associated with alpha-synuclein, optionally wherein the method is performed at multiple time points in matched samples between the treatment and placebo groups in order to monitor the effectiveness of the candidate therapy over a defined time period or comprises contacting samples from the one or more treated subjects and the subjects not treated with the therapy with an antibody or antigen-binding fragment of claim 72 prior to treatment, with the therapy or placebo respectively, to determine base levels of alpha-synuclein.

90. The method according to claim 86 wherein the alpha-synuclein comprises, consists essentially of or consists of pathological alpha-synuclein or aggregated alpha-synuclein and wherein the disease, disorder or condition associated with alpha-synuclein is Parkinson's disease (PD) (sporadic, familial with alpha-synuclein mutations, familial with mutations other than alpha-synuclein, pure autonomic failure and Lewy body dysphagia), Lewy Body dementia (LBD; dementia with Lewy bodies (DLB) (“pure” Lewy body dementia), Parkinson's disease dementia (PDD)), Diffuse Lewy Body Disease (DLBD), sporadic Alzheimer's disease, familial Alzheimer's disease with APP mutations, familial Alzheimer's disease with PS-1, PS-2 or other mutations, familial British dementia, Lewy body variant of Alzheimer's disease, multiple system atrophy (MSA) (Shy-Drager syndrome, striatonigral degeneration and olivopontocerebellar atrophy), inclusion-body myositis, traumatic brain injury, chronic traumatic encephalopathy, dementia pugilistica, tauopathies (Pick's disease, frontotemporal dementia, progressive supranuclear palsy, corticobasal degeneration, Frontotemporal dementia with Parkinsonism linked to chromosome 17 and Niemann-Pick type C1 disease), Down syndrome, Creutzfeldt-Jakob disease, Huntington's disease, motor neuron disease, amyotrophic lateral sclerosis (sporadic, familial and ALS-dementia complex of Guam), neuroaxonal dystrophy, neurodegeneration with brain iron accumulation type 1 (Hallervorden-Spatz syndrome), prion diseases, Gerstmann-Straussler-Scheinker disease, ataxia telangiectatica, Meige's syndrome, subacute sclerosing panencephalitis, Gaucher disease, Krabbe disease as well as other lysosomal storage disorders (including Kufor-Rakeb syndrome and Sanfilippo syndrome), or rapid eye movement (REM) sleep behavior disorder.

91. The alpha-synuclein antibody or antigen-binding fragment thereof of claim 72 for research use, in particular as an analytical tool or reference molecule.

92. A pharmaceutical composition comprising the alpha-synuclein binding molecule of claim 72 and a pharmaceutically acceptable carrier or excipient.

93. A nucleic acid, recombinant expression vector comprising a nucleic acid, host cell comprising a nucleic acid or cell-free expression system comprising a recombinant expression vector which comprises a nucleic acid, wherein the nucleic acid encodes the alpha-synuclein binding molecule of claim 72, optionally wherein the nucleic acid comprises a nucleotide sequence as provided in SEQ ID NO: 618, SEQ ID NO: 619, SEQ ID NO: 628, SEQ ID NO: 629, SEQ ID NO: 638, SEQ ID NO: 639, SEQ ID NO: 648, SEQ ID NO: 649, SEQ ID NO: 658, SEQ ID NO: 659, SEQ ID NO: 668, SEQ ID NO: 669, SEQ ID NO: 678, SEQ ID NO: 679, SEQ ID NO: 688, SEQ ID NO: 689, SEQ ID NO: 698, SEQ ID NO: 699, SEQ ID NO: 708, SEQ ID NO: 709, SEQ ID NO: 718, SEQ ID NO: 719, SEQ ID NO: 728, SEQ ID NO: 729, SEQ ID NO: 738, SEQ ID NO: 739, SEQ ID NO: 748, SEQ ID NO: 749, SEQ ID NO: 758, SEQ ID NO: 759, SEQ ID NO: 768, SEQ ID NO: 769, SEQ ID NO: 778, SEQ ID NO: 779, SEQ ID NO: 789, SEQ ID NO: 798, SEQ ID NO: 799, SEQ ID NO: 808, SEQ ID NO: 809, SEQ ID NO: 818, SEQ ID NO: 819, SEQ ID NO: 828, SEQ ID NO: 829, SEQ ID NO: 838, SEQ ID NO: 839, SEQ ID NO: 848 or SEQ ID NO: 849, optionally wherein the nucleic acid is a part of a viral vector for targeted delivery to the blood brain barrier or any other cell type in the CNS, optionally wherein the viral vector is a recombinant adeno-associated viral vector (rAAV), preferably a recombinant adeno-associated viral vector selected from AAV1 to AAV12.

94. A method for producing an isolated alpha-synuclein binding molecule, in particular an antibody or antigen-binding fragment thereof, comprising the steps of:
a. culturing the host cell or the cell-free expression system of claim 93 under conditions suitable for producing the alpha-synuclein binding molecule, in particular the antibody or antigen-binding fragment thereof, and
b. isolating the alpha-synuclein binding molecule, in particular the antibody or antigen-binding fragment thereof.

95. A kit for diagnosis of a disease, disorder or abnormality associated with alpha-synuclein, comprising an alpha-synuclein binding molecule according to claim 72, optionally further comprising a container.


SEQUENCE LISTING (851 sequences)

SEQ ID NO: 1 - protein, 140 aa, Unknown
  FL-alpha-synuclein of the class Mammalia
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 5 aa, Unknown
  Binding epitope of the class Mammalia
GVLYV

SEQ ID NO: 3 - protein, 7 aa, Unknown
  Binding epitope of the class Mammalia
GVATVAE

SEQ ID NO: 4 - protein, 10 aa, Unknown
  Binding epitope of the class Mammalia
NVGGAVVTGV

SEQ ID NO: 5 - protein, 17 aa, Unknown
  Binding epitope of the class Mammalia
NVGGAVVTGVTAVAQKT

SEQ ID NO: 6 - skipped

SEQ ID NO: 7 - protein, 8 aa, Unknown
  Binding epitope of the class Mammalia
AYEMPSEE

SEQ ID NO: 8 - protein, 8 aa, Unknown
  Binding epitope of the class Mammalia
PSEEGYQD

SEQ ID NO: 9 - protein, 10 aa, Unknown
  Binding epitope of the class Mammalia
EGYQDYEPEA

SEQ ID NO: 10 - protein, 121 aa, Artificial Sequence
  ACI-7067-1101C8-Ab2 1101C8F7 VH
EVQLVESGGGLVQPKGSLKLSCAASGFSFNIYAMNWVRQAPGKGLEWVARIRSKSNNYATYYADSVKDRFTISRADSESMLYLQMNNLKTEDTAMYYCVRVGLRFYAMDYWGQGTSVTVSS

SEQ ID NO: 11 - protein, 5 aa, Artificial Sequence
  ACI-7067-1101C8-Ab2 1101C8F7 VH CDR1
IYAMN

SEQ ID NO: 12 - protein, 19 aa, Artificial Sequence
  ACI-7067-1101C8-Ab2 1101C8F7 VH CDR2
RIRSKSNNYATYYADSVKD

SEQ ID NO: 13 - protein, 10 aa, Artificial Sequence
  ACI-7067-1101C8-Ab2 1101C8F7 VH CDR3
VGLRFYAMDY

SEQ ID NO: 14 - protein, 112 aa, Artificial Sequence
  ACI-7067-1101C8-Ab2 1101C8F7 VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSQGPLTFGAGTKLELK

SEQ ID NO: 15 - protein, 16 aa, Artificial Sequence
  ACI-7067-1101C8-Ab2 1101C8F7 VL CDR1
RSSQSIVHSNGNTYLE

SEQ ID NO: 16 - protein, 7 aa, Artificial Sequence
  ACI-7067-1101C8-Ab2 1101C8F7 VL CDR2
KVSNRFS

SEQ ID NO: 17 - protein, 9 aa, Artificial Sequence
  ACI-7067-1101C8-Ab2 1101C8F7 VL CDR3
FQGSQGPLT

SEQ ID NO: 18 - DNA, 363 bp, Artificial Sequence
  ACI-7067-1101C8-Ab2 1101C8F7 VH
gaggtgcagcttgttgagtctggtggaggattggtgcagcctaaagggtcattgaaactctcatgtgcagcctctggattcagcttcaatatctacgccatgaactgggtccgccaggctccaggaaagggtttggaatgggttgctcgcataagaagtaaaagtaataattatgcaacatattatgccgattcagtgaaagacagattcaccatctccagagctgattcagaaagcatgctctatctgcaaatgaacaacttgaaaactgaggacacagccatgtattactgtgtaagggtgggcctacggttctatgctatggactactggggtcaaggcacctcagtcaccgtctcctca

SEQ ID NO: 19 - DNA, 336 bp, Artificial Sequence
  ACI-7067-1101C8-Ab2 1101C8F7 VL
gatgttttgatgacccaaactccactctccctgcctgtcagtcttggagatcaagcctccatctcttgcagatctagtcagagcattgtacatagtaatggaaacacctatttagaatggtacttgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacaaggtccgctcacgttcggtgctgggaccaagctggagctgaaa

SEQ ID NO: 20 - protein, 114 aa, Artificial Sequence
  ACI-7067-1102G3-Ab1 1102G3F2 VH
EVKLEESGGGLVQPGGSMKLSCAASGFTFSDAWMNWVRQSPEKGLEWVAEIRNKAHNHATYYAESVKGRFTISGDDSKSSVYLQMNNLRAEDTGIYYCTIYSYWGQGTLVTVSA

SEQ ID NO: 21 - protein, 4 aa, Artificial Sequence
  ACI-7067-1102G3-Ab1 1102G3F2 VH CDR1
DAWM

SEQ ID NO: 22 - protein, 19 aa, Artificial Sequence
  ACI-7067-1102G3-Ab1 1102G3F2 VH CDR2
EIRNKAHNHATYYAESVKG

SEQ ID NO: 23 - skipped

SEQ ID NO: 24 - protein, 107 aa, Artificial Sequence
  ACI-7067-1102G3-Ab1 1102G3F2 VL
SIVMTQTPKFLLVSAGDRVTITCKASQSVTKDVAWYQQKPGQSPKLLIYSTSNRYSGVPDRFTGSGYGTDFTFTINTVQTEDLAVYFCQQDYRIPYTFGGGTKLEIK

SEQ ID NO: 25 - protein, 11 aa, Artificial Sequence
  ACI-7067-1102G3-Ab1 1102G3F2 VL CDR1
KASQSVTKDVA

SEQ ID NO: 26 - protein, 7 aa, Artificial Sequence
  ACI-7067-1102G3-Ab1 1102G3F2 VL CDR2
STSNRYS

SEQ ID NO: 27 - protein, 9 aa, Artificial Sequence
  ACI-7067-1102G3-Ab1 1102G3F2 VL CDR3
QQDYRIPYT

SEQ ID NO: 28 - DNA, 342 bp, Artificial Sequence
  ACI-7067-1102G3-Ab1 1102G3F2 VH
gaagtgaagcttgaggagtctggaggaggcttggtgcaacctggaggatccatgaaactctcttgtgctgcctctggattcacttttagtgacgcctggatgaactgggtccgccagtctccagagaaggggcttgagtgggttgctgaaattagaaacaaagctcataatcatgcaacatactatgctgagtctgtgaaagggaggttcaccatctcaggagatgattccaaaagtagtgtctacctgcaaatgaacaacttaagagctgaagacactggcatttattactgtaccatttactcttattggggccaagggactctggtcactgtctctgca

SEQ ID NO: 29 - DNA, 321 bp, Artificial Sequence
  ACI-7067-1102G3-Ab1 1102G3F2 VL
agtattgtgatgacccagactcccaaattcctgcttgtatcagcaggagacagggttaccataacctgcaaggccagtcagagtgtgactaaagatgtagcttggtaccaacagaagccagggcagtctcctaaactgctgatatactctacatccaatcgctacagtggagtccctgatcgcttcactggcagtggatatgggacggatttcactttcaccatcaatactgtgcagactgaagacctggcagtttatttctgtcagcaggattacaggattccgtacacgttcggaggggggaccaagctggaaataaaa

SEQ ID NO: 30 - protein, 120 aa, Artificial Sequence
  ACI-7067-1106A8-Ab2 1106A8H3 VH
EVQLVESGGGLVQPKGSLKLSCAASGFTFNTYAMHWVRQAPGKGLEWVARIRSKGSNYATNYADSVKDRFTISRDDSQSMLYLQMNNLKTEDTAMYYCVRGHGSSYFSYWGQGTLVTVSA

SEQ ID NO: 31 - protein, 5 aa, Artificial Sequence
  ACI-7067-1106A8-Ab2 1106A8H3 VH CDR1
TYAMH

SEQ ID NO: 32 - protein, 19 aa, Artificial Sequence
  ACI-7067-1106A8-Ab2 1106A8H3 VH CDR2
RIRSKGSNYATNYADSVKD

SEQ ID NO: 33 - protein, 9 aa, Artificial Sequence
  ACI-7067-1106A8-Ab2 1106A8H3 VH CDR3
GHGSSYFSY

SEQ ID NO: 34 - protein, 106 aa, Artificial Sequence
  ACI-7067-1106A8-Ab2 1106A8H3 VL
QIVLTQSPAIMSASPGEKVTMTCSASSSVSYMHWYQQKSGTSPKRWIYDTSNLASGVPARFSGSGSGTSYSLTISSMEAEDAATYYCQQWNSHPPTFGAGTKLELK

SEQ ID NO: 35 - protein, 8 aa, Artificial Sequence
  ACI-7067-1106A8-Ab2 1106A8H3 VL CDR1
SASSSVSY

SEQ ID NO: 36 - protein, 7 aa, Artificial Sequence
  ACI-7067-1106A8-Ab2 1106A8H3 VL CDR2
DTSNLAS

SEQ ID NO: 37 - protein, 9 aa, Artificial Sequence
  ACI-7067-1106A8-Ab2 1106A8H3 VL CDR3
QQWNSHPPT

SEQ ID NO: 38 - DNA, 360 bp, Artificial Sequence
  ACI-7067-1106A8-Ab2 1106A8H3 VH
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaggtagtaattatgcaacaaattatgccgattcagtgaaagacagattcaccatctccagagatgattcgcaaagcatgctctatctgcaaatgaacaacctgaaaactgaggacacagccatgtattactgtgtgagaggacacggtagtagctacttttcttactggggccaagggactctggtcactgtctctgca

SEQ ID NO: 39 - DNA, 318 bp, Artificial Sequence
  ACI-7067-1106A8-Ab2 1106A8H3 VL
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaatctggcttctggagtccctgctcgcttcagtggcagtgggtctgggacctcttactctctcacaatcagcagcatggaggctgaagatgctgccacttattactgccagcagtggaatagtcacccacccacgttcggtgctgggaccaagctggaactgaaa

SEQ ID NO: 40 - protein, 113 aa, Artificial Sequence
  ACI-7067-1107G5-Ab2 1107G5B6 VH
QVQLQQPGTELVKPGASVKLSCKASGYTFTKYWMHWVKQRPGQGLEWIGNINPNNGDTNYNEKFKSKATLTVDKSSSTAYMQLSSLTSEDSAVYYCAIAMDYWGQGTSVTVSS

SEQ ID NO: 41 - protein, 5 aa, Artificial Sequence
  ACI-7067-1107G5-Ab2 1107G5B6 VH CDR1
KYWMH

SEQ ID NO: 42 - protein, 17 aa, Artificial Sequence
  ACI-7067-1107G5-Ab2 1107G5B6 VH CDR2
NINPNNGDTNYNEKFKS

SEQ ID NO: 43 - protein, 4 aa, Artificial Sequence
  ACI-7067-1107G5-Ab2 1107G5B6 VH CDR3
AMDY

SEQ ID NO: 44 - protein, 107 aa, Artificial Sequence
  ACI-7067-1107G5-Ab2 1107G5B6 VL
DIQMTQSPSSLSAFLGERVSLTCRASQDIGNNLNWFQQEPDGTIKRLIYATSSLDSGVPKRFSGSRSGSEYSLTISSLESEDFVDYYCLQFGSSPLTFGAGTKLELK

SEQ ID NO: 45 - protein, 11 aa, Artificial Sequence
  ACI-7067-1107G5-Ab2 1107G5B6 VL CDR1
RASQDIGNNLN

SEQ ID NO: 46 - protein, 7 aa, Artificial Sequence
  ACI-7067-1107G5-Ab2 1107G5B6 VL CDR2
ATSSLDS

SEQ ID NO: 47 - protein, 9 aa, Artificial Sequence
  ACI-7067-1107G5-Ab2 1107G5B6 VL CDR3
LQFGSSPLT

SEQ ID NO: 48 - DNA, 339 bp, Artificial Sequence
  ACI-7067-1107G5-Ab2 1107G5B6 VH
caggtccaactgcagcagcctgggactgaactggtgaagcctggggcttcagtgaagctgtcctgcaaggcttctggctacaccttcaccaaatactggatgcactgggtgaagcagaggcctggacaaggccttgagtggattggaaatattaatcctaacaatggtgatactaactacaatgagaagttcaagagcaaggccacactgactgtagacaaatcctccagcacagcctacatgcagctcagcagtctgacatctgaggactctgcggtctattattgtgcaattgctatggactactggggtcaaggaacctcagtcaccgtctcctca

SEQ ID NO: 49 - DNA, 321 bp, Artificial Sequence
  ACI-7067-1107G5-Ab2 1107G5B6 VL
gacatccagatgacccagtctccatcctccttatctgcctttctgggagaaagagtcagtctcacttgtcgggcaagtcaggacattggtaataacttaaactggtttcagcaggaaccagatggaactattaaacgtctgatctacgccacatccagtttagattctggtgtccccaaaaggttcagtggcagtaggtctgggtcagaatattctctcaccatcagcagccttgagtctgaagattttgtagactattactgtctacaatttggtagttctccgctcacgttcggtgctgggaccaagctggagctgaaa

SEQ ID NO: 50 - protein, 114 aa, Artificial Sequence
  ACI-7067-1108H1-Ab1 1108H1E1 VH
EVKLVESGGGLVQPGGSMKLSCTASGFTFSDAWMNWVRQSPEKGLEWVAEIRNKAHNHATNYAESVKGRFTISGDDSKSSVYLQMNNLRAEDTGIYYCTIYSFWGQGTLVTVSA

SEQ ID NO: 51 - protein, 4 aa, Artificial Sequence
  ACI-7067-1108H1-Ab1 1108H1E1 VH CDR1
DAWM

SEQ ID NO: 52 - protein, 19 aa, Artificial Sequence
  ACI-7067-1108H1-Ab1 1108H1E1 VH CDR2
EIRNKAHNHATNYAESVKG

SEQ ID NO: 53 - skipped

SEQ ID NO: 54 - protein, 107 aa, Artificial Sequence
  ACI-7067-1108H1-Ab1 1108H1E1 VL
SIVMTQTPKFLLVSAGDRVTITCKASQSVTNYVAWYHQKPGQSPKLLIYSASNRYSGVPDRFTGSGYGTDFTFTINTVQTEDLAVYFCQQDYRIPYTFGGGTKLEIK

SEQ ID NO: 55 - protein, 11 aa, Artificial Sequence
  ACI-7067-1108H1-Ab1 1108H1E1 VL CDR1
KASQSVTNYVA

SEQ ID NO: 56 - protein, 7 aa, Artificial Sequence
  ACI-7067-1108H1-Ab1 1108H1E1 VL CDR2
SASNRYS

SEQ ID NO: 57 - protein, 9 aa, Artificial Sequence
  ACI-7067-1108H1-Ab1 1108H1E1 VL CDR3
QQDYRIPYT

SEQ ID NO: 58 - DNA, 342 bp, Artificial Sequence
  ACI-7067-1108H1-Ab1 1108H1E1 VH
gaggtgaagctggtggagtctggaggaggcttggtgcaacctggaggatccatgaaactctcttgtactgcctctggattcacttttagtgacgcctggatgaactgggtccgccagtctccagagaaggggcttgagtgggttgctgaaattagaaacaaagctcataatcatgcaacaaactatgctgagtctgtgaaggggaggttcaccatctcaggagatgattccaaaagtagtgtctacctgcaaatgaacaacttaagagctgaagacactggcatttattactgtaccatttactctttttggggccaagggactctggtcactgtctctgca

SEQ ID NO: 59 - DNA, 321 bp, Artificial Sequence
  ACI-7067-1108H1-Ab1 1108H1E1 VL
agtattgtgatgacccagactcccaaattcctgcttgtatcagcaggagacagggttaccataacctgcaaggccagtcagagtgtgactaattatgtagcttggtaccatcagaagccagggcagtctcctaaactgctgatatactctgcatccaatcgctacagtggagtccctgatcgcttcactggcagtggatatgggacggatttcactttcaccatcaatactgtgcagactgaagacctggcagtttatttctgtcagcaggattacaggattccgtacacgttcggaggggggactaagctggaaataaaa

SEQ ID NO: 60 - protein, 113 aa, Artificial Sequence
  ACI-7067-1111B12-Ab2 1111B12H10 VH
QVQLLQPGTALVMPGASVKLSCKASGYTFTTYWMHWVKQRPGQGLEWIGNINPINGGSNYNEKFKSKASLTVDKSSSTAYMQLSSLTSEDSAVYYCVIAMDYWGQGTSVTVSS

SEQ ID NO: 61 - protein, 5 aa, Artificial Sequence
  ACI-7067-1111B12-Ab2 1111B12H10 VH CDR1
TYWMH

SEQ ID NO: 62 - protein, 17 aa, Artificial Sequence
  ACI-7067-1111B12-Ab2 1111B12H10 VH CDR2
NINPINGGSNYNEKFKS

SEQ ID NO: 63 - protein, 4 aa, Artificial Sequence
  ACI-7067-1111B12-Ab2 1111B12H10 VH CDR3
AMDY

SEQ ID NO: 64 - protein, 107 aa, Artificial Sequence
  ACI-7067-1111B12-Ab2 1111B12H10 VL
DIQMTQSPSSLSASLGERVSLTCRASQDIGISLNWFQQEPDGTIKRLIYATSSLDSGVPKRFSGNRSGSDYSLTISSLESEDFADYYCLQFASSPLTFGAGTKLELK

SEQ ID NO: 65 - protein, 11 aa, Artificial Sequence
  ACI-7067-1111B12-Ab2 1111B12H10 VL CDR1
RASQDIGISLN

SEQ ID NO: 66 - protein, 7 aa, Artificial Sequence
  ACI-7067-1111B12-Ab2 1111B12H10 VL CDR2
ATSSLDS

SEQ ID NO: 67 - protein, 9 aa, Artificial Sequence
  ACI-7067-1111B12-Ab2 1111B12H10 VL CDR3
LQFASSPLT

SEQ ID NO: 68 - DNA, 339 bp, Artificial Sequence
  ACI-7067-1111B12-Ab2 1111B12H10 VH
caggtccaactgctgcagcctgggactgcactggtgatgcctggggcttcagtgaagctgtcctgcaaggcttctggctacaccttcaccacctactggatgcactgggtgaagcagaggcctggacaaggccttgagtggattggaaatattaatcctatcaatggtggtagtaactacaatgagaagttcaagagcaaggcctcactgactgtagacaagtcctccagcacagcctacatgcagctcagcagcctgacatctgaggactctgcggtctattattgtgtcattgctatggactactggggtcaaggaacctcagtcaccgtctcctca

SEQ ID NO: 69 - DNA, 321 bp, Artificial Sequence
  ACI-7067-1111B12-Ab2 1111B12H10 VL
gacatccagatgacccagtctccatcctccttatctgcctctctgggagaaagagtcagtctcacatgtcgggcaagtcaggacattggtattagcttaaactggtttcagcaggaaccagatggaactattaaacgcctgatctacgccacatccagtttagattctggtgtccccaaaaggttcagtggcaataggtctgggtcagattattctctcaccatcagtagccttgagtctgaagattttgcagactattactgtctacaatttgctagttctccgctcacgttcggtgctgggaccaagctggagctgaaa

SEQ ID NO: 70 - protein, 114 aa, Artificial Sequence
  ACI-7067-1112H8-Ab2 1112H8C12 VH
EVKLEESGGGLVQPGGSMKLSCAASGFTFTDAWMNWVRQSPEKGLEWIAEIRNKAHNYATYYAESVKGRFDISGDDSKSSVYLQMNNLRVEDTGIYYCTIYSYWGPGTLVTVSA

SEQ ID NO: 71 - protein, 4 aa, Artificial Sequence
  ACI-7067-1112H8-Ab2 1112H8C12 VH CDR1
DAWM

SEQ ID NO: 72 - protein, 19 aa, Artificial Sequence
  ACI-7067-1112H8-Ab2 1112H8C12 VH CDR2
EIRNKAHNYATYYAESVKG

SEQ ID NO: 73 - skipped

SEQ ID NO: 74 - protein, 107 aa, Artificial Sequence
  ACI-7067-1112H8-Ab2 1112H8C12 VL
SIVMTQTPKFLLMSPGDRVTMTCTASQSVSNYVAWYQQKPGQSPKLLIYSASNRFTGVPDRFTGSGYGTDFTFTINTVQTEDMAVYFCQQDYTSPYTFGGGTKLEIK

SEQ ID NO: 75 - protein, 11 aa, Artificial Sequence
  ACI-7067-1112H8-Ab2 1112H8C12 VL CDR1
TASQSVSNYVA

SEQ ID NO: 76 - protein, 7 aa, Artificial Sequence
  ACI-7067-1112H8-Ab2 1112H8C12 VL CDR2
SASNRFT

SEQ ID NO: 77 - protein, 9 aa, Artificial Sequence
  ACI-7067-1112H8-Ab2 1112H8C12 VL CDR3
QQDYTSPYT

SEQ ID NO: 78 - DNA, 342 bp, Artificial Sequence
  ACI-7067-1112H8-Ab2 1112H8C12 VH
gaagtgaagcttgaggagtctggaggaggcttggtgcaacctggaggatccatgaaactctcttgtgctgcctctggattcacttttactgacgcctggatgaactgggtccgccagtctccagaaaaggggcttgagtggattgctgaaattagaaacaaagctcataattatgcaacatactatgctgagtctgtgaaagggaggttcgacatctcaggagatgattccaaaagtagtgtctacctgcaaatgaacaacttgagagttgaagacactggcatttattactgtaccatttactcttactggggcccagggactctggtcactgtctctgca

SEQ ID NO: 79 - DNA, 321 bp, Artificial Sequence
  ACI-7067-1112H8-Ab2 1112H8C12 VL
agtattgtgatgacccagactcccaaattcctgcttatgtcaccaggagacagggttaccatgacctgcacggccagtcagagtgtgagtaattatgtggcttggtaccaacagaagccagggcagtctcctaaactgctgatatactctgcatccaatcgcttcactggagtccctgatcgcttcactggcagtggatatgggacggatttcactttcaccatcaacactgtgcagactgaagacatggcagtttatttctgtcagcaggattacacctctccgtacacgttcgggggggggaccaagctggaaataaaa

SEQ ID NO: 80 - protein, 120 aa, Artificial Sequence; identical to SEQ ID NO: 30
  ACI-7067-1108B11-Ab2 1108B11D3 VH

SEQ ID NO: 81 - protein, 5 aa, Artificial Sequence
  ACI-7067-1108B11-Ab2 1108B11D3 VH CDR1
TYAMH

SEQ ID NO: 82 - protein, 19 aa, Artificial Sequence
  ACI-7067-1108B11-Ab2 1108B11D3 VH CDR2
RIRSKGSNYATNYADSVKD

SEQ ID NO: 83 - protein, 9 aa, Artificial Sequence
  ACI-7067-1108B11-Ab2 1108B11D3 VH CDR3
GHGSSYFSY

SEQ ID NO: 84 - protein, 106 aa, Artificial Sequence
  ACI-7067-1108B11-Ab2 1108B11D3 VL
QIVLTQSPAIMSASPGERITMTCSANSSVTYMHWYQQKSGTSPKRWIYDTSNLASGVPARFSGSGSGTSYSLTISSMEAEDAATYYCQQWKSHPPTFGAGTKLELK

SEQ ID NO: 85 - protein, 10 aa, Artificial Sequence
  ACI-7067-1108B11-Ab2 1108B11D3 VL CDR1
SANSSVTYMH

SEQ ID NO: 86 - protein, 7 aa, Artificial Sequence
  ACI-7067-1108B11-Ab2 1108B11D3 VL CDR2
DTSNLAS

SEQ ID NO: 87 - protein, 9 aa, Artificial Sequence
  ACI-7067-1108B11-Ab2 1108B11D3 VL CDR3
QQWKSHPPT

SEQ ID NO: 88 - DNA, 360 bp, Artificial Sequence; identical to SEQ ID NO: 38
  ACI-7067-1108B11-Ab2 1108B11D3 VH

SEQ ID NO: 89 - DNA, 318 bp, Artificial Sequence
  ACI-7067-1108B11-Ab2 1108B11D3 VL
caaattgttctcacccagtctccagcaatcatgtctgcatctccaggggagaggatcaccatgacctgcagtgccaactcaagtgttacttacatgcactggtaccagcagaagtcaggcacctcccccaaaagatggatttatgacacatccaatctggcttctggagtccctgctcgcttcagtggcagtgggtctgggacctcttactctctcacaatcagcagcatggaggctgaagatgctgccacttattactgccagcagtggaaaagtcacccacccacgttcggtgctgggaccaagctggaactgaaa

SEQ ID NO: 90 - protein, 119 aa, Artificial Sequence
  ACI-7067-1113D10-Ab1 1113D10E3D5 VH
EVQLVESGGGLVQPKGSLKLSCAASGFTFNTYALHWVRQAPGKGLEWVARIRSKSSNYATYYADSVKDRFTISRDDSQSMLYLQMNNLKTEDTGMYYCVRGGVSPFDYWGQGTTLTVSS

SEQ ID NO: 91 - protein, 5 aa, Artificial Sequence
  ACI-7067-1113D10-Ab1 1113D10E3D5 VH CDR1
TYALH

SEQ ID NO: 92 - protein, 19 aa, Artificial Sequence
  ACI-7067-1113D10-Ab1 1113D10E3D5 VH CDR2
RIRSKSSNYATYYADSVKD

SEQ ID NO: 93 - protein, 8 aa, Artificial Sequence
  ACI-7067-1113D10-Ab1 1113D10E3D5 VH CDR3
GGVSPFDY

SEQ ID NO: 94 - protein, 106 aa, Artificial Sequence
  ACI-7067-1113D10-Ab1 1113D10E3D5 VL
QIVLTQSPAIMSASPGEKVTMTCSASSSVSYMHWYQQKSGTSPKRWIYDTSKLASGVPARFSGSGSGTSYSLTISSMEAEDSATYYCQQWSNNPPTFGGGTKLEIK

SEQ ID NO: 95 - protein, 10 aa, Artificial Sequence
  ACI-7067-1113D10-Ab1 1113D10E3D5 VL CDR1
SASSSVSYMH

SEQ ID NO: 96 - protein, 7 aa, Artificial Sequence
  ACI-7067-1113D10-Ab1 1113D10E3D5 VL CDR2
DTSKLAS

SEQ ID NO: 97 - protein, 9 aa, Artificial Sequence
  ACI-7067-1113D10-Ab1 1113D10E3D5 VL CDR3
QQWSNNPPT

SEQ ID NO: 98 - DNA, 357 bp, Artificial Sequence
  ACI-7067-1113D10-Ab1 1113D10E3D5 VH
gaggtgcagcttgttgagtctggtggaggattggtgcagcctaaaggatcattgaaactctcatgtgccgcctctggtttcaccttcaatacctatgccctgcactgggtccgccaggctccaggaaagggtttggaatgggttgctcgcataagaagtaaaagtagtaattatgcaacatattatgccgattcagtgaaagacagattcaccatctccagagatgattcacaaagcatgctctatctgcaaatgaacaacctgaaaactgaggacacaggcatgtattactgtgtaagagggggtgtttctccctttgactactggggccaaggcaccactctcacagtctcctca

SEQ ID NO: 99 - DNA, 318 bp, Artificial Sequence
  ACI-7067-1113D10-Ab1 1113D10E3D5 VL
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagattctgccacttattactgccagcagtggagtaataacccaccgacgttcggtggaggcaccaagctggaaatcaaa

SEQ ID NO: 100 - protein, 113 aa, Artificial Sequence
  ACI-7067-1116F2-Ab1 1116F2A2 VH
DVQLQESGPGFVKPSQSLSLTCSVTGYSITRGFYWNWIRQFPGNKLEWMGYISDDGNSNYNPSLKNRISITRDTFKNQVFLRLNSVTTEDTATYYCTRGDLLWGQGTTLTVSS

SEQ ID NO: 101 - protein, 6 aa, Artificial Sequence
  ACI-7067-1116F2-Ab1 1116F2A2 VH CDR1
RGFYWN

SEQ ID NO: 102 - protein, 16 aa, Artificial Sequence
  ACI-7067-1116F2-Ab1 1116F2A2 VH CDR2
YISDDGNSNYNPSLKN

SEQ ID NO: 103 - protein, 4 aa, Artificial Sequence
  ACI-7067-1116F2-Ab1 1116F2A2 VH CDR3
GDLL

SEQ ID NO: 104 - protein, 112 aa, Artificial Sequence
  ACI-7067-1116F2-Ab1 1116F2A2 VL
DVVMTQTALTLSVTIGQPASISCKSSQSLLDSDGETYLNWLLQRPGQSPKRLIYLVSKLDSGVPDRFTGSGSGTDFALKISRVEAEDLGIYYCWQGTHFPQTFGGGTKLEIK

SEQ ID NO: 105 - protein, 16 aa, Artificial Sequence
  ACI-7067-1116F2-Ab1 1116F2A2 VL CDR1
KSSQSLLDSDGETYLN

SEQ ID NO: 106 - protein, 7 aa, Artificial Sequence
  ACI-7067-1116F2-Ab1 1116F2A2 VL CDR2
LVSKLDS

SEQ ID NO: 107 - protein, 9 aa, Artificial Sequence
  ACI-7067-1116F2-Ab1 1116F2A2 VL CDR3
WQGTHFPQT

SEQ ID NO: 108 - DNA, 339 bp, Artificial Sequence
  ACI-7067-1116F2-Ab1 1116F2A2 VH
gatgtacaacttcaggagtcaggacctggcttcgtgaaaccttctcagtctctgtctctcacctgctctgtcactggctactcaataaccagaggtttttactggaactggatccgacagtttccaggaaacaaactggaatggatgggctacataagtgacgatggtaatagtaactacaatccctctctcaaaaatcgaatctccatcactcgtgacacatttaagaatcaggttttcctgaggttgaactctgtgactactgaggacactgccacatactattgtacaagaggagatctactttggggccaaggcaccactctcacagtctcctca

SEQ ID NO: 109 - DNA, 336 bp, Artificial Sequence
  ACI-7067-1116F2-Ab1 1116F2A2 VL
gatgttgtgatgacccagactgcactcactttgtcggttaccattggacaaccagcctccatctcttgcaagtcaagtcaaagcctcttagatagtgatggagagacatatttgaattggttgttacagaggccaggccagtctccaaagcgcctaatctatctggtgtctaaactggactctggagtccctgacaggttcactggtagtggatcagggacagatttcgcactgaaaatcagcagagtggaggctgaggacttgggaatttattattgctggcaaggtacacattttcctcagacgttcggtggaggcaccaagctggaaatcaaa

SEQ ID NO: 110 - protein, 123 aa, Artificial Sequence
  ACI-7067-1206E5-Ab1 1206E5D2 VH
QVQLQQSGPELVKPGASVKMSCKASGYTFTDYVISWVKQGTGQGLEWIGEIYPGNDSTYYNEKFKGKATLTADKSSNTAYMQLSSLTSEDSAVYFCAREGVSNGYLYLSMDYWGQGTSVTVSS

SEQ ID NO: 111 - protein, 5 aa, Artificial Sequence
  ACI-7067-1206E5-Ab1 1206E5D2 VH CDR1
DYVIS

SEQ ID NO: 112 - protein, 17 aa, Artificial Sequence
  ACI-7067-1206E5-Ab1 1206E5D2 VH CDR2
EIYPGNDSTYYNEKFKG

SEQ ID NO: 113 - protein, 14 aa, Artificial Sequence
  ACI-7067-1206E5-Ab1 1206E5D2 VH CDR3
EGVSNGYLYLSMDY

SEQ ID NO: 114 - protein, 112 aa, Artificial Sequence
  ACI-7067-1206E5-Ab1 1206E5D2 VL
DVLMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPWTFGGGTKLEIK

SEQ ID NO: 115 - protein, 16 aa, Artificial Sequence
  ACI-7067-1206E5-Ab1 1206E5D2 VL CDR1
KSSQSLLYSNGKTYLN

SEQ ID NO: 116 - protein, 7 aa, Artificial Sequence
  ACI-7067-1206E5-Ab1 1206E5D2 VL CDR2
LVSKLDS

SEQ ID NO: 117 - protein, 9 aa, Artificial Sequence
  ACI-7067-1206E5-Ab1 1206E5D2 VL CDR3
VQGTHFPWT

SEQ ID NO: 118 - DNA, 369 bp, Artificial Sequence
  ACI-7067-1206E5-Ab1 1206E5D2 VH
caggttcagctgcagcagtctggacctgagctggtgaagcctggggcttcagtgaagatgtcctgcaaggcttctggatacacattcactgactatgttataagctgggtgaagcagggaactggacagggccttgagtggattggagagatttatcctggaaatgatagtacttactacaatgagaagttcaagggcaaggccacactgactgcagacaaatcctccaacacagcctacatgcagctcagcagcctgacatctgaggactctgcggtctatttctgtgcaagagagggggtctctaatggttacctatatttgtctatggactactggggtcaaggaacctcagtcaccgtctcctca

SEQ ID NO: 119 - DNA, 336 bp, Artificial Sequence
  ACI-7067-1206E5-Ab1 1206E5D2 VL
gatgttttgatgacccaaactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacattttccgtggacgttcggtggaggcaccaagctggaaatcaaa

SEQ ID NO: 120 - protein, 14 aa, Unknown
  Artificial peptide dervied from the class Mammalia
MDVFMKGLSKAKEG

SEQ ID NO: 121 - protein, 15 aa, Unknown
  Artificial peptide dervied from the class Mammalia
MDVFMKGLSKAKEGV

SEQ ID NO: 122 - protein, 15 aa, Unknown
  Artificial peptide dervied from the class Mammalia
KAKEGVVAAAEKTKQ

SEQ ID NO: 123 - protein, 15 aa, Unknown
  Artificial peptide dervied from the class Mammalia
AEKTKQGVAEAAGKT

SEQ ID NO: 124 - protein, 15 aa, Unknown
  Artificial peptide dervied from the class Mammalia
EAAGKTKEGVLYVGS

SEQ ID NO: 125 - protein, 15 aa, Unknown
  Artificial peptide dervied from the class Mammalia
VLYVGSKTKEGVVHG

SEQ ID NO: 126 - protein, 15 aa, Unknown
  Artificial peptide dervied from the class Mammalia
EGVVHGVATVAEKTK

SEQ ID NO: 127 - protein, 15 aa, Unknown
  Artificial peptide derived from the class Mammalia
VAEKTKEQVTNVGGA

SEQ ID NO: 128 - protein, 15 aa, Unknown
  Binding epitope from the class Mammalia
TNVGGAVVTGVTAVA

SEQ ID NO: 129 - protein, 15 aa, Unknown
  Binding epitope from the class Mammalia
GVTAVAQKTVEGAGS

SEQ ID NO: 130 - protein, 15 aa, Unknown
  Binding epitope from the class Mammalia
VEGAGSIAAATGFVK

SEQ ID NO: 131 - protein, 15 aa, Unknown
  Binding epitope from the class Mammalia
ATGFVKKDQLGKNEE

SEQ ID NO: 132 - protein, 15 aa, Unknown
  Artificial peptide derived from the class Mammalia
LGKNEEGAPQEGILE

SEQ ID NO: 133 - protein, 15 aa, Unknown
  Artificial peptide derived from the class Mammalia
QEGILEDMPVDPDNE

SEQ ID NO: 134 - protein, 15 aa, Unknown
  Binding epitope from the class Mammalia
VDPDNEAYEMPSEEG

SEQ ID NO: 135 - protein, 14 aa, Unknown
  Binding epitope from the class Mammalia
MPSEEGYQDYEPEA

SEQ ID NO: 136 - protein, 8 aa, Unknown
  Binding epitope from the class Mammalia
GVATVAEK

SEQ ID NO: 137 - protein, 40 aa, Unknown
  Binding epitope from the class Mammalia
TVEGAGSIAAATGFVKKDQLGKNEEGAPQEGILEDMPVDP

SEQ ID NO: 138 - protein, 31 aa, Unknown
  Binding epitope from the class Mammalia
VVAAAEKTKQGVAEAAGKTKEGVLYVGSKTK

SEQ ID NO: 139 - protein, 23 aa, Unknown
  Binding epitope from the class Mammalia
EAAGKTKEGVLYVGSKTKEGVVH

SEQ ID NO: 140 - protein, 121 aa, Artificial Sequence
  ACI-7079-2501G2-Ab2 2501G2E5 VH
EVQLVESGGGLVQPKGSLKLSCAASGFNFNTYAMNWVRQAPGKGLEWVARIRTKSNNFATYYAHSVKDRFTISRDDSESMLYLQMNNLKTEDTAMYYCVRQGLAYYAMDYWGQGTSVTVSS

SEQ ID NO: 141 - protein, 5 aa, Artificial Sequence
  ACI-7079-2501G2-Ab2 2501G2E5 VH CDR1
TYAMN

SEQ ID NO: 142 - protein, 19 aa, Artificial Sequence
  ACI-7079-2501G2-Ab2 2501G2E5 VH CDR2
RIRTKSNNFATYYAHSVKD

SEQ ID NO: 143 - protein, 10 aa, Artificial Sequence
  ACI-7079-2501G2-Ab2 2501G2E5 VH CDR3
QGLAYYAMDY

SEQ ID NO: 144 - protein, 112 aa, Artificial Sequence
  ACI-7079-2501G2-Ab2 2501G2E5 VL
DVLMTQTPLSLPVSLGDQVSISCRSSQTIVHSNGNTYLEWYLQKPGQSPKLLIYKVSNRFSGVPDRFSGSGSGTDFTLKISRVEAEDLGVYYCFQGSQGPLTFGAGTKLELK

SEQ ID NO: 145 - protein, 16 aa, Artificial Sequence
  ACI-7079-2501G2-Ab2 2501G2E5 VL CDR1
RSSQTIVHSNGNTYLE

SEQ ID NO: 146 - protein, 30 aa, Unknown
  Binding epitope from the class Mammalia
GKTKEGVLYVGSKTKEGVVHGVATVAEKTK

SEQ ID NO: 147 - protein, 45 aa, Unknown
  Binding epitope from the class Mammalia
KKDQLGKNEEGAPQEGILEDMPVDPDNEAYEMPSEEGYQDYEPEA

SEQ ID NO: 148 - DNA, 363 bp, Artificial Sequence
  ACI-7079-2501G2-Ab2 2501G2E5 VH
gaggtgcagcttgttgagtctggtggaggattggtgcagcctaaagggtcattgaaactctcatgtgcagcctctggattcaacttcaatacctatgccatgaactgggtccgccaggctccaggaaagggtttggaatgggttgctcgcataagaactaaaagtaataattttgcaacatattatgcccattcagtgaaagacagattcaccatctccagagatgattcagaaagcatgctctatctgcaaatgaacaacttgaaaactgaggacacagccatgtattactgtgtgagacagggactagcctactatgctatggactactggggtcaaggaacctcagtcaccgtctcctca

SEQ ID NO: 149 - DNA, 336 bp, Artificial Sequence
  ACI-7079-2501G2-Ab2 2501G2E5 VL
gatgttttgatgacccaaactccactctccctgcctgtcagtcttggagatcaagtctccatctcttgcagatctagtcaaaccattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacaaggtccgctcacgttcggtgctgggaccaaactggagctgaaa

SEQ ID NO: 150 - protein, 113 aa, Artificial Sequence
  ACI-7079-2503C6-Ab1 2503C6H9 VH
DVQLQESGPGLVKPSQSLSLTCSVTGYSITSGYYWNWIRLFPGNKLEWLGYINYDGSNNFNPSLKNRISITRDTSKNQFFLKLNSVTSEDTATYFCLRGDWDWGQGTLVTVSA

SEQ ID NO: 151 - protein, 6 aa, Artificial Sequence
  ACI-7079-2503C6-Ab1 2503C6H9 VH CDR1
SGYYWN

SEQ ID NO: 152 - protein, 16 aa, Artificial Sequence
  ACI-7079-2503C6-Ab1 2503C6H9 VH CDR2
YINYDGSNNFNPSLKN

SEQ ID NO: 153 - protein, 4 aa, Artificial Sequence
  ACI-7079-2503C6-Ab1 2503C6H9 VH CDR3
GDWD

SEQ ID NO: 154 - protein, 112 aa, Artificial Sequence
  ACI-7079-2503C6-Ab1 2503C6H9 VL
DVVMTQTPLTLSVTIGQPASISCKSSQSLLDSDGETYLNWLLQRPGQSPKRLICLVSKLDSGVPDRFTGSGSGTDFTLKISRVEAEDLGVYYCWQGTHFPQTFGGGTRLEIK

SEQ ID NO: 155 - protein, 16 aa, Artificial Sequence
  ACI-7079-2503C6-Ab1 2503C6H9 VL CDR1
KSSQSLLDSDGETYLN

SEQ ID NO: 156 - protein, 7 aa, Artificial Sequence
  ACI-7079-2503C6-Ab1 2503C6H9 VL CDR2
LVSKLDS

SEQ ID NO: 157 - protein, 9 aa, Artificial Sequence
  ACI-7079-2503C6-Ab1 2503C6H9 VL CDR3
WQGTHFPQT

SEQ ID NO: 158 - DNA, 339 bp, Artificial Sequence
  ACI-7079-2503C6-Ab1 2503C6H9 VH
gatgtacagcttcaggagtcaggacctggcctcgtgaaaccttctcagtctctgtctctcacctgctctgtcactggctactccatcaccagtggttattactggaactggatccgactatttccaggaaacaaactggaatggctgggctacataaactacgatggtagcaataacttcaacccatctctcaaaaatcgaatctccatcactcgtgacacatctaagaaccagtttttcctgaaattgaattctgtgacttctgaggacacagccacatatttctgtttaagaggggactgggactggggccaagggactctggtcactgtctctgca

SEQ ID NO: 159 - DNA, 336 bp, Artificial Sequence
  ACI-7079-2503C6-Ab1 2503C6H9 VL
gatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctgtctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaggctggaaatcaaa

SEQ ID NO: 160 - protein, 114 aa, Artificial Sequence
  ACI-7079-2504A6-Ab1 2504A6C8 VH
QVQLQQSGVELARPGASVKLSCKASGYTFTSYGISWVKQRTGQGLKWIGEIYPGSGNTYYNEKFKGKATLTADKSSSTAYMELRSLTSEDSAVYFCATDYDAYWGQGTTLTVSS

SEQ ID NO: 161 - protein, 5 aa, Artificial Sequence
  ACI-7079-2504A6-Ab1 2504A6C8 VH CDR1
SYGIS

SEQ ID NO: 162 - protein, 17 aa, Artificial Sequence
  ACI-7079-2504A6-Ab1 2504A6C8 VH CDR2
EIYPGSGNTYYNEKFKG

SEQ ID NO: 163 - protein, 5 aa, Artificial Sequence
  ACI-7079-2504A6-Ab1 2504A6C8 VH CDR3
DYDAY

SEQ ID NO: 164 - protein, 112 aa, Artificial Sequence
  ACI-7079-2504A6-Ab1 2504A6C8 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 165 - protein, 16 aa, Artificial Sequence
  ACI-7079-2504A6-Ab1 2504A6C8 VL CDR1
RSSQSLVHSNGNTYLH

SEQ ID NO: 166 - protein, 7 aa, Artificial Sequence
  ACI-7079-2504A6-Ab1 2504A6C8 VL CDR2
KVSNRFS

SEQ ID NO: 167 - protein, 9 aa, Artificial Sequence
  ACI-7079-2504A6-Ab1 2504A6C8 VL CDR3
SQSTHVPLT

SEQ ID NO: 168 - DNA, 342 bp, Artificial Sequence
  ACI-7079-2504A6-Ab1 2504A6C8 VH
caggttcagctgcagcagtctggagttgagctggcgaggcctggggcttcagtgaaactgtcctgcaaggcttctggctacaccttcacaagctatggtataagctgggtgaagcagagaactggacagggccttaagtggattggagagatttatcctggaagtggtaatacttactacaatgagaagttcaagggcaaggccacactgactgcagacaaatcctccagcacagcgtacatggagctccgcagcctgacgtctgaggactctgcggtctatttctgtgcaaccgattacgacgcctactggggccaaggcaccactctcacagtctcctca

SEQ ID NO: 169 - DNA, 336 bp, Artificial Sequence
  ACI-7079-2504A6-Ab1 2504A6C8 VL
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaa

SEQ ID NO: 170 - protein, 120 aa, Artificial Sequence
  ACI-7079-2506E2-Ab2 2506E2G4 VH
QVQLQQSGPELVRPGASVKISCKASGYAFSNSWMNWVKQRPGKGLEWIGRIFPGDGDTYYDGKFKGKVKLTTDKFSNTAYMQLRSLTSEDSAVYFCARWGGTNDEWFAHWGQGTLVTVSV

SEQ ID NO: 171 - protein, 5 aa, Artificial Sequence
  ACI-7079-2506E2-Ab2 2506E2G4 VH CDR1
NSWMN

SEQ ID NO: 172 - protein, 17 aa, Artificial Sequence
  ACI-7079-2506E2-Ab2 2506E2G4 VH CDR2
RIFPGDGDTYYDGKFKG

SEQ ID NO: 173 - protein, 11 aa, Artificial Sequence
  ACI-7079-2506E2-Ab2 2506E2G4 VH CDR3
WGGTNDEWFAH

SEQ ID NO: 174 - protein, 111 aa, Artificial Sequence
  ACI-7079-2506E2-Ab2 2506E2G4 VL
DIVLTQSPASLTVSLGQRATISCRASQSVSTSRNSYMHWYQQKPRQPPKLLIKYASNLESGVPARFSGSGSGADFTLNIHPVEEEDTATYYCQHSWDIPLTFGTGTKLELS

SEQ ID NO: 175 - protein, 15 aa, Artificial Sequence
  ACI-7079-2506E2-Ab2 2506E2G4 VL CDR1
RASQSVSTSRNSYMH

SEQ ID NO: 176 - protein, 7 aa, Artificial Sequence
  ACI-7079-2506E2-Ab2 2506E2G4 VL CDR2
YASNLES

SEQ ID NO: 177 - protein, 9 aa, Artificial Sequence
  ACI-7079-2506E2-Ab2 2506E2G4 VL CDR3
QHSWDIPLT

SEQ ID NO: 178 - DNA, 360 bp, Artificial Sequence
  ACI-7079-2506E2-Ab2 2506E2G4 VH
caggttcagttgcagcagtctggacctgagctggtgaggcctggggcctcagtgaagatttcctgcaaggcttctggctacgcattcagtaactcctggatgaactgggtgaagcagaggcctggaaagggtcttgagtggattggacggatttttcctggagatggagatacttactacgatgggaagttcaagggcaaggtcaaactgacaacagacaaattctccaacacagcctacatgcaactccgcagcctgacatctgaggactctgcggtctacttctgtgcaagatgggggggtactaacgatgagtggtttgctcactggggccaagggactctggtcactgtctctgta

SEQ ID NO: 179 - DNA, 333 bp, Artificial Sequence
  ACI-7079-2506E2-Ab2 2506E2G4 VL
gacattgtgctgacacagtctcctgcttccttaactgtatctctggggcagagggccaccatctcatgcagggccagccaaagtgtcagtacatctaggaatagttatatgcactggtaccaacagaaaccaagacagccacccaaactcctcatcaagtatgcatccaacctagaatctggggtccctgccaggttcagtggcagtgggtctggggcagacttcaccctcaacatccatcctgtggaggaggaggatactgcaacatattactgtcagcacagttgggatattccgctcacgttcggtactgggaccaagctggagctgagt

SEQ ID NO: 180 - protein, 114 aa, Artificial Sequence
  ACI-7079-2506F3-Ab1 2506F3E12 VH
QVQLQQPGAELVKPGASVKLSCKASGYTFTTYWMQWVKQRPGQGLEWIGEIDPSDSYINYNQKFKGKATLTVDTSSSTAFMQLSSLTSEDSAVYYCARGMMDYWGQGTSVTVSS

SEQ ID NO: 181 - protein, 5 aa, Artificial Sequence
  ACI-7079-2506F3-Ab1 2506F3E12 VH CDR1
TYWMQ

SEQ ID NO: 182 - protein, 17 aa, Artificial Sequence
  ACI-7079-2506F3-Ab1 2506F3E12 VH CDR2
EIDPSDSYINYNQKFKG

SEQ ID NO: 183 - protein, 5 aa, Artificial Sequence
  ACI-7079-2506F3-Ab1 2506F3E12 VH CDR3
GMMDY

SEQ ID NO: 184 - protein, 112 aa, Artificial Sequence
  ACI-7079-2506F3-Ab1 2506F3E12 VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFKGSHVPYTFGGGTKLEIK

SEQ ID NO: 185 - protein, 16 aa, Artificial Sequence
  ACI-7079-2506F3-Ab1 2506F3E12 VL CDR1
RSSQSIVHSNGNTYLE

SEQ ID NO: 186 - protein, 7 aa, Artificial Sequence
  ACI-7079-2506F3-Ab1 2506F3E12 VL CDR2
KVSNRFS

SEQ ID NO: 187 - protein, 9 aa, Artificial Sequence
  ACI-7079-2506F3-Ab1 2506F3E12 VL CDR3
FKGSHVPYT

SEQ ID NO: 188 - DNA, 342 bp, Artificial Sequence
  ACI-7079-2506F3-Ab1 2506F3E12 VH
caggtccaactgcagcagcctggggctgagcttgtgaagcctggggcttcagtgaagctgtcctgcaaggcttctggctacaccttcaccacctactggatgcagtgggtaaaacagaggcctggacagggccttgagtggatcggagagattgatccttctgatagctatattaactacaatcaaaagttcaagggcaaggccacattgactgtagacacatcctccagcacagccttcatgcagctcagcagcctgacatctgaggactctgcggtctattactgtgcaagggggatgatggactactggggtcaaggaacctcagtcaccgtctcctca

SEQ ID NO: 189 - DNA, 336 bp, Artificial Sequence
  ACI-7079-2506F3-Ab1 2506F3E12 VL
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttaaaggttcacatgttccgtacacgttcggaggggggaccaagctggaaataaaa

SEQ ID NO: 190 - protein, 120 aa, Artificial Sequence
  ACI-7079-2507B3-Ab1 2507B3G8 VH
EVQLVESGGGLVQPKGSLKVSCAASGFTFKTYAMHWVRQAPGKGLEWVARIRSENSNFAKYYADSVKDRFTISRDDSQSMLYLQMHTLKTEDTAIYYCVRGYNGSSLDYWGQGTTLTVSS

SEQ ID NO: 191 - protein, 5 aa, Artificial Sequence
  ACI-7079-2507B3-Ab1 2507B3G8 VH CDR1
TYAMH

SEQ ID NO: 192 - protein, 19 aa, Artificial Sequence
  ACI-7079-2507B3-Ab1 2507B3G8 VH CDR2
RIRSENSNFAKYYADSVKD

SEQ ID NO: 193 - protein, 9 aa, Artificial Sequence
  ACI-7079-2507B3-Ab1 2507B3G8 VH CDR3
GYNGSSLDY

SEQ ID NO: 194 - protein, 106 aa, Artificial Sequence
  ACI-7079-2507B3-Ab1 2507B3G8 VL
QIVLTQSPAIMSAFPGERVTMTCSASSSVNYMHWYQQKSGTSPKRWIYDTSKLASGVPARFSGGGSGTSYSLTISNMEAEDAATYYCQQWRSNPPTFGGGTKLEIK

SEQ ID NO: 195 - protein, 10 aa, Artificial Sequence
  ACI-7079-2507B3-Ab1 2507B3G8 VL CDR1
SASSSVNYMH

SEQ ID NO: 196 - protein, 7 aa, Artificial Sequence
  ACI-7079-2507B3-Ab1 2507B3G8 VL CDR2
DTSKLAS

SEQ ID NO: 197 - protein, 9 aa, Artificial Sequence
  ACI-7079-2507B3-Ab1 2507B3G8 VL CDR3
QQWRSNPPT

SEQ ID NO: 198 - DNA, 360 bp, Artificial Sequence
  ACI-7079-2507B3-Ab1 2507B3G8 VH
gaggtgcagcttgttgagtctggtggaggattggtgcagcctaaaggatcattgaaagtctcatgtgccgcctctggtttcaccttcaagacctatgccatgcactgggtccgccaggctccgggaaagggtttggaatgggttgctcgcataagaagtgaaaacagtaattttgcaaaatattatgccgattcagtgaaagacagattcaccatctccagagatgattcacaaagtatgctctatctgcaaatgcacaccctgaaaactgaggacacagccatctattattgtgtaaggggatataacggcagtagccttgactactggggccaaggcaccactctcacagtctcctca

SEQ ID NO: 199 - DNA, 318 bp, Artificial Sequence
  ACI-7079-2507B3-Ab1 2507B3G8 VL
caaattgttctcacccagtctccagcaatcatgtctgcatttccaggggagagggtcaccatgacctgcagtgccagctcaagtgtaaattacatgcactggtaccagcagaagtccggcacctcccccaaaagatggatttatgacacatccaaactggcttctggagtccctgctcgcttcagtggcggtgggtctgggacctcttactctctcacaatcagcaacatggaggctgaagatgctgccacttattactgccagcagtggagaagtaatccacccactttcggaggggggaccaagctggaaataaaa

SEQ ID NO: 200 - protein, 113 aa, Artificial Sequence
  ACI-7079-2511B3-Ab3 2511B3B12 VH
DVQLQESGPGLVKPSQSLSLTCSVTGFSITSYYYWNWIRQFPGNKLEWMAYISYDGSNNYNPSLKNRISITRDTSKNQFFLKLNSVTTEDTATYYCTRGDWDWGQGTLVTVSA

SEQ ID NO: 201 - protein, 6 aa, Artificial Sequence
  ACI-7079-2511B3-Ab3 2511B3B12 VH CDR1
SYYYWN

SEQ ID NO: 202 - protein, 16 aa, Artificial Sequence
  ACI-7079-2511B3-Ab3 2511B3B12 VH CDR2
YISYDGSNNYNPSLKN

SEQ ID NO: 203 - protein, 4 aa, Artificial Sequence
  ACI-7079-2511B3-Ab3 2511B3B12 VH CDR3
GDWD

SEQ ID NO: 204 - protein, 112 aa, Artificial Sequence
  ACI-7079-2511B3-Ab3 2511B3B12 VL
DVVMTQTPLTLSLTIGQPASISCKSSQSLLDSDGETYLNWLLQRPGQSPKRLIYLVSKLESGVPDRFTGSGSGTVFTLKISRVEAEDLGVYYCWQGTHFPQTFGGGTKLEIK

SEQ ID NO: 205 - protein, 16 aa, Artificial Sequence
  ACI-7079-2511B3-Ab3 2511B3B12 VL CDR1
KSSQSLLDSDGETYLN

SEQ ID NO: 206 - protein, 7 aa, Artificial Sequence
  ACI-7079-2511B3-Ab3 2511B3B12 VL CDR2
LVSKLES

SEQ ID NO: 207 - protein, 9 aa, Artificial Sequence
  ACI-7079-2511B3-Ab3 2511B3B12 VL CDR3
WQGTHFPQT

SEQ ID NO: 208 - DNA, 339 bp, Artificial Sequence
  ACI-7079-2511B3-Ab3- 2511B3B12 VH
gatgtacagcttcaggaatcaggacctggcctcgtgaaaccttctcagtctctgtctctcacctgctctgtcactggcttctccatcaccagttattattactggaactggatccggcagtttccaggaaacaaactggaatggatggcctacataagctacgatggtagcaataactacaacccatctctcaaaaatcgaatctccatcactcgtgacacatctaagaaccagtttttcctgaagttgaattctgtgactactgaggacacagccacatattactgtacaagaggggactgggactggggccaagggactctggtcactgtctctgca

SEQ ID NO: 209 - DNA, 336 bp, Artificial Sequence
  ACI-7079-2511B3-Ab3- 2511B3B12 VL
gatgttgtgatgacccagactccactcactttgtcgcttaccattggacaaccagcctccatctcttgcaagtcaagtcagagcctcttagatagtgatggagagacatatttgaattggttgttacagaggccaggtcagtctccaaagcgcctaatctatctggtgtctaaactggaatctggagtccctgacaggttcactggcagtggatcagggacagttttcacactgaaaatcagcagagtggaggctgaggatttgggagtttattattgctggcaagggacacattttcctcagacgttcggtggaggcaccaagctggaaatcaaa

SEQ ID NO: 210 - protein, 113 aa, Artificial Sequence
  ACI-7079-2601B6-Ab1 2601B6D2 VH
EIQLQQSGAELVRPGASVKLSCTTSGFNIKDDYIHWVKQRPEQGLEWIGWIDPENGDTDYASKFQGKATITADTSSNTAYLHLSSLTSEDAAVYFCTTRGFGYWGQGTLVTVS

SEQ ID NO: 211 - protein, 5 aa, Artificial Sequence
  ACI-7079-2601B6-Ab1 2601B6D2 VH CDR1
DDYIH

SEQ ID NO: 212 - protein, 17 aa, Artificial Sequence
  ACI-7079-2601B6-Ab1 2601B6D2 VH CDR2
WIDPENGDTDYASKFQG

SEQ ID NO: 213 - protein, 5 aa, Artificial Sequence
  ACI-7079-2601B6-Ab1 2601B6D2 VH CDR3
RGFGY

SEQ ID NO: 214 - protein, 107 aa, Artificial Sequence
  ACI-7079-2601B6-Ab1 2601B6D2 VL
DIVMTQSHKFMSTSVGDRVSITCKASQDVGNVVAWYQQKPGQSPKLLIYWASSRHTGVPDRFTGSGSGTEFTLTISNVQSEDLADYFCQQYSSYPLTFGAGTKLELK

SEQ ID NO: 215 - protein, 11 aa, Artificial Sequence
  ACI-7079-2601B6-Ab1 2601B6D2 VL CDR1
KASQDVGNVVA

SEQ ID NO: 216 - protein, 7 aa, Artificial Sequence
  ACI-7079-2601B6-Ab1 2601B6D2 VL CDR2
WASSRHT

SEQ ID NO: 217 - protein, 9 aa, Artificial Sequence
  ACI-7079-2601B6-Ab1 2601B6D2 VL CDR3
QQYSSYPLT

SEQ ID NO: 218 - DNA, 339 bp, Artificial Sequence
  ACI-7079-2601B6-Ab1 2601B6D2 VH
gagattcaactgcagcagtctggggctgagcttgtgaggccaggggcctcagtcaagttgtcctgcacaacttccggctttaacattaaagacgactatattcactgggtgaagcagaggcctgaacagggcctggagtggattggatggattgatcctgagaatggtgatactgattatgcctcgaagttccagggcaaggccactataacagcagacacatcctccaacacagcctacctgcacctcagcagcctgacatcagaggacgctgccgtctatttctgtactacaagaggatttggttactggggccaagggactctggtcactgtctct

SEQ ID NO: 219 - DNA, 321 bp, Artificial Sequence
  ACI-7079-2601B6-Ab1 2601B6D2 VL
gacattgtgatgacccagtctcacaaattcatgtccacatcagtaggagacagggtcagcatcacctgcaaggccagtcaggatgtgggtaatgttgttgcctggtatcaacagaaaccaggacaatctcctaaactactgatttactgggcatcctcccggcacactggagtccctgatcgcttcacaggcagtggatctgggacagaattcactctcaccattagcaatgtgcagtctgaagacttggcagattatttctgtcagcaatatagcagctatccgctcacgttcggtgctgggaccaagctggagctgaag

SEQ ID NO: 220 - protein, 120 aa, Artificial Sequence
  ACI-7079-2602G4-Ab4 2602G4H1 VH
EVQLVESGGGLVQPKGSLKLSCAASGFTFKTYAMHWVRQAPGKGLEWVARIRSKGSDYATYYADSVKDRFTISRDDSQSMLYLQMNNLKTEDTAMYFCVRGGADSWFAYWGQGTLVTVST

SEQ ID NO: 221 - protein, 5 aa, Artificial Sequence
  ACI-7079-2602G4-Ab4 2602G4H1 VH CDR1
TYAMH

SEQ ID NO: 222 - protein, 19 aa, Artificial Sequence
  ACI-7079-2602G4-Ab4 2602G4H1 VH CDR2
RIRSKGSDYATYYADSVKD

SEQ ID NO: 223 - protein, 9 aa, Artificial Sequence
  ACI-7079-2602G4-Ab4 2602G4H1 VH CDR3
GGADSWFAY

SEQ ID NO: 224 - protein, 106 aa, Artificial Sequence
  ACI-7079-2602G4-Ab4 2602G4H1 VL
QIVLTQSPAIMSASPGERITMTCTASSSVSYMHWYQQKSGTSPKRWIYDTSKLASGVPARFSGSGSGASYTLTISSMEAEDAATYYCQQWNRNPPTFGGGTQLAIK

SEQ ID NO: 225 - protein, 10 aa, Artificial Sequence
  ACI-7079-2602G4-Ab4 2602G4H1 VL CDR1
TASSSVSYMH

SEQ ID NO: 226 - protein, 7 aa, Artificial Sequence
  ACI-7079-2602G4-Ab4 2602G4H1 VL CDR2
DTSKLAS

SEQ ID NO: 227 - protein, 9 aa, Artificial Sequence
  ACI-7079-2602G4-Ab4 2602G4H1 VL CDR3
QQWNRNPPT

SEQ ID NO: 228 - DNA, 360 bp, Artificial Sequence
  ACI-7079-2602G4-Ab4 2602G4H1 VH
gaggtgcagcttgttgagtctggtggaggattggtgcagcctaaaggatcattgaaactctcatgtgccgcctctggtttcaccttcaagacctatgccatgcactgggtccgccaggctccaggaaagggtttggaatgggttgctcgcataagaagtaaaggtagtgattatgcaacatattatgccgattcagtgaaggacagattcaccatctccagagatgattcacaaagcatgctctatctgcaaatgaacaacctgaaaactgaggatacagccatgtatttctgtgtgagagggggtgctgactcctggtttgcttactggggccaagggactctggtcactgtctctaca

SEQ ID NO: 229 - DNA, 318 bp, Artificial Sequence
  ACI-7079-2602G4-Ab4 2602G4H1 VL
caaattgttctcacccagtctccagcaatcatgtctgcatctccaggggagaggatcaccatgacctgcactgccagctcaagtgtaagttacatgcactggtaccagcagaagtcaggcacctcccccaaaagatggatttatgacacatccaaactggcttctggagtccctgctcgcttcagtggcagtgggtctggggcctcttatactctcacaatcagcagcatggaggctgaagatgctgccacttattactgccagcagtggaatcgtaacccaccgacgttcggtggaggcacccagctggcaatcaaa

SEQ ID NO: 230 - protein, 114 aa, Artificial Sequence
  ACI-7079-2603C1-Ab3 2603C1H6 VH
QVQLQQPGADLVKPGASVKLSCKASGYTFTSYWMQWTKQRPGQGLEWIGEIDPSDSYANYNQKFKGKATLTVDKYSSTAYMQLNSLTSEDSAVYYCALYDGPSYWGQGTLVTVS

SEQ ID NO: 231 - protein, 5 aa, Artificial Sequence
  ACI-7079-2603C1-Ab3 2603C1H6 VH CDR1
SYWMQ

SEQ ID NO: 232 - protein, 17 aa, Artificial Sequence
  ACI-7079-2603C1-Ab3 2603C1H6 VH CDR2
EIDPSDSYANYNQKFKG

SEQ ID NO: 233 - protein, 6 aa, Artificial Sequence
  ACI-7079-2603C1-Ab3 2603C1H6 VH CDR3
YDGPSY

SEQ ID NO: 234 - protein, 106 aa, Artificial Sequence
  ACI-7079-2603C1-Ab3 2603C1H6 VL
ENVLTQSPAIMSASPGEKVTMTCSAGSSVSYMHWFQQKSSTSPKLWIYDTSKLPSGVPGRFSGSGSGNSYSLTISSMEAEDVATYYCFQGSGYPYTFGGGTKLEIK

SEQ ID NO: 235 - protein, 10 aa, Artificial Sequence
  ACI-7079-2603C1-Ab3 2603C1H6 VL CDR1
SAGSSVSYMH

SEQ ID NO: 236 - protein, 7 aa, Artificial Sequence
  ACI-7079-2603C1-Ab3 2603C1H6 VL CDR2
DTSKLPS

SEQ ID NO: 237 - protein, 9 aa, Artificial Sequence
  ACI-7079-2603C1-Ab3 2603C1H6 VL CDR3
FQGSGYPYT

SEQ ID NO: 238 - DNA, 342 bp, Artificial Sequence
  ACI-7079-2603C1-Ab3 2603C1H6 VH
caggtccaactgcagcaacctggggctgaccttgtgaagcctggggcttcagtgaagctgtcctgtaaggcttctggctacaccttcaccagttactggatgcagtggacaaaacagaggcctggacagggccttgagtggatcggagagattgatccttctgatagctatgctaactacaatcaaaagttcaagggcaaggccacattgactgttgacaaatattccagcacagcctacatgcagctcaacagcctgacatctgaggactctgcggtctattactgtgccctctatgatggtccctcttactggggccaagggactctggtcactgtctct

SEQ ID NO: 239 - DNA, 318 bp, Artificial Sequence
  ACI-7079-2603C1-Ab3 2603C1H6 VL
gaaaatgttctcacccagtctccagcaatcatgtctgcatctccaggggaaaaggtcaccatgacctgcagtgccggctcaagtgtaagttacatgcactggttccaacagaagtcaagcacctcccccaaactctggatttatgacacatccaaactgccttctggagtcccaggtcgcttcagtggcagtgggtctggaaactcttactctctcacgatcagcagcatggaggctgaagatgttgccacttattactgttttcaggggagtgggtacccgtacacgttcggaggggggaccaagctggaaataaaa

SEQ ID NO: 240 - protein, 120 aa, Artificial Sequence
  ACI-7079-2603F3-Ab1 2603F3H3 VH
EVQLVESGGGLVQPKGSLKLSCAASGFTFNTYAMHWVRQAPGKGLEWVARIRSKGSNYATYYADSVKDRFTISRDDSQSMLYLQMNNLKTEDTAMYYCVRGGGDSWFAYWGQGTLVTVSA

SEQ ID NO: 241 - protein, 5 aa, Artificial Sequence
  ACI-7079-2603F3-Ab1 2603F3H3 VH CDR1
TYAMH

SEQ ID NO: 242 - protein, 19 aa, Artificial Sequence
  ACI-7079-2603F3-Ab1 2603F3H3 VH CDR2
RIRSKGSNYATYYADSVKD

SEQ ID NO: 243 - protein, 9 aa, Artificial Sequence
  ACI-7079-2603F3-Ab1 2603F3H3 VH CDR3
GGGDSWFAY

SEQ ID NO: 244 - protein, 106 aa, Artificial Sequence
  ACI-7079-2603F3-Ab1 2603F3H3 VL
QIVLTQSPAIMSASPGERVTMTCTASSSVSYMHWYQQKSGTSPKRWIYDTSKLASGVPARFSGSGSGASYTLTISSMEAEDAATYYCQQWNSNPPTFGGGTQLAIK

SEQ ID NO: 245 - protein, 10 aa, Artificial Sequence
  ACI-7079-2603F3-Ab1 2603F3H3 VL CDR1
TASSSVSYMH

SEQ ID NO: 246 - protein, 7 aa, Artificial Sequence
  ACI-7079-2603F3-Ab1 2603F3H3 VL CDR2
DTSKLAS

SEQ ID NO: 247 - protein, 9 aa, Artificial Sequence
  ACI-7079-2603F3-Ab1 2603F3H3 VL CDR3
QQWNSNPPT

SEQ ID NO: 248 - DNA, 360 bp, Artificial Sequence
  ACI-7079-2603F3-Ab1 2603F3H3 VH
gaggtgcagcttgttgagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaggtagtaattatgcaacatattatgccgattcagtgaaagacagattcaccatctccagagatgattcacaaagcatgctctatctgcaaatgaacaacctgaaaactgaggacacagccatgtattactgtgtgagagggggtggtgactcctggtttgcttactggggccaagggactctggtcactgtctctgca

SEQ ID NO: 249 - DNA, 318 bp, Artificial Sequence
  ACI-7079-2603F3-Ab1 2603F3H3 VL
caaattgttctcacccagtctccagcaatcatgtctgcatctccaggggagagggtcaccatgacctgcactgccagctcaagtgtaagttacatgcactggtaccagcagaagtcaggcacctcccccaaaagatggatttatgacacatccaaactggcttctggagtccctgctcgcttcagtggcagtgggtctggggcctcttatactctcacaatcagcagcatggaggctgaagatgctgccacttattactgccagcagtggaatagtaacccaccgacgttcggtggaggcacccagctggcaatcaaa

SEQ ID NO: 250 - protein, 120 aa, Artificial Sequence
  ACI-7079-2605B3-Ab2 2605B3D1 VH
EVQLVESGGGLVQPKGSLKLSCAASGFIFKTYAMHWVRQAPGKGLEWVARIRSKGGNYATYFADSVKDRFTISRDDSQNMLYLQVNNLKIEDTAMYFCVRGGNYSWFAYWGQGTLVTVSA

SEQ ID NO: 251 - protein, 5 aa, Artificial Sequence
  ACI-7079-2605B3-Ab2 2605B3D1 VH CDR1
TYAMH

SEQ ID NO: 252 - protein, 19 aa, Artificial Sequence
  ACI-7079-2605B3-Ab2 2605B3D1 VH CDR2
RIRSKGGNYATYFADSVKD

SEQ ID NO: 253 - protein, 9 aa, Artificial Sequence
  ACI-7079-2605B3-Ab2 2605B3D1 VH CDR3
GGNYSWFAY

SEQ ID NO: 254 - protein, 106 aa, Artificial Sequence
  ACI-7079-2605B3-Ab2 2605B3D1 VL
QIVLTQSPAIMSASPGEKVTMTCSASSSVTYMHWYQQKSGTSPKRWIYDTSQLASGVPARFSGSGSGTSHSLTISSMETEDAATYYCQQWTRNPPTFGGGTKLAIK

SEQ ID NO: 255 - protein, 10 aa, Artificial Sequence
  ACI-7079-2605B3-Ab2 2605B3D1 VL CDR1
SASSSVTYMH

SEQ ID NO: 256 - protein, 7 aa, Artificial Sequence
  ACI-7079-2605B3-Ab2 2605B3D1 VL CDR2
DTSQLAS

SEQ ID NO: 257 - protein, 8 aa, Artificial Sequence
  ACI-7079-2605B3-Ab2 2605B3D1 VL CDR3
QQWTRNPP

SEQ ID NO: 258 - DNA, 360 bp, Unknown
  ACI-7079-2605B3-Ab2 2605B3D1 VH from the class Mammalia
gaggtgcagcttgttgagtctggtggaggattggtgcagcctaaaggatcattgaaactctcatgtgccgcctctggtttcatctttaaaacctatgccatgcattgggtccgccaggctccaggaaagggtttggaatgggttgctcgaataagaagtaaaggtggtaattatgcaacatattttgccgattcagtgaaagacagattcaccatctccagagatgattcacaaaatatgctctatctgcaagtgaacaacctgaaaattgaggacacagccatgtatttctgtgtgagagggggtaattactcctggtttgcttactggggccaagggactctggtcactgtctctgca

SEQ ID NO: 259 - DNA, 318 bp, Unknown
  ACI-7079-2605B3-Ab2 2605B3D1 VL from the class Mammalia
caaattgttctcacccagtctccagcaatcatgtctgcttctccaggggagaaggtcaccatgacctgcagtgccagctcaagtgtaacttacatgcattggtaccagcagaagtcaggcacctcccccaaaagatggatttatgacacatcccaactggcttctggagtccctgctcgcttcagtggcagtgggtctgggacctctcactctctcacaatcagcagcatggagactgaagatgctgccacttattactgccaacaatggactagaaacccaccgacgttcggtggaggcaccaagctggcaatcaaa

SEQ ID NO: 260 - protein, 119 aa, Artificial Sequence
  ACI-7079-2606A6-Ab2 2606A6D5 VH
QVQLQQSGPELVKPGASVKISCKASGFAFSSSWMNWVKQRPGKGLEWVGRIFPGDGDTNYDRKFKDKATLTADKSSSTAYMQLSSLTSEDSAVYFCARWTGGYDWFAYWGQGTLVTVSA

SEQ ID NO: 261 - protein, 5 aa, Artificial Sequence
  ACI-7079-2606A6-Ab2 2606A6D5 VH CDR1
SSWMN

SEQ ID NO: 262 - protein, 17 aa, Artificial Sequence
  ACI-7079-2606A6-Ab2 2606A6D5 VH CDR2
RIFPGDGDTNYDRKFKD

SEQ ID NO: 263 - protein, 10 aa, Artificial Sequence
  ACI-7079-2606A6-Ab2 2606A6D5 VH CDR3
WTGGYDWFAY

SEQ ID NO: 264 - protein, 111 aa, Artificial Sequence
  ACI-7079-2606A6-Ab2 2606A6D5 VL
DIVLTQSPASLAVSLGQRATISCRASQSVSTSNYNYLHWYQQKPGQPPKLLITYASNLESGVPARFSGSGSGTDFTLNIHPVEEGDTATYYCQHSWEIPLTFGAGTKLELK

SEQ ID NO: 265 - protein, 15 aa, Artificial Sequence
  ACI-7079-2606A6-Ab2 2606A6D5 VL CDR1
RASQSVSTSNYNYLH

SEQ ID NO: 266 - protein, 7 aa, Artificial Sequence
  ACI-7079-2606A6-Ab2 2606A6D5 VL CDR2
YASNLES

SEQ ID NO: 267 - protein, 9 aa, Artificial Sequence
  ACI-7079-2606A6-Ab2 2606A6D5 VL CDR3
QHSWEIPLT

SEQ ID NO: 268 - DNA, 357 bp, Artificial Sequence
  ACI-7079-2606A6-Ab2 2606A6D5 VH
caggttcagctgcaacagtctggacctgagctggtgaagcctggggcctcagtgaagatttcctgcaaggcttctggcttcgcattcagtagctcctggatgaactgggtgaagcagaggcctggaaagggtcttgagtgggttggacggatttttcctggagatggagatactaactacgataggaagttcaaggacaaggccacactgactgcagacaaatcctccagcacagcctacatgcaactcagcagcctgacatctgaggactctgcggtctacttctgtgcaagatggacggggggttacgactggtttgcttactggggccaagggactctggtcactgtctctgca

SEQ ID NO: 269 - DNA, 333 bp, Artificial Sequence
  ACI-7079-2606A6-Ab2 2606A6D5 VL
gacattgtgctgacacagtctcctgcttccttagctgtatctctggggcagagggccaccatctcatgcagggccagccaaagtgtcagtacatctaactataattatcttcactggtaccaacagaaaccaggacagccacccaaactcctcatcacgtatgcatccaacctagaatctggggtccctgccaggttcagtggcagtgggtctgggacagacttcaccctcaacatccatcctgtggaggagggagatactgcaacatattactgtcaacacagttgggagattccgctcacgttcggtgctgggaccaagctggagctgaaa

SEQ ID NO: 270 - protein, 117 aa, Artificial Sequence
  ACI-7079-2509E5-Ab2 2509E5E5 VH
EVQLQQSGPELVKPGASLKMSCKASGYSFTDYNMHWVKQSRGKSLEWIGYINPNNGVPTYKQKFKGRATLTVNQSSSTAYMEIRSLTSEDSAVYYCTRGGDHRFAYWGQGTLVTVSA

SEQ ID NO: 271 - protein, 5 aa, Artificial Sequence
  ACI-7079-2509E5-Ab2 2509E5E5 VH CDR1
DYNMH

SEQ ID NO: 272 - protein, 17 aa, Artificial Sequence
  ACI-7079-2509E5-Ab2 2509E5E5 VH CDR2
YINPNNGVPTYKQKFKG

SEQ ID NO: 273 - protein, 8 aa, Artificial Sequence
  ACI-7079-2509E5-Ab2 2509E5E5 VH CDR3
GGDHRFAY

SEQ ID NO: 274 - protein, 111 aa, Artificial Sequence
  ACI-7079-2509E5-Ab2 2509E5E5 VL
DIVLTQSPASLAVSLGQRATISCRASESVDYYGFSFVNWFQQKPGQPPKLLIYSASYKGSGVPVRFSGSGSGTDFSLSIHPMEADDTAMYFCQQNKEVPLTFGAGTKLELK

SEQ ID NO: 275 - protein, 15 aa, Artificial Sequence
  ACI-7079-2509E5-Ab2 2509E5E5 VL CDR1
RASESVDYYGFSFVN

SEQ ID NO: 276 - protein, 7 aa, Artificial Sequence
  ACI-7079-2509E5-Ab2 2509E5E5 VL CDR2
SASYKGS

SEQ ID NO: 277 - protein, 9 aa, Artificial Sequence
  ACI-7079-2509E5-Ab2 2509E5E5 VL CDR3
QQNKEVPLT

SEQ ID NO: 278 - DNA, 351 bp, Artificial Sequence
  ACI-7079-2509E5-Ab2 2509E5E5 VH
gaggtccagctgcaacagtctggacctgaactggtgaagcctggggcttcgctgaagatgtcctgcaaggcttctggatactcattcactgactacaacatgcactgggtgaaacagagccgtggaaagagccttgagtggattggatatattaaccctaacaatggtgttcccacgtataagcagaagttcaagggcagggccaccttgactgtaaaccagtcctccagcacagcctacatggagatccgcagcctgacatcggaagattctgcagtctattactgtacaagagggggtgatcaccggtttgcttactggggccaagggactctggtcactgtctctgca

SEQ ID NO: 279 - DNA, 333 bp, Artificial Sequence
  ACI-7079-2509E5-Ab2 2509E5E5 VL
gacattgtgctgacccaatctccagcttctttggctgtgtctctagggcagagggccaccatctcctgcagagccagcgaaagtgttgattattatggctttagttttgtgaactggttccaacagaaaccaggacagccacccaaactcctcatctatagtgcgtcctacaaaggatccggggtccctgtcaggttcagtggcagtgggtctgggacagacttcagtctcagcatccatcctatggaggcggatgatactgcaatgtatttctgtcagcaaaataaggaggttccgctcacgttcggtgctgggaccaagctggagctgaaa

SEQ ID NO: 280 - protein, 120 aa, Artificial Sequence
  ACI-7079-2501B11-Ab3 2501B11C7 VH
QVQLQQSGPELVKPGASVKISCKASGYAFSSFWMNWMKQRPGKGLEWIGRIYPGDGDAHYNGEFKGRATLTADKSSSTAYMQLSSLTSEDSAVYFCARKGDFYGSNYDYWGQGTTLTVSS

SEQ ID NO: 281 - protein, 4 aa, Artificial Sequence
  ACI-7079-2501B11-Ab3 2501B11C7 VH CDR1
SFWM

SEQ ID NO: 282 - protein, 17 aa, Artificial Sequence
  ACI-7079-2501B11-Ab3 2501B11C7 VH CDR2
RIYPGDGDAHYNGEFKG

SEQ ID NO: 283 - protein, 11 aa, Artificial Sequence
  ACI-7079-2501B11-Ab3 2501B11C7 VH CDR3
KGDFYGSNYDY

SEQ ID NO: 284 - protein, 109 aa, Artificial Sequence
  ACI-7079-2501B11-Ab3 2501B11C7 VL
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTRLTVL

SEQ ID NO: 285 - protein, 14 aa, Artificial Sequence
  ACI-7079-2501B11-Ab3 2501B11C7 VL CDR1
RSSTGAVTTSNYAN

SEQ ID NO: 286 - protein, 7 aa, Artificial Sequence
  ACI-7079-2501B11-Ab3 2501B11C7 VL CDR2
GTNNRAP

SEQ ID NO: 287 - protein, 9 aa, Artificial Sequence
  ACI-7079-2501B11-Ab3 2501B11C7 VL CDR3
ALWYSNHLV

SEQ ID NO: 288 - DNA, 360 bp, Artificial Sequence
  ACI-7079-2501B11-Ab3 2501B11C7 VH
caggttcagctgcagcagtctggacctgagctggtgaagcctggggcctcagtgaagatttcctgcaaggcttctggctacgcattcagtagtttctggatgaactggatgaaacagaggcctggaaagggtcttgagtggattggacggatttatcctggagatggagatgctcactacaatggggagttcaagggcagggccacactgactgcagacaaatcctccagcacagcctacatgcaactcagcagcctgacatctgaggactctgcggtctacttctgtgcaagaaagggggatttctacggtagtaactacgactattggggccaaggcaccactctcacagtctcctca

SEQ ID NO: 289 - DNA, 327 bp, Artificial Sequence
  ACI-7079-2501B11-Ab3 2501B11C7 VL
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccagactgactgtccta

SEQ ID NO: 290 - protein, 120 aa, Artificial Sequence
  ACI-7079-2501D10-Ab1 2501D10C3 VH
EVQLVESGGGLVQPKGSLKVSCAASGFTFKTYAMHWVRQAPGKGLEWVARIRSENSNFAKYYADSVKDRFTISRDDSQSMLYLQMNNLKTEDTAMYYCVRGYNGSSLDYWGQGTTLTVSS

SEQ ID NO: 291 - protein, 5 aa, Artificial Sequence
  ACI-7079-2501D10-Ab1 2501D10C3 VH CDR1
TYAMH

SEQ ID NO: 292 - protein, 19 aa, Artificial Sequence
  ACI-7079-2501D10-Ab1 2501D10C3 VH CDR2
RIRSENSNFAKYYADSVKD

SEQ ID NO: 293 - protein, 9 aa, Artificial Sequence
  ACI-7079-2501D10-Ab1 2501D10C3 VH CDR3
GYNGSSLDY

SEQ ID NO: 294 - protein, 106 aa, Artificial Sequence; identical to SEQ ID NO: 194
  ACI-7079-2501D10-Ab1 2501D10C3 VL

SEQ ID NO: 295 - protein, 10 aa, Artificial Sequence
  ACI-7079-2501D10-Ab1 2501D10C3 VL CDR1
SASSSVNYMH

SEQ ID NO: 296 - protein, 7 aa, Artificial Sequence
  ACI-7079-2501D10-Ab1 2501D10C3 VL CDR2
DTSKLAS

SEQ ID NO: 297 - protein, 9 aa, Artificial Sequence
  ACI-7079-2501D10-Ab1 2501D10C3 VL CDR3
QQWRSNPPT

SEQ ID NO: 298 - DNA, 360 bp, Artificial Sequence
  ACI-7079-2501D10-Ab1 2501D10C3 VH
gaggtgcagcttgttgagtctggtggaggattggtgcagcctaaaggatcattgaaagtctcatgtgccgcctctggtttcaccttcaagacctatgccatgcactgggtccgccaggctccgggaaagggtttggaatgggttgctcgcataagaagtgaaaacagtaattttgcaaaatattatgccgattcagtgaaggacagattcaccatctccagagatgattcacaaagtatgctctatctgcaaatgaacaacctgaaaactgaggacacagccatgtattattgtgtaaggggatataacggcagtagccttgactactggggccaaggcaccactctcacagtctcctca

SEQ ID NO: 299 - DNA, 318 bp, Artificial Sequence; identical to SEQ ID NO: 199
  ACI-7079-2501D10-Ab1 2501D10C3 VL

SEQ ID NO: 300 - protein, 117 aa, Artificial Sequence
  ACI-7087-4119E10-Ab2 VH
QVQLQQSGAELVRPGASVTLSCKASGYTFSDYEMNWVKQTPVHGLEWIGAIDPETGGTAYNQKFKGKAILTSDKSSSTAYMELRSLTSEDSAVYYCTRFLLIDFDYWGQGTTLTVSS

SEQ ID NO: 301 - protein, 5 aa, Artificial Sequence
  ACI-7087-4119E10-Ab2 VH CDR1
DYEMN

SEQ ID NO: 302 - protein, 17 aa, Artificial Sequence
  ACI-7087-4119E10-Ab2 VH CDR2
AIDPETGGTAYNQKFKG

SEQ ID NO: 303 - protein, 8 aa, Artificial Sequence
  ACI-7087-4119E10-Ab2 VH CDR3
FLLIDFDY

SEQ ID NO: 304 - protein, 112 aa, Artificial Sequence
  ACI-7087-4119E10-Ab2 VL
DVLMTQTPLSLPVSLGDQASISCRSSQSIVHSNGNTYLEWYLKKAGQSPKVLIYKVSNRFSGVPDRFSGSGSGTDFTLKISRVEAEDLGVYYCFQGSHVPYTFGGGTELEIK

SEQ ID NO: 305-306 - skipped (2 entries)

SEQ ID NO: 307 - protein, 9 aa, Artificial Sequence
  ACI-7087-4119E10-Ab2 VL CDR3
FQGSHVPYT

SEQ ID NO: 308 - DNA, 351 bp, Artificial Sequence
  ACI-7087-4119E10-Ab2 VH
caggttcaactgcagcagtctggggctgagctggtgaggcctggggcttcagtgacgctgtcctgcaaggcttcgggctacacattttctgactatgaaatgaactgggtgaagcagacacctgtgcatggcctggaatggattggagctattgatcctgaaactggtggtactgcctacaatcagaagttcaagggcaaggccatactgacttcagacaaatcctccagcacagcctacatggagctccgcagcctgacatctgaggactctgccgtctattactgtacaagattcctgttaatcgactttgactattggggccaaggcaccactctcacagtctcctca

SEQ ID NO: 309 - DNA, 336 bp, Artificial Sequence
  ACI-7087-4119E10-Ab2 VL
gatgtcttgatgacccaaactccactctccctgcctgtcagtcttggagatcaagcctccatctcttgcagatctagtcagagcattgtacatagtaatggaaacacctatttagaatggtacctgaagaaagcaggccagtctccaaaggtcctgatctacaaagtttccaaccgattttctggggtcccagacaggttcagtggcagtggatcagggacagatttcacactcaaaatcagcagggtggaggctgaggatctgggagtttattactgctttcaaggttcacatgttccgtacacattcggaggggggaccgagctggaaataaaa

SEQ ID NO: 310 - protein, 113 aa, Artificial Sequence
  ACI-7087-4125E6-Ab1 VH
QVQLQQPGTELVKPGASVRLSCKASGYAFTSYWMHWVKQRPGQGLEWIGNINPSNGGTNYNEKFKNKATLTVDKSSSTAYMQLSGLTSEDSAVYYCATGLHYWGQGTTLTVSS

SEQ ID NO: 311 - protein, 5 aa, Artificial Sequence
  ACI-7087-4125E6-Ab1 VH CDR1
SYWMH

SEQ ID NO: 312 - protein, 17 aa, Artificial Sequence
  ACI-7087-4125E6-Ab1 VH CDR2
NINPSNGGTNYNEKFKN

SEQ ID NO: 313 - protein, 4 aa, Artificial Sequence
  ACI-7087-4125E6-Ab1 VH CDR3
GLHY

SEQ ID NO: 314 - protein, 107 aa, Artificial Sequence
  ACI-7087-4125E6-Ab1 VL
DIQMTQSPSSLSASLGERVTLTCRASQDIGNYLNWLQQEPDGTIKRLIYATSSLDSGVPKRFSGSRSGSDYSLTISSLESEDFVDYYCLQFASSPLTFGPGTKLELK

SEQ ID NO: 315 - protein, 11 aa, Artificial Sequence
  ACI-7087-4125E6-Ab1 VL CDR1
RASQDIGNYLN

SEQ ID NO: 316-317 - skipped (2 entries)

SEQ ID NO: 318 - DNA, 339 bp, Artificial Sequence
  ACI-7087-4125E6-Ab1 VH
caggtccaactgcagcagcctgggactgaactggtgaagcctggggcttcagtgaggctgtcctgcaaggcttctggctacgccttcaccagctactggatgcactgggtgaagcagaggcctggacaaggccttgagtggattggaaatattaatcctagcaatggtggtactaactacaatgagaagttcaagaacaaggccacactgactgtagacaaatcctccagcacagcctatatgcagctcagcggcctgacatctgaggactctgcggtctattattgtgcaacgggccttcactactggggccaaggcaccactctcacagtctcctca

SEQ ID NO: 319 - DNA, 321 bp, Artificial Sequence
  ACI-7087-4125E6-Ab1 VL
gacatccagatgacccagtctccatcctccttatctgcctctctgggagaaagagtcactctcacttgtcgggcaagtcaggacattggtaattacttaaactggcttcagcaggaaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacaatttgctagttctccgctcacgttcggtcctgggaccaaactggaactgaaa

SEQ ID NO: 320 - protein, 117 aa, Artificial Sequence
  ACI-7088-4301D5-Ab2 VH
QVQLQQSGPELVRPGASVKISCKASGYRFTSYYIHWVKQRPGQGLEWIGWIYPGSDNTKHNDKFKGKATLTADTSSSTAYMQLSSLTSEDSAVYFCARDYDVGFGYWGQGTLVTVSS

SEQ ID NO: 321 - protein, 5 aa, Artificial Sequence
  ACI-7088-4301D5-Ab2 VH CDR1
SYYIH

SEQ ID NO: 322 - protein, 17 aa, Artificial Sequence
  ACI-7088-4301D5-Ab2 VH CDR2
WIYPGSDNTKHNDKFKG

SEQ ID NO: 323 - protein, 8 aa, Artificial Sequence
  ACI-7088-4301D5-Ab2 VH CDR3
DYDVGFGY

SEQ ID NO: 324 - protein, 111 aa, Artificial Sequence
  ACI-7088-4301D5-Ab2 VL
DIVLTQSPASLAVSLGQRATISCRASESVDNYGISFMNWFQQKPGQPPKLLIYAASNQGSGVPARFSGIGSGTDFSLNIHPMEEDDTAMYFCQQSQEVPLTFGAGTKLELK

SEQ ID NO: 325 - protein, 15 aa, Artificial Sequence
  ACI-7088-4301D5-Ab2 VL CDR1
RASESVDNYGISFMN

SEQ ID NO: 326 - protein, 7 aa, Artificial Sequence
  ACI-7088-4301D5-Ab2 VL CDR2
AASNQGS

SEQ ID NO: 327 - protein, 9 aa, Artificial Sequence
  ACI-7088-4301D5-Ab2 VL CDR3
QQSQEVPLT

SEQ ID NO: 328 - DNA, 351 bp, Artificial Sequence
  ACI-7088-4301D5-Ab2 VH
caggtccagctgcagcagtctggacctgagctggtgaggcctggggcttcagtgaagatatcctgcaaggcttctggctacaggttcacaagctactatatacactgggtgaagcagaggcctggacagggacttgagtggattggatggatttatcctggaagtgataatactaagcacaatgacaagttcaagggcaaggccacactgacggcagacacatcctccagcactgcctacatgcagctcagcagcctaacatctgaggactctgcggtctatttctgtgcaagagactacgacgtggggtttggttactggggccaagggactctggtcactgtctctgca

SEQ ID NO: 329 - DNA, 333 bp, Artificial Sequence
  ACI-7088-4301D5-Ab2 VL
gacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcattgggtctgggacagacttcagcctcaacatccatcctatggaggaggatgatactgcaatgtatttctgtcagcaaagtcaggaggttccgctcacgttcggtgctgggaccaagctggagctgaaa

SEQ ID NO: 330 - protein, 113 aa, Artificial Sequence
  ACI-7088-4301E12-Ab2 VH
DVQLQESGPGLVKPSQSLSLTCSVTGYSITSGYYWNWIRQFPGNKLEWMGYISDDGSKNYNPSLKNRISITRDTSKNQLFMKLNSVTTEDTATYYCARGDSRLGQGTLVTVSA

SEQ ID NO: 331 - skipped

SEQ ID NO: 332 - protein, 16 aa, Artificial Sequence
  ACI-7088-4301E12-Ab2 VH CDR2
YISDDGSKNYNPSLKN

SEQ ID NO: 333 - protein, 4 aa, Artificial Sequence
  ACI-7088-4301E12-Ab2 VH CDR3
GDSR

SEQ ID NO: 334 - protein, 112 aa, Artificial Sequence
  ACI-7088-4301E12-Ab2 VL
DVVLTQTPLTLSVTIGQPASISCRSSQNLLDSDGETYLNWLLQRPGQSPKRLIYLVSELDSGVPDRFTGSGSGTDFTLKISRVEAEDLGVYYCWQGTHFPQTFGGGTKLEII

SEQ ID NO: 335 - protein, 16 aa, Artificial Sequence
  ACI-7088-4301E12-Ab2 VL CDR1
RSSQNLLDSDGETYLN

SEQ ID NO: 336 - protein, 7 aa, Artificial Sequence
  ACI-7088-4301E12-Ab2 VL CDR2
LVSELDS

SEQ ID NO: 337 - skipped

SEQ ID NO: 338 - DNA, 339 bp, Artificial Sequence
  ACI-7088-4301E12-Ab2 VH
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagcgacgatggtagtaaaaattacaacccatctctcaaaaatcgaatctccatcactcgtgacacatctaagaaccagcttttcatgaagttgaattctgtgactactgaggacacagccacatattactgtgcaagaggcgattcccgcctgggccaagggactctggtcactgtctctgca

SEQ ID NO: 339 - DNA, 336 bp, Artificial Sequence
  ACI-7088-4301E12-Ab2 VL
gatgttgtgttgacccagactccactcactttgtcggttaccattggacaaccagcctccatctcttgcaggtcaagtcagaacctcttagatagtgatggagagacatatttgaattggttgttacagaggccaggccagtctccaaagcgcctaatctatctggtgtctgagctggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcatt

SEQ ID NO: 340 - protein, 117 aa, Artificial Sequence
  ACI-7088-4301H3-Ab2 VH
EVQLQQSGPELVKPGASVKISCKASGYTFADYFMNWVKQSHGKSLEWIGDINPNNGGTTYNQKFKGKATLTVDKSSNTAYMELRSLTSEDSAVYYCARGRNYAMDYWGQGTSVTVSS

SEQ ID NO: 341 - protein, 5 aa, Artificial Sequence
  ACI-7088-4301H3-Ab2 VH CDR1
DYFMN

SEQ ID NO: 342 - protein, 17 aa, Artificial Sequence
  ACI-7088-4301H3-Ab2 VH CDR2
DINPNNGGTTYNQKFKG

SEQ ID NO: 343 - protein, 8 aa, Artificial Sequence
  ACI-7088-4301H3-Ab2 VH CDR3
GRNYAMDY

SEQ ID NO: 344 - protein, 112 aa, Artificial Sequence
  ACI-7088-4301H3-Ab2 VL
DIVMSQSPSSLAVSAGEKVTMSCKSSQSLLNSRTRKNYLAWYQQKPGQSPKLLIYSASTRESGVPDRFTGSGFGTDFTLTISSVQAEDLAVYYCKQSYDLWTFGGGTKLEIK

SEQ ID NO: 345 - protein, 17 aa, Artificial Sequence
  ACI-7088-4301H3-Ab2 VL CDR1
KSSQSLLNSRTRKNYLA

SEQ ID NO: 346 - protein, 7 aa, Artificial Sequence
  ACI-7088-4301H3-Ab2 VL CDR2
SASTRES

SEQ ID NO: 347 - protein, 8 aa, Artificial Sequence
  ACI-7088-4301H3-Ab2 VL CDR3
KQSYDLWT

SEQ ID NO: 348 - DNA, 351 bp, Artificial Sequence
  ACI-7088-4301H3-Ab2 VH
gaggtccagctgcaacaatctggacctgaactggtgaagcctggggcttcagtgaagatatcttgtaaggcttctggatacacgttcgctgactacttcatgaactgggtgaagcagagccatggaaagagccttgagtggattggagatattaatcctaacaatggtggtactacctacaaccagaagttcaagggcaaggccacattgactgtagacaagtcctccaacacagcctacatggagctccgcagcctgacatctgaggactctgcagtctactactgtgcaagaggtagaaactacgctatggactactggggtcaaggaacctcagtcaccgtctcctca

SEQ ID NO: 349 - DNA, 336 bp, Artificial Sequence
  ACI-7088-4301H3-Ab2 VL
gacattgtgatgtcacagtctccatcctccctggctgtgtcagcaggagagaaggtcactatgagctgcaaatccagtcagagtctcctcaacagtagaacccgaaagaattatttggcttggtaccagcagaaaccagggcagtctcctaaattgttgatctactcggcatccactagggaatctggggtccctgatcgcttcacaggcagtggatttgggacagatttcactctcaccatcagcagtgtgcaggctgaggacctggcagtttattactgcaagcaatcttatgatctgtggacgttcggtggaggcaccaagctggaaatcaaa

SEQ ID NO: 350 - protein, 119 aa, Artificial Sequence
  ACI-7088-4303A1-Ab1 VH
EVQLQQSGAELVRPGASVKLSCTASGFNIKDDYMHWVKQRPEQGLEWIGWIDPENGDSEYASKFQGKATMTADTSSNTAYLQLSSLTSEDTAVYYCKTWGTAQALFPYWGQGTLVTVSA

SEQ ID NO: 351 - protein, 5 aa, Artificial Sequence
  ACI-7088-4303A1-Ab1 VH CDR1
DDYMH

SEQ ID NO: 352 - protein, 17 aa, Artificial Sequence
  ACI-7088-4303A1-Ab1 VH CDR2
WIDPENGDSEYASKFQG

SEQ ID NO: 353 - protein, 10 aa, Artificial Sequence
  ACI-7088-4303A1-Ab1 VH CDR3
WGTAQALFPY

SEQ ID NO: 354 - protein, 107 aa, Artificial Sequence
  ACI-7088-4303A1-Ab1 VL
DIVMTQSQKFMSTSVGDRVSITCKASQNVGTSVGWYQQKAGQSPKLLIHSASNRYTGVPDRFTGSGSGTDFTLTINNMQSEDLADYFCQQYRSYPLTFGAGTKLELK

SEQ ID NO: 355 - protein, 11 aa, Artificial Sequence
  ACI-7088-4303A1-Ab1 VL CDR1
KASQNVGTSVG

SEQ ID NO: 356 - protein, 7 aa, Artificial Sequence
  ACI-7088-4303A1-Ab1 VL CDR2
SASNRYT

SEQ ID NO: 357 - protein, 9 aa, Artificial Sequence
  ACI-7088-4303A1-Ab1 VL CDR3
QQYRSYPLT

SEQ ID NO: 358 - DNA, 357 bp, Artificial Sequence
  ACI-7088-4303A1-Ab1 VH
gaggttcagctgcagcagtctggggctgaacttgtgaggccaggggcctcagtcaagttgtcctgcacagcttctggctttaacattaaagacgactatatgcactgggtgaaacagaggcctgaacagggcctggagtggattggatggattgatcctgagaatggtgattctgaatatgcctcgaagttccagggcaaggccactatgacagcagacacatcctccaacacagcctacctgcaactcagcagcctgacatctgaggacactgccgtctattattgtaaaacatgggggacagctcaggccctctttccttactggggccaagggactctggtcactgtctctgca

SEQ ID NO: 359 - DNA, 321 bp, Artificial Sequence
  ACI-7088-4303A1-Ab1 VL
gacattgtgatgacccagtctcaaaaattcatgtccacatcagtaggagacagggtcagcatcacctgcaaggccagtcagaatgtgggtacttctgtaggctggtatcaacaaaaagcaggacaatctcctaaactactgattcactcggcatctaatcggtacactggagtccctgatcgcttcacaggcagtggatctgggacagatttcactctcaccatcaacaatatgcagtctgaagacctggcagattatttctgccagcaatatagaagttatccgctcacgttcggtgctgggaccaagctggagctgaaa

SEQ ID NO: 360 - protein, 117 aa, Artificial Sequence
  ACI-7088-4303A3-Ab1 VH
QVQLQQSGAELVRPGASVTLSCKASGYTFTDYEMHWVKQTPVHGLEWIGVIDPETGGAVQNQKFKGKAILTADNSSSTAYMDLRSLTSEDSAVYNCAMGAALRLAYWGQGTLVTVSS

SEQ ID NO: 361 - protein, 5 aa, Artificial Sequence
  ACI-7088-4303A3-Ab1 VH CDR1
DYEMH

SEQ ID NO: 362 - protein, 17 aa, Artificial Sequence
  ACI-7088-4303A3-Ab1 VH CDR2
VIDPETGGAVQNQKFKG

SEQ ID NO: 363 - protein, 8 aa, Artificial Sequence
  ACI-7088-4303A3-Ab1 VH CDR3
GAALRLAY

SEQ ID NO: 364 - protein, 112 aa, Artificial Sequence
  ACI-7088-4303A3-Ab1 VL
DVLMTQTPLSLPVSLGDQASISCRSSQSIVHSNGNSYLEWYLQKPGQSPKLLIYKVSNRFSGVPDRFSGSGSGTDFTLKINRVEAEDLGVYYCFQGSHVPFTFGSGTKLEIK

SEQ ID NO: 365 - protein, 16 aa, Artificial Sequence
  ACI-7088-4303A3-Ab1 VL CDR1
RSSQSIVHSNGNSYLE

SEQ ID NO: 366 - skipped

SEQ ID NO: 367 - protein, 9 aa, Artificial Sequence
  ACI-7088-4303A3-Ab1 VL CDR3
FQGSHVPFT

SEQ ID NO: 368 - DNA, 351 bp, Artificial Sequence
  ACI-7088-4303A3-Ab1 VH
caggttcaactgcagcagtctggggctgagctggtgaggcctggggcttcagtgacgctgtcctgcaaggcttcgggctacacatttactgactatgaaatgcactgggtgaaacagacacctgtgcatggcctggagtggattggagttattgatcctgaaactggtggtgctgtccagaatcagaagttcaagggcaaggccatactgactgcagacaattcctccagcacagcctacatggacctccgcagcctgacatctgaggactctgccgtctataactgtgcaatgggtgcggcattacggcttgcttactggggccaagggactctggtcactgtctctgca

SEQ ID NO: 369 - DNA, 336 bp, Artificial Sequence
  ACI-7088-4303A3-Ab1 VL
gatgttttgatgacccaaactccactctccctgcctgtcagtcttggagatcaagcctccatctcttgcagatctagtcagagcattgtacatagtaatggaaactcctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccattcacgttcggctcggggacaaagttggaaataaaa

SEQ ID NO: 370 - protein, 123 aa, Artificial Sequence
  ACI-7088-4303B6-Ab2 VH
EVQLQQSGPELVKPGASVKISCKASGYTFTDYYMNWVKQSHGKSLEWIGDINPNNGGTTYNQKFKDKATLTVDRSSSTAYMELRSLTSGDSAVYYCARSGYSGSRLYYAMDYWSQGSSVTVSS

SEQ ID NO: 371 - protein, 5 aa, Artificial Sequence
  ACI-7088-4303B6-Ab2 VH CDR1
DYYMN

SEQ ID NO: 372 - protein, 17 aa, Artificial Sequence
  ACI-7088-4303B6-Ab2 VH CDR2
DINPNNGGTTYNQKFKD

SEQ ID NO: 373 - protein, 14 aa, Artificial Sequence
  ACI-7088-4303B6-Ab2 VH CDR3
SGYSGSRLYYAMDY

SEQ ID NO: 374 - protein, 112 aa, Artificial Sequence
  ACI-7088-4303B6-Ab2 VL
DIVMSQSPSSLAVSAREKVTMSCKSSQSLLNSRTRKNYLAWYQQKPGQSPKLLIFWASTRESGVPDRFTGSGSGTDFTLTISSVQAEDLAVYYCKQSYDLWTFGGGTKLEIK

SEQ ID NO: 375 - skipped

SEQ ID NO: 376 - protein, 7 aa, Artificial Sequence
  ACI-7088-4303B6-Ab2 VL CDR2
WASTRES

SEQ ID NO: 377 - skipped

SEQ ID NO: 378 - DNA, 369 bp, Artificial Sequence
  ACI-7088-4303B6-Ab2 VH
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactacctacaaccagaagttcaaggacaaggccacattgactgtggacaggtcctccagcacagcctacatggaactccgcagcctgacatctggggactctgcagtctattactgtgcaagatcggggtactccggtagtcgcctctactatgctatggactactggagtcaaggatcctcagtcaccgtctcctca

SEQ ID NO: 379 - DNA, 336 bp, Artificial Sequence
  ACI-7088-4303B6-Ab2 VL
gacattgtgatgtcacagtctccatcctccctggctgtgtcagcacgagagaaggtcactatgagctgcaaatccagtcagagtctgctcaacagtagaacccgaaagaactacttggcttggtaccagcagaaaccagggcagtctcctaaactgctgatcttctgggcttccactagggaatctggggtccctgatcgcttcactggcagtggatctgggacagatttcactctcaccatcagcagtgtgcaggctgaagacctggcagtttattactgcaaacaatcttatgatctgtggacgttcggtggcggcaccaagctggaaatcaaa

SEQ ID NO: 380 - protein, 119 aa, Artificial Sequence
  ACI-7088-4303H6-Ab1 VH
EVQLQQSGAELVRPGASVKLSCTASGFNIQDDYMHWVKQRPEQGLEWIGWIDPENGDTEYASKFQGKATLTADTSSNTAYLQLSRLTSEDTAVYYCTTAGSGVQLFDYWGQGTTLTVSA

SEQ ID NO: 381 - skipped

SEQ ID NO: 382 - protein, 17 aa, Artificial Sequence
  ACI-7088-4303H6-Ab1 VH CDR2
WIDPENGDTEYASKFQG

SEQ ID NO: 383 - protein, 10 aa, Artificial Sequence
  ACI-7088-4303H6-Ab1 VH CDR3
AGSGVQLFDY

SEQ ID NO: 384 - protein, 107 aa, Artificial Sequence
  ACI-7088-4303H6-Ab1 VL
DILMTQSQKFMSTSVGDRVSVTCKASQNVGTNVAWYQQKPGQSPKPLISSASSRYSGVPDRFTGSGSGTDFTLTISNVQSEDLADYFCQQYNRYPLTFGAGTKLELK

SEQ ID NO: 385 - protein, 11 aa, Artificial Sequence
  ACI-7088-4303H6-Ab1 VL CDR1
KASQNVGTNVA

SEQ ID NO: 386 - protein, 7 aa, Artificial Sequence
  ACI-7088-4303H6-Ab1 VL CDR2
SASSRYS

SEQ ID NO: 387 - protein, 9 aa, Artificial Sequence
  ACI-7088-4303H6-Ab1 VL CDR3
QQYNRYPLT

SEQ ID NO: 388 - DNA, 357 bp, Artificial Sequence
  ACI-7088-4303H6-Ab1 VH
gaggttcagctgcagcagtctggggctgagcttgtgaggccaggggcctcagtcaagttgtcctgcacagcttctggctttaacattcaagacgactatatgcactgggtgaagcagaggcctgaacagggcctggagtggattggttggattgatcctgagaatggtgatactgaatatgcctcgaaattccagggcaaggccactttaacagcagacacatcctccaacacagcctacctgcagctcagcagactgacatctgaggacactgccgtctattactgtactacagcgggctcaggcgtccaactctttgactactggggccaaggcaccactctcacagtctcctca

SEQ ID NO: 389 - DNA, 321 bp, Artificial Sequence
  ACI-7088-4303H6-Ab1 VL
gacattttgatgacccagtctcaaaaattcatgtccacatcagtaggagacagggtcagcgtcacctgcaaggccagtcagaatgtgggtactaatgtagcctggtatcaacagaaaccagggcaatctcctaaaccactgatttcctcggcatcctcccggtacagtggcgtccctgatcgcttcacaggcagtggatctgggacagatttcactctcaccatcagcaatgtgcagtctgaagacttggcagactatttctgtcagcaatataaccgctatcctctcacgttcggtgctgggaccaagctggagctgaaa

SEQ ID NO: 390 - protein, 119 aa, Artificial Sequence
  ACI-7088-4305H7-Ab1 VH
EVQLQQSGAELVRPGASVKLSCTASGFNIKDDYMHWVKQRPEQGLEWIGWIDPENGDTEYASKFQGKATMIADTSSNTAYLQLSSLTSEDTAVYYCKTWGTTQALFPYWGQGTLVTVSS

SEQ ID NO: 391-392 - skipped (2 entries)

SEQ ID NO: 393 - protein, 10 aa, Artificial Sequence
  ACI-7088-4305H7-Ab1 VH CDR3
WGTTQALFPY

SEQ ID NO: 394 - protein, 107 aa, Artificial Sequence
  ACI-7088-4305H7-Ab1 VL
DIVMTQSQKFMSTSVGDRVSITCKASQNVGTAVGWYQQKAGQSPKLLIHSASNRYTGVPDRFTGSGSGTDFTLTINNMQSEDLADYFCQQYRSYPLTFGAGTKLELK

SEQ ID NO: 395 - protein, 11 aa, Artificial Sequence
  ACI-7088-4305H7-Ab1 VL CDR1
KASQNVGTAVG

SEQ ID NO: 396-397 - skipped (2 entries)

SEQ ID NO: 398 - DNA, 357 bp, Artificial Sequence
  ACI-7088-4305H7-Ab1 VH
gaggttcagctgcagcagtctggggctgaacttgtgaggccaggggcctcagtcaagttgtcctgcacagcttctggctttaacattaaagacgactatatgcactgggtgaagcagaggcctgaacagggcctggagtggattggatggattgatcctgagaatggtgatactgaatatgcctcgaagttccagggcaaggccactatgatagcagacacatcctccaacacagcctacctgcaactcagcagcctgacatctgaggacactgccgtctattattgtaaaacatgggggacaactcaggccctctttccttactggggccaagggactctggtcactgtctctgca

SEQ ID NO: 399 - DNA, 321 bp, Artificial Sequence
  ACI-7088-4305H7-Ab1 VL
gacattgtgatgacccagtctcaaaaattcatgtccacatcagtaggagacagggtcagcatcacctgcaaggccagtcagaatgtgggtactgctgtaggctggtatcaacaaaaagcaggacaatctcctaaactactgattcactcggcatccaatcggtacactggagtccctgatcgcttcacaggcagtggatctgggacagatttcactctcaccatcaacaatatgcagtctgaagacctggcagattatttctgccagcaatatagaagttatccgctcacgttcggtgctgggaccaagctggagctgaaa

SEQ ID NO: 400 - protein, 119 aa, Artificial Sequence
  ACI-7088-4317A4-Ab1 VH
EVQLQQSGAELVRPGASVKLSCTASGFNIKDDYMHWVKQRPEQGLEWIGWIDPENGDTEYASKFQGKATMTADTSSNTAYLQLSSLTSEDTAVYYCKTWGTTQALFPYWGQGTLVTVSA

SEQ ID NO: 401-403 - skipped (3 entries)

SEQ ID NO: 404 - protein, 107 aa, Artificial Sequence
  ACI-7088-4317A4-Ab1 VL
DIVMTQSQKFMYTSVGDRVSITCKASQNVGNAVGWYQQKAGQSPKLLIHSASNRYTGVPDRFTGTGSGTDFTLTINNMQSEDLADYFCQQYRSYPLTFGAGTKLELK

SEQ ID NO: 405 - protein, 11 aa, Artificial Sequence
  ACI-7088-4317A4-Ab1 VL CDR1
KASQNVGNAVG

SEQ ID NO: 406-407 - skipped (2 entries)

SEQ ID NO: 408 - DNA, 357 bp, Artificial Sequence
  ACI-7088-4317A4-Ab1 VH
gaggttcagctgcagcagtctggggctgaacttgtgaggccaggggcctcagtcaagttgtcctgcacagcttctggctttaacattaaagacgactatatgcactgggtgaagcagaggcctgaacagggcctggagtggattggatggattgatcctgagaatggtgatactgaatatgcctcgaagttccagggcaaggccactatgacagcagacacatcctccaacacagcctacctgcaactcagcagcctgacatctgaggacactgccgtctattattgtaaaacatgggggacaactcaggccctctttccttactggggccaagggactctggtcactgtctctgca

SEQ ID NO: 409 - DNA, 321 bp, Artificial Sequence
  ACI-7088-4317A4-Ab1 VL
gacattgtgatgacccagtctcaaaagttcatgtacacatcagtgggagacagggtcagcatcacctgcaaggccagtcagaatgtgggtaatgctgtaggctggtatcaacaaaaagcaggacaatctcctaaactactgattcactcggcatccaatcggtacactggagtccctgatcgcttcacaggcactggatctgggacagatttcactctcaccatcaacaatatgcagtctgaagacctggcagattatttctgccagcaatatagaagttatccgctcacgttcggtgctgggaccaagctggagctgaaa

SEQ ID NO: 410 - protein, 113 aa, Artificial Sequence
  ACI-7089-4409F1-Ab1 VH
DVQLQESGPGLVKPSQSLSLTCSVTGYSITRGYYWNWIRQFPGNKLEWMGYISYDGSNNYNPSLRNRISITRDTSKNQFFLKLKSVTTEDTATYFCARGDSNWGQGTTLTVSA

SEQ ID NO: 411 - protein, 6 aa, Artificial Sequence
  ACI-7089-4409F1-Ab1 VH CDR1
RGYYWN

SEQ ID NO: 412 - protein, 16 aa, Artificial Sequence
  ACI-7089-4409F1-Ab1 VH CDR2
YISYDGSNNYNPSLRN

SEQ ID NO: 413 - protein, 4 aa, Artificial Sequence
  ACI-7089-4409F1-Ab1 VH CDR3
GDSN

SEQ ID NO: 414 - protein, 112 aa, Artificial Sequence
  ACI-7089-4409F1-Ab1 VL
DVVMTQTPLTLSVTIGQPASISCKSSQSLLDSDGETYLNWLLQRPGQSPKRLIYLVSKLDSGVPDRFTGSGSGTDFTLKISRVEAEDLGVYYCWQGTHFPQTFGGGTKLEIK

SEQ ID NO: 415-417 - skipped (3 entries)

SEQ ID NO: 418 - DNA, 339 bp, Artificial Sequence
  ACI-7089-4409F1-Ab1 VH
gatgtacagcttcaggagtcaggacctggcctcgtgaaaccttctcagtctctgtctctcacctgctctgtcactggctactccatcaccaggggttattactggaactggatccggcagtttccaggaaacaaactggaatggatgggctacataagctacgatggtagcaataactacaacccatctctcagaaatcgaatctccatcactcgtgacacatctaagaaccagtttttcctgaagttgaaatctgtgactactgaggacacagccacatatttctgtgcaagaggggatagtaactggggccaaggcaccactctcacagtctcctca

SEQ ID NO: 419 - DNA, 336 bp, Artificial Sequence
  ACI-7089-4409F1-Ab1 VL
gatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctatctggtgtctaaactggactctggagtccctgacaggttcactggtagtggatcagggacagatttcacactgaaaatcagcagagtggaggctgaggatttgggagtttattattgctggcaaggtacacattttcctcagacgttcggtggaggcaccaagttggaaatcaaa

SEQ ID NO: 420 - protein, 112 aa, Artificial Sequence
  ACI-7089-4415G5-Ab1 VH
QVQLQQSGAELARPGASVKVSCKASGYTFTSSGISWLKHRTGQGLEWIGDIYPRSGNTYYNEKFKDKATLTADKSSSTAYMELRSLTSEDSAVYFCASGNYWGQGTTLTVSA

SEQ ID NO: 421 - protein, 5 aa, Artificial Sequence
  ACI-7089-4415G5-Ab1 VH CDR1
SSGIS

SEQ ID NO: 422 - protein, 17 aa, Artificial Sequence
  ACI-7089-4415G5-Ab1 VH CDR2
DIYPRSGNTYYNEKFKD

SEQ ID NO: 423 - skipped

SEQ ID NO: 424 - protein, 112 aa, Artificial Sequence
  ACI-7089-4415G5-Ab1 VL
DIVITQDDLSNPVTSGESVSISCRSSKSLLYKDGKTYLNWFLQRPGQSPQLLIYLMSTRASGVSDRFSGSGSGTDFTLEISRVKAEDVGVYYCQQLLEYPLTFGAGTKLELK

SEQ ID NO: 425 - protein, 16 aa, Artificial Sequence
  ACI-7089-4415G5-Ab1 VL CDR1
RSSKSLLYKDGKTYLN

SEQ ID NO: 426 - protein, 7 aa, Artificial Sequence
  ACI-7089-4415G5-Ab1 VL CDR2
LMSTRAS

SEQ ID NO: 427 - protein, 9 aa, Artificial Sequence
  ACI-7089-4415G5-Ab1 VL CDR3
QQLLEYPLT

SEQ ID NO: 428 - DNA, 336 bp, Artificial Sequence
  ACI-7089-4415G5-Ab1 VH
caggttcagctgcagcagtctggagctgagctggcgaggcctggggcttcagtgaaggtgtcctgcaaggcttctggctacaccttcacaagctctggtataagctggttgaagcacagaactggacagggccttgagtggattggagacatttatcctagaagtggtaatacttactacaatgagaaattcaaggacaaggccacactgactgcagacaaatcctccagcacggcgtacatggagctccgcagcctgacatctgaggactctgcggtctatttctgtgcaagtggtaactactggggccaaggcaccactctcacagtctcctca

SEQ ID NO: 429 - DNA, 336 bp, Artificial Sequence
  ACI-7089-4415G5-Ab1 VL
gatattgtgataacccaggatgacctctccaatcctgtcacttctggagaatcagtttccatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaacttttagagtatccgctcacgttcggtgctgggaccaagctggagctgaaa

SEQ ID NO: 430 - protein, 114 aa, Artificial Sequence
  ACI-7089-4417G6-Ab1 VH
QVQLQQSGAELVRPGASVTLSCKASGYTFTGYEMHKQTPVHGLEWIGAIDPETGGTAYIQKFKGKATLTADKSSSTAYMELRSLTSEDSAVYYCTRGWDYFDYWGQGTTLTVSA

SEQ ID NO: 431 - protein, 5 aa, Artificial Sequence
  ACI-7089-4417G6-Ab1 VH CDR1
GYEMH

SEQ ID NO: 432 - protein, 17 aa, Artificial Sequence
  ACI-7089-4417G6-Ab1 VH CDR2
AIDPETGGTAYIQKFKG

SEQ ID NO: 433 - protein, 7 aa, Artificial Sequence
  ACI-7089-4417G6-Ab1 VH CDR3
GWDYFDY

SEQ ID NO: 434 - protein, 112 aa, Artificial Sequence
  ACI-7089-4417G6-Ab1 VL
DVVMTQTPLSLPVSLGDQASISCRSSQSLLHSNGFTYLHWYLQKPGQSPKLLIYRVSNRFSGVPDRFSGSGSGTDFTLKISRVEAEDLGVYFCSQSTHVPYTFGGGTKLEIK

SEQ ID NO: 435 - protein, 16 aa, Artificial Sequence
  ACI-7089-4417G6-Ab1 VL CDR1
RSSQSLLHSNGFTYLH

SEQ ID NO: 436 - protein, 7 aa, Artificial Sequence
  ACI-7089-4417G6-Ab1 VL CDR2
RVSNRFS

SEQ ID NO: 437 - protein, 9 aa, Artificial Sequence
  ACI-7089-4417G6-Ab1 VL CDR3
SQSTHVPYT

SEQ ID NO: 438 - DNA, 348 bp, Artificial Sequence
  ACI-7089-4417G6-Ab1 VH
caggttcaactgcagcagtctggggctgagttggtgaggcctggggcttcagtgacgctgtcctgcaaggcttcgggctacacatttactggctatgaaatgcactgggtgaagcagacacctgtgcatggcctggaatggattggagctattgatcctgaaaccggtggaactgcctatattcagaagttcaagggcaaggccacactgactgcagacaaatcctccagcacagcctacatggagctccgcagcctgacatctgaggactctgccgtctattactgtacaagaggctgggactattttgactactggggccaaggcaccactctcacagtctcctca

SEQ ID NO: 439 - DNA, 336 bp, Artificial Sequence
  ACI-7089-4417G6-Ab1 VL
gatgttgtgatgacccaaactccactctccctgcctgtcagtcttggagatcaagcctccatctcttgcagatctagtcagagccttctacacagtaatggattcacctatttacattggtacctgcagaagccaggccagtctccaaagctcctgatctacagagtttccaatcgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagctggaaataaaa

SEQ ID NO: 440 - protein, 113 aa, Artificial Sequence
  ACI-7089-4418C5-Ab1 VH
DGQLQESGPGLVKPSQSLSLTCSVTGYSITSGYYWNWIRQFPGNKLEWMGYINYDGSNNYNPSLKNRISITRDTSKNQFFLKFNFVTTEDTATYYCVRGDVYWGQGTTLTVSS

SEQ ID NO: 441 - skipped

SEQ ID NO: 442 - protein, 16 aa, Artificial Sequence
  ACI-7089-4418C5-Ab1 VH CDR2
YINYDGSNNYNPSLKN

SEQ ID NO: 443 - protein, 4 aa, Artificial Sequence
  ACI-7089-4418C5-Ab1 VH CDR3
GDVY

SEQ ID NO: 444-447 - skipped (4 entries)

SEQ ID NO: 448 - DNA, 339 bp, Artificial Sequence
  ACI-7089-4418C5-Ab1 VH
gatggacaacttcaggagtcaggacctggcctcgtgaaaccttctcagtctctgtctctcacctgctctgtcactggctactccatcaccagtggatattactggaactggatccggcagtttccaggaaacaaactggaatggatgggctacataaactacgatggtagcaataactacaacccatctctcaaaaatcgaatctccatcactcgtgacacatctaagaatcagtttttcctgaagttcaattttgtgactactgaggacacagccacatattactgtgtgaggggggacgtctactggggccaaggcaccactctcacagtctcctca

SEQ ID NO: 449 - DNA, 336 bp, Artificial Sequence
  ACI-7089-4418C5-Ab1 VL
gatgttgtgatgacccagactccactcactttgtcggttaccattggacaaccagcctccatctcttgcaagtcaagtcagagcctcttagatagtgatggagagacatatttgaattggttattacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 450 - protein, 112 aa, Artificial Sequence
  ACI-7089-4418F6-Ab1 VH
QVQLQQSGAELARPGASVKVSCKASGYTFTSSGISWLKHRTGQGLEWIGDIYPRSGNTYYNEKFKDKATLTADKSSSTAYMELRSLTSEDSAVYFCSSGNYWGQGTTLTVSS

SEQ ID NO: 451-457 - skipped (7 entries)

SEQ ID NO: 458 - DNA, 336 bp, Artificial Sequence
  ACI-7089-4418F6-Ab1 VH
caggttcagctgcagcagtctggagctgagctggcgaggcctggggcttcagtgaaggtgtcctgcaaggcttctggctacaccttcacaagttctggtataagctggttgaagcacagaactggacagggccttgagtggattggagacatttatcctagaagtggtaatacttactacaatgagaaattcaaggacaaggccacactgactgcagacaaatcctccagcacggcgtacatggagctccgcagcctgacatctgaggactctgcggtctatttctgttcaagtggtaactactggggccaaggcaccactctcacagtctcctca

SEQ ID NO: 459 - DNA, 336 bp, Artificial Sequence
  ACI-7089-4418F6-Ab1 VL
gatattgtgataacccaggatgacctctccaatcctgtcacttctggagaatcagtttccatctcctgtaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtagagtgaaggctgaggatgtgggtgtgtattactgtcaacaacttttagagtatccgctcacgttcggtgctgggaccaagctggagctgaaa

SEQ ID NO: 460 - protein, 114 aa, Artificial Sequence
  ACI-8033-5A12-Ab1 VH
QVHLKQSGADLVRPGASVKLSCKASGYTFTDYYINWVKQRPGQGLEWIARIYPGSGNTYYNEKFKGRATLSAEKSSTTAYMQLSSLTSEDSAVYFCVVGYYGAWGQGTTLTVSS

SEQ ID NO: 461 - protein, 5 aa, Artificial Sequence
  ACI-8033-5A12-Ab1 VH CDR1
DYYIN

SEQ ID NO: 462 - protein, 17 aa, Artificial Sequence
  ACI-8033-5A12-Ab1 VH CDR2
RIYPGSGNTYYNEKFKG

SEQ ID NO: 463 - protein, 5 aa, Artificial Sequence
  ACI-8033-5A12-Ab1 VH CDR3
GYYGA

SEQ ID NO: 464 - protein, 112 aa, Artificial Sequence
  ACI-8033-5A12-Ab1 VL
DVVMTQTPLSLPVSLGDQASISCRSSQSLVHSNGKTHLHWYLQKPGQSPKLLIYKVSNRFSGVPDRFSGSGSGTDFTLKISRVEAEDLGVYFCSQSTHVPWTFGGGTKLEIK

SEQ ID NO: 465 - protein, 16 aa, Artificial Sequence
  ACI-8033-5A12-Ab1 VL CDR1
RSSQSLVHSNGKTHLH

SEQ ID NO: 466 - skipped

SEQ ID NO: 467 - protein, 9 aa, Artificial Sequence
  ACI-8033-5A12-Ab1 VL CDR3
SQSTHVPWT

SEQ ID NO: 468 - DNA, 342 bp, Artificial Sequence
  ACI-8033-5A12-Ab1 VH
caggtccacctgaagcagtctggggctgacctggtgaggcctggggcttcagtgaagctgtcctgcaaggcgtctggctacactttcactgactactatataaactgggtgaagcagaggcctggacagggacttgagtggattgcaaggatttatcctggaagtggtaatacttactacaatgagaagttcaagggcagggccacactgagtgcagaaaaatcctccaccactgcctacatgcagctcagcagcctgacatctgaggactctgctgtctatttctgtgtagtggggtactacggtgcctggggccaaggcaccactctcacagtctcctca

SEQ ID NO: 469 - DNA, 336 bp, Artificial Sequence
  ACI-8033-5A12-Ab1 VL
gatgttgtgatgacccaaactccactctccctgcctgtcagtcttggagatcaagcctccatctcttgtagatctagtcagagccttgtacacagtaatggaaaaacccatttacattggtacctgcagaagccaggccagtctccaaagctcctgatctataaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 470 - protein, 115 aa, Artificial Sequence
  ACI-8033-25A3-Ab1 VH
EVQLVESGGGLVQPKGSLKLSCAASGFSFNTYAMNWVRQAPGKGLEWVARIRSKSNNFATYYADSVKDRFTISRDESESMLYLQMNNLKTEDTAMYYCVRSFDYWGQGTTLTVSS

SEQ ID NO: 471 - skipped

SEQ ID NO: 472 - protein, 19 aa, Artificial Sequence
  ACI-8033-25A3-Ab1 VH CDR2
RIRSKSNNFATYYADSVKD

SEQ ID NO: 473 - protein, 4 aa, Artificial Sequence
  ACI-8033-25A3-Ab1 VH CDR3
SFDY

SEQ ID NO: 474 - protein, 107 aa, Artificial Sequence
  ACI-8033-25A3-Ab1 VL
DIKMTQSPSSMYASLGERVTITCKASQDINSYLSWFQQKPGKSPKTLIYRAKRLVDGVPSRFSGSGSGQDYSLTISSLEYEDMGIYYCLQYDEFPFTFGSGTKLEIK

SEQ ID NO: 475 - protein, 11 aa, Artificial Sequence
  ACI-8033-25A3-Ab1 VL CDR1
KASQDINSYLS

SEQ ID NO: 476 - protein, 7 aa, Artificial Sequence
  ACI-8033-25A3-Ab1 VL CDR2
RAKRLVD

SEQ ID NO: 477 - protein, 9 aa, Artificial Sequence
  ACI-8033-25A3-Ab1 VL CDR3
LQYDEFPFT

SEQ ID NO: 478 - DNA, 345 bp, Artificial Sequence
  ACI-8033-25A3-Ab1 VH
gaggtgcagcttgttgagtctggtggaggattggtgcagcctaaagggtcattgaaactctcatgtgcagcctctggattcagcttcaatacctacgccatgaactgggtccgccaggctccaggaaagggtttggaatgggttgctcgcataagaagtaaaagtaataattttgcaacatattatgccgattcagtgaaagacagattcaccatctccagagatgaatcagaaagcatgctctatctgcaaatgaacaacttgaaaactgaggacacagccatgtattactgtgtgaggtcctttgactactggggccaaggcaccactctcacagtctcctca

SEQ ID NO: 479 - DNA, 321 bp, Artificial Sequence
  ACI-8033-25A3-Ab1 VL
gacatcaagatgacccagtctccatcttccatgtatgcatctctaggagagagagtcactatcacttgcaaggcgagtcaggacattaatagctatttaagctggttccagcagaaaccagggaaatctcctaagaccctaatctatcgtgcaaaaagattggtagatggggtcccatcaaggttcagtggcagtggatctgggcaagattattctctcaccatcagcagcctggagtatgaagatatgggaatttattattgtctacagtatgatgagtttccattcacgttcggctcggggacaaagttggaaataaaa

SEQ ID NO: 480 - protein, 119 aa, Artificial Sequence
  ACI-8033-1G10-Ab1 VH
DVQLQESGPGLVKPSQTVFLTCTVTGISITTGNYRWSWIRQFPGNKLEWIGYIYYSGTITYNPSLTSRTTITRDTPKNQFFLEMNSLTAEDTATYYCARIYYGNAMDYWGQGTSVTVSS

SEQ ID NO: 481 - protein, 7 aa, Artificial Sequence
  ACI-8033-1G10-Ab1 VH CDR1
TGNYRWS

SEQ ID NO: 482 - protein, 16 aa, Artificial Sequence
  ACI-8033-1G10-Ab1 VH CDR2
YIYYSGTITYNPSLTS

SEQ ID NO: 483 - protein, 9 aa, Artificial Sequence
  ACI-8033-1G10-Ab1 VH CDR3
IYYGNAMDY

SEQ ID NO: 484 - protein, 112 aa, Artificial Sequence
  ACI-8033-1G10-Ab1 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPHTFGGGTKLEIK

SEQ ID NO: 485-486 - skipped (2 entries)

SEQ ID NO: 487 - protein, 9 aa, Artificial Sequence
  ACI-8033-1G10-Ab1 VL CDR3
SQSTHVPHT

SEQ ID NO: 488 - DNA, 357 bp, Artificial Sequence
  ACI-8033-1G10-Ab1 VH
gatgtgcagcttcaggagtcaggacctggcctggtgaaaccttctcagacagtgttcctcacctgcactgtcactggcatttccatcaccactggaaattacaggtggagctggatccggcagtttccaggaaacaaactggagtggatagggtacatatactacagtggtaccattacctacaatccatctctcacaagtcgaaccaccatcactagagacactcccaagaaccagttcttcctggaaatgaactctttgactgctgaggacacagccacatactactgtgcacggatttactacggtaatgctatggactactggggtcaaggaacctcagtcaccgtctcctca

SEQ ID NO: 489 - DNA, 336 bp, Artificial Sequence
  ACI-8033-1G10-Ab1 VL
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctcacacgttcggaggggggaccaagctggaaataaaa

SEQ ID NO: 490 - protein, 115 aa, Artificial Sequence
  ACI-8033-19A2-Ab1 VH
EVQLVESGGGLVQPKGSLKLSCAASGFSFNTYAMNWVRQAPGKGLEWVARIRSKSNNYATYYVDSVKDRFTISRDDSESMLYLQMNNLKTEDTALYYCVSESAYWGQGTLVTVSA

SEQ ID NO: 491 - skipped

SEQ ID NO: 492 - protein, 19 aa, Artificial Sequence
  ACI-8033-19A2-Ab1 VH CDR2
RIRSKSNNYATYYVDSVKD

SEQ ID NO: 493 - protein, 4 aa, Artificial Sequence
  ACI-8033-19A2-Ab1 VH CDR3
ESAY

SEQ ID NO: 494 - protein, 112 aa, Artificial Sequence
  ACI-8033-19A2-Ab1 VL
DVVMTQTPLSLPVSLGDQASISCRSSQSLVHSNGNTYLYWYLQKPGQSPKLLIYKVSNRLSGVPDRFSGSGSGTDFTLKISRVEAEDLGVYFCSQSTHVPFTFGSGTKLEIK

SEQ ID NO: 495 - protein, 16 aa, Artificial Sequence
  ACI-8033-19A2-Ab1 VL CDR1
RSSQSLVHSNGNTYLY

SEQ ID NO: 496 - protein, 7 aa, Artificial Sequence
  ACI-8033-19A2-Ab1 VL CDR2
KVSNRLS

SEQ ID NO: 497 - protein, 9 aa, Artificial Sequence
  ACI-8033-19A2-Ab1 VL CDR3
SQSTHVPFT

SEQ ID NO: 498 - DNA, 345 bp, Artificial Sequence
  ACI-8033-19A2-Ab1 VH
gaggtgcaacttgttgagtctggtggaggattggtgcagcctaaagggtcattgaaactctcatgtgcagcctctggattcagcttcaatacctacgccatgaactgggtccgccaggctccaggaaagggtttggaatgggttgctcgcataagaagtaaaagtaataattatgcaacatattatgtcgattcagtgaaagacagattcaccatctccagagatgattcagaaagcatgctctatctgcaaatgaacaacttgaaaactgaggacacagccctgtattactgtgtgagcgaatccgcttactggggccaagggactctggtcactgtctctgca

SEQ ID NO: 499 - DNA, 336 bp, Artificial Sequence
  ACI-8033-19A2-Ab1 VL
gatgttgtgatgacccaaactccactctccctgcctgtcagtcttggagatcaagcctccatctcttgcagatctagtcagagccttgtacacagtaatggaaacacctatttatattggtacctgcagaagccaggccagtctccaaagctcctgatctacaaagtttccaaccgactttctggggtcccagacaggttcagtggcagtggatcagggacagatttcacactcaagatcagcagagtggaggctgaggatctgggagtttatttctgctctcaaagtacacatgttccattcacgttcggctcggggacaaagttggaaataaaa

SEQ ID NO: 500 - protein, 113 aa, Artificial Sequence
  ACI-8033-8C10-Ab1 VH
DVQLQESGPGLVKPSQSLSLTCSVTGQSITSGYYWNWIRQFPGNKLEWMGYISNDGSSKTNPSLTNRISVTRDTSKNQVFLKLKSVTTEDTATYYCVRGDQHWGQGTALTVSS

SEQ ID NO: 501 - skipped

SEQ ID NO: 502 - protein, 16 aa, Artificial Sequence
  ACI-8033-8C10-Ab1 VH CDR2
YISNDGSSKTNPSLTN

SEQ ID NO: 503 - protein, 4 aa, Artificial Sequence
  ACI-8033-8C10-Ab1 VH CDR3
GDQH

SEQ ID NO: 504 - protein, 112 aa, Artificial Sequence
  ACI-8033-8C10-Ab1 VL
DVVLTQTPLTLSVTIGQPASISCKSSQSLLDSDGETYLNWLLQRPGQSPKRLIYLVSELDSGVSDRFTGSGSGTDFTLKISRLEAEDLGVYYCWQGTHFPQTFGGGTKLEIK

SEQ ID NO: 505-507 - skipped (3 entries)

SEQ ID NO: 508 - DNA, 339 bp, Artificial Sequence
  ACI-8033-8C10-Ab1 VH
gatgtacagcttcaggagtcaggacctggcctcgtgaaaccttctcagtctctgtctctcacctgctctgtcactggccaatccatcaccagtggttattactggaactggatccggcaatttccaggaaacaaactggaatggatgggctacataagcaacgatggtagcagtaaaaccaacccatctctcacaaatcgaatctccgtcactcgtgacacatctaagaaccaggttttcctgaagttgaaatctgtgactactgaggacacagccacatattactgtgtaagaggggaccagcactggggccaaggcaccgctctcacagtctcctca

SEQ ID NO: 509 - DNA, 336 bp, Artificial Sequence
  ACI-8033-8C10-Ab1 VL
gatgttgtgttgacccagactccactcactttgtcagttaccattgggcaaccagcctccatctcttgcaagtcaagtcagagcctcttagatagtgatggagagacatatttgaattggttgttacagaggccaggccagtctccaaagcgcctaatctatctggtgtctgaactggactctggagtctctgacaggttcactggcagtggttcagggacagatttcacactgaaaatcagcagactggaggctgaggatttgggagtttattattgctggcaaggtacacattttcctcagacgttcggtggaggcaccaagctggaaatcaaa

SEQ ID NO: 510 - protein, 120 aa, Artificial Sequence
  ACI-8033-7A2-Ab1 VH
QVQLQQPGTELVKPGASVNLPCKASGYTFTSYWMHWVKQRPGQGLDWIGNVNPNNSDSNYNEKFKRKATLTVDKSSSTAYMHLSSLTSEDSAVYYCARSPYYGGRYLDYWGQGTTLTVSS

SEQ ID NO: 511 - skipped

SEQ ID NO: 512 - protein, 17 aa, Artificial Sequence
  ACI-8033-7A2-Ab1 VH CDR2
NVNPNNSDSNYNEKFKR

SEQ ID NO: 513 - protein, 11 aa, Artificial Sequence
  ACI-8033-7A2-Ab1 VH CDR3
SPYYGGRYLDY

SEQ ID NO: 514 - protein, 113 aa, Artificial Sequence
  ACI-8033-7A2-Ab1 VL
DIVMSQSPSSLAVSVGEKVTMTCKSSQSLLYRSNQKNYLAWYQQKPGQSPKLLIYWAFTRESGVPDRFTGSGSGTDFTLTISSVKAEDLAVYYCQQYYSYPLTFGAGTKLELK

SEQ ID NO: 515 - protein, 17 aa, Artificial Sequence
  ACI-8033-7A2-Ab1 VL CDR1
KSSQSLLYRSNQKNYLA

SEQ ID NO: 516 - protein, 7 aa, Artificial Sequence
  ACI-8033-7A2-Ab1 VL CDR2
WAFTRES

SEQ ID NO: 517 - protein, 9 aa, Artificial Sequence
  ACI-8033-7A2-Ab1 VL CDR3
QQYYSYPLT

SEQ ID NO: 518 - DNA, 360 bp, Artificial Sequence
  ACI-8033-7A2-Ab1 VH
caggtccaactacagcagcctgggactgaactggtgaagcctggggcttcagtgaacctgccctgcaaggcttctggctacaccttcaccagctactggatgcactgggtgaagcagaggcctggtcaaggccttgattggattggaaatgttaatcctaacaatagtgatagtaattacaatgagaagttcaagaggaaggccacactgactgtagacaaatcctccagcacagcctacatgcacctcagcagcctgacatctgaggactctgcggtctattattgtgcaagatctccttactacggtggccgttaccttgactactggggccaaggcaccactctcacagtctcctca

SEQ ID NO: 519 - DNA, 339 bp, Artificial Sequence
  ACI-8033-7A2-Ab1 VL
gacattgtgatgtcacagtctccatcctccctagctgtgtcagttggagagaaggttactatgacctgcaagtccagtcagagccttttatatagaagcaatcaaaagaactacttggcctggtaccagcagaaaccaggacagtctcctaaactgttgatttactgggcattcactagggaatctggggtccctgatcgcttcacaggcagtggatctgggacagatttcactctcaccatcagcagtgtgaaggctgaagacctggcagtttattactgtcagcaatattatagctatcctctcacgttcggtgctgggaccaagctggagctgaaa

SEQ ID NO: 520 - protein, 114 aa, Artificial Sequence
  ACI-8033-1A12-Ab1 VH
QVHLKQSGADLVRPGASVKLSCKASGYSFTDFYINWVKQTPGQGLEWIARIYPGNNNTFYNEKFKGKATLSAEKSSTTAYMQLSSLTSEDSAVYFCVVGYYGAWGQGTTLTVSS

SEQ ID NO: 521 - protein, 5 aa, Artificial Sequence
  ACI-8033-1A12-Ab1 VH CDR1
DFYIN

SEQ ID NO: 522 - protein, 17 aa, Artificial Sequence
  ACI-8033-1A12-Ab1 VH CDR2
RIYPGNNNTFYNEKFKG

SEQ ID NO: 523 - skipped

SEQ ID NO: 524 - protein, 112 aa, Artificial Sequence
  ACI-8033-1A12-Ab1 VL
DVVMTQTPLSLPVSLGDQASISCRSSQSLVHSNGNTHLHWYLQKPGQSPKLLIYKVSNRFSGVPDRFSGSGSGTDFTLKISRVEAEDLGFYFCSQSTHVPWTFGGGTKLEIK

SEQ ID NO: 525 - protein, 16 aa, Artificial Sequence
  ACI-8033-1A12-Ab1 VL CDR1
RSSQSLVHSNGNTHLH

SEQ ID NO: 526-527 - skipped (2 entries)

SEQ ID NO: 528 - DNA, 342 bp, Artificial Sequence
  ACI-8033-1A12-Ab1 VH
caggtccacctgaagcagtctggggctgacctggtgaggcctggggcttcagtgaagctgtcctgcaaggcgtctggctacagtttcactgacttttatataaattgggtgaagcagacgcctggacagggacttgagtggattgcgaggatttatcctggaaataataatactttctacaatgagaaattcaagggcaaggccacactgagtgcagaaaaatcctccaccactgcctacatgcagctcagcagcctgacatctgaggactctgctgtctatttctgtgtagtggggtactacggtgcctggggccaaggcaccactctcacagtctcctca

SEQ ID NO: 529 - DNA, 336 bp, Artificial Sequence
  ACI-8033-1A12-Ab1 VL
gatgttgtgatgacccaaactccactctccctgcctgtcagtcttggagatcaagcctccatctcttgcagatctagtcagagccttgtacacagtaatggaaacacccatttgcattggtacctgcagaagccaggccagtctccaaagctcctgatctataaagtttccaaccgattttctggggtcccagacaggttcagtggcagtggatcagggacagatttcacactcaagatcagcagagtggaggctgaggatctaggattttatttctgctctcaaagtacacatgttccgtggacgttcggtggaggcaccaagctggaaatcaaa

SEQ ID NO: 530 - protein, 125 aa, Artificial Sequence
  ACI-8033-4F3-Ab1 VH
EVQLQQSGPELVKPGASVKISCKASGYTFTDYYMNWVKQSHGKSLEWIGDINPNTGTNSYNQKFKGRASLTVDKFSSAAYMELRSLTSEDSAVYYCARTGYGDPISSYYYALDYWGQGTSVTVSS

SEQ ID NO: 531 - skipped

SEQ ID NO: 532 - protein, 17 aa, Artificial Sequence
  ACI-8033-4F3-Ab1 VH CDR2
DINPNTGTNSYNQKFKG

SEQ ID NO: 533 - protein, 16 aa, Artificial Sequence
  ACI-8033-4F3-Ab1 VH CDR3
TGYGDPISSYYYALDY

SEQ ID NO: 534 - protein, 112 aa, Artificial Sequence
  ACI-8033-4F3-Ab1 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEIK

SEQ ID NO: 535-536 - skipped (2 entries)

SEQ ID NO: 537 - protein, 8 aa, Artificial Sequence
  ACI-8033-4F3-Ab1 VL CDR3
KQSYNLWT

SEQ ID NO: 538 - DNA, 375 bp, Artificial Sequence
  ACI-8033-4F3-Ab1 VH
gaggtccagctgcaacaatctggacctgagctggtgaagcctggggcttcagtgaagatatcctgtaaggcttctggatacacgttcactgactactacatgaactgggtgaagcagagccatggaaagagccttgaatggattggagatattaatcctaacactggtactaatagctacaaccagaagttcaagggcagggcctcactgactgtagacaagttctccagcgcagcctacatggagctccgcagcctgacatctgaggactctgcagtctattactgtgcaagaaccggctatggcgaccctatttcctcatattactatgctctggactactggggtcaaggaacctcagtcaccgtctcctca

SEQ ID NO: 539 - DNA, 336 bp, Artificial Sequence
  ACI-8033-4F3-Ab1 VL
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttataatctgtggacgttcggtggaggcaccaagctggaaatcaaa

SEQ ID NO: 540 - protein, 117 aa, Artificial Sequence
  ACI-8033-17F5-Ab1 VH
EVQLQQSGPELVKPGASVKISCKASGYTFTDYFMNWVKQSHGKSLEWIGDINPNIDVTNYNQKFKGKATLTVDKSSSTAYMELRSLTSEDSAVYYCARGRDYAMDFWGQGTSVTVSS

SEQ ID NO: 541 - skipped

SEQ ID NO: 542 - protein, 17 aa, Artificial Sequence
  ACI-8033-17F5-Ab1 VH CDR2
DINPNIDVTNYNQKFKG

SEQ ID NO: 543 - protein, 8 aa, Artificial Sequence
  ACI-8033-17F5-Ab1 VH CDR3
GRDYAMDF

SEQ ID NO: 544 - protein, 112 aa, Artificial Sequence
  ACI-8033-17F5-Ab1 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYDLWTFGGGTKLEIK

SEQ ID NO: 545-547 - skipped (3 entries)

SEQ ID NO: 548 - DNA, 351 bp, Artificial Sequence
  ACI-8033-17F5-Ab1 VH
gaggtccaactgcaacaatctggacctgagctggtgaagcctggggcttcagtgaagatatcctgtaaggcttctggatacacgttcactgactacttcatgaactgggtgaagcagagccatggaaagagccttgagtggattggagatattaatcctaacattgatgttactaactacaaccagaagttcaagggcaaggccacattgactgtagacaagtcctccagcacagcctacatggagctccgcagcctgacatctgaggactctgcagtctattactgtgcaagagggcgggactatgctatggacttctggggtcaaggaacctcagtcaccgtctcctca

SEQ ID NO: 549 - DNA, 336 bp, Artificial Sequence
  ACI-8033-17F5-Ab1 VL
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcaccctcaccatcagcagtgtgcaggctgaagacctggcagtttattactgcaagcaatcttatgatctgtggacgttcggtggaggcaccaagctggaaatcaaa

SEQ ID NO: 550 - protein, 116 aa, Artificial Sequence
  ACI-8033-18C11-Ab1 VH
QVQLQQPGAELVKPGASVKMSCKAAGYTFSSYWITWVRQRPGQGLDWIGDIYPGGGVTNYNEKFKTKATLTVDTSSSTAYMQLSSLTSEDSAVYYCATAQTTFAYWGQGTLVTVSA

SEQ ID NO: 551 - protein, 5 aa, Artificial Sequence
  ACI-8033-18C11-Ab1 VH CDR1
SYWIT

SEQ ID NO: 552 - protein, 17 aa, Artificial Sequence
  ACI-8033-18C11-Ab1 VH CDR2
DIYPGGGVTNYNEKFKT

SEQ ID NO: 553 - protein, 7 aa, Artificial Sequence
  ACI-8033-18C11-Ab1 VH CDR3
AQTTFAY

SEQ ID NO: 554 - protein, 112 aa, Artificial Sequence
  ACI-8033-18C11-Ab1 VL
DVLMTQTPLSLPVSLGDQASISCRSSQNIVHNNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 555 - protein, 16 aa, Artificial Sequence
  ACI-8033-18C11-Ab1 VL CDR1
RSSQNIVHNNGNTYLE

SEQ ID NO: 556 - skipped

SEQ ID NO: 557 - protein, 9 aa, Artificial Sequence
  ACI-8033-18C11-Ab1 VL CDR3
FQGSHVPRT

SEQ ID NO: 558 - DNA, 348 bp, Artificial Sequence
  ACI-8033-18C11-Ab1 VH
caggtccaactccagcagcctggggctgagcttgtgaagcctggggcttcagtgaagatgtcctgcaaggctgctggctacaccttcagcagctactggataacctgggtgaggcagaggcctggacaaggccttgactggattggagatatttatcctggtggaggtgttactaactacaatgagaagttcaagaccaaggccacactgactgtagacacatcctccagcacagcctacatgcagctcagcagcctgacatctgaggactctgcggtctattactgtgcgacagctcagactacgtttgcttactggggccaagggactctggtcactgtctctgca

SEQ ID NO: 559 - DNA, 336 bp, Artificial Sequence
  ACI-8033-18C11-Ab1 VL
gatgttttgatgacccaaactccactgtccctgcctgtcagtcttggagatcaagcctccatctcttgcagatctagtcagaacattgtacataataatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaa

SEQ ID NO: 560 - protein, 116 aa, Artificial Sequence
  ACI-8033-18D12-Ab1 VH
QVQLQQPGAELVKPGASVKMSCKASGYTFTSYWITWVRQRPGQGLDWIGDIYPGGGVTNYNEKFKTKATLTVDTSSSTAYMHLSSLTSEDSAVYFCATAQTTFAHWGQGTLVTVSA

SEQ ID NO: 561-562 - skipped (2 entries)

SEQ ID NO: 563 - protein, 7 aa, Artificial Sequence
  ACI-8033-18D12-Ab1 VH CDR3
AQTTFAH

SEQ ID NO: 564 - protein, 112 aa, Artificial Sequence
  ACI-8033-18D12-Ab1 VL
DVLMTQTPLSLPVSLGDQASISCRSSQNIAHNNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 565 - protein, 16 aa, Artificial Sequence
  ACI-8033-18D12-Ab1 VL CDR1
RSSQNIAHNNGNTYLE

SEQ ID NO: 566-567 - skipped (2 entries)

SEQ ID NO: 568 - DNA, 348 bp, Artificial Sequence
  ACI-8033-18D12-Ab1 VH
caggtccaactccagcagcctggggctgagcttgtgaagcctggggcttcagtgaagatgtcctgcaaggcttctggctacaccttcaccagctactggataacctgggtgaggcagaggcctggacaaggccttgactggattggagatatttatcctggtggaggtgttactaactacaatgagaagttcaagaccaaggccacactgactgtagacacatcctccagcacagcctacatgcacctcagcagcctgacatctgaggactctgcggtctatttctgtgcgacagctcagactacgtttgctcactggggccaagggactctggtcactgtctctgca

SEQ ID NO: 569 - DNA, 336 bp, Artificial Sequence
  ACI-8033-18D12-Ab1 VL
gatgttttgatgacccaaactccactctccctgcccgtcagtcttggagatcaagcctccatctcttgcagatctagtcagaatattgcacataataatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaa

SEQ ID NO: 570 - protein, 113 aa, Artificial Sequence
  ACI-8033-1F8-Ab1 VH
DVQLQESGPGLVKPSQSLSLTCSVTGYSITSGFYWNWIRQFPGNKLEWMGYISYDGSNNYNPSLKNRISIIRDTSKNQFFLKLKSVTSEDTATYYCVRGDVDWGQGTTLTVSS

SEQ ID NO: 571 - protein, 6 aa, Artificial Sequence
  ACI-8033-1F8-Ab1 VH CDR1
SGFYWN

SEQ ID NO: 572 - skipped

SEQ ID NO: 573 - protein, 4 aa, Artificial Sequence
  ACI-8033-1F8-Ab1 VH CDR3
GDVD

SEQ ID NO: 574 - protein, 112 aa, Artificial Sequence
  ACI-8033-1F8-Ab1 VL
DVVMTQTPLTLSVTIGQPASISCKSSQSLLDSDGETYLNWLFQRPGQSPKRLIYLVSKLDSGVPDRFTGSGSGTDFTLKISRVEPEDLGVYYCWQGTHFPQTLGGGTKLEIK

SEQ ID NO: 575-577 - skipped (3 entries)

SEQ ID NO: 578 - DNA, 339 bp, Artificial Sequence
  ACI-8033-1F8-Ab1 VH
gatgtacagcttcaggagtcaggacctggcctcgtgaaaccttctcagtctctgtctctcacctgctctgtcactggctactccatcaccagtgggttttactggaactggatccggcaatttccaggaaataaactggaatggatgggctacataagctacgatggtagcaataactacaacccatctctcaaaaatcgaatctccattattcgtgacacatctaagaaccagtttttcctgaagttgaaatctgtgacttctgaggacacagccacatattattgtgtaagaggggacgtcgactggggccaaggcaccactctcactgtctcctca

SEQ ID NO: 579 - DNA, 336 bp, Artificial Sequence
  ACI-8033-1F8-Ab1 VL
gatgttgtgatgacccagactccactcactttgtcggtcaccattggacaaccagcctccatctcttgcaagtcaagtcagagcctcttagatagtgatggagagacatatttgaattggttgtttcagaggccaggccagtctccaaagcgcctaatctatctggtgtctaaactggactctggagtccctgacaggttcactggcagtggatcagggacagatttcacactgaaaatcagcagagtggagcctgaggatttgggagtttattattgctggcaaggtacacattttcctcagacgctcggtggaggcaccaagctggaaatcaaa

SEQ ID NO: 580 - protein, 120 aa, Artificial Sequence
  ACI-8033-22E5-Ab1 VH
EVQLVESGGDLVKPGGSLKLSCAASGFTFSSYGMSWVRQTPDKRLEWVATISNGGSYTYYPDSVKGRFTISRDNAKNTLYLQMSSLKSEDTAMYYCARQLRRDGWYFDVWGTGTTVTVSS

SEQ ID NO: 581 - protein, 5 aa, Artificial Sequence
  ACI-8033-22E5-Ab1 VH CDR1
SYGMS

SEQ ID NO: 582 - protein, 17 aa, Artificial Sequence
  ACI-8033-22E5-Ab1 VH CDR2
TISNGGSYTYYPDSVKG

SEQ ID NO: 583 - protein, 11 aa, Artificial Sequence
  ACI-8033-22E5-Ab1 VH CDR3
QLRRDGWYFDV

SEQ ID NO: 584 - protein, 108 aa, Artificial Sequence
  ACI-8033-22E5-Ab1 VL
EIVLTQSPALMAASPGEKVTITCSVSSSISSSKLHWYQQKSETSPKLWIYGTSNLASGVPVRFSGSGSGTSYSLTISSMEAEDAATYYCQQWSSYPLTFGAGTKLELK

SEQ ID NO: 585 - protein, 12 aa, Artificial Sequence
  ACI-8033-22E5-Ab1 VL CDR1
SVSSSISSSKLH

SEQ ID NO: 586 - protein, 7 aa, Artificial Sequence
  ACI-8033-22E5-Ab1 VL CDR2
GTSNLAS

SEQ ID NO: 587 - protein, 9 aa, Artificial Sequence
  ACI-8033-22E5-Ab1 VL CDR3
QQWSSYPLT

SEQ ID NO: 588 - DNA, 360 bp, Artificial Sequence
  ACI-8033-22E5-Ab1 VH
gaggtgcaactagtggagtctgggggagacttagtgaagcctggagggtccctgaaactctcctgtgcagcctctggattcactttcagtagctatggcatgtcttgggttcgccagactccagacaagaggctggagtgggtcgcaaccattagtaatggtggtagttacacctactatccagacagtgtgaaggggcgattcaccatctccagagacaatgccaagaacaccctgtacctgcaaatgagcagtctgaagtctgaggacacagccatgtattactgtgcaagacaattacgacgggacggttggtacttcgatgtctggggcacagggaccacggtcaccgtctcctca

SEQ ID NO: 589 - DNA, 324 bp, Artificial Sequence
  ACI-8033-22E5-Ab1 VL
gaaattgtgctcacccagtctccagcactcatggctgcatctccaggggagaaggtcaccatcacctgcagtgtcagctcaagtataagttccagcaagttgcactggtaccagcagaagtcagaaacctcccccaaactctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaa

SEQ ID NO: 590 - protein, 115 aa, Artificial Sequence
  ACI-8033-27D8-Ab1 VH
EVQLVESGGGLVQPKGSLKLSCAASGFTFNTYAMNWVRQAPGKGLEWVARIRSKSNNFATYYADSVKDRFTISRDESESMLYLQMNNLKAEDTAMYYCVRSFDYWGQGTTLTVSS

SEQ ID NO: 591-597 - skipped (7 entries)

SEQ ID NO: 598 - DNA, 345 bp, Artificial Sequence
  ACI-8033-27D8-Ab1 VH
gaggtgcagcttgttgagtctggtggaggattggtgcagcctaaagggtcattgaaactctcatgtgcagcctctggattcaccttcaatacctacgccatgaactgggtccgccaggctccaggaaagggtttggaatgggttgctcgcataagaagtaaaagtaataattttgcaacatattatgccgattcagtgaaagacagattcaccatctccagagatgaatcagaaagcatgctctatctgcaaatgaacaacttgaaagctgaggacacagccatgtattactgtgtgaggtcctttgactactggggccaaggcaccactctcacagtctcctca

SEQ ID NO: 599 - skipped

SEQ ID NO: 600 - protein, 116 aa, Artificial Sequence
  ACI-8033-21C8-Ab1 VH
QVQLQQPGAELVKPGASVKMSCKASGYTFTSYWITWVRQRPGQGLDWIGDIYPGGGVTNYNEKFKTKATLTVDTSSSTAYMHLSSLTSEDSAVYFCATAQTTFAYWGQGTLVTVSA

SEQ ID NO: 601-607 - skipped (7 entries)

SEQ ID NO: 608 - DNA, 348 bp, Artificial Sequence
  ACI-8033-21C8-Ab1 VH
caggtccaactccagcagcctggggctgagcttgtgaagcctggggcttcagtgaagatgtcctgcaaggcttctggctacaccttcaccagctactggataacctgggtgaggcagaggcctggacaaggccttgactggattggagatatttatcctggtggaggtgttactaactacaatgagaagttcaagaccaaggccacactgactgtagacacatcctccagcacagcctacatgcacctcagcagcctgacatctgaggactctgcggtctatttctgtgcgacagctcagactacgtttgcttactggggccaagggactctggtcactgtctctgca

SEQ ID NO: 609 - DNA, 336 bp, Artificial Sequence
  ACI-8033-21C8-Ab1 VL
gatgttttgatgacccaaactccactctccctgcctgtcagtcttggagatcaagcctccatctcttgcagatctagtcagaacattgtacataataatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaa

SEQ ID NO: 610 - protein, 119 aa, Artificial Sequence
  ACI-7079-3101E3-Ab1 HCVR
QVQLKESGPGLVAPSQSLSITCTVSGFSLTRYIINWVRQPPGKGLEWLGVIWTGGGTDYNSALKSRLSISKDNSKSQVFLKMNSLQTDDTARYYCAREYGYDGAWFAYWGQGTLVTVSA

SEQ ID NO: 611 - protein, 5 aa, Artificial Sequence
  ACI-7079-3101E3-Ab1 VH-CDR1
RYIIN

SEQ ID NO: 612 - protein, 16 aa, Artificial Sequence
  ACI-7079-3101E3-Ab1 VH-CDR2
VIWTGGGTDYNSALKS

SEQ ID NO: 613 - protein, 11 aa, Artificial Sequence
  ACI-7079-3101E3-Ab1 VH CDR3
EYGYDGAWFAY

SEQ ID NO: 614 - protein, 112 aa, Artificial Sequence
  ACI-7079-3101E3-Ab1 LCVR
DVLMTQTPLSLPVSLGDQASISCRSSQTIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 615 - protein, 16 aa, Artificial Sequence
  ACI-7079-3101E3-Ab1 VL CDR1
RSSQTIVHSNGNTYLE

SEQ ID NO: 616 - protein, 7 aa, Artificial Sequence
  ACI-7079-3101E3-Ab1 VL CDR2
KVSNRFS

SEQ ID NO: 617 - protein, 9 aa, Artificial Sequence
  ACI-7079-3101E3-Ab1 VL CDR3
FQGSHVPYT

SEQ ID NO: 618 - DNA, 357 bp, Artificial Sequence
  ACI-7079-3101E3-Ab1 VH
caggtgcagctgaaggagtcaggacctggcctggtggcgccctcacagagcctgtccatcacatgcactgtctctggcttctcattaaccaggtatattataaattgggttcgccagccaccaggaaagggtctggagtggcttggagtaatctggactggtggaggcacagattataattcagctctcaaatccagactgagcatcagcaaagacaactccaagagtcaagttttcttaaaaatgaacagtctgcaaactgatgacacagccaggtactactgtgccagagagtatggttacgacggggcctggtttgcttactggggccaagggactcttgtcactgtctctgca

SEQ ID NO: 619 - DNA, 336 bp, Artificial Sequence
  ACI-7079-3101E3-Ab1 VL
gatgttttgatgacccaaactccactctccctgcctgtcagtcttggagatcaagcctccatctcttgcagatctagtcagaccattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 620 - protein, 123 aa, Artificial Sequence
  ACI-7079-3103D9-Ab1 HCVR
QVSLKESGPGILQPSQTLSLTCSFSGFSLSTFGMGVGWIRQPSGKGLDWLTHIWWDDDKYYNPGLKSRLTISKDTSKNQVFLEIANVDTADTATYYCARIGDYYGTRGYFDVWGTGTTVTVSS

SEQ ID NO: 621 - protein, 7 aa, Artificial Sequence
  ACI-7079-3103D9-Ab1 VH CDR1
TFGMGVG

SEQ ID NO: 622 - protein, 16 aa, Artificial Sequence
  ACI-7079-3103D9-Ab1 VH CDR2
HIWWDDDKYYNPGLKS

SEQ ID NO: 623 - protein, 13 aa, Artificial Sequence
  ACI-7079-3103D9-Ab1 VH CDR3
IGDYYGTRGYFDV

SEQ ID NO: 624 - protein, 113 aa, Artificial Sequence
  ACI-7079-3103D9-Ab1 LCVR
DIVMTQSPSSLTMSLGQTVTMSCKSSQSLLNSSNQKNHLAWYQQRPGQSPKLLVYFASTRESGVPERFIGGGSGTDFTLTISNVQSEDLTDYFCQQHYSTPYTFGGGTKLEIK

SEQ ID NO: 625 - protein, 17 aa, Artificial Sequence
  ACI-7079-3103D9-Ab1 VL CDR1
KSSQSLLNSSNQKNHLA

SEQ ID NO: 626 - protein, 7 aa, Artificial Sequence
  ACI-7079-3103D9-Ab1 VL CDR2
FASTRES

SEQ ID NO: 627 - protein, 9 aa, Artificial Sequence
  ACI-7079-3103D9-Ab1 VL CDR3
QQHYSTPYT

SEQ ID NO: 628 - DNA, 369 bp, Artificial Sequence
  ACI-7079-3103D9-Ab1 VH
caggtttctctgaaagagtctggccctgggatattgcagccctcccagaccctcagtctgacttgctctttctctgggttttcactgagcacttttggtatgggtgtaggctggattcgtcagccttcagggaagggtctggactggctgacacacatttggtgggatgatgataagtactataatccaggcctgaagagtcgcctcacaatctccaaggatacctccaaaaaccaggtattcctcgagatcgccaatgtggacactgcagatactgccacatattactgtgcgcgaataggagattactacggtactagagggtacttcgatgtctggggcacagggaccacggtcaccgtctcctca

SEQ ID NO: 629 - DNA, 339 bp, Artificial Sequence
  ACI-7079-3103D9-Ab1 VL
gacattgtgatgacacagtctccatcctccctgactatgtcactaggacagacggtcactatgagctgcaagtccagtcagagccttttaaatagtagcaatcaaaagaaccatctggcctggtaccagcagagaccaggacagtctcccaaacttctggtatactttgcatccactagggaatctggggtccctgagcgcttcataggcggtggatctgggacagatttcactcttaccatcagcaatgtgcagtctgaagacctgacagattacttctgtcagcaacattatagcactccgtacacgttcggaggggggaccaagctggaaataaaa

SEQ ID NO: 630 - protein, 118 aa, Artificial Sequence
  ACI-7079-3103G12-Ab2 HCVR
DVQLQESGPGMVKPSQSLSLTCTVTGYSITSGYDWHWIRHFPGNKLEWMGYITYSGSTNYNPSLKSRISITHDTSKNHFFLKLTSVTTEDTATYYCARDTVDAWFAYWGQGTLVTVSA

SEQ ID NO: 631 - protein, 6 aa, Artificial Sequence
  ACI-7079-3103G12-Ab2 VH CDR1
SGYDWH

SEQ ID NO: 632 - protein, 16 aa, Artificial Sequence
  ACI-7079-3103G12-Ab2 VH CDR2
YITYSGSTNYNPSLKS

SEQ ID NO: 633 - protein, 9 aa, Artificial Sequence
  ACI-7079-3103G12-Ab2 VH CDR3
DTVDAWFAY

SEQ ID NO: 634 - protein, 112 aa, Artificial Sequence
  ACI-7079-3103G12-Ab2 LCVR
DVLMTQAPLSLPVSLGDQASISCRSSQSIVHSNGDTYLEWYLQKPGQSPKLLIYKVSNRFSGVPDRFSGSGSGTDFTLKISRVEAEDLGVYYCFQGSHVPPTFGGGTKLEIK

SEQ ID NO: 635 - protein, 16 aa, Artificial Sequence
  ACI-7079-3103G12-Ab2 VL-CDR1
RSSQSIVHSNGDTYLE

SEQ ID NO: 636 - skipped

SEQ ID NO: 637 - protein, 9 aa, Artificial Sequence
  ACI-7079-3103G12-Ab2 VL-CDR3
FQGSHVPPT

SEQ ID NO: 638 - DNA, 354 bp, Artificial Sequence
  ACI-7079-3103G12-Ab2 VH
gatgtgcagcttcaggagtcaggacctggcatggtgaaaccttctcagtcactttccctcacctgcactgtcactggctactccatcaccagtggttatgactggcactggatccgacattttccaggaaacaaactggagtggatgggctacataacctacagtggtagcactaactacaacccatccctcaaaagtcgaatctccatcactcatgacacatctaagaaccatttcttcctgaagttgacttctgtgactactgaggacacagccacatattactgtgcaagagacacggtagacgcctggtttgcttactggggccaggggactctggtcactgtctctgca

SEQ ID NO: 639 - DNA, 336 bp, Artificial Sequence
  ACI-7079-3103G12-Ab2 VL
gatgttttgatgacccaagctccactctccctgcctgtcagtcttggagatcaagcctccatctcgtgcagatctagtcagagcattgtacatagtaatggagacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 640 - protein, 123 aa, Artificial Sequence
  ACI-7079-3104F12-Ab2 HCVR
QVTLKESGPGIVQPSQTLSLTCSFSGFSLNTFGLGVGWIRQPSGKGLDWLTHIWWDDDKYYNPALKSRLTISKDTSKNQVFLKIANVDTADTATYYCARIGDFYGSRGYFDVWGTGTTVTVSS

SEQ ID NO: 641 - protein, 7 aa, Artificial Sequence
  ACI-7079-3104F12-Ab2 VH-CDR1
TFGLGVG

SEQ ID NO: 642 - protein, 16 aa, Artificial Sequence
  ACI-7079-3104F12-Ab2 VH-CDR2
HIWWDDDKYYNPALKS

SEQ ID NO: 643 - protein, 13 aa, Artificial Sequence
  ACI-7079-3104F12-Ab2 VH-CDR3
IGDFYGSRGYFDV

SEQ ID NO: 644 - protein, 113 aa, Artificial Sequence
  ACI-7079-3104F12-Ab2 LCVR
DIVMTQSPSSLAMSVGQKVTLRCKSSQSLLNSSNQKNHLAWYQQKPGQSPKLLLCFASTRESGVPDRFIGSGSGTDFTLTISSVQAEDLADYFCQQHYSTPYTFGGGTKLEIK

SEQ ID NO: 645-647 - skipped (3 entries)

SEQ ID NO: 648 - DNA, 369 bp, Artificial Sequence
  ACI-7079-3104F12-Ab2 VH
caggttactctgaaagagtctggccctggaatagtgcagccctcccagaccctcagtctgacttgctctttctctggattttcactgaacacttttggtttgggtgtaggctggattcgtcagccttcagggaagggtctggactggctgacacacatttggtgggatgatgataagtactataatccagccctgaagagtcggctcacaatctccaaggatacctccaaaaaccaggtattcctcaagatcgccaatgtggacactgcagatactgccacatactactgtgctcgaataggcgatttctacggtagtagaggatatttcgatgtctggggcacagggaccacggtcaccgtctcctca

SEQ ID NO: 649 - DNA, 339 bp, Artificial Sequence
  ACI-7079-3104F12-Ab2 VL
gacattgtgatgacacagtctccatcctccctggctatgtcagttggacagaaggtcactttgaggtgcaagtccagtcagagccttttaaatagtagtaatcaaaagaaccatttggcctggtaccagcagaaaccaggacagtctcctaaacttctgctatgctttgcatccactagggaatctggggtccctgatcgcttcataggcagtggatctgggacagatttcactcttaccatcagtagtgtgcaggctgaagacctggcagattacttctgtcagcaacattatagcactccgtacacgttcggaggggggaccaagctggaaataaaa

SEQ ID NO: 650 - protein, 123 aa, Artificial Sequence
  ACI-7079-3106C5-Ab1 HCVR
QVTLKESGPGILQPSQTLSLTCSFSGFSLSTFGMGVGWIRQPSGKGLEWLTHIWWDDDKYYNPALKSRLTISRDPSKNQVFLKIANVDTADTATYYCARIGDYYGSRGYFDVWGTGTTVTVSS

SEQ ID NO: 651-652 - skipped (2 entries)

SEQ ID NO: 653 - protein, 13 aa, Artificial Sequence
  ACI-7079-3106C5-Ab1 VH-CDR3
IGDYYGSRGYFDV

SEQ ID NO: 654 - protein, 113 aa, Artificial Sequence
  ACI-7079-3106C5-Ab1 LCVR
DIVMTQSPSSLAMSVGQKVTMSCKSRQSLLNSSNQKNHLAWYQQKPGQSPKLLVYFASTRESGVPDRFIGSGSGTDFTLTISSVQAEDLADYFCQQHYSTPYTFGGGTKLEIK

SEQ ID NO: 655 - protein, 17 aa, Artificial Sequence
  ACI-7079-3106C5-Ab1 VL-CDR1
KSRQSLLNSSNQKNHLA

SEQ ID NO: 656-657 - skipped (2 entries)

SEQ ID NO: 658 - DNA, 368 bp, Artificial Sequence
  ACI-7079-3106C5-Ab1 VH
caggttactctgaaagagtctggccctgggatattgcagccctcccagaccctcagtctgacttgttctttctctgggttttcactgagtacttttggtatgggtgtaggctggattcgtcagccttcagggaagggtctggagtggctaacacacatttggtgggatgatgataagtactataacccagccctgaagagtcggctcacaatctccagggatccctccaaaaaccaggtattctcaagatcgccaatgtggacactgcagatactgccacatactactgtgctcgaataggggattactacggtagtagaggatacttcgatgtctggggcacagggaccacggtcaccgtctcctca

SEQ ID NO: 659 - DNA, 339 bp, Artificial Sequence
  ACI-7079-3106C5-Ab1 VL
gacattgtgatgacacagtctccatcctccctggctatgtcagtaggacagaaggtcactatgagctgcaagtccaggcagagccttttaaatagtagtaatcaaaagaatcatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattatagcactccgtacacgttcgggggggggaccaagctggaaataaaa

SEQ ID NO: 660 - protein, 119 aa, Artificial Sequence
  ACI-7079-3106F2-Ab1 HCVR
EVQLQQSGAELVRPGASVKLSCTASGFNVKDDYMHWVKQRPEQGLEWIGWIDPENGDTEYASKFQGKATLTADTSSNTAYLQLSGLTSVDTAVYYCTTAGTSPYYFDYWGQGTTLTVSS

SEQ ID NO: 661 - protein, 5 aa, Artificial Sequence
  ACI-7079-3106F2-Ab1 VH-CDR1
DDYMH

SEQ ID NO: 662 - protein, 17 aa, Artificial Sequence
  ACI-7079-3106F2-Ab1 VH-CDR2
WIDPENGDTEYASKFQG

SEQ ID NO: 663 - protein, 10 aa, Artificial Sequence
  ACI-7079-3106F2-Ab1VH-CDR3
AGTSPYYFDY

SEQ ID NO: 664 - protein, 107 aa, Artificial Sequence
  ACI-7079-3106F2-Ab1 LCVR
DIVMTQSQKFMSTSVGDRVSVTCKASQNVGTNVAWYQQKVGQSPKALIYSASYRYSGVPDRFTGSGSGTDFTLTISNVQSEDLADYFCQQYNSYPLTFGSGTKLEIK

SEQ ID NO: 665 - protein, 11 aa, Artificial Sequence
  ACI-7079-3106F2-Ab1 VL-CDR1
KASQNVGTNVA

SEQ ID NO: 666 - protein, 7 aa, Artificial Sequence
  ACI-7079-3106F2-Ab1 VL-CDR2
SASYRYS

SEQ ID NO: 667 - protein, 9 aa, Artificial Sequence
  ACI-7079-3106F2-Ab1 VL-CDR3
QQYNSYPLT

SEQ ID NO: 668 - DNA, 357 bp, Artificial Sequence
  ACI-7079-3106F2-Ab1 VH
gaggttcagctgcagcagtctggggctgagcttgtgaggccaggggcctcagtcaagttgtcctgcacagcttctggctttaacgttaaagacgactatatgcactgggtgaagcagaggcctgaacagggcctggagtggattggatggattgatcctgagaatggtgatactgaatatgcctcgaagttccagggcaaggccactttaacagcagacacatcctccaacacagcctacctgcagctcagcggcctgacatctgtggacactgccgtctattactgtactacagccggtactagcccgtactactttgactactggggccaaggcaccactctcacagtctcctcc

SEQ ID NO: 669 - DNA, 321 bp, Artificial Sequence
  ACI-7079-3106F2-Ab1 VL
gacattgtgatgacccagtctcaaaaattcatgtccacatcagtaggagacagggtcagcgtcacctgcaaggccagtcagaatgtgggaactaatgtagcctggtatcaacagaaagtaggacaatctcctaaagcactgatttactcggcatcctaccggtacagtggagtccctgatcgcttcacaggcagtggatctgggacagatttcactctcaccatcagcaatgtgcagtctgaagacttggcagactatttctgtcagcaatataacagctatcctctcacgttcggctcggggacaaagttggaaataaaa

SEQ ID NO: 670 - protein, 117 aa, Artificial Sequence
  ACI-7079-3112H1-Ab1 VH
EVQLQQSGPELVKPGASVKISCKASGYTFSDYFMNWVKQSHGKSLEWIGDINPNNGGSSYIQKFKGKATLTVDKSSTTAYMELRSLTSEDSAVYYCARGSNYAMDSWGQGTSVTVSS

SEQ ID NO: 671 - protein, 5 aa, Artificial Sequence
  ACI-7079-3112H1-Ab1 VH CDR1
DYFMN

SEQ ID NO: 672 - protein, 17 aa, Artificial Sequence
  ACI-7079-3112H1-Ab1 VH CDR2
DINPNNGGSSYIQKFKG

SEQ ID NO: 673 - protein, 8 aa, Artificial Sequence
  ACI-7079-3112H1-Ab1 VH CDR3
GSNYAMDS

SEQ ID NO: 674 - protein, 112 aa, Artificial Sequence; identical to SEQ ID NO: 544
  ACI-7079-3112H1-Ab1 VL

SEQ ID NO: 675 - protein, 17 aa, Artificial Sequence
  ACI-7079-3112H1-Ab1 VL CDR1
KSSQSLLNSRTRKNYLA

SEQ ID NO: 676 - protein, 7 aa, Artificial Sequence
  ACI-7079-3112H1-Ab1 VL CDR2
WASTRES

SEQ ID NO: 677 - protein, 8 aa, Artificial Sequence
  ACI-7079-3112H1-Ab1 VL CDR3
KQSYDLWT

SEQ ID NO: 678 - DNA, 351 bp, Artificial Sequence
  ACI-7079-3112H1-Ab1 VH
gaggtccagctgcaacaatctggacctgaactggtgaagcctggggcttcagtgaagatatcctgtaaggcttctggatacacgttcagtgactacttcatgaactgggtgaagcagagtcatggaaaaagccttgagtggattggagatattaatcctaacaatggtggttctagctacatccagaagttcaagggcaaggccacattgactgtagacaagtcctccaccacagcctatatggagctccgcagcctgacatctgaggactcagcagtctattactgtgcaagaggtagcaactatgctatggactcctggggtcagggaacctcagtcaccgtctcctca

SEQ ID NO: 679 - DNA, 336 bp, Artificial Sequence
  ACI-7079-3112H1-Ab1 VL
gacattgtgatgtcacagtctccatcctccctggctgtgtcagcaggagagaaggtcactatgagctgcaaatccagtcagagtctgctcaacagtagaacccggaagaactacttggcttggtaccagcagaaaccaggacagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttatgatctgtggacgttcggtgggggcaccaagctggaaatcaaa

SEQ ID NO: 680 - protein, 123 aa, Artificial Sequence
  ACI-7079-3107E6-Ab1 VH
QVTLKESGPGILQPSQTLSLTCSFSGFSLSTFGMGVGWIRQPSGKGLEWLTHIWWDDDKYYNPALKSRLTISKDASKNQMFLKIANVDTADTATYFCARIGDYYGSRGFFDVWGTGTTVTVSS

SEQ ID NO: 681-682 - skipped (2 entries)

SEQ ID NO: 683 - protein, 13 aa, Artificial Sequence
  ACI-7079-3107E6-Ab1 VH CDR3
IGDYYGSRGFFDV

SEQ ID NO: 684 - protein, 113 aa, Artificial Sequence
  ACI-7079-3107E6-Ab1 VL
DIVMTQSPSSLAMSVGQKVTMSCKSSQSLLNSSNQKNHLAWYQQKPGQSPKLLVYFASTRDSGVPDRFIGSGSGTDFTLTISSVQAEDLADYFCQQHYSTPYTFGGGTKLEIK

SEQ ID NO: 685 - skipped

SEQ ID NO: 686 - protein, 7 aa, Artificial Sequence
  ACI-7079-3107E6-Ab1 VL CDR2
FASTRDS

SEQ ID NO: 687 - skipped

SEQ ID NO: 688 - DNA, 369 bp, Artificial Sequence
  ACI-7079-3107E6-Ab1 VH
caggttactctgaaagagtctggccctgggatattgcagccctcccagaccctcagtctgacttgttctttctctgggttttcactgagcacttttggtatgggtgtaggctggattcgtcagccttcagggaagggtctggagtggctgacacacatttggtgggatgatgataaatactataacccagcccttaagagtcggctcacaatctccaaggatgcctccaaaaaccagatgttcctcaagatcgccaatgtggacactgcagatactgccacatacttctgtgctcgaataggggattactacggtagtagagggttcttcgatgtctggggcacagggaccacggtcaccgtctcctca

SEQ ID NO: 689 - DNA, 339 bp, Artificial Sequence
  ACI-7079-3107E6-Ab1 VL
gacattgtgatgacacagtctccatcctccctggccatgtcagtaggacagaaggtcactatgagctgcaagtccagtcagagccttttaaatagtagcaatcaaaagaaccatttggcctggtatcagcagaaaccaggacagtctcctaaacttctggtatactttgcatccactagggactctggggtccctgatcgcttcataggcagtggatctgggacagatttcactcttaccatcagcagtgtgcaggctgaggacctggcagattacttctgtcagcaacattatagcactccgtacacgttcggaggggggaccaagctggaaataaaa

SEQ ID NO: 690 - protein, 114 aa, Artificial Sequence
  ACI-7079-3108C10-Ab2 VH
ELQLVESGGGLVRPGGSLKLSCATSGFTFRNYAMSWVRQTPAKRLEWVATISDGGTYTFYSDNVKGRFTISRDNVKNTLYLQMSHLKSEDTAMYYCSRAGSGHWGQGTTLTVSS

SEQ ID NO: 691 - protein, 5 aa, Artificial Sequence
  ACI-7079-3108C10-Ab2 VH CDR1
NYAMS

SEQ ID NO: 692 - protein, 17 aa, Artificial Sequence
  ACI-7079-3108C10-Ab2 CDR2
TISDGGTYTFYSDNVKG

SEQ ID NO: 693 - protein, 5 aa, Artificial Sequence
  ACI-7079-3108C10-Ab2 VH CDR3
AGSGH

SEQ ID NO: 694 - protein, 112 aa, Artificial Sequence
  ACI-7079-3108C10-Ab2 VL
DVVMTQTPRTLSVIIGQPASISCKSSQSLLDSDGKTYLSWLLQRPGQSPKRLIYLVSKVDSGVPDRFTGSGSGTDFTLKISRVEAEDLGVYYCWQGTYFPRTFGGGTKLEIK

SEQ ID NO: 695 - protein, 16 aa, Artificial Sequence
  ACI-7079-3108C10-Ab2 VL CDR1
KSSQSLLDSDGKTYLS

SEQ ID NO: 696 - protein, 7 aa, Artificial Sequence
  ACI-7079-3108C10-Ab2 VL CDR2
LVSKVDS

SEQ ID NO: 697 - protein, 9 aa, Artificial Sequence
  ACI-7079-3108C10-Ab2 VL CDR3
WQGTYFPRT

SEQ ID NO: 698 - DNA, 342 bp, Artificial Sequence
  ACI-7079-3108C10-Ab2 VH
gaactgcagctggtggagtctgggggaggcttagtgaggcctggagggtccctgaaactctcctgtgcaacctctggattcactttcagaaactatgccatgtcttgggttcgccagactccggcaaagaggctggagtgggtcgcaaccattagtgatggtggtacttacaccttctattcagacaatgtaaagggccgattcaccatctccagagacaatgtcaagaacaccttgtaccttcaaatgagccatctgaagtctgaggacacagccatgtattactgttcaagagccggctctggccactggggccaaggcaccactctcacagtctcctca

SEQ ID NO: 699 - DNA, 336 bp, Artificial Sequence
  ACI-7079-3108C10-Ab2 VL
gatgttgtgatgacccagactccacgcactttgtcggtaatcattggacaaccagcctccatctcttgcaagtcaagtcagagcctcttagatagtgatggaaagacatacttgagttggttgttacagaggccaggccagtctccaaagcgcctaatctatctggtgtctaaagtggactctggagtccctgacaggttcactggcagtggatcagggacagatttcacactgaaaatcagcagagtggaggctgaggatttgggagtttattattgctggcaaggtacatattttcctcggacgttcggtggaggcaccaagctggaaatcaaa

SEQ ID NO: 700 - protein, 117 aa, Artificial Sequence
  ACI-8030-6106F5-Ab1 VH
QVQLQQSGPELVKPGASVKISCKASGYSFTTYYIHWMKQRPGQGPEWIGWIYPGRGYTKYNEKFKDKAAQTADTSSNTAYMQLSSLRSEDSAVYYCARDWETGFPYWGQGTLVTVSA

SEQ ID NO: 701 - protein, 5 aa, Artificial Sequence
  ACI-8030-6106F5-Ab1 VH CDR1
TYYIH

SEQ ID NO: 702 - protein, 17 aa, Artificial Sequence
  ACI-8030-6106F5-Ab1 VH CDR2
WIYPGRGYTKYNEKFKD

SEQ ID NO: 703 - protein, 8 aa, Artificial Sequence
  ACI-8030-6106F5-Ab1 VH CDR3
DWETGFPY

SEQ ID NO: 704 - protein, 111 aa, Artificial Sequence
  ACI-8030-6106F5-Ab1 VL
DIVLTQSPASLAVSLGQRATISCKASQSVDYGGDSWLNWYQQKPGQPPKLLIYAASNLDSGIPARFGGSGSGTDFTLNIHPVEEEDAATYYCQQSGEDPFTFGGGTNLEIK

SEQ ID NO: 705 - protein, 15 aa, Artificial Sequence
  ACI-8030-6106F5-Ab1 VL CDR1
KASQSVDYGGDSWLN

SEQ ID NO: 706 - protein, 7 aa, Artificial Sequence
  ACI-8030-6106F5-Ab1 VL CDR2
AASNLDS

SEQ ID NO: 707 - protein, 9 aa, Artificial Sequence
  ACI-8030-6106F5-Ab1 VL CDR3
QQSGEDPFT

SEQ ID NO: 708 - DNA, 351 bp, Artificial Sequence
  ACI-8030-6106F5-Ab1 VH
caggtccagctgcagcagtctggacctgaactggtgaagcctggggcttcagtgaagatatcctgcaaggcttctggctacagcttcacaacctactatatacactggatgaagcagaggcctggacagggacctgagtggattggatggatttatcctggaaggggttatactaagtacaatgagaagttcaaggacaaggccgcacagacggcagacacatcctccaacactgcctacatgcagctcagcagcctaagatctgaggactctgcggtctattactgtgcaagagactgggaaaccggatttccttactggggccaaggtactctggtcactgtctctgca

SEQ ID NO: 709 - DNA, 333 bp, Artificial Sequence
  ACI-8030-6106F5-Ab1 VL
gacattgtgctgacccaatctccagcttctttggctgtgtctctagggcagagggccaccatctcctgcaaggccagccaaagtgttgattatggtggtgatagttggctgaactggtaccaacagaaaccaggacagccacccaaactcctcatctatgctgcatccaatctagattctgggatcccagccaggtttggtggcagtgggtctgggacagacttcaccctcaacatccatcctgtggaggaggaggatgctgcaacctattactgtcaacaaagtggagaggatccgttcacgttcggaggggggaccaatctggaaataaaa

SEQ ID NO: 710 - protein, 114 aa, Artificial Sequence
  ACI-8031-6207G10-Ab1 VH
QVQLQQSGAELVKPGASVKISCKASGYAFSSYWMNWVKQRPGKGLEWIGQIYPGDGDTNYNGKFKGKATLTADRSSSTAYMQLSSLTSEDSAVYFCAITHYDYWGQGTTLTVSS

SEQ ID NO: 711 - protein, 5 aa, Artificial Sequence
  ACI-8031-6207G10-Ab1 VH CDR1
SYWMN

SEQ ID NO: 712 - protein, 17 aa, Artificial Sequence
  ACI-8031-6207G10-Ab1 VH CDR2
QIYPGDGDTNYNGKFKG

SEQ ID NO: 713 - protein, 5 aa, Artificial Sequence
  ACI-8031-6207G10-Ab1 VH CDR3
THYDY

SEQ ID NO: 714 - protein, 107 aa, Artificial Sequence
  ACI-8031-6207G10-Ab1 VL
DILLTQSPAILSVSPGDRVSFSCRASQSIGTRIHWYQQRTNGSPRLLIKYASESISGTPSRFSGSGSGTDFTLSINSVESEDIADYYCQQSNSWPLTFGAGTKLELK

SEQ ID NO: 715 - protein, 11 aa, Artificial Sequence
  ACI-8031-6207G10-Ab1 VL CDR1
RASQSIGTRIH

SEQ ID NO: 716 - protein, 7 aa, Artificial Sequence
  ACI-8031-6207G10-Ab1 VL CDR2
YASESIS

SEQ ID NO: 717 - protein, 9 aa, Artificial Sequence
  ACI-8031-6207G10-Ab1 VL CDR3
QQSNSWPLT

SEQ ID NO: 718 - DNA, 342 bp, Artificial Sequence
  ACI-8031-6207G10-Ab1 VH
caggttcagctgcagcagtctggggctgagctggtgaagcctggggcctcagtgaagatttcctgcaaagcttctggctacgcattcagtagctactggatgaactgggtgaagcagaggcctggaaagggtcttgagtggattggacagatttatcctggagatggtgatactaactacaacggaaagttcaagggcaaggccacactgactgcagacagatcctccagcacagcctacatgcagctcagcagcctgacctctgaggactctgcggtctatttctgtgcaattacgcactatgactactggggccaaggcaccactctcacagtctcctca

SEQ ID NO: 719 - DNA, 321 bp, Artificial Sequence
  ACI-8031-6207G10-Ab1 VL
gacatcttgctgactcagtctccagccatcctgtctgtgagtccaggagatagagtcagtttctcctgcagggccagtcagagcattggcacaaggatacactggtatcagcaaagaacaaatggttctccaaggcttctcataaagtatgcttctgagtctatctctgggaccccttccaggtttagtggcagtggatcagggacagattttactcttagcatcaacagtgtggagtctgaagatattgcagattattactgtcaacaaagtaatagttggccactcacgttcggtgctgggaccaagctggagctgaaa

SEQ ID NO: 720 - protein, 122 aa, Artificial Sequence
  ACI-8032-6301A10-Ab2 VH
QVQLQQSGAELARPGASVKLSCKASGYTFTSYGISWVKQRTGQGLEWIGEIYPRSGNTYYNEKFKGKATLTADKSSSTAYMELRSLTSEDSAVYFCASESYYGDYDWYFDVWGTGTTVTVSS

SEQ ID NO: 721 - protein, 5 aa, Artificial Sequence
  ACI-8032-6301A10-Ab2 VH CDR1
SYGIS

SEQ ID NO: 722 - protein, 17 aa, Artificial Sequence
  ACI-8032-6301A10-Ab2 VH CDR2
EIYPRSGNTYYNEKFKG

SEQ ID NO: 723 - protein, 13 aa, Artificial Sequence
  ACI-8032-6301A10-Ab2 VH CDR3
ESYYGDYDWYFDV

SEQ ID NO: 724 - protein, 112 aa, Artificial Sequence
  ACI-8032-6301A10-Ab2 VL
DVLMTQTPLSLPVSLGDQASISCRSSHSIEHSNGNTYLEWCLQKPGQPPKFLIYKVSNRFSGVPDRFSGSGSGTDFTLRISRVEAEDLGVYYCFQGSHVPPTFGGGTKLEIK

SEQ ID NO: 725 - protein, 16 aa, Artificial Sequence
  ACI-8032-6301A10-Ab2 VL CDR1
RSSHSIEHSNGNTYLE

SEQ ID NO: 726-727 - skipped (2 entries)

SEQ ID NO: 728 - DNA, 366 bp, Artificial Sequence
  ACI-8032-6301A10-Ab2 VH
caggttcagctgcagcagtctggagctgaactggcgaggcctggggcttcagtgaagctgtcctgcaaggcttctggctacaccttcacaagctatggtatcagctgggtgaagcagagaactggacagggccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagtgagagctactatggtgactacgactggtacttcgatgtctggggcactgggaccacggtcaccgtctcctca

SEQ ID NO: 729 - DNA, 336 bp, Artificial Sequence
  ACI-8032-6301A10-Ab2 VL
gatgttttgatgacccaaactccactctccctgcctgtcagtcttggagatcaagcctccatctcttgcagatctagtcacagcattgaacatagtaatggaaacacctatttagaatggtgcctgcagaaaccaggccagcctccaaagttcctgatctacaaagtttccaaccgattttctggggtcccagacaggttcagtggcagtggatcagggacagatttcacactcaggatcagtagagtggaggctgaggatctgggagtttattactgctttcaaggttcacatgttcctccgacgttcggtggaggcaccaagctggaaatcaaa

SEQ ID NO: 730 - protein, 122 aa, Artificial Sequence
  ACI-8032-6301C8-Ab2 VH
QVQLQQSGAELARPGTSVKLSCKASGYTFTTYGISWVKQRTGQGLEWIGEIYPRSGNTYYNEKFKGKATLTADKPSSTAYMELRSLTSEDSAVYFCASESYYGDYAWYFDVWGTGTTVTVSS

SEQ ID NO: 731 - protein, 5 aa, Artificial Sequence
  ACI-8032-6301C8-Ab2 VH CDR1
TYGIS

SEQ ID NO: 732 - skipped

SEQ ID NO: 733 - protein, 13 aa, Artificial Sequence
  ACI-8032-6301C8-Ab2 VH CDR3
ESYYGDYAWYFDV

SEQ ID NO: 734 - protein, 112 aa, Artificial Sequence
  ACI-8032-6301C8-Ab2 VL
DVLMTQIPLSLPVSLGDQASISCRSSQSIVHSNGNTYLEWHLQKPGQSPKLLIYEVSNRFSGVPDRFSGSGSGTDFTLNISRVEAEDLGVYYCFQGSHVPPTFGGGTKLEIK

SEQ ID NO: 735 - protein, 16 aa, Artificial Sequence
  ACI-8032-6301C8-Ab2 VL CDR1
RSSQSIVHSNGNTYLE

SEQ ID NO: 736 - protein, 7 aa, Artificial Sequence
  ACI-8032-6301C8-Ab2 VL CDR2
EVSNRFS

SEQ ID NO: 737 - skipped

SEQ ID NO: 738 - DNA, 366 bp, Artificial Sequence
  ACI-8032-6301C8-Ab2 VH
caggttcagctgcagcagtctggagctgaactggcgaggcctgggacttcagtgaagctgtcctgcaaggcttctggctacaccttcacaacctatggtataagctgggtgaagcagagaactggacagggccttgagtggattggagagatttatcctagaagtggtaatacttactacaatgagaaattcaagggcaaggccacactgactgcagacaagccctccagcacagcgtacatggagctccgcagcctgacatctgaggactctgcggtctatttctgtgcaagtgagagttactatggtgactacgcctggtacttcgatgtctggggcacagggaccacggtcaccgtctcctca

SEQ ID NO: 739 - DNA, 336 bp, Artificial Sequence
  ACI-8032-6301C8-Ab2 VL
gatgttttgatgacccaaattccactctccctgcctgtcagtcttggagatcaagcctccatctcttgcagatctagtcagagcattgtacatagtaatggaaacacctatttagaatggcacctgcagaaaccaggccagtctccaaagctcctgatctacgaagtttccaaccgattttctggggtcccagacaggttcagtggcagtggatcagggacagatttcacactcaatatcagcagagtggaggctgaggatctgggagtttattactgctttcaaggttcacatgttcctccgacgttcggtggaggcaccaagctggaaatcaaa

SEQ ID NO: 740 - protein, 65 aa, Artificial Sequence
  ACI-8032-6301G2-Ab2 VH
TNYNPKFKDRATLTVDKSSSSAYMELRSLTSEDSTVYYCARGGGSNPLYYAMDYWGQGTSVTVSS

SEQ ID NO: 741 - skipped

SEQ ID NO: 742 - protein, 17 aa, Artificial Sequence
  ACI-8032-6301G2-Ab2 VH CDR2
DINPNIGVTNYNPKFKD

SEQ ID NO: 743 - protein, 13 aa, Artificial Sequence
  ACI-8032-6301G2-Ab2 VH CDR3
GGGSNPLYYAMDY

SEQ ID NO: 744 - protein, 112 aa, Artificial Sequence
  ACI-8032-6301G2-Ab2 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYFCKQSYDLWTFGGGTKLEIK

SEQ ID NO: 745-747 - skipped (3 entries)

SEQ ID NO: 748 - DNA, 366 bp, Artificial Sequence
  ACI-8032-6301G2-Ab2 VH
gaggtccaactgcaacaatctggacctgagctggtgaagcctggggcttcagtgaagatatcctgtaaggcttctggatacacgttcactgactacttcatgaactgggtgaagcagagccatggaaagagccttgagtggattggagatattaatcctaacattggtgttactaactacaacccgaagttcaaggacagggccacattgactgtagacaagtcctccagctcagcctacatggagctccgcagcctgacatctgaggactctacagtctattactgtgcaagagggggagggagtaatcccctttactatgctatggactactggggtcaaggaacctcagtcaccgtctcctca

SEQ ID NO: 749 - DNA, 336 bp, Artificial Sequence
  ACI-8032-6301G2-Ab2 VL
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttatttctgcaagcaatcttatgatctgtggacgttcggtggaggcaccaagctggaaatcaaa

SEQ ID NO: 750 - protein, 122 aa, Artificial Sequence
  ACI-8032 VH
QVQLQQSGAELARPGASVKLSCKASGYTFTSYGVSWVKQRTGQGLEWIGEIYPRSGNTYYNEKFKGKATLTADKSSSTAYMELRSLTSEDSAVYFCASESYYGDYDWYFDVWGTGTTVTVSS

SEQ ID NO: 751 - protein, 5 aa, Artificial Sequence
  ACI-8032 VH CDR1
SYGVS

SEQ ID NO: 752-753 - skipped (2 entries)

SEQ ID NO: 754 - protein, 112 aa, Artificial Sequence
  ACI-8032-6304F3-Ab1 VL
DVLMTQIPLSLPVSPGDQVSISCRSSQSIVHSNGNTYLEWYLQKPGQSPKPLIYKVSNRFSGVPDRFSGSGSGTDFTLKISRVEAEDLGVYYCFQGSHVPPTFGGGTKLEIK

SEQ ID NO: 755-757 - skipped (3 entries)

SEQ ID NO: 758 - DNA, 366 bp, Artificial Sequence
  ACI-8032 VH
caggttcagctgcagcagtctggagctgagctggcgagacctggggcttcagtgaagctgtcctgcaaggcttctggctacaccttcacaagctatggtgtaagctgggtgaagcagagaactggacagggccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagtgagagctactatggtgactacgactggtatttcgatgtctggggcacagggaccacggtcaccgtctcctca

SEQ ID NO: 759 - DNA, 336 bp, Artificial Sequence
  ACI-8032-6304F3-Ab1 VL
gatgttttgatgacccaaattccactctccctgcctgtcagtcctggagatcaagtttccatctcttgcagatctagtcagagcattgtacatagtaatggaaacacctatttagaatggtacctgcagaaaccaggccagtctccaaagcccctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 760 - protein, 122 aa, Artificial Sequence
  ACI-8032-6307F1-Ab2 VH
QVQLQQSGAELARPGASVKLSCKASGYTFTSYGMSWVKQRTGQGLEWIGEIYPRSGNTYYNEKFKGKATLTSDKSSSTSYMELRSLTSEDSAVYFCASESYYGDYAWYFDVWGTGTTVTVSS

SEQ ID NO: 761 - protein, 5 aa, Artificial Sequence
  ACI-8032-6307F1-Ab2 VH CDR1
SYGMS

SEQ ID NO: 762-763 - skipped (2 entries)

SEQ ID NO: 764 - protein, 112 aa, Artificial Sequence
  ACI-8032-6307F1-Ab2 VL
DVLMTQTPLSLPVSLGDQASISCRSSQSVVHSNGITYLEWYLQKPGQSPKLLIYKVSNRFSGVPDRFSGSGSGTYFTLKISRVEAEDLGVYYCFQGSHVPPTFGGGTKLEIK

SEQ ID NO: 765 - protein, 16 aa, Artificial Sequence
  ACI-8032-6307F1-Ab2 VL CDR1
RSSQSVVHSNGITYLE

SEQ ID NO: 766-767 - skipped (2 entries)

SEQ ID NO: 768 - DNA, 366 bp, Artificial Sequence
  ACI-8032-6307F1-Ab2 VH
caggttcagctgcagcagtctggagctgagctggcgaggcctggggcttcagtgaagctgtcctgcaaggcttctggctacaccttcacaagctatggtatgagttgggtgaaacagagaactggacagggccttgagtggattggagagatttatcctagaagtggtaatacttactacaatgagaagttcaagggcaaggccacactgacttcagacaaatcctccagcacatcatacatggagctccgcagcctgacatctgaggactctgcggtctatttctgtgcaagtgagagctactatggtgactacgcctggtacttcgatgtctggggcacagggaccacggtcaccgtctcctca

SEQ ID NO: 769 - DNA, 336 bp, Artificial Sequence
  ACI-8032-6307F1-Ab2 VL
gatgttttgatgacccaaactccactctccctgcctgtcagtcttggagatcaagcctccatctcttgcagatctagtcagagcgttgtacatagtaatggaatcacctatttagaatggtacctgcagaaaccaggccagtctccaaagctcctgatctacaaagtttccaaccgattttctggggtcccagacaggttcagtggcagtggatcagggacatatttcacactcaagatcagcagagtggaggctgaggatcttggagtttattactgctttcaaggttcacatgttcctccgacgttcggtggaggcaccaagctggaaatcaaa

SEQ ID NO: 770 - protein, 123 aa, Artificial Sequence
  ACI-8032-6313G2-Ab1 VH
EVQLQQSGPELVKPGASVKISCKASGYTFTDYYMNWVKQSHGKSLEWIGDIDPNNGVTSYSQKFKGKATLTVDKSSSTAYMELRSLTSEDSAVYYCARTGYYGTSLYYAMDYWGQGTSVTVSS

SEQ ID NO: 771 - protein, 5 aa, Artificial Sequence
  ACI-8032-6313G2-Ab1 VH CDR1
DYYMN

SEQ ID NO: 772 - protein, 17 aa, Artificial Sequence
  ACI-8032-6313G2-Ab1 VH CDR2
DIDPNNGVTSYSQKFKG

SEQ ID NO: 773 - protein, 14 aa, Artificial Sequence
  ACI-8032-6313G2-Ab1 VH CDR3
TGYYGTSLYYAMDY

SEQ ID NO: 774 - protein, 112 aa, Artificial Sequence
  ACI-8032-6313G2-Ab1 VL
DIVMSQSPSSLAVSAGEKVTMNCKSSQSLLNSRTRKNYLAWYQQKPGQSPKLLIYWASTRESGVPDRFIGSGSGTGFTLTISSVQAEDLAVYYCKQSYDLWTFGGGTKLEIK

SEQ ID NO: 775-777 - skipped (3 entries)

SEQ ID NO: 778 - DNA, 369 bp, Artificial Sequence
  ACI-8032-6313G2-Ab1 VH
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattgatcctaacaatggtgttactagttacagccagaagttcaagggcaaggccacattgactgtagacaagtcctccagcacagcctacatggagctccgcagcctgacatctgaagactctgcagtctattactgtgcaaggacggggtactacggtacttctctatactatgctatggactactggggtcaaggaacctcagtcaccgtctcctca

SEQ ID NO: 779 - DNA, 336 bp, Artificial Sequence
  ACI-8032-6313G2-Ab1 VL
gacattgtgatgtcacagtcgccatcctccctggctgtgtcagcaggagagaaggtcactatgaactgcaaatccagtcagagtctgctcaacagtagaacccgaaagaactacttggcttggtaccagcagaaaccagggcagtctccaaaactactgatctactgggcatccactagggaatctggggtccctgatcgcttcataggcagtggatctgggacaggtttcactctcaccattagcagtgtgcaggctgaagacctggcagtttattactgcaagcaatcttatgatctgtggacgttcggtggaggcaccaagctggaaatcaaa

SEQ ID NO: 780-783 - skipped (4 entries)

SEQ ID NO: 784 - protein, 112 aa, Artificial Sequence
  ACI-8032-6314A3-Ab3 VL
DVLMTQIPLSLPVSPGDQVFISCRSSQSIVHSNGNTYLEWYLQKPGQSPKPLIYKVSNRFSGVPDRFSGSGSGTDFTLKISRVEAEDLGVYYCFQGSHVPPTFGGGTKLEIK

SEQ ID NO: 785-788 - skipped (4 entries)

SEQ ID NO: 789 - DNA, 336 bp, Artificial Sequence
  ACI-8032-6314A3-Ab3 VL
gatgttttgatgacccaaattccactctccctgcctgtcagtcctggagatcaagttttcatctcttgcagatctagtcagagcattgtacatagtaatggaaacacctatttagaatggtacctgcagaaaccaggccagtctccaaagcccctgatctacaaagtttccaaccgattttctggggtcccagacaggttcagtggcagtggatcagggacagatttcacactcaagatcagcagagtggaggctgaggatctgggagtttattactgctttcaaggttcacatgttcctccgacgttcggtggaggcaccaagctggaaatcaaa

SEQ ID NO: 790 - protein, 116 aa, Artificial Sequence
  ACI-8033-6401F2-Ab1 VH
QVQLQQPGAELVKPGASVKMSCKASGYTFTNYWITWVKQRPGQGLEWIGDIYPGNGDVDYSEIFKNKAKMTVDTSSTTAYMQLSSLTSEDSAVYHCVLGQTSFGHWGQGTLVTVSA

SEQ ID NO: 791 - protein, 5 aa, Artificial Sequence
  ACI-8033-6401F2-Ab1 VH CDR1
NYWIT

SEQ ID NO: 792 - protein, 17 aa, Artificial Sequence
  ACI-8033-6401F2-Ab1 VH CDR2
DIYPGNGDVDYSEIFKN

SEQ ID NO: 793 - protein, 7 aa, Artificial Sequence
  ACI-8033-6401F2-Ab1 VH CDR3
GQTSFGH

SEQ ID NO: 794 - protein, 112 aa, Artificial Sequence
  ACI-8033-6401F2-Ab1 VL
DVLMTQTPLSLPVSLGDQASISCRSSQSVVHVNGNTYLDWYLQKPGQSPKLLIYKVSNRFSGVPDRFSGSGSGTDFTLRITRVEAEDLGIYYCFQGSHVPRTFGGGTKLEIK

SEQ ID NO: 795 - protein, 16 aa, Artificial Sequence
  ACI-8033-6401F2-Ab1 VL CDR1
RSSQSVVHVNGNTYLD

SEQ ID NO: 796 - skipped

SEQ ID NO: 797 - protein, 9 aa, Artificial Sequence
  ACI-8033-6401F2-Ab1 VL CDR3
FQGSHVPRT

SEQ ID NO: 798 - DNA, 348 bp, Artificial Sequence
  ACI-8033-6401F2-Ab1 VH
caggtccaactgcagcagcctggggctgagcttgtgaagcctggggcttcagtgaagatgtcctgcaaggcttctggctacaccttcaccaactactggattacctgggtaaaacagaggcctggacaaggccttgagtggattggggatatttatcctggtaatggtgatgttgactacagtgagatcttcaagaataaggccaaaatgactgtagacacatcctccaccacagcctacatgcagctcagcagcctgacatctgaggactctgcggtctatcactgtgtcttgggtcagacttcctttggtcattggggccaagggactctggtcactgtctctgca

SEQ ID NO: 799 - DNA, 336 bp, Artificial Sequence
  ACI-8033-6401F2-Ab1 VL
gatgttttgatgacccaaactccactctccctgcctgtcagtcttggagatcaagcctccatctcttgcagatctagtcagagtgttgtgcatgttaatggaaacacctatttagattggtacctgcagaaaccaggccagtctccaaagctcctgatctacaaagtttccaaccgattttctggggtcccagacaggttcagtggcagtggatcagggacagatttcacgctcaggatcaccagagtggaggctgaggatctgggaatttattactgttttcaaggttcacatgttcctcggacgttcggtggaggcaccaagctggaaatcaaa

SEQ ID NO: 800 - protein, 119 aa, Artificial Sequence
  ACI-8033-6402E2-Ab2 VH
EVQLQHSGPDLVKPGASVKISCKASGYTFTDYYMNWVKQSHGKSLEWIGDINPNNGDTTYNQKFKGRATLTVDKSSSTAYMELRSLTSEDSAVYYCARSGSNYYAMDSWGQGTSVTVSS

SEQ ID NO: 801 - skipped

SEQ ID NO: 802 - protein, 17 aa, Artificial Sequence
  ACI-8033-6402E2-Ab2 VH CDR2
DINPNNGDTTYNQKFKG

SEQ ID NO: 803 - protein, 10 aa, Artificial Sequence
  ACI-8033-6402E2-Ab2 VH CDR3
SGSNYYAMDS

SEQ ID NO: 804 - protein, 112 aa, Artificial Sequence
  ACI-8033-6402E2-Ab2 VL
DIVMSQSPSSLAVSAGERVTMSCKSSQSLFNSRTRKNYLAWYQQKPGQSPKLLIYWASTRESGVPDRFTGSGSGTDFSLNISSVQAEDLAVYYCKQSYDLWTFGGGTKLEIK

SEQ ID NO: 805 - protein, 17 aa, Artificial Sequence
  ACI-8033-6402E2-Ab2 VL CDR1
KSSQSLFNSRTRKNYLA

SEQ ID NO: 806-807 - skipped (2 entries)

SEQ ID NO: 808 - DNA, 357 bp, Artificial Sequence
  ACI-8033-6402E2-Ab2 VH
gaggtccagctgcaacattctggacctgacctggtgaagcctggggcttcagtgaagatatcctgtaaggcttctggatacacgttcactgactactacatgaactgggtgaaacagagccatggaaagagccttgagtggattggagatattaatcctaacaatggtgatactacctacaaccagaagttcaagggcagggccacattgactgtagacaagtcctccagcacagcctacatggagctccgcagcctgacatctgaggactctgcagtctattactgtgcaagatcggggagtaattactatgctatggactcctggggtcaaggaacctcagtcaccgtctcctca

SEQ ID NO: 809 - DNA, 336 bp, Artificial Sequence
  ACI-8033-6402E2-Ab2 VL
gacattgtgatgtcacagtctccatcctccctggctgtgtcagcaggagagagggtcactatgagctgcaaatccagtcagagtctgttcaacagtagaacccgaaagaactacttggcttggtaccagcagaaaccagggcagtctcctaaactgctgatctactgggcatccactagggaatctggggtccctgatcgcttcacaggcagtggatctgggacagatttcagtctcaacatcagcagtgtgcaggctgaagacctggcagtttattactgcaagcaatcttatgatctgtggacgttcggtggaggcaccaagctggaaatcaaa

SEQ ID NO: 810 - protein, 113 aa, Artificial Sequence
  ACI-8033-6402E10-Ab1 VH
DVQLQESGPGLVKPVQSLSLTCSVTGYSITSGYYWNWIRQFPGNKLEWMGYISSDGSNNYNPSLKNRISISRDTSKNQFFLKLDSVTTEDTATYYCVRADNYWGQGTTLTVSS

SEQ ID NO: 811 - protein, 6 aa, Artificial Sequence
  ACI-8033-6402E10-Ab1 VH CDR1
SGYYWN

SEQ ID NO: 812 - protein, 16 aa, Artificial Sequence
  ACI-8033-6402E10-Ab1 VH CDR2
YISSDGSNNYNPSLKN

SEQ ID NO: 813 - protein, 4 aa, Artificial Sequence
  ACI-8033-6402E10-Ab1 VH CDR3
ADNY

SEQ ID NO: 814 - protein, 112 aa, Artificial Sequence
  ACI-8033-6402E10-Ab1 VL
DVVMTQTPLTLSVTIGQPASISCKSSQSLLDSDGETYLNWLLQRPGQSPKRLIYLVSKVDSGVPDRFTGRGSGTDFTLKISRVEAEDLGVYYCWQGTHFPQTFGGGTKLEIK

SEQ ID NO: 815 - protein, 16 aa, Artificial Sequence
  ACI-8033-6402E10-Ab1 VL CDR1
KSSQSLLDSDGETYLN

SEQ ID NO: 816 - skipped

SEQ ID NO: 817 - protein, 9 aa, Artificial Sequence
  ACI-8033 VL CDR3
WQGTHFPQT

SEQ ID NO: 818 - DNA, 339 bp, Artificial Sequence
  ACI-8033-6402E10-Ab1 VH
gatgtacagcttcaggagtcaggacctggcctcgtgaaacctgttcagtctttgtctctcacctgctctgtcactggctactccatcaccagtggttattactggaactggatccggcagtttccaggaaacaaactggaatggatgggctacataagctccgatggtagcaataactacaacccatctctcaaaaatcgaatctccatctctcgtgacacatctaagaaccagtttttcctgaagttggattctgtgactactgaggacacagccacatattactgtgtaagagcggacaactactggggccaaggcaccactctcacagtctcctcg

SEQ ID NO: 819 - DNA, 336 bp, Artificial Sequence
  ACI-8033-6402E10-Ab1 VL
gatgttgtgatgacccagactccactcactttgtcggttaccattggacaaccagcctccatctcttgcaagtcaagtcagagcctcttagatagtgatggagagacatatttgaattggttgttacagaggccaggccagtctccaaagcgcctaatctatctggtgtctaaagtggactctggagtccctgacaggttcactggcagaggatcagggacagatttcacactgaaaatcagcagagtggaggctgaggatttgggagtttattattgctggcaaggcacacattttcctcagacgttcggtggaggcaccaagctggaaatcaag

SEQ ID NO: 820 - protein, 115 aa, Artificial Sequence
  ACI-8033-6403A4-Ab1 VH
EVQLVESGGGLVQPKGSLKLSCAASGFSFNTYAMNWVRQAPGKGLEWVARIRSKSNNYTTYYADSVKDRFTISRDDSESMLYLQMNNLKTEDTAMYYCVRTMDYWGQGTSVTVSS

SEQ ID NO: 821 - protein, 5 aa, Artificial Sequence
  ACI-8033-6403A4-Ab1 VH CDR1
TYAMN

SEQ ID NO: 822 - protein, 19 aa, Artificial Sequence
  ACI-8033-6403A4-Ab1 VH CDR2
RIRSKSNNYTTYYADSVKD

SEQ ID NO: 823 - protein, 4 aa, Artificial Sequence
  ACI-8033-6403A4-Ab1 VH CDR3
TMDY

SEQ ID NO: 824 - protein, 105 aa, Artificial Sequence
  ACI-8033-6403A4-Ab1 VL
QIVLTQSPAIMSASLGEEITLTCSASSSISYMHWYQQKSGTSPKLLIHSTFNLASGVPSRFSGSGSGTFYSLTISSVEAEDAADYYCHQWSSYYTFGGGTKLEIK

SEQ ID NO: 825 - protein, 10 aa, Artificial Sequence
  ACI-8033-6403A4-Ab1 VL CDR1
SASSSISYMH

SEQ ID NO: 826 - protein, 7 aa, Artificial Sequence
  ACI-8033-6403A4-Ab1 VL CDR2
STFNLAS

SEQ ID NO: 827 - protein, 8 aa, Artificial Sequence
  ACI-8033-6403A4-Ab1 VL CDR3
HQWSSYYT

SEQ ID NO: 828 - DNA, 345 bp, Artificial Sequence
  ACI-8033-6403A4-Ab1 VH
gaggtgcagcttgttgagtctggtggaggattggtgcagcctaaagggtcattgaaactctcatgtgcagcctctggattcagtttcaatacctacgccatgaactgggtccgccaggctccaggaaagggtttggaatgggttgctcgcataagaagtaaaagtaataattatacaacatattatgccgattcagtgaaagacagattcaccatctccagagatgattcagaaagcatgctctatctgcaaatgaacaacttgaaaactgaggacacagccatgtattactgtgtgagaactatggactactggggtcaaggaacctcagtcaccgtctcctca

SEQ ID NO: 829 - DNA, 315 bp, Artificial Sequence
  ACI-8033-6403A4-Ab1 VL
caaattgttctcacccagtctccagcaatcatgtctgcatctctaggggaggagatcaccctaacctgcagtgccagttcgagtataagttacatgcactggtaccagcagaagtcaggcacttctcccaaactcttgattcatagcacattcaacctggcttctggagtcccttctcgcttcagtggcagtgggtctgggaccttttattctctcacaatcagcagtgtggaggctgaagatgctgccgattattactgccatcagtggagtagttattacacgttcggaggggggaccaagctggaaataaaa

SEQ ID NO: 830 - protein, 113 aa, Artificial Sequence
  ACI-8033-6403E11-Ab2 VH
DVQLQESGPGLVKPSQSLSLTCSVTGYSITSAYYWNWIRQFPGNKLEWMGYISYDGSNNYIPSLKNRISISRDTSKNQFFLKLNSVTTEDTATYYCIRGDVNWGQGTTLTVSS

SEQ ID NO: 831 - protein, 6 aa, Artificial Sequence
  ACI-8033-6403E11-Ab2 VH CDR1
SAYYWN

SEQ ID NO: 832 - protein, 16 aa, Artificial Sequence
  ACI-8033-6403E11-Ab2 VH CDR2
YISYDGSNNYIPSLKN

SEQ ID NO: 833 - protein, 4 aa, Artificial Sequence
  ACI-8033-6403E11-Ab2 VH CDR3
GDVN

SEQ ID NO: 834 - protein, 112 aa, Artificial Sequence
  ACI-8033-6403E11-Ab2 VL
DVVMTQTPLTLSVTIGQPASISCKSSQSLLDGDGETYLNWLLQRPGQSPKRLIYLVSKLDSGVPDRFTGSGSGTDFTLKISRVEAEDLGVYYCWQGTHFPQTFGGGTKLEIK

SEQ ID NO: 835 - protein, 16 aa, Artificial Sequence
  ACI-8033-6403E11-Ab2 VL CDR1
KSSQSLLDGDGETYLN

SEQ ID NO: 836 - protein, 7 aa, Artificial Sequence
  ACI-8033-6403E11-Ab2 VL CDR2
LVSKLDS

SEQ ID NO: 837 - skipped

SEQ ID NO: 838 - DNA, 339 bp, Artificial Sequence
  ACI-8033-6403E11-Ab2 VH
gatgtacagcttcaggagtcaggacctggcctcgtgaaaccttctcagtctctgtctctcacctgctctgtcactggctactccatcaccagtgcttattactggaactggatccggcagtttccaggaaacaaactggaatggatgggctacataagctacgatggtagcaataactacatcccttctctcaaaaatcgaatctccatcagtcgtgacacatctaagaaccagtttttcctgaagttgaattctgtgactactgaggacacagccacatattactgtataagaggggatgttaactggggccaaggcaccactctcacagtctcctca

SEQ ID NO: 839 - DNA, 336 bp, Artificial Sequence
  ACI-8033-6403E11-Ab2 VL
gatgttgtgatgacccagactccactcactttgtcggtcaccattggacaaccagcctccatctcttgcaagtcaagtcagagcctcttagatggtgatggagagacatatttgaattggttgttacagaggccaggccagtctccaaagcgcctaatctatctggtatctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaa

SEQ ID NO: 840 - protein, 119 aa, Artificial Sequence
  ACI-7067-4813-R4A-G7-rec1 VH
EVQLEESGGGLVHPKGSLKLSCAASGFTFNSYAMHWVRQAPGKGLEWVARIRSKRSNYATYYADSVKDRFTISRDDSQSMVYLEMNNLEAEDTATYYCVRGGREAMDFWGQGTSVTVSS

SEQ ID NO: 841 - protein, 5 aa, Artificial Sequence
  ACI-7067-4813-R4A-G7-rec1 VH CDR1
SYAMH

SEQ ID NO: 842 - protein, 19 aa, Artificial Sequence
  ACI-7067-4813-R4A-G7-rec1 VH CDR2
RIRSKRSNYATYYADSVKD

SEQ ID NO: 843 - protein, 8 aa, Artificial Sequence
  ACI-7067-4813-R4A-G7-rec1 VH CDR3
GGREAMDF

SEQ ID NO: 844 - protein, 109 aa, Artificial Sequence
  ACI-7067-4813-R4A-G7-rec1 VL
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 845 - protein, 14 aa, Artificial Sequence
  ACI-7067-4813-R4A-G7-rec1 VL CDR1
RSSTGAVTTSNYAN

SEQ ID NO: 846 - protein, 7 aa, Artificial Sequence
  ACI-7067-4813-R4A-G7-rec1 VL CDR2
GTNNRAP

SEQ ID NO: 847 - protein, 7 aa, Artificial Sequence
  ACI-7067-4813-R4A-G7-rec1 VL CDR3
ALWYSNH

SEQ ID NO: 848 - DNA, 357 bp, Artificial Sequence
  ACI-7067-4813-R4A-G7-rec1 VH
gaggtgcagctggaggagtctggtggaggattggtgcaccctaaaggatcattgaaactctcatgtgccgcctctggtttcaccttcaattcctatgccatgcactgggtccgccaggctccaggaaagggtttggaatgggttgctcgcataagaagtaaaagaagtaattatgcaacatattatgccgattcagtgaaagacagattcaccatctccagagatgattcacaaagcatggtctatctggaaatgaacaacctggaagctgaggacacagccacgtattactgtgtgagagggggacgagaggctatggacttctggggtcaaggaacctcagtcaccgtctcctca

SEQ ID NO: 849 - DNA, 327 bp, Artificial Sequence
  ACI-7067-4813-R4A-G7-rec1 VL
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 850 - protein, 60 aa, Unknown
  Binding epitope from the class Mammalia
MDVFMKGLSKAKEGVVAAAEKTKQGVAEAAGKTKEGVLYVGSKTKEGVVHGVATVAEKTK

SEQ ID NO: 851 - protein, 35 aa, Unknown
  Binding epitope from the class Mammalia
EQVTNVGGAVVTGVTAVAQKTVEGAGSIAAATGFV